(19) United States
(12) Patent Application Publication
(10) Pub. No.: US 2025/0032643 A1
(43) Pub. Date: Jan. 30, 2025

(54) SYNTHETIC RECEPTORS

(71) Applicant: UCL BUSINESS LTD, London (GB)

(72) Inventors: Dimitri KULLMANN, London (GB); Andreas LIEB, Innsbruck (AT); Steven O DEVENISH, London (GB); Teresa KASERER, Silz (AT)

(21) Appl. No.: 18/559,778

(22) PCT Filed: May 11, 2022
(86) PCT No.: PCT/EP2022/062850

§ 371 Date: Nov. 8, 2023

(30) Foreign Application Priority Data
May 12, 2021 (GB) 2106754.1

(51) Int. Cl. A61K 48/00; A61K 38/00; C07K 14/705; C12N 15/86

(52) U.S. Cl. CPC A61K 48/0058; A61K 48/0075; C07K 14/705; C12N 15/86; A61K 38/00; C12N 2750/14143; C12N 2830/008

(57) ABSTRACT

The invention relates to modified G-protein coupled receptors (GPCRs) which (i) have decreased responsiveness to an endogenous activating ligand, and (ii) may be activated by exogenous agonists, which may be relatively benign over the counter drugs such as antihistamines. The modifications comprise mutations at particular amino acid positions, relative to the unmodified GPCRs. The invention also provides methods of use comprising administration of the modified GPCRs, for example in treating a neurological circuit disorder.

### STATEMENT REGARDING THE SEQUENCE LISTING

This application includes a sequence listing in .txt format titled “008510687_ST25.txt”, which is 375,144 bytes in size and was created on Jun. 27, 2024. The sequence listing is electronically submitted with this application via Patent Center and is incorporated herein by reference in its entirety.

### TECHNICAL FIELD

The present invention relates generally to synthetic G-protein coupled receptors for use in therapy, and methods and materials relating to the same.

### BACKGROUND ART

Gene therapy using synthetic G-protein coupled receptors (GPCRs) has great promise in conferring sensitivity of neurons to exogenous drugs in order to achieve controllable manipulation of neural circuits. For example an inhibitory human M4 (hM4) muscarinic receptor has been mutated to render it insensitive to its endogenous ligand, acetylcholine, but made sensitive to a series of molecules including the antipsychotic agents clozapine and olanzapine.

Receptors modified in this way have been called DREADDs (Designer Receptors Exclusively Activated by Designer Drugs) and RASSLs (Receptors Activated Solely by Synthetic Ligands) in the literature.

Expression of the hM4 DREADD (hm4D) using an adeno-associated viral (AAV) vector in the brain of experimental animals is well tolerated and has no effect in the absence of the exogenous ligand.

WO2015/136247 (UCL Business Ltd) describes how the hM4-derived DREADD (hM4D (Gi)), when expressed in the epileptogenic area of the rodent brain, allowed seizures to be suppressed on demand upon administration of clozapine, olanzapine or clozapine-N-oxide, a metabolite of clozapine. Other DREADDs are also described.

WO2018045178A1 (RUTGERS, THE STATE UNIVERSITY) relates to DREADDs for use in treating a disease or disorder of the nervous system in a subject.

WO2018/175443 (UNIVERSITY OF PITTSBURGH-OF THE COMMONWEALTH SYSTEM OF HIGHER EDUCATION relates to modified ligand-gated ion channel proteins for use in excitable cells or secretory cells for treatment of a disease or disorder associated with the nervous system.

Further developments in the technology are described by Avaliani, N., et al. “DREADDs suppress seizure-like activity in a mouse model of pharmacoresistant epileptic brain tissue.” Gene therapy 23.10 (2016): 760-7661; Wicker, Evan, and Patrick A. Forcelli. “Chemogenetic silencing of the midline and intralaminar thalamus blocks amygdala-kindled seizures.” Experimental neurology 283 (2016): 404-412; Berglind, Fredrik, My Andersson, and Merab Kokaia. “Dynamic interaction of local and transhemispheric networks is necessary for progressive intensification of hippocampal seizures.” Scientific reports 8.1 (2018): 1-15; Desloovere, Jana, et al. “Long-term chemogenetic suppression of spontaneous seizures in a mouse model for temporal lobe epilepsy.” Epilepsia 60.11 (2019): 2314-2324; Weston, Mikail, et al. “Olanzapine: a potent agonist at the hM4D (Gi) DREADD amenable to clinical translation of chemogenetics.” Science advances 5.4 (2019): eaaw1567.

Given their therapeutic utility, it can be seen that providing novel DREADDs which have different activation characteristics to those known in the art would provide useful technical contributions in this field.

### DISCLOSURE OF THE INVENTION

The present inventors have observed that the drugs used to activate known DREADDs may be sub-optimal in some contexts. For example both olanzapine and clozapine have numerous pharmacological targets including histaminergic, muscarinic and dopaminergic receptors, which contribute to their anti-psychotic effects. They are both prescription-only medicines.

Although olanzapine is relatively well tolerated, it is mildly sedating and associated with mild weight gain, antimuscarinic side effects, eosinophilia and sexual dysfunction. Clozapine additionally can be pro-epileptic and is associated with white cell abnormalities that require frequent blood tests, making it less attractive as the activating ligand.

***

The inventors have used innovative approaches to define residues in GPCRs that modify their activation characteristics such that they are activatable by different types of drugs to those used in the DREADDs in the prior art. These new DREADDS may be activated by ligands which are relatively benign over the counter drugs such as antihistamines.

By way of non-limiting example, the inventors have modified residues in the hM4D (Gi) receptor, allowing it to be activated by the well tolerated anti-histamine drug diphenhydramine.

They have further identified residues in other G-protein coupled receptors which correspondingly modify their pharmacology.

These modifications provide a combination of desirable properties i.e. relatively high potency and relatively high efficacy with the relevant drug, and low basal activity in the absence of ligand. These designer receptors that can be activated by over-the-counter drugs may be referred to herein, purely for brevity, as “GRANPAs” (G-protein coupled Receptors Activated by Non-Prescription Agents).

GRANPAs may be used to affect or elicit G protein-mediated cellular responses in target cells in subjects, for example in neurons. For example populations of cells can be transformed with the vector encoding the GRANPA. Thus GRANPAs have utility in treating a wide range of indications, particularly neurological circuit disorders.

More specifically, as described below, the present inventors have further modified a muscarinic type 4 DREADD (hM4D) which was derived from an M4 receptor (CHRM4) acetylcholine receptor, also known as the cholinergic receptor, by incorporating the previously known Y113C and A203G substitutions. The wild type amino acid sequence of the M4 receptor is shown in SEQ ID NO: 1. Unless stated otherwise all numbering refers to this M4 receptor sequence.

Extensive mutagenesis of hM4D and a variety of screens for activity with novel “over the counter” ligands identified several novel mutants imparting beneficial properties.

As with known DREADDs the GRANPAs have (i) a decreased responsiveness to an endogenous activating ligand (ii) a retained or enhanced responsiveness to an exogenous agonist, of the type described herein. “Responsiveness” as described herein relates to the potency and/or efficacy of the ligand or exogenous agonist.

For example S85V and Y416F improved potency with the ligand diphenhydramine while V120I improved efficacy.

“Potency” as used herein is the concentration of drug required for its half maximal effect (EC50) on the investigated protein.

“Efficacy” as used herein is the maximum effect which can be achieved with a drug (Emax) on the investigated protein, in comparison to a control compound.

Combinations of these mutations had (at least) additive effects.

Mutation at these positions, and in preferred embodiments, these specific substitutions, (S85V, Y416F and V120I) individually and in combination, form aspects of the present invention.

A preferred hM4D GRANPA incorporates S85V+Y113C+V120I+A203G+Y416F.

Other mutations imparting beneficial properties are also described herein e.g. L123T, L123C, L123S, L123V or L123I, F128I, F128L or F128V, M121F, A200T, F204Y, W413L, and I410V.

Mutation at these positions, and in preferred embodiments these specific substitutions, individually and in combination, also form aspects of the present invention.

Unless stated otherwise, any of these novel modifications (i.e. not including Y113C and A203G substitutions) may be referred to herein for brevity as a “modification of the invention”.

For example a further referred preferred hM4D GRANPA incorporates L123T in combination with S85V, Y416F and/or V120I.

The hM4D derived GRANPA is coupled to the G, alpha subunit (or G/Go or Gi protein) and activates G protein-coupled inwardly-rectifying potassium channels (GIRKs).

As noted above, the present inventors have identified corresponding residues in other G-protein coupled receptors which can therefore likewise modify their pharmacology. Thus the present invention has wide applicability to GPCRs. Unless stated otherwise, wherever a modification of the invention is described with reference to the M4 receptor sequence, it will be understood that the disclosure applies mutatis mutandis to the corresponding modification in the GPCRs discussed herein. Identification of “corresponding” positions and modifications is described in detail hereinafter, and such corresponding modifications are therefore also to be understood to be “modifications of the invention”.

Thus in one aspect of the invention there is provided a modified G-protein coupled receptor (GPCR) wherein the modified GPCR has:
(i) a decreased responsiveness to an endogenous activating ligand, and
(ii) a retained or enhanced responsiveness to an exogenous agonist;
compared to a parent which is the native GPCR,
wherein the modified GPCR comprises one, more preferably two, three or four, modifications compared to the parent GPCR at positions selected from:
(i) 85
(ii) 416
(iii) 120
(iv) 123
plus optionally one or more modifications compared to the parent GPCR at positions selected from:
(v) 128
(vi) 121
(vii) 200
(viii) 204
(viii) 410, and
(vix) 413

In all cases herein (unless context demands otherwise) the amino acid positions given for the modified GPCR are numbered by correspondence with the amino acid sequence of SEQ ID NO:1 (i.e. the amino acid positions are those which correspond to that numbering in SEQ ID NO:1). As described in more detail below, the actual amino acid numbering may therefore differ for the GPCR in question compared to SEQ ID NO:1.

As explained above, the modified GPCR may comprise the following residues at the following positions:
(a) 113C or 113N, and
(b) 203G.

The modified GPCR may comprise one or more of the following substitutions at the following positions:
(a) Y113C or Y113N, and
(b) A203G.

The modified GPCR may comprise one or more of the following residues at the following positions:
(i) 85V or 85C
(ii) 120I.
(iii) 416F
(iv) 123C, 123I, 123T or 123S,

The modified GPCR may comprise one or more of the following substitutions at the following positions:
(i) S85V or S85C, most preferably S85V
(ii) V120I
(iii) Y416F
(iv) L123C, L123I, L123T, or L123S.

The modified GPCR, in addition to the residues or modifications (a) and (b), may comprise residues or substitutions: (i); (i), and (ii); (i), (ii), and (iii); (i), (ii), (iii), and (iv) above.

In addition to the residues or modifications described above, the modified GPCR may comprise one or more of the following residues at the following positions:
(v) 128I, 128L or 128V
(vi) 121F
(vii) 200T
(viii) 204Y (viii) 410V
(ix) 413L.

The modified GPCR may comprise one or more of the following substitutions at the following positions:
(v) F128I, F128L or F128V
(vi) M121F
(vii) A200T
(viii) F204Y
(viii) I410V
(ix) W413L.

In one embodiment the modified GPCR comprises Y113C+A203G+S85V+L123T+V120I+Y416F.

In one embodiment, the modified GPCR comprises Y113C+A203G+S85V+Y416F.

In some embodiments, the modified GPCR comprises:
Y113C+A203G,
Y113C+A203G+S85V+Y416F,
Y113C+A203G+S85V+Y416F+V120I;
Y113C+A203G+S85V+Y416F+L123T;
Y113C+A203G+S85V+Y416F+V120I+L123T;
Y113C+A203G+S85V+Y416F+L123C;
Y113C+A203G+S85V+Y416F+V120I+L123C,
Y113C+A203G+S85V+Y416F+L123S;
Y113C+A203G+S85V+Y416F+V120I+L123S;
Y113C+A203G+S85V+Y416F+L123I;
Y113C+A203G+S85V+Y416F+V120|+L123I; or
Y113C+A203G+S85V+Y416F+L123V.

***

In another aspect there is provided a method of increasing the potency and/or efficacy of an exogenous agonist which is an antihistamine to a parent modified G-protein coupled receptor (GPCR) wherein the parent modified GPCR comprises modified residues at the following positions:
(a) 113, and
(b) 203,
the method comprising making further modifications at one or more further positions in the parent modified GPCR, the further modifications being as described above,
   wherein the amino acid positions of the parent modified GPCR are numbered by correspondence with the amino acid sequence of SEQ ID NO:1

In one embodiment the exogenous agonist (or exogenous ligand) is diphenhydramine or an analog thereof. In other embodiments the exogenous agonist or ligand is selected from Table 1 or Table 2 e.g. selected from: diphenhydramine, cyproheptadine, diphenylpyraline, desloratadine, benzatropine.

***

### GPCRs

“GPCR” as used herein means a receptor that, upon binding of its natural ligand and activation of the receptor, transduces G protein-mediated signal(s) that result in a cellular response. GPCRs form a large family of evolutionarily related proteins (see WO97/35478). Proteins that are members of the GPCR family are structurally related and generally composed of seven putative transmembrane domains.

As explained at www.addgene.org/guides/chemogenetics
(the contents of which is incorporated herein by cross reference) GPCRs are the largest group of membrane receptors in eukaryotes, and the largest class of signal-transducing molecules in the brain. GPCRs are cell surface receptors that can intercept a variety of extracellular signals, including light, peptides, sugars, and lipids, and relay signaling to an intracellular G protein. The intracellular G proteins that associate with GPCRs are comprised of three subunits: the alpha, beta, and gamma subunits. In the resting state, the heterotrimeric G protein is bound to the GPCR and, in particular, the alpha subunit is in its inactive, GDP-bound state. Once a signal is received by a GPCR, it undergoes a conformational shift that activates the G protein, causing the exchange of GDP with GTP. The trimeric G protein now dissociates into two parts: the active, GTP-bound alpha subunit and the beta-gamma dimer complex, both of which can then diffuse laterally (remaining bound to the plasma membrane) and signal to other membrane proteins.

Activated G proteins can signal to a variety of other proteins, and can activate production of second messengers.

Each of the G protein subunits has different versions that have different binding partners, and thus, functions. There have been 5 beta-subunits, 11 gamma-subunits, and 20 alpha-subunits identified in mammals. Some of these G proteins activate their targets, while others can have inhibitory effects, and the combination of various G protein subunits to compose a G protein produces a diverse repertoire of G proteins and GPCR signaling within an organism. For example, the alpha subunit as (Gas) activates adenylate cyclase, causing production of the common second messenger cAMP. In neurons, cAMP elevation activates neuronal firing, while in smooth muscle, cAMP elevation causes muscle relaxation. Alternatively, the alpha subunit ai (Gai) inhibits adenylate cyclase, and as a result can have an opposing effect (neuronal inhibition and smooth muscle contraction, respectively). Different alpha subunits can also have similar phenotypic outcomes. For example, the alpha subunit aq (Gaq) also causes smooth muscle contraction, but does so through the activation of phospholipase C. Thus, G proteins can activate a wide array of signaling pathways and lead to a variety of cellular responses.

The present invention has utilities with both inhibitory and excitatory GPCRs.

GPCRs typically have a preference for one G protein subtype, but are capable of coupling to multiple subtypes. For example, the human muscarinic receptor M1 predominantly activates Gaq, but has also been shown to couple to Gai and Gas pathways. The human muscarinic receptor M3, however, has only been shown to couple with Gaq.

Non-limiting Examples of DREADDs and their activity in neurons is given at www.addgene.org/guides/chemogenetics

| | Original | | | Outcome |
|---|---|---|---|---|
| DREADD | receptor | Effector | Effect | (in neurons) |
| hM3Dq | Human M3 | Gαq | Increase Ca2+ | Neuronal |
| | muscarinic | | | burst firing |
| hM4D(Gi) | Human M4 | Gαi | Decrease cAMP | Neuronal |
| | muscarinic | | β/γ-GIRK | inhibition |
| | | | activation | |
| GsD | Rat M3 | Gαs | Increase cAMP | Neuronal |
| | muscarinic | | | burst firing |
| | & turkey β1- | | | |
| | adrenergic | | | |
| Rq(R165L) | Human M3 | Arrestin- | Increase Arrestin | Arrestin |
| | muscarinic | 2/-3 | translocation | signalling |

Another mechanism by which a GPCR may modify neuronal excitability and hence neurotransmission is through coupling via G-proteins to G protein-coupled inwardly-rectifying potassium channels (GIRKs). The G protein-coupled cellular response here is thus membrane hyperpolarization and neuronal inhibition.

As used herein a “G protein-coupled cellular response” means a cellular response or signalling pathway that occurs upon ligand binding by a GPCRG. Such G protein-coupled cellular responses relevant to the present invention are those which modify neuronal excitability and hence neurotransmission. One response is an inhibitory response whereby activation of the receptor with the ligand causes synaptic silencing or inhibition.

As described herein the present inventors have used a variety of assays including arrestin recruitment, the Gi cascade (to verify the ability to inhibit cAMP production) and an electrophysiology assay to test the G-protein dependent opening of Kir3.1 and Kir3.2 GIRKs).

In one embodiment the GPCR is a Gi-coupled GPCR.

In one embodiment the GPCR is coupled via a G-protein to an ion channel, wherein the ion channel is optionally inwardly rectifying and/or wherein the ion channel is optionally a potassium channel, which is preferably a protein-coupled inwardly-rectifying potassium channel.

In one embodiment the GPCR is a Gq-coupled or Gs coupled GPCR.

In one embodiment the GPCR is selected from a cholinergic receptors muscarinic receptor (CHRM); a histamine receptor (HRH); a 5-Hydroxytryptamine (serotonin) receptor (HTR); a dopamine receptor (DRD); an alpha adrenergic receptor (ADRA); a beta adrenergic receptor (B1-4 adrenoceptor) (ADRB).

In one embodiment the GPCR is selected from: CHRM4, CHRM3, CHRM1, CHRM2, CHRM5, HRH1, HRH2, HRH3, HRH4, 5HTR-1A, 5HTR-1B, 5HTR-1D, 5HTR-1E, 5HTR-1F, 5HTR-2A, 5HTR-2B, 5HTR-2C, 5HTR-4, 5HTR-5A, 5HTR-6, 5HTR-7, DRD-1, DRD-2, DRD-3, DRD-4, DRD-5, ADRA-1A, ADRA-1B, ADRA-1D, ADRA-2A, ADRA-2B, ADRA-2C, ADRB-1, ADRB-2, ADRB-3.

In one embodiment the GPCR is selected from a GPCR identified in Table 3 below.

***

Where the invention is utilised with non-hM4 GPCRs, residues “corresponding” to those numbered with respect to the M4 receptor (CHRM4) can be readily identified based on the disclosure herein.

Example 13 provides alignments of GPCRs to explicitly show the corresponding positions of the mutations of the invention.

It will be appreciated that the native residue in these GPCRs may not always be identical to that given in CHRM4 used for the reference numbering. Nevertheless due to the high degree of conservation between the aminergic GPCRs (see e.g. Example 13, FIGS. 23 and 24) the present invention may likewise be applied to these other GPCRs.

By way of the example, the native residues corresponding to the CHRM4 amino acids in selected GPCRs are as follows:

S85V: CHRMs(S), HRH3 (C), HRH4 (S). Thus, again by way of example, the corresponding substitution in HRH3 would be C->V. The corresponding position is 87. So this would be C87V in HRH3.

V120I: CHRMs (V), HRH3 (A), HRH4 (V). Thus, by way of example, the corresponding substitution in HRH3 would be A->I. The corresponding position is 122. So this would be A1221.

Further corresponding positions and native residues are listed in Example 13.

***

Alternatively, in any aspect or embodiment of the invention, the “Ballesteros-Weinstein numbering system” may be used to identify “corresponding” positions and residues. This class A GPCR residue numbering system (Ballesteros J A, Weinstein H. Integrated methods for the construction of three-dimensional models and computational probing of structure-function relations in G protein-coupled receptors. Methods in neurosciences. 1995; 25:366-428) is well understood in the art, and had more than 1100 citations by 2015 (see Isberg, Vignir, et al. “Generic GPCR residue numbers-aligning topology maps while minding the gaps.” Trends in pharmacological sciences 36.1 (2015): 22-31.)

Briefly, the Ballesteros-Weinstein numbering scheme is based on the presence of highly conserved residues in each of the seven transmembrane (TM) helices of GPCRs. It consists of two numbers where the first denotes the helix, 1-7, and the second the residue position relative to the most conserved residue, defined as number 50. For example, 5.42 denotes a residue located in TM5, eight residues before the most conserved residue, Pro5.50.

Using this system residues described herein with reference to CHRM4 have the following Ballesteros-Weinstein numbering:
S85—(2.57)
Y113—(3.33)
V120—(3.40)
M121—(3.41)
L123—(3.43)
F128—(3.48)
A200—(5.43)
A203—(5.46)
F204—(5.47)
I410—(6.45)
W413—(6.48)
Y416—(6.51)

Although the GRANPAs of the invention are themselves functional mutants of DREADDs or native GPCRs, it will be understood by those skilled in the art that further variants derived from the GRANPAs described herein may likewise be employed in the present invention.

For example GRANPAs may comprise further modifications (relative to the wild type) that nevertheless do not substantially affect their activity or utility. In accordance with the present invention, preferred further changes in the agent are commonly known as “conservative” or “safe” substitutions. Conservative amino acid substitutions are those with amino acids having sufficiently similar chemical properties, in order to preserve the structure and the biological function of the agent. It is clear that insertions and deletions of amino acids may also be made in the above defined sequences without altering their function, particularly if the insertions or deletions only involve a few amino acids, e.g. under ten and preferably under five, and do not remove or displace amino acids which are critical to the functional confirmation of the agent (e.g. agonist binding pocket). The literature provide many models on which the selection of conservative amino acids substitutions can be performed on the basis of statistical and physico-chemical studies on the sequence and/or the structure of a natural protein. In such cases the GRANPA will retain the properties in the terms defined above e.g. targeted cellular activation in the presence of the agonist, but not the natural ligand.

Furthermore, due to the degeneracy of the genetic code, it is clear that any nucleic acid sequence could be varied or changed without substantially affecting the sequence of the agent protein encoded thereby, to provide a functional variant thereof. Suitable nucleotide variants are those having a sequence altered by the substitution of different codons that encode the same amino acid within the sequence, thus producing a silent change.

***

Modification of GPCRs, and expression of GRANPAs, may be performed by those skilled in the art in the light of the present disclosure through conventional molecular biology techniques (see, e.g., Sambrook et al, Molecular Cloning: Cold Spring Harbor Laboratory Press). Example vectors and promoters are described hereinafter.

Embodiments of the invention are further directed to nucleic acids or isolated nucleic acids encoding the GRANPAs described herein. Further embodiments are directed to an expression vector comprising a nucleic acid or isolated nucleic acid described herein operably linked to a regulatory sequence.

Even further embodiments are directed to a host cell comprising an expression vector described herein, or nucleic acids encoding the GRANPAs described herein.

Still further embodiments are directed to methods of producing a GRANPA described herein comprising: stably transforming a host cell with an expression vector comprising a polynucleotide encoding the GRANPA; culturing the transformed host cell under suitable conditions to produce the GRANPA; and recovering the GRANPA.

In some embodiments, the host cell is a bacterial cell or a fungal cell. These may be useful for producing GRANPA proteins e.g. for structural analysis or raising antibodies.

In some embodiments, the host cell is a mammalian cell, for example a subject being treated by the methods of the present invention, or a stem cell. Suitable vectors for this purpose are described hereinafter.

For stable expression of the GRANPA protein, suitable expression hosts are bacterial expression host genera including Escherichia (e.g., E. coli), Pseudomonas (e.g., P. fluorescens or P. stutzerei), Proteus (e.g., P. mirabilis), Ralstonia (e.g., R. eutropha), Streptomyces, Staphylococcus (e.g., S. carnosus), Lactococcus (e.g., L. lactis), or Bacillus (subtilis, megaterium, licheniformis, etc.). Also particularly suitable are yeast expression hosts such as S. cerevisiae, S. pombe, Y. lipolytica, H. polymorpha, K. lactis or P. pastoris.

Also suited are mammalian expression hosts such as mouse (e.g., NS0), Chinese Hamster Ovary (CHO), HEK, or Baby Hamster Kidney (BHK) cell lines. Other eukaryotic hosts such as insect cells or viral expression systems (e.g., bacteriophages such as M13, T7 phage or Lambda, or viruses such as Baculovirus) are also suitable for producing recombinant polypeptides such as GRANPAs.

***

GRANPAs are variant polypeptides that may be “substantially similar” to wild type reference GPCRs or DREADDs from which they are derived, and may have at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity with a reference GPCR from which they were derived.

For example they may be substantially similar to any one of polypeptide sequences SEQ ID Nos 1-35 identified in Table 8.

For example in one embodiment the modified GPCR has at least 70% sequence identity with its native parent GPCR of any one of SEQ ID 1-35 of Table 8.

The modified GPCR may comprise a sequence shown in any one of Tables 3-6 comprising said modifications.

The term “variant polynucleotide” refers to a polynucleotide that encodes a GRANPA and has a specified degree of homology/identity with a parent polynucleotide, or hybridizes under stringent conditions to a parent polynucleotide or the complement thereof. For example, a variant polynucleotide has at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% nucleotide sequence identity with a parent polynucleotide.

For example they may be substantially similar to any one of polynucleotide sequences SEQ ID Nos 36-70 identified in Table 8.

For example the invention provides a polynucleotide comprising a nucleic acid sequence encoding the modified GPCR described herein.

The nucleic acid may have at least 70% sequence identity with its native parent GPCR of any one of SEQ ID 36-70 of Table 8.

Calculation of percentage identities between different amino acid/polypeptide/nucleic acid sequences may be carried out as follows. A multiple alignment is first generated by the ClustaIX program (pair wise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix Gonnet series, DNA weight IUB; Protein gap parameters, residue-specific penalties on, hydrophilic penalties on, hydrophilic residues GPSNDQERK (SEQ ID NO:212), gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

### Exogenous Agonists

As explained above, the inventors have used an innovative approach to define residues in G-protein coupled receptors (GPCRs) that modify their activation characteristics such that they are activatable by different types of drugs to those used in the DREADDs in the prior art, which can be over the counter drugs such as antihistamines.

Thus GRANPAs are activated by the presence of an exogenous agonist. The exogenous agonist (or ‘drug’, ligand, or small molecule, the terms are generally used interchangeably herein) is one which can be delivered directly or indirectly to the target cells expressing the GRANPA. Modes of administration are discussed in more detail below. The ligand is exogenous in that it is generally absent from the target cell, or present in sufficiently low basal concentrations that it does not activate the GRANPA.

Suitable target cells in which the GRANPA may be is expressed are discussed in more detail below.

In one embodiment the target cell is in the brain, and the agonist is administered directly or is able to penetrate the blood-brain barrier, either passively or via active transport. Typically molecules that cross the blood brain barrier are less charged than peptide molecules. Synthetic drugs can be made that do, or do not cross the blood-brain barrier depending on the number of charged groups on the molecule (see, e.g., Freidinger, 1993, Prog. Drug Res. 40:33-98). Smaller molecules, e.g., less than 4000 Da, are also more likely to cross the blood-brain barrier.

Ligands may be natural products, but preferably the ligand is synthetic i.e. not naturally occurring. Preferred ligand(s) are those possessing minimal or benign biological activities other than GRANPA activation. Preferably the ligand is an “over-the-counter” drug as described herein, for example an antihistamine or structural analog thereof. Any of these ligands as described herein may be referred to for brevity as “agonists of the invention”.

One preferred ligand is the antihistamine drug diphenhydramine (DPH). This has been used as an active ingredient or component of several over-the-counter medications used as mildly sedating anti-allergy or anti-motion sickness treatments available in the UK, including Nytol Original, Nytol One-a-Night, Sleepeaze, Benylin Chesty Coughs, Covonia Night Time Formula and Histergan. In the USA it is sold as Benadryl and Nytol among other popular brands, and is a component of many other non-prescription medications including Excedrin, Sudafed, Motrin PE and Robitussin Night Time Cough and Cold.

DPH provides a number of benefits as a ligand for DREADDs. For example:
(i) it is well tolerated and safe;
(ii) its pharmacology is well known, with a good separation between the concentrations at which it acts as an H1 antagonist and concentrations at which it acts on different receptors
(iii) the fact that it is effective as a mildly sedating agent with use in motion sickness confirms that it penetrates the CNS in humans

Many native GPCRs show a relatively low binding affinity for DPH, compared to clozapine used in existing DREADDs:

| Receptor | CLZ | DPH |
|---|---|---|
| H1 | 1.13 | 9.6-16 |
| M1 | 6.17 | 80-100 |
| M2 | 36.67 | 120-490 |
| M3 | 19.25 | 84-229 |
| M4 | 15.33 | 53-112 |
| M5 | 15.5 | 30-260 |
| α1A | 1.62 | >1000 |
| 5-HT2A | 5.35 | 260 |
| α2C | 6 | >1000 |
| α1B | 7 | >1000 |
| 5-HT2C | 9.44 | 780 |
| 5-HT6 | 13.49 | >1000 |
| 5-HT7 | 17.95 | >1000 |
| D4 | 26.36 | >1000 |
| α2B | 26.5 | >1000 |
| α2A | 37 | >1000 |

The modifications described herein may be used to enhance the potency and/or efficacy of DPH or related compounds, thereby making them viable agonists for therapeutic GRANPAS.

Alternative ligands may be selected from the following:

Table 1—Agents Having Chemical Similarity to Diphenhydramine and Example Salts Thereof (−)-cetirizine, Alprazolam, Amitriptyline, Amoxapine, Antazoline, Benzatropine, Bibenzonium bromide, Biperiden, Bromazine, Bromodiphenhydramine, Bromodiphenhydramine Hydrochloride, Brompheniramine, Buclizine, Butorphanol, Butriptyline, Captodiame, Carbinoxamine, Carbinoxamine Maleat, Cetirizine, Chlorcyclizine, Chlorphenamine, Chlorphenoxamine, Cinnarizine, Cinoxacin, Clomipramine, Cloperastine, Cyamemazine, Cyclizine, Cyclobenzaprine, Cyclopentolate, Cycrimine, Demexiptiline, Desipramine, Desvenlafaxine, Dexbrompheniramine, Dimenhydrinate, Dimetindene, Diphenhydramine Citrate, Diphenhydramine Hydrochloride, Diphenhydramine Methylbromide, Diphenhydramine Salicylate, Diphenylpyraline, Doxepin, Doxylamine, Embramine, Emedastine, Felbamate, Fendiline, Fluconazole, Flumexadol, Flunarizine, Fluorescein, Fosphenytoin, Glycopyrrolate, Hydroxyzine, Imipramine, Levallorphan, Levocetirizine, Mazindol, Meclizine, Mepyramine, Mequitazine, Methdilazine, Metixene, Mianserin, Moxastine, Nefopam, Nortriptyline, Olanzapine, Orphenadrine, Orphenadrine Citrate, Orphenadrine Hydrochloride, Oxitriptyline, Paroxetine, Perhexiline, Phenindamine, Pheniramine, Procyclidine, Progabide, Protriptyline, Rotoxamine, Trihexyphenidyl, Trimipramine, Tripelennamine, Triprolidine, Tymazoline.

### Table 2—Other Antihistamines

Acrivastine, Alimemazine, Alimemazine Tartrate, Antazoline, Astemizole, Azatadine, Azelastine, Bepotastine, Bilastine, Bromazine, Bromodiphenhydramine, Brompheniramine, Buclizine, Carbinoxamine, Cetirizine, Chlorcyclizine, Chlorodiphenhydramine, Chloropyramine, Chlorphenamine, Chlorpheniramine, Cinnarizine, Clemastine, Clofedanol, Cyclizine, Cyproheptadine, Desloratadine, Dexbrompheniramine, Dexchlorpheniramine, Dexchlorpheniramine maleate, Dextromethorphan, Dimenhydrinate, Dimetindene Maleate, Dimetindene, Diphenylpyraline, Dosulepin, Doxylamine, Ebastine, Embramine, Emedastine, Epinastine, Fexofenadine, Hydroxyzine, Ketotifen, Levocabastine, Levocetirizine, Loratadine, Meclizine, Mepyramine, Mirtazapine, Mizolastine, Naphazoline, Olopatadine, Orphenadrine, Phenindamine, Pheniramine, Phenylpropanolamine, Phenyltoloxamine, Pizotifen, Promethazine, Propiomazine, Pseudoephedrine, Pyrilamine, Quetiapine, Quifenadine, Rupatadine, Sertraline, Terfenadine, Thonzylamine, Trazodone, Trimeprazine, Tripelennamine, Triprolidine, Xylomeazoline,

***

It will be understood that the GRANPAs used herein are modified with respect to their corresponding native GPCR in that the GRANPA exhibits binding for a selected natural ligand that is decreased, preferably substantially decreased, more preferably substantially eliminated, relative to binding of the natural ligand by its corresponding native GPCR. Therefore GRANPA activity is relatively unaffected by natural fluctuations of the selected natural ligand (e.g. acetylcholine). Preferably GRANPA binding of the selected natural ligand is decreased by at least 5-fold, preferably 10-fold, more preferably 50-fold, still more preferably 75-fold, and may be decreased 100-fold or more relative to binding by the GRANPA's corresponding native G protein-coupled receptor.

GRANPAs can also be characterized by the ratio of synthetic ligand binding (for example, antihistamine or other drugs described above) affinity to binding affinity of a selected natural ligand. Preferably, GRANPAs of the invention exhibit a high synthetic ligand binding to selected natural ligand binding ratio, and exhibit synthetic ligand: selected natural ligand binding ratios of at least 0.1, 0.2, 0.3, 0.4, 0.5, 0.6, 0.7, 0.8, 0.9, preferably at least 1.0, more preferably at least 5, even more preferably 10, still more preferably 100 or higher.

Preferably, GRANPAs exhibit binding ratios that are 2-fold greater, preferably 5-fold greater, more preferably 10-fold greater, even more preferably 50- to 100-fold greater than the synthetic ligand: selected natural ligand binding ratio of a native G protein-coupled receptor.

GRANPAs can also be characterized by the ratios of the level of activation by exposure to synthetic ligand to the level of activation by exposure to a selected natural ligand (“activation ratio”). Activation levels can be measured as described in the Examples herein. Preferably, GRANPAs of the invention exhibit a synthetic ligand activation to selected natural ligand activation ratio, and exhibit synthetic ligand: selected natural ligand activation ratios of at least 0.8, preferably at least 1.0, more preferably at least 5, even more preferably 10, still more preferably 100 or higher. Preferably, GRANPAs exhibit activation ratios that are 2-fold greater, preferably 5-fold greater, more preferably 10-fold greater, even more preferably 50- to 100-fold greater than the synthetic molecule ligand: selected natural ligand activation ratio of a native G protein-coupled receptor.

***

The present invention provides methods of producing variant or modified GPCRs by modifying the peptides or nucleic acid encoding therefor, with one or more of the amino acid modifications of the invention described herein. These may be used to increase the potency and/or efficacy of an exogenous agonist (such as an antihistamine).

The present invention provides methods of producing the GRANPAs described herein by expression from nucleic acids encoding therefore.

The present invention provides methods of increasing the potency and/or efficacy of an exogenous agonist (such as an antihistamine) to a modified G-protein coupled receptor (GPCR) wherein the modified GPCR comprises modified residues at the following positions:
(a) 113, and
(b) 203,
the method comprising making additional modifications of the invention at one or more further positions in the modified GPCR. Such methods may typically be performed by modification of nucleic acids encoding therefore.

The present invention provides uses of modifications of the invention described herein to achieve novel technical effects. Such modifications are one or more amino acids introduced into a modified G-protein coupled receptor (GPCR) wherein the modified GPCR comprises modified residues at the following positions:
(a) 113, and
(b) 203,
to increase the potency and/or efficacy of an exogenous agonist (such as an antihistamine). This use may comprise modification of nucleic acids encoding therefore.

Thus in one aspect there is provided a process for producing a modified G-protein coupled receptor (GPCR) having modified responsiveness to an exogenous agonist, which process comprises modifying a parent GPCR with two, three or four, modifications compared to the parent GPCR at positions selected from:
(i) 85
(ii) 416
(iii) 120
(iv) 123
plus optionally one or more modifications compared to the parent GPCR at positions selected from:
(v) 128
(vi) 121
(vii) 200
(viii) 204
(viii) 410
(ix) 413
wherein the amino acid positions of the modified GPCR are numbered by correspondence with the amino acid sequence of SEQ ID NO:1.

The process may be to improve the potency and/or efficacy of the exogenous agonist in the modified GPCR compared to the parent GPCR.

The parent GPCR to be modified may be derived from a native GPCR which includes certain of the residues or substitutions described above (e.g. at 113 and/or 203).

Also provided are modified GPCRs obtained or obtainable by the processes or methods described herein.

Another aspect of the invention provides use of a modification as described herein to increase the potency and/or efficacy of an exogenous agonist such as an antihistamine, which modification is a further amino acid modification introduced into a parent modified G-protein coupled receptor (GPCR) wherein the parent modified GPCR comprises modified residues at the following positions: (a) 113, and (b) 203, wherein the amino acid positions of the modified GPCR are numbered by correspondence with the amino acid sequence of SEQ ID NO:1.

### Utility and Methods of Use

In one aspect the invention provides a method of selectively modifying G-protein activation, or activating a G-protein, in a cell of a subject or organism, the method comprising the steps of:

(i) expressing the GRANPA in the cell; and

(ii) administering to the subject or organism an agonist of the invention to the expressed GRANPA.

The GRANPA will typically be expressed in the cell prior to administration of the agonist. Such methods can be used to alter G-protein activation in the cell in a region- and time-specific manner.

The subject or organism may therefore have been previously administered the polynucleotide, prior to performance of the method. In those cases the polynucleotide comprising a nucleic acid sequence encoding the heterologous GRANPA is already in the cell of subject or organism.

Once activated the G-protein may then inhibit or stimulate further signaling pathways and cellular processes or responses, for example affecting the excitability or other characteristic of the cell, tissue, subject or organism (see discussion of Gas, Gai and Gaq proteins, and corresponding modified GPCRs above).

### Subject or Organism

The mammal may be a human subject.

The mammal may be a non-human mammal e.g. a test animal such as a rodent (e.g. mouse, rat) or primate. The mammal may be a transgenic mammal.

The subject or organism may be a bird, fish, reptile or amphibian.

Such test animals (not humans) form further aspects of the invention.

### Target Cell Types

As described herein the methods have utility in a wide variety of target-cell types, and the methods or modes of expression (e.g. cell specific expression) and administration are adopted according to the subject and desired target cell type.

Preferably the cell is an “excitable cell” such as a neuron of the CNS or PNS, muscle cell including striated and smooth muscle, or endocrine cell.

GRANPAs may have utility in manipulating the autonomic nervous system and heart, since hM3D (Gq) has been used previously in this way (Agulhon C, Boyt K M, Xie A X, Friocourt F, Roth B L, McCarthy K D. Modulation of the autonomic nervous system and behaviour by acute glial cell Gq protein-coupled receptor activation in vivo. J Physiol. 2013 Nov. 15: 591 (22): 5599-609. doi: 10.1113/jphysiol.2013.261289. Epub 2013 Sep. 16. PMID: 24042499; PMCID: PMC3853498); Kaiser E, Tian Q. Wagner M, Barth M, Xian W. Schröder L, Ruppenthal S, Kaestner L, Boehm U, Wartenberg P. Lu H, McMillin S M, Bone D B J, Wess J, Lipp P. DREADD technology reveals major impact of Gq signalling on cardiac electrophysiology. Cardiovasc Res. 2019 May 1: 115 (6): 1052-1066. doi: 10.1093/cvr/cvy251. PMID: 30321287; PMCID: PMC6736079.

GRANPAs may have utility in altering pancreatic function, since hM4D (Gi) has been used in this way previously to manipulate pancreatic alpha cells Zhu L, Dattaroy D, Pham J, Wang L, Barella L F, Cui Y, Wilkins K J, Roth B L, Hochgeschwender U, Matschinsky F M, Kaestner K H, Doliba N M, Wess J. Intra-islet glucagon signaling is critical for maintaining glucose homeostasis. JCI Insight. 2019 Apr. 23; 5 (10): e127994. doi: 10.1172/jci.insight. 127994. PMID: 31012868; PMCID: PMC6542600.

In other embodiments the cells are “non-excitable” cells e.g. hepatocytes. For example it is believed that hM4D (Gi) activation in hepatocytes worsens glucose control and that deletion of Gi in hepatocytes improves glucose control (Rossi M, Zhu L, McMillin S M, Pydi S P, Jain S, Wang L, Cui Y, Lee R J, Cohen A H, Kaneto H, Birnbaum M J, Ma Y, Rotman Y, Liu J, Cyphert T J, Finkel T, McGuinness O P, Wess J. Hepatic Gi signaling regulates whole-body glucose homeostasis. J Clin Invest. 2018 Feb. 1; 128 (2): 746-759. doi: 10.1172/JCI94505. Epub 2018 Jan. 16. PMID: 29337301; PMCID: PMC5785257). Accordingly Gs-coupled GRANPAs may have utility in improving glucose control.

***

In one aspect of the invention there is provided a method of selectively modifying the excitability of neurons in the CNS (e.g. brain) of a mammal in a region- and time-specific manner, the method comprising the steps of:
a. administering an effective amount of polynucleotide comprising a nucleic acid sequence encoding the GRANPA to the subject
b. expressing the GRANPA of step (a) prior to administration of an agonist to the GRANPA; and
c. administering to the subject an agonist to the expressed GRANPA.

As explained above, in preferred embodiments activation of said GRANPA alters the excitability of the neurons in the nervous system of the subject.

Typically the GRANPA is expressed in the central nervous system (brain or spinal cord). Suitable vectors and promoters for this purpose are described hereinafter.

Activation of the GRANPA may inhibit neurotransmission by excitatory neurons, or activate inhibitory neurons. Activation of inhibitory neurons may lead to an inhibitory response such as synaptic silencing or inhibition.

In other embodiments the activation of the GRANPA may activate excitatory neurons.

In other embodiments the activation of the GRANPA may inhibit excitatory neurons.

In other embodiments the activation of the GRANPA may inhibit inhibitory neurons.

Thus in one aspect the invention provides a method of selectively modifying the excitability of neurons in the brain of a mammal in a region- and time-specific manner, the method comprising the steps of in a subject, comprising the steps of:
(i) expressing the GRANPA prior to administration of an agonist to the GRANPA; and
(ii) administering to the subject an agonist of the invention to the expressed GRANPA.

### Modes of Administration

Exogenous agonists may be administered by any appropriate method known in the art, provided that they are thereby distributed to the target cells comprising the GRANPAs.

Non-limiting routes of administration include the following:
a) Oral;
b) Parenteral by intravenous or intra-muscular route;
c) Sub-cutaneous injection (which may be preferred in palliative care, for example
where the GRANPA is used for the relief of pain, nausea or anxiety);
d) Sub-lingual, buccal, intranasal, rectal, for rapid absorption, a route used for benzodiazepines in acute seizure treatment in adults or children (e.g. rectal diazepam, buccal or nasal midazolam);
e) Via an inhaler or nebulizer (which may be preferred in the treatment of chronic obstructive airways disease, cystic fibrosis, emphysema, bronchiolitis or bronchiectasis, by analogy with salbutamol, a beta 2 adrenergic agonist, used to treat COAD);
f) Topical: Eye, which may be preferred for treatment of eye disease such as glaucoma, by analogy with timolol (alpha 2 adrenergic agonist) eyedrops which are used to treat open-angle glaucoma;
g) Topical: Skin (trans-dermal), by analogy with topical clonidine gel (alpha 2 adrenergic agonist) which has been used to treat neuropathic pain
h) Other topical routes: otic, intrathecal, intravaginal, intravesicular

Systemic modes of administration may be preferred.

Exogenous agonists may be adapted for the route of administration according to methods known in the art. For example oral, injectable and topical formulations of diphenhydramine are known in the art.

***

The novel GRANPAs described herein can have utility in the gene therapy of a wide range of diseases or disorders, for example of the nervous system, for example neurological circuit disorders. These include neuropsychiatric disorders, neurodegenerative diseases, chronic pain, cerebrovascular accident (CVA) or stroke. Examples of diseases in which GRANPAs may show utility are given hereinafter.

In a particular embodiment, the GRANPA is based on hM4D (Gi) (human M4 muscarinic cholinergic Gi-coupled DREADD). In a particular embodiment, the DREADD is human muscarinic acetylcholine receptor M4, including the modifications of the invention described herein.

By way of non-limiting example, controlled suppression of activation neurotransmission (by inhibition of excitatory neurons, e.g. by Gi-coupled receptors, or activation of inhibitory neurons) has utility in epilepsy and other diseases characterized by episodes of abnormal cellular activity such as migraine, cluster headache, trigeminal neuralgia, post-herpetic neuralgia, paroxysmal movement disorders, and uni- or bipolar affective disorders.

In a particular embodiment, the GRANPA is coupled with Gq. In a particular embodiment, the GRANPA is based on the Gq-coupled human M3 muscarinic receptor (hM3Dq) (see, e.g., Alexander et al. (2009) Neuron 63 (1): 27-39; Armbruster et al. (2007) Proc. Natl. Acad. Set, 104 (12): 5163-5168) including the modifications of the invention described herein.

Activation of excitatory (e.g. Gs-coupled and Gq-coupled) GPCRs may be useful in other mental health disorders such as Parkinson's Disease, and other diseases where some neurological circuits are thought to be underactive.

***

In one aspect of the invention there is provided a method of treating a disease or disorder in a subject, the method comprising the steps of:
(i) expressing in a target cell or organ of the subject a GRANPA; and
(ii) administering to the subject an agonist to the expressed GRANPA.

In one aspect of the invention there is provided a method of treating a disease or disorder in a subject, the method comprising the steps of:
(a) administering to the subject an effective amount of a polynucleotide or vector as described herein encoding a GRANPA e.g. by direct injection;
(b) expressing the GRANPA of step (a) in a target cell or organ of the subject; and
(c) administering to the subject exogenous agonist to the expressed GRANPA.

In one aspect of the invention there is provided a method of treating a disease or disorder of the nervous system in a subject, comprising the steps of:
a. administering an effective amount of polynucleotide comprising a nucleic acid sequence encoding the GRANPA to the subject
b. expressing the GRANPA of step (a) prior to administration of an agonist to the GRANPA; and
c. administering to the subject an agonist to the expressed GRANPA.

As explained above, typically the GRANPA is expressed in the central nervous system (brain or spinal cord).

In one aspect there is a provide a method of treating a disease or disorder of the nervous system in a subject, comprising the steps of:
(i) expressing the GRANPA prior to administration of an agonist to the GRANPA; and
(ii) administering to the subject an agonist to the expressed GRANPA.

***

As explained in WO2015/136247, the use of DREADDs potentially allows fine-tuning of the therapeutic effect, so that the optimal modulation of circuit function can be achieved with minimal off-target effects on normal brain function. The treatments can be targeted both to the brain region where the viral vector is introduced and to the cell type within that region and so the effect when the ligand is delivered can be effectively localised, and in the absence the ligand there would not be expected to be any effect on brain function. Thus the therapy is both targeted and temporally limited. However the leading inhibitory DREADD hM4D (Gi) is limited by the side effect profiles of activating ligands.

Thus in one aspect there is provided a method of treating a seizure disorder in a patient suffering from said disorder, which method comprises:
(a) administering to said patient a vector encoding a GRANPA, wherein said GRANPA is expressed neurons of a seizure focus in brain of the patient;
(b) administering to said patient said exogenous agonist,
whereby the presence of said agonist in the brain of the patient activates said GRANPA, whereby activation of said GRANPA reversibly alters the excitability of the neurons in the seizure focus.

There is also provided a method of treating a seizure disorder in a patient suffering from said disorder,
wherein said patient has previously been administered a vector encoding a GRANPA,
wherein said GRANPA is expressed neurons of a seizure focus in brain of the patient;
which method comprises administering to said patient said exogenous agonist,
whereby the presence of said agonist in the brain of the patient activates said GRANPA,
whereby activation of said GRANPA reversibly alters the excitability of the neurons in the seizure focus.

In the context of the present invention, the GRANPA may be used to treat a seizure disorder in a subject suffering from said disorder. In such treatments the the presence of said agonist in the brain of the patient activates said GRANPA, thereby reversibly altering, preferably inhibiting, the excitability of the neurons in the seizure focus.

For example the activation of said GRANPA (i) reversibly inhibits the excitability of and neurotransmission by excitatory neurons in the seizure focus, or (ii) reversibly excites inhibitory neurons in the seizure focus.

In preferred embodiments, the seizure disorder is epilepsy, for example idiopathic, symptomatic and cryptogenic epilepsy. The methods described herein may be used to quench or blocking epileptogenic activity. The methods may be used for raising the seizure threshold in brain or neural tissue of a patient in need thereof, or reducing epileptic bursting in brain cells of the patient.

The combined chemical-genetic (also known as chemogenetic) methods of the present invention may be used for the treatment of epilepsy via the suppression of seizures in a region- and time-specific manner.

In one embodiment, the epilepsy is generalized epilepsy. It has been reported (Wicker, Evan, and Patrick A. Forcelli. “Chemogenetic silencing of the midline and intralaminar thalamus blocks amygdala-kindled seizures.” Experimental neurology 283 (2016): 404-412) that that seizures could be worsened by silencing inhibitory interneurons, suggesting that other manipulations (silencing thalamocortical excitatory cells) would achieve on-demand seizure suppression.

In one embodiment, the epilepsy is human focal epilepsy.

The patient may be one who has been diagnosed as having well defined focal epilepsy affecting a single area of the neocortex of the brain. Focal epilepsy can arise, for example, from developmental abnormalities or following strokes, tumours, penetrating brain injuries or infections.

However the invention may also be used to treat multiple epileptic foci simultaneously by injection directly into the multiple identified loci.

The patient may be one who has been diagnosed as having drug-resistant or medically-refractory epilepsy, by which is meant that epileptic seizures continue despite adequate administration of antiepileptic drugs.

The patient may be one who is under an existing treatment with anti-epileptic drugs, wherein the method has the purpose of permitting the existing treatment to be discontinued or the drug regime to be reduced.

The patient may be one who has been diagnosed as having epilepsia partialis continua.

The treatments of the present invention have particular utility where a permanent reduction in neuronal excitability (as could be achieved with potassium channel overexpression, for instance) is undesirable, for example because it represents too great a risk to normal brain function. Even if the epileptogenic zone is in the cortical regions responsible for language or motor function, there would be no effect on these functions except when the ligand was administered. Patients with intractable focal epilepsy are likely to consider this an acceptable side effect.

Although the invention has particular utility for seizure disorders characterized by focal onset, such as temporal lobe epilepsy and focal neocortical epilepsy, it may also be applied to more generalised forms epilepsy, particularly as a second-line indication. In these cases the target for delivery will be chosen as appropriate to the condition e.g. delivery may be bilaterally to the thalamus. Thus other disorders to which the invention may be applied include infantile spasms, myoclonic and “minor motor” seizures, as well as tonic-clonic seizures and partial complex seizures.

Furthermore, in principle, the invention could be used prophylactically by causing continued alteration of neuronal excitability for a fixed period with the purpose of ‘resetting’ epileptogenic circuits in some circumstances, bringing about a persistent reduction in seizures that outlasts the administration of the ligand.

Thus in different embodiments of the invention:
(i) the seizure disorder is epilepsy, which is optionally focal epilepsy or generalized epilepsy, and/or
(ii) the subject has been diagnosed as having drug-resistant or medically-refractory epilepsy, and/or
(iii) wherein the subject is under an existing treatment with anti-epileptic drugs, wherein the method has the purpose of permitting the existing treatment to be discontinued or the drug regime to be reduced, and/or
(iv) the subject has been diagnosed as having epilepsia partialis continua, and/or
(v) the exogenous ligand is administered to the subject prior to the patient having an epileptic seizure, and/or
(vi) the exogenous ligand is administered to the subject during an epileptic seizure, and/or
(vii) the exogenous ligand is administered to the subject after having an epileptic seizure, and/or
(viii) the exogenous ligand is administered to the subject within 30 minutes before or 24 hours after the human has an epileptic seizure, and/or
(ix) the exogenous ligand is administered to the subject automatically either (i) by a device that is either coupled to an automated seizure detection mechanism, or (ii) in response to a predicted seizure by EEG analysis, and/or
(x) the exogenous ligand is administered in a combination therapy with one or more other agents for treating the seizure disorder.

***

Study of diphenhydramine overdoses concluded that the commonest adverse events were tachycardia, hallucinations, somnolence, agitation and mydriasis, with a much lower occurrence of seizures (Palmer et al., 2019 doi.org/10.1080/15563650.2019.1609683). The effect on lowering seizure threshold is a rare side-effect shared with other anti-histamine drugs, but is generally thought to be related to an anti-muscarinic effect. Our pharmacological studies suggest that diphenhydramine activates the GRANPA at lower concentrations than it blocks muscarinic receptors.

***

In addition to the diseases discussed above, DREADDs (and hence GRANPAs) have potential in other disorders. For example, in rodent models, DREADDs have demonstrated the ability to control neuronal activity to ameliorate disease phenotypes in conditions as diverse as Parkinson disease,⁶ Down syndrome,⁷ and autism.¹⁰ In addition, DREADD-based approaches modulate behaviours as diverse as addiction,^{11,12} sleep,¹³aggression,¹⁴breathing,¹⁵ and feeding.¹⁶⁻¹⁸DREADDS have also enhanced and silenced learning and memory and have been used to create artificial memories.¹⁹⁻²¹

Other CNS applications of DREADD-based therapeutics suggested in the art include psychostimulant (Ferguson et al., 2011) and ethanol (Pleil et al., 2015) abuse, depression (Urban et al., 2015), post-traumatic stress disorder (Zhu et al., 2014), intractable seizures (Kätzel et al., 2014), and many other disorders (English and Roth, 2015).

In addition to the well established effects DREADDs have on the nervous system, a number of studies have also identified potential therapeutic strategies using DREADDs on other organs. Animal models of such diseases/disorders treated by DREADDs include but are not limited to diabetes (Jain, S. et al., “Chronic activation of a designer G(q)-coupled receptor improves B cell function” J Clin Invest. 2013; 123:1750-1762), metabolic disorders (Li, J, et al., “A novel experimental strategy to assess the metabolic effects of selective activation of a G(q)-coupled receptor in hepatocytes in vivo” Endocrinology. 2013; 154:3539-3551), inflammatory disorders (Park, J. et al., “Synthetic control of mammalian-cell motility by engineering chemotaxis to an orthogonal bioinert chemical signal” Proc Natl Acad Sci USA. 2014; 111:5896-5901), and respiratory disorders (Curado, T. et al., “DREADD approach to sleep disordered breathing” Am J Respir Crit Care Med. Ahead of print 10.1164/rccm.202002-0321OC).

Specific examples of target circuits that can be manipulated with GRANPAs for therapeutic benefit are described in the following studies that have used preclinical models of neurological and neuropsychiatric circuit disorders:
pain (Weir et al, PMID: 28969375)
spasticity and spinal cord injury (Asboth et al PMID: 29556028, Chen et al PMID: 30033363)
Parkinson's disease, dystonia, chorea, Huntington's disease (Assaf & Schiller PMID: 30536778)
Alzheimer's disease and frontotemporal degeneration (Rorabaugh et al PMID: 29053824, Yuan et al PMID: 26758850)
amyotrophic lateral sclerosis (Khademullah et al PMID: 32203578, Alami et al PMID: 32900826)
attention deficit/hyperactivity disorder, obsessive-compulsive disorder, impulse control disorder, Tourette syndrome and autistic spectrum disorder (Rapanelli et al PMID: 28584117)
schizophrenia (Boekhoudt et al PMID: 27712862, Kätzel et al PMID: 33178010, Katzel & Kullmann “Optogenetic and Chemogenetic Tools for Drug Discovery in Schizophrenia” in doi.org/10.1039/9781782622499-00234)
major depressive disorder, bipolar disorder (Biselli et al PMID: 31633833, Muir et al PMID: 30555161)
anxiety and generalized anxiety disorder (Hirschberg et al PMID: 29027903; Jiang et al PMID: 29410218)
insomnia and sleep disorders (Sasaki et al PMID: 21647372)
anorexia, bulimia and other eating disorders (Krashes, NBK453150, Xu et al PMID: 29670283)

In other embodiments of the invention the disease or disorder is a non-CNS and/or non-PNS disorder.

### Vectors

As explained above, the GRANPAs of the invention are typically expressed in vivo to provide their medical benefit. This is achieved by use of polynucleotides comprising a nucleic acid sequence encoding the GRANPA, which are operably linked to suitable promoters. Typically the polynucleotide is in the form of, or comprised within, a genetic construct comprising an open reading frame encoding the GRANPA under transcriptional control of transcriptional control elements governing cell-specific expression, for example in CNS neurons or other excitable cells.

Examples of target CNS neurons include spinal cord cells, such as dorsal horn cells and/or brain cells, including and without limitation a brainstem, hindbrain, midbrain or forebrain excitatory or inhibitory cell population.

In methods of delivering nucleic acids encoding GRANPAs according to any aspect described herein to a cell or to a patient, the nucleic acid may be delivered by any useful method, in any useful form, as is recognized by those of ordinary skill in the field of genetic therapies. The nucleic acid may be naked nucleic acid, such as a plasmid, deposited, for example and without limitation, by a colloidal drug delivery method, such as liposomes, e.g., cationic liposomes, or nanoparticles, or as part of a recombinant viral genome, as are broadly-known.

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

For example the polynucleotide may be in the form of, or comprised within, a viral vector comprising a promoter operably linked to the nucleic acid sequence encoding the GRANPA, and optionally a 3′ untranslated region.

Any of a variety of vectors can be used in accordance with the invention to produce GRANPA-expressing cells. A vector for use in the therapies of the present invention will be suitable for in vivo gene therapy protocols. The vector may be a stable integrating vector or a stable non-integrating vector. A preferred vector is viral vector, such as a lentiviral or AAV (Adeno-associated virus) vector.

The use of both these types of viral vector is well known in the art for gene therapy. By way of example only, WO2008011381 describes the use of these and other vectors for expressing receptors in a subject. The content of that application, in respect of its description of the preparation and characteristics of AAV and lentiviral vectors is specifically incorporated herein by reference.

Briefly, as described in WO2008011381, AAV is a defective parvovirus and is a preferred vector because it can infect many cell types and is nonpathogenic to humans. AAV type vectors can transport about 4 to 5 kb and wild type AAV is known to stably insert into chromosome 19.

In another type of AAV vector, the AAV contains a pair of inverted terminal repeats (ITRs) which flank at least one cassette containing a promoter which directs cell-specific expression operably linked to a heterologous gene (here: a GRANPA). Further information can be found in U.S. Pat. No. 6,261,834. AAV vectors are discussed in WO2018/175443.

Viral vectors are well known in the art, and commercially available e.g. from Viralgen, Parque Científico y Tecnológico de Gipuzkoa, Paseo Mikeletegi 83, 20009 San Sebastián, Spain.

Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses. Typically, a lentiviral vector contains the 5′ and 3′ LTR regions of a lentivirus, such as SIV and HIV. Lentiviral vectors also typically contain the Rev Responsive Element (RRE) of a lentivirus, such as SIV and HIV. Examples of lentiviral vectors include those of Dull, T. et al., “A Third-generation lentivirus vector with a conditional packaging system” J. Virol 72 (11): 8463-71 (1998).

***

The vectors described herein can be delivered locally to the target cells in a variety of access methods known in the art.

For example liposomes or nanoparticles comprising the nucleic acid may be injected at a desired site, such as in or adjacent to specific neuronal tissue. In other aspects, a recombinant viral particle (transducing particle), is delivered, for example, injected, at a desired site, such as in or adjacent to, or otherwise targeting specific neuronal tissue. The nucleic acid may be injected once or more than once in order to establish sufficient expression of the GRANPA in the target neuron.

In particular delivery can be via direct injection into the brain using known methodologies, such as direct interstitial infusion, burr-hole craniotomy and stereotactic injection (see e.g. “Stereotactic and Functional Neurosurgery” Editors: Nikkhah & Pinsker; Acta Neurochirurgica Supplement Volume 117, 2013).

For the treatment of seizure disorders, the injection will be targeted to a seizure focus where that has been defined (e.g. in focal epilepsy) or more generally into areas of the brain suspected of overactivity in other seizure diseases.

Vectors may be used to effect permanent transformation, or may be only be transiently expressed in the brain.

Thus in one embodiment there is provided an expression vector comprising the polynucleotide of the invention described above. The vector may be a viral vector e.g. an adenovirus vector and/or an adeno-associated vector (AAV), which is optionally selected from the group consisting of: AAV1, AAV2, AAV3, AAV4, AAV5, AAV6, AAV7, AAV8, AAV9, AAV10, AAV11, AAV 12, and hybrids thereof. Alternatively, the vector may be a herpes virus vector, a retrovirus vector, or a lentivirus vector

***

Preferably, the DNA construct contains a promoter to facilitate expression of the GRANPA-encoding DNA within the target cell.

Promoters may be any known in the art suitable for gene therapy-see e.g. Papadakis, E. D., et al. “Promoters and control elements: designing expression cassettes for gene therapy.” Current gene therapy 4.1 (2004): 89-113; and Joshi C R, Labhasetwar V, Ghorpade A. “Destination Brain: the Past, Present, and Future of Therapeutic Gene Delivery.” J Neuroimmune Pharmacol. 2017; 12 (1): 51-83. Promoters may be natural nucleotide sequences, or synthetic combinations of minimal promoter sequences together with other regulatory elements such as enhancers. Examples of commonly used promoters include hSyn, mdl, CBA, Ef1a, TH, CMV, mDIx5/6, DRD2, Drd1a.

However specificity can be achieved by regional and cell-type specific expression of the receptor exclusively e.g. using a tissue or region specific promoter.

For example the promoter may direct cell-specific expression in CNS neurons, such as dorsal horn neurons, spinal cord cells, or brain cells, or in inhibitory neurons or nerve cells.

A promoter is “specific” to specified cells (e.g. excitable cells or secretory cells) if it causes gene expression in those cells of a gene to a sufficient extent for production of useful or therapeutically effective amounts of the described GRANPAs in the specified cells, and insignificant expression elsewhere in the context of the use, e.g. therapeutic use.

An example is the Camk2a (alpha CaM kinase II gene) promoter, which drives expression in relatively specifically in the forebrain-see e.g. Sakurada et al (2005) “Neuronal cell type-specific promoter of the alpha CaM kinase II gene is activated by Zic2, a Zic family zinc finger protein.” Neurosci Res. 2005 November; 53 (3): 323-30. Epub 2005 Sep. 12.

Other neuronal cell type-specific promoters include the NSE promoter (Liu H. et al., Journal of Neuroscience. 23 (18): 7143-54, 2003); tyrosine hydroxylase promoter (Kessler M A. et al., Brain Research. Molecular Brain Research. 112 (1-2): 8-23, 2003); myelin basic protein promoter (Kessler M A. et al Biochemical & Biophysical Research Communications. 288 (4): 809-18, 2001); glial fibrillary acidic protein promoter (Nolte C. et al., GLIA. 33(I): 72-86, 2001); neurofilaments gene (heavy, medium, light) promoters (Yaworsky P J. et al., Journal of Biological Chemistry. 272 (40): 25112-20, 1997) (All of which are herein incorporated by reference at least for the sequence of the promoters and related sequences.) The NSE promoter is disclosed in Peel A L. et al., Gene Therapy.

4 (1): 16-24, 1997) (SEQ ID NO:69) (pTR-NT3myc; Powell Gene Therapy Center, University of Florida, Gainesville F L). A further suitable promoter is the Synapsin1 promoter (see Kügler et al “Human synapsin 1 gene promoter confers highly neuron-specific long-term transgene expression from an adenoviral vector in the adult rat brain depending on the transduced area.” Gene Ther. 2003 February; 10 (4): 337-47). A further suitable promoter is the cd68 promoter, expressed in microglia. Promoters suitable for general expression include the EF1a or CAG promoters.

In one embodiment a vector encoding a GRANPA may comprise any of these promoters.

In one embodiment the nucleic acid encoding the modified GPCR is operably linked to a tissue or cell specific promoter e.g. a neuronal cell type-specific promoter. In one embodiment the promoter is the CaMk2A promoter.

In yet another embodiment, the neuron-specific promoter is preprotachykinin-1 promoter (TAC-1).

***

While it is possible for the ligand to be used (e.g., administered) alone, it is often preferable to present it as a composition or formulation e.g. with a pharmaceutically acceptable carrier or diluent.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition (e.g., formulation, preparation, medicament) comprising, or consisting essentially of, or consisting of as a sole active ingredient, a ligand as described herein, and a pharmaceutically acceptable carrier, diluent, or excipient.

As described in WO2008096268, in gene therapy embodiments employing viral delivery of the GRANPA, the unit dose may be calculated in terms of the dose of viral particles being administered. Viral doses include a particular number of virus particles or plaque forming units (pfu). For embodiments involving AAV, particular unit doses include 10³, 10⁴, 10⁵, 10⁶, 10⁷, 10⁸, 10⁹, 10¹⁰, 10¹¹, 10¹², 10¹³ or 10¹⁴ pfu or vector genomes. Particle doses may be somewhat higher (10 to 100 fold) due to the presence of infection-defective particles.

In one embodiment a vector is injected as 500 microL of a suspension of 5×10¹¹vg/ml (=2.5×10¹¹ viral genomes).

***

For ligands used with GRANPAs in the treatment of disease, an appropriate dosage can be utilised based on half-life and other pharmacokinetic and pharmacodynamic parameters. For example for DPH, based on a half life of around ˜9 hours, it may be preferred that would imply taking it at 3-4× daily. A typical dose based on comparable affinities at H1 and GRANPA may be around 25 to 50 mg (orally) 3 to 4× daily in an adult. However other dosages are also envisaged, based on the discretion of the physician.

Dosage forms may be extended or slow release (see e.g. Krowczynski, Laezek. Extended-release dosage forms. CRC press, 2020) or immediate release forms (see e.g. Nyol, Sandeep, and M. M. Gupta. “Immediate drug release dosage form: A review.” Journal of Drug Delivery and Therapeutics 3.2 (2013).

In one embodiment diphenhydramine is administered at 50-100 mg/day in divided doses.

***

In some embodiments the methods or treatments of the present invention may be combined with other therapies, whether symptomatic or disease modifying.

The term “treatment” includes combination treatments and therapies, in which two or more treatments or therapies are combined, for example, sequentially or simultaneously.

For example it may be beneficial to combine treatment with a compound as described herein with one or more other (e.g., 1, 2, 3, 4) agents or therapies.

Appropriate examples of co-therapeutics will be known to those skilled in the art on the basis of the disclosure herein. Typically the co-therapeutic may be any known in the art which it is believed may give therapeutic effect in treating the diseases described herein, subject to the diagnosis of the individual being treated. For example epilepsy can sometimes be ameliorated by directly treating the underlying etiology, but anticonvulsant drugs, such as phenytoin, gabapentin, lamotrigine, levetiracetam, carbamazepine and clobazam, and topiramate, and others, which suppress the abnormal electrical discharges and seizures, are the mainstay of conventional treatment (Rho & Sankar, 1999, Epilepsia 40:1471-1483).

The particular combination would be at the discretion of the physician who would also select dosages using his/her common general knowledge and dosing regimens known to a skilled practitioner.

The agents (i.e. the GRANPA and ligand, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

***

Where aspects of the invention comprises methods of treating a disease or disorder by use of a GRANPA, or polynucleotide comprising a nucleic acid sequence encoding the GRANPA, and/or agonist, there is also provided:
(i) a GRANPA or polynucleotide comprising a nucleic acid sequence encoding the GRANPA (e.g. vector as described herein), and/or agonist, for use in such methods;
(i) use of a GRANPA or polynucleotide comprising a nucleic acid sequence encoding the GRANPA (e.g. vector as described herein), and/or agonist, in the preparation of a medicament for such treatments.

***

The invention also provides a vector encoding a GRANPA, and an exogenous agonist for said receptor, for use in a method of treatment of a seizure disorder in a patient suffering from said disorder, which treatment comprises:
(a) administering to said patient said vector, wherein said GRANPA is expressed in neurons of a seizure focus in brain of the patient;
(b) administering to said patient said exogenous agonist,
whereby the presence of said agonist in the brain of the patient activates said GRANPA, whereby activation of said GRANPA reversibly alters the excitability of the neurons in the seizure focus.

The invention also provides a vector encoding a GRANPA for use in a method of treatment of a seizure disorder in a patient suffering from said disorder, which treatment comprises:
(a) administering to said patient said vector, wherein said GRANPA is expressed in neurons of a seizure focus in brain of the patient;
(b) administering to said patient said exogenous agonist,
whereby the presence of said agonist in the brain of the patient activates said GRANPA,
whereby activation of said GRANPA reversibly alters the excitability of the neurons in the seizure focus.

The invention also provides an exogenous agonist for use in a method of treatment of a seizure disorder in a patient suffering from said disorder, which treatment comprises:
(a) administering to said patient a vector encoding a GRANPA, wherein said GRANPA is expressed in neurons of a seizure focus in brain of the patient;
(b) administering to said patient said exogenous agonist,
whereby the presence of said agonist in the brain of the patient activates said GRANPA, whereby activation of said GRANPA reversibly alters the excitability of the neurons in the seizure focus.

The invention also provides an exogenous agonist for use in a method of treatment of a seizure disorder in a patient suffering from said disorder, wherein said patient has previously been administered a vector encoding a GRANPA, wherein said GRANPA is expressed in neurons of a seizure focus in brain of the patient;
which treatment comprises administering to said patient said exogenous agonist,
whereby the presence of said agonist in the brain of the patient activates said GRANPA,
whereby activation of said GRANPA reversibly alters the excitability of the neurons in the seizure focus

The invention also provides a vector and/or agonist as defined for use in these methods of treating seizure disorders.

The invention also provides a use of a GRANPA and/or vector and/or polynucleotide and/or agonist as defined herein in the preparation of a medicament for use in a method of treatment or therapy as described herein.

***

The present invention also provides kits comprising one or more components including, but not limited to, the viral vectors, promoter, and GRANPA, as discussed, in association with one or more additional components including, but not limited to, a pharmaceutically acceptable carrier and the GRANPA agonist.

The viral vectors, promoter, GRANPA composition and/or the GRANPA agonist can be formulated as pure compositions or in combination with a pharmaceutically acceptable carrier, in a pharmaceutical composition.

Kits may also include primers, buffers, and probes along with instructions for use in the methods described herein.

In one embodiment, a kit includes a viral vector, a promoter, a GRANPA composition of the invention or a pharmaceutical composition thereof in one container and a GRANPA agonist or a pharmaceutical composition thereof in another container (e.g., in a sterile glass or plastic vial).

If the kit includes a pharmaceutical composition for parenteral administration to a subject, the kit can include a device for performing such administration. For example, the kit can include one or more hypodermic needles or other injection devices.

### General Statements

As used herein, a “polypeptide” refers to a molecule comprising a plurality of amino acids linked through peptide bonds. The terms “polypeptide,” “peptide,” and “protein” are used interchangeably. Proteins may optionally be modified (e.g., glycosylated, phosphorylated, acylated, farnesylated, prenylated, and sulfonated) to add functionality. The conventional one-letter or three-letter codes for amino acid residues are used, with amino acid sequences being presented in the standard amino-to-carboxy terminal orientation (i.e., N→C).

The terms “polynucleotide” encompasses DNA, RNA, heteroduplexes, and synthetic molecules capable of encoding a polypeptide. Nucleic acids may be single-stranded or double-stranded, and may have chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence. Unless otherwise indicated, nucleic acid sequences are presented in a 5′-to-3′ orientation.

As used herein, the terms “wild-type”, “native”, or “reference” refer to polypeptides or polynucleotides that are found in nature. The terms, with respect to a polypeptide, refer to a naturally-occurring polypeptide that does not include a man-made substitution, insertion, or deletion at one or more amino acid positions. The terms with respect to a polynucleotide, refer to a naturally-occurring polynucleotide that does not include a man-made substitution, insertion, or deletion at one or more nucleosides. However, note that a polynucleotide encoding a wild-type or native or reference polypeptide is not limited to a naturally-occurring polynucleotide, and encompasses any polynucleotide encoding that polypeptide.

The term “derived from” encompasses the terms “originated from,” “obtained from,” “obtainable from,” “isolated from,” and “created from” and generally indicates that one specified material find its origin in another specified material or has features that can be described with reference to the another specified material (which may be termed “reference” or “parent”). The GRANPAs herein may be derived from reference or parent sequences, which may be wild type GPCR or DREADDs of the prior art

The term “hybridization” refers to the process by which a strand of nucleic acid joins with a complementary strand through base pairing, as known in the art. The term “hybridization conditions” refers to the conditions under which hybridization reactions are conducted. These conditions are typically classified by degree of “stringency” of the conditions under which hybridization is measured. The degree of stringency can be based, for example, on the melting temperature (Tₘ) of the nucleic acid binding complex or probe. For example, “maximum stringency” typically occurs at about Tₘ−5° C. (5° C. below the Tₘ of the probe); “high stringency” at about 5-10° C. below the Tₘ; “intermediate stringency” at about 10-20° C. below the Tₘ of the probe; and “low stringency” at about 20-25° C. below the Tₘ. Alternatively, or in addition, hybridization conditions can be based upon the salt or ionic strength conditions of hybridization and/or one or more stringency washes, e.g., 6X Saline Sodium Citrate (SSC)=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used).

The terms “substantially similar” and “substantially identical” in the context of at least two nucleic acids or polypeptides means that a polynucleotide or polypeptide comprises either a sequence that has at least about 60%, 70%, 80%, 90%, 91%, 92%, 93%, 94%, 95%, 96%, 97%, 98%, or 99% identity to a parent or reference sequence, or a sequence that includes amino acid substitutions, insertions, deletions, or modifications made only to circumvent the present description without adding functionality.

The term “expression vector” refers to a DNA construct containing a DNA sequence that encodes the specified polypeptide and is operably linked to a suitable control sequence capable of effecting the expression of the polypeptides in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself.

The term “recombinant” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally, recombinant nucleic acids, polypeptides, and cells based thereon, have been manipulated by man such that they are not identical to related nucleic acids, polypeptides, and cells found in nature.

“Receptor-ligand binding,” “ligand binding,” and “binding” are used interchangeably herein to mean physical interaction between a receptor (e.g., a native GPCR or GRANPA) and a ligand (e.g., a natural ligand, (e.g., peptide ligand) or synthetic ligand (e.g., synthetic small molecule ligand)). Ligand binding can be measured by a variety of methods known in the art (e.g., detection of association with a radioactively labeled ligand).

“Signaling” means the generation of a biochemical or physiological response as a result of ligand binding (e.g., as a result of synthetic ligand binding to a GRANPA).

“Receptor activation,” “GRANPA activation,” and “GPCR activation” mean binding of a ligand (e.g., a natural or synthetic ligand) to a receptor in a manner that elicits G protein-mediated signaling, and a physiological or biochemical response associated with G protein-mediated signaling. Activation can be measured by measuring a biological signal associated with G protein-related signals (e.g. using electrophysiology or other assays described herein).

“Targeted cellular activation” and “target cell activation” are used interchangeably herein to mean GRANPA mediated activation of a specific G protein-mediated physiological response in a target cell, where GRANPA-mediated activation occurs by binding of a synthetic small molecule to the GRANPA. As used herein, cellular activation includes (without limitation) inhibitory responses such as synaptic silencing or inhibition, and activation of G proteins in both inhibitory and stimulatory cells.

“Natural ligand” and “naturally occurring ligand” and “endogenous ligand” of a native GPCR are used interchangeably herein to mean a biomolecule endogenous to a mammalian host, which biomolecule binds to a native GPCR to elicit a G protein-coupled cellular response. An example is acetylcholine.

“Synthetic small molecule”, “synthetic small molecule ligand,” “synthetic ligand”, and “synthetic agonist” and the like are used interchangeably herein to mean any compound made exogenously by natural or chemical means that can bind within the transmembrane domains of a GPCR or modified GPCR (i.e., GRANPA) and facilitate activation of the receptor and receptor-mediated response.

The terms “transfect”, “transfection”, “transfected”, and like terms refer to the introduction of a gene into a eukaryotic cell, such as a neuron or keratinocyte, and includes “transduction,” which is viral-mediated gene transfer, for example, by use of recombinant AAV, adenovirus (Ad), retrovirus (e.g., lentivirus), or any other applicable viral-mediated gene transfer platform.

“Transformation” means a transient or permanent genetic change induced in a cell following incorporation of new DNA (i.e., DNA exogenous to the cell). Where the cell is a mammalian cell, a permanent genetic change is generally achieved by introduction of the DNA into the genome of the cell.

“Promoter” means a minimal DNA sequence sufficient to direct transcription of a DNA sequence to which it is operably linked. “Promoter” is also meant to encompass those promoter elements sufficient for promoter-dependent gene expression controllable for cell-type specific, tissue specific or inducible by external signals or agents; such elements may be located in the 5′ or 3′ regions of the native gene.

A “subject” may be a human or animal, e.g., vertebrates or mammals, including rat, mouse, rabbit, pig, monkey, chimpanzee, cat, dog, horse, goat, guinea pig, and birds.

The subject may be a “patient”.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress (prolonged survival), a halt in the rate of progress, regression of the condition, amelioration of the condition, and cure of the condition.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen.

The invention also embraces treatment as a prophylactic measure is also included and “treating” will be understood accordingly. Prophylactic treatment may utilise a “prophylactically effective amount,” which, where used herein, pertains to that amount of an agent which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

***

Wherever a method of treatment employing an agent is described herein, it will be appreciated that an agent for use in that method is also described, as is an agent for use in the manufacture of a medicament for treating the relevant disease.

Wherever a composition is described herein, it will be appreciated that the same composition for use in the therapeutic methods (including prophylactic methods) described herein is also envisaged, as is the composition for use in the manufacture of a medicament for treating the relevant disease.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. A) Comparison of diphenhydramine (DPH, blue) and iperoxo (orange, PDB entry 4MQS (Kruse, Ring et al. 2013) (iperoxo bound to the M2 muscarinic receptor)) binding mode suggests that the tyrosine is involved in hydrophobic interactions with iperoxo, but would clash with DPH. B). The N-Methyl group (highlighted by the dotted circle) of methysergide (red sticks) would clash with A225 (golden sticks) in the active 5-HT2B conformation (gold, PDB entry 6DRY (McCorvy et al., Nat. Struct. Mol. Biol. 2018, 25, 787-796, doi.org/10.1038/s41594-018-0116-7)), but is compatible with the A225G mutant (green, PDB entry 6DRZ (McCorvy et al., Nat. Struct. Mol. Biol. 2018, 25, 787-796, doi.org/10.1038/s41594-018-0116-7). Please note, for better visibility of the binding site, residues 180-198 and 132-138 are not shown.

FIG. 2. A) Wild type hM4 (WT CHRM4) activation by DPH. Y113C in combination with A203G, but not A203N, converts DPH from an antagonist to a low-potency agonist. B) Y113N can substitute for Y113C, but several other Y113 substitutions reduce DPH efficacy without substantially altering potency. C) Yet other Y113C substitutions abolish DPH-dependent activation. For reference, the double mutant A203G+Y113C (hM4D originally reported by Armbruster et al 2007) is shown in blue.

FIG. 3. S85 is involved in an interaction network with D112, Y443, and S116 in the active (left) and D112 and Y443 in the inactive (right) conformation.

FIG. 4. Effects of S85 mutations on DPH dependent activation in combination with A203G+Y113C. Double mutant A203G+Y113C is shown in blue for comparison.

FIG. 5. Y416 is involved in hydrogen bond interactions with olanzapine (left panel), but creates a polar binding site less compatible with binding of the hydrophobic DPH phenyl ring (right panel). The Y416F mutation makes the binding site hydrophobic in this area.

FIG. 6: A) Effect of Y416 mutations on DPH-dependent activation in combination with A203G+Y113C. Double mutant A203G+Y113C is shown in blue for reference. B) Effect of Y416F in combination with S85V, as well as A203G+Y113C.

FIG. 7. Aligned models of olanzapine (green) and DPH (blue) bound to hM4D (Gi) suggest that the methyl-group of IIe compensates for loss of the methyl-group in olanzapine, which is missing in DPH.

FIG. 8. A) Effect of V120 mutations on DPH dependent activation in combination with A203G+Y113C. Double mutant A203G+Y113C is shown in blue for reference. B) V120I also increases efficacy of DPH in combination with Y113C+A203G+S85V+Y416F described in FIG. 6B. For reference, construct Y113C+A203G+S85V+Y416F is shown in green.

FIG. 9. Mutation of L123 can stabilize a network of interactions present in active conformation (left panel), but cannot be formed in inactive conformation (right panel, PDB entry 5DSG (hM4 bound to tiotropium (Thal et al., Nature 2016, 531:335-340, DOI: 10.1038/nature17188).

FIG. 10. A) Effect of L123 mutations on DPH dependent activation in combination with A203G+Y113C. Double mutant A203G+Y113C is shown in blue for reference. B) Addition of L123 mutants leads to an increased efficacy/potency of DPH also in additional constructs. For reference construct Y113C+A203G+S85V+Y416F is shown in green and construct Y113C+A203G+S85V+Y416F+V120I is shown in red.

FIG. 11. Addition of F128 mutants leads to an increased efficacy/potency of DPH also in additional constructs. For reference construct Y113C+A203G+S85V+Y416F is shown in green, and double mutant A203G+Y113C is shown in blue.

FIG. 12. Addition of the M121F mutant leads to increased potency of DPH. For reference the double mutant A203G+Y113C is shown in blue.

FIG. 13. The A200T mutation introduces a hydrogen bond with N417 and thus stabilizes the active conformation of the receptor.

FIG. 14. Addition of the A200T mutant leads to an increased potency of DPH. For reference the double mutant A203G+Y113C is shown in blue.

FIG. 15. The F204Y mutation introduces a new hydrogen bond with T414 and thus stabilizes the active conformation of the receptor.

FIG. 16. Addition of the F204Y mutant leads to an increased potency of DPH. For reference the double mutant A203G+Y113C is shown in blue.

FIG. 17A. Addition of the W413L mutant leads to an increased potency of DPH. For reference the double mutant A203G+Y113C is shown in blue.

FIG. 17B. Addition of the I410V mutant leads to an increased efficacy of DPH. For reference the double mutant A203G+Y113C is shown in blue.

FIG. 18. Gi coupled activity of hM4D (Gi) (Y113C+A203G) was measured with the GloSensor assay. The addition of mutations S85V, V120I and Y416F shifts the EC50 for DPH from 1.4 UM to 3.7 nM.

FIG. 19. Gi coupled activity of hM4D (Gi) (Y113C+A203G) and mutants were measured with the GloSensor assay.

FIG. 20. hM4D (Gi) and mutants were tested for effects on basal cAMP levels using the GloSensor assay.

FIG. 21. Electrophysiology-based screen of hM4D (Gi) activation. A) Left: Representative traces of Kir3.1 and Kir3.2 currents with (+DPH 100 nM, bottom) and without (baseline, top) agonist application in a cell expressing Y113C+A203G+S85V+V120I+Y416F+L123T. Middle: Mean current measured during the time indicated by the grey area in the left panel, plotted against holding voltage. The red line indicates the calculation of the membrane leak conductance, obtained from a linear fit between 0 and +50 mV. Right: Leak-subtracted Kir3.1/Kir3.2-mediated currents, together with a linear fit to currents at negative potentials (blue). The slope of the current-voltage relationship (k) was used for subsequent analysis of mutant activation.
B) Left: DPH acts as a potent agonist on both Y113C+A203G+S85V+V120I+Y416F and Y113C+A203G+S85V+V120I+Y416F+L123T mutants. Right: EC50 (nM) of Y113C+A203G+S85V+V120I+Y416F and Y113C+A203G+S85V+V120I+Y416F+L123T mutants.
C) Without agonist application the Y113C+A203G+S85V+V120I+Y416F mutant shows significant basal activity in comparison to GFP-only transfection and the Y113C+A203G+S85V+V120I+Y416F+L123T mutant (GFP vs. Y113C+A203G+S85V+V120I+Y416F p=0.008; GFP vs. Y113C+A203G+S85V+V120|+Y416F+L123T p=0.777, One-way ANOVA with Bonferroni post hoc test).

D) In contrast to FIG. 10B, the Y113C+A203G+S85V+V120I+Y416F+L123T mutant does not show lower DPH efficacy than Y113C+A203G+S85V+V120I+Y416F in Kir3.1 and Kir3.2 activation (p=0.513, unpaired t-test).

FIG. 22. Other agents can be used as the activating ligand. A) Other drugs ordinarily show weak activation of hM4D (Gi) relative to DPH. B) Their potency and/or efficacy is improved upon the addition of mutations S85V, V120I and Y416F.

FIG. 23. Alignment of closely related GPCR family members shows high structural conservation. Alignment of all structures (a) and pairwise alignment of CHRM4 (red, PDB entry 5DSG (Thal et al., Nature 2016, 531:335-340) with (b) CHRM2 (orange, PDB entry 5ZKC (Suno et al., Nat Chem Biol 2018, 14:1150-1158)), (c) ADRB2 (cyan, PDB entry 3PDS (Rosenbaum et al., Nature 2011, 469:236-240)), (d) HRH1 (blue, PDB entry 3RZE (Shimamura et al., Nature 2011, 475:65-70). Please note the high structural similarity between diphenhydramine and the co-crystallized ligand doxepine, (e) DRD3 (violet, PDB entry 3PBL (Chien et al., Science 2010, 330:1091-1095), and (f) HTR2A (dark blue, PDB entry 6A93 (Kimura et al., Nat Struct Mol Biol 2019, 26:121-128). Figures and RMSD values have been obtained using the default alignment function in PyMOL Molecular Graphics System, Version 1.8.0.0 Schrödinger, LLC.

FIG. 24. Comparison of GRANPA residues in CHRM4 (red, PDB entry 5DSG (Thal et al., Nature 2016, 531:335-340)) and (a) CHRM2 (orange, PDB entry 5ZKC (Suno et al., Nat Chem Biol 2018, 14:1150-1158)), (b) ADRB2 (cyan, PDB entry 3PDS (Rosenbaum et al., Nature 2011, 469:236-240)), (c) HRH1 (blue, PDB entry 3RZE (Shimamura et al., Nature 2011, 475:65-70). (d) DRD3 (violet, PDB entry 3PBL (Chien et al., Science 2010, 330:1091-1095), and (e) 5-HT2A (dark blue, PDB entry 6A93 (Kimura et al., Nat Struct Mol Biol 2019, 26:121-128). Residues are labeled according to CHRM4 (bold) and CHRM3 (a), ADRB2 (b), HRH1 (c), DRD3 (d), and HTR2A (e) numbering. Please note that L119W and M164W mutants were introduced into the DRD3 (PDB entry 6PBL) and HTR2A (PDB entry 6A93) crystallization constructs. These have been mutated back to the wild type residues using PyMOL version 1.8.0.0.

FIG. 25. GRANPA activation with diphenhydramine in vivo alters behaviour.

### EXAMPLES

### Methods

For comparison of the hM4D (Gi) DREADD and the related beta-adrenoceptors and histamine receptors, the PDB entries 3PDS (Rosenbaum, Zhang et al. 2011) (beta-2 adrenergic receptor in complex with an irreversible agonist) and 3RZE (Shimamura, Shiroishi et al. 2011) (H1 histamine receptor in complex with doxepin) were aligned to the olanzapine-DREADD model reported in Weston et al. (Weston, Kaserer et al. 2019) and manually inspected using PyMOL version 0.99rc6 (The 2014).

A crude diphenhydramine (DPH)-DREADD binding model was generated by aligning DPH to the olanzapine binding pose using the RDKit Open 3D Alignment node (Masson, Ellis et al. 1992, Young, Fong et al. 2014) in KNIME version 4.0.0. (Berthold, Cebron et al. 2007). For refinement, the torsion angles were adjusted and the model was minimized in UCSF-Chimera 1.13.1 (Pettersen, Goddard et al. 2004). The DPH-DREADD model was aligned to the M4 muscarinic receptor in complex with tiotropium (PDB entry 5DSG (Thal, Sun et al. 2016)) to compare the active and inactive states and the structures were manually analysed using PyMOL version 1.8.0.0 (Hausser 2014). Polypeptide alignment of monoamine neurotransmitter receptors to identify non-consensus amino acids of CHRM4 was conducted using MEGA X version 10.1.8¹¹.

### Production of Mutants

The Gi-coupled human muscarinic receptor “hM4D (Gi)” has been made sensitive to the orally bioavailable and normally inert metabolite of clozapine, clozapine-N-oxide (CNO). This modified GPCR includes the following mutations: Y113C/A203G.

The modified receptor hM4D (Gi) was originally described by B. N. Armbruster, X. Li, M. H. Pausch, S. Herlitze, B. L. Roth, “Evolving the lock to fit the key to create a family of G protein-coupled receptors potently activated by an inert ligand”, Proc. Natl. Acad. Sci. U.S.A. 104, 5163-5168 (2007). Those authors describe a general, validated and unbiased approach for generating GPCRs with defined ligand specificities, which was utilised to create a family of muscarinic ACh receptor (mAChR) DREADDs. The content of that publication, in respect of its description of the preparation and characteristics of these DREADDs, is specifically incorporated herein by reference.

The preparation of a human M4 DREADD is also described in Nawaratne, V., Leach, K., Suratman, N., Loiacono, R. E., Felder, C. C., Armbruster, B. N., & Christopoulos, A. (2008). “New insights into the function of M4 muscarinic acetylcholine receptors gained using a novel allosteric modulator and a DREADD (designer receptor exclusively activated by a designer drug)”. Molecular pharmacology, 74 (4), 1119-1131.

A plasmid encoding hM4D (Gi) is available commercially as plasmid 45548: pcDNA5/FRT-HA-hM4D (Gi) from Addgene, Cambridge, MA 02139 (www.addgene.org/45548/).

A plasmid encoding hM3Dq is also available commercially from Addgene (www.addgene.org/44361). This receptor is sensitive to perlapine (27)

Manipulation of DNA was conducted through conventional molecular biology techniques (see, e.g., Sambrook et al, Molecular Cloning: Cold Spring Harbor Laboratory Press), or with commercial kits according to the manufacturer's protocol. Site directed mutagenesis was performed using the QuikChange II XL kit (Agilent). Other modifications of DNA were performed using the Q5 site directed mutagenesis and Hi-Fi assembly kits (New England Biolabs). Plasmid DNA was purified using the Monarch plasmid miniprep (New England Biolabs) or NucleoBond Xtra midi kits (Thermo Fisher). DNA was quantified by absorbance spectrophotometry using a Nanodrop 1000 (Thermo Fisher), and underwent Sanger sequencing by Source BioScience Limited, UK.

Substitutions were as Follows:

253-255—S85: TCC
V - GTT/GTC/GTA/GTG
337-339—Y113 TAC
C - TGT/TGC
N - AAT/AAC
358-360—V120 GTC
I - ATT/ATC/ATA
361-363—M121 ATG
F - TTT/TTC
367-369—L123 CTT
C - TGT/TGC
I - ATT/ATC/ACA
S - TCT/TCC/TCA/TCG/AGT/AGC
T - ACT/ACC/ACA/ACG
V - GTT/GTC/GTA/GTG
382-384—F128 TTT
I - ATT/ATC/ATA
L - TTA/TTG/CTT/CTC/CTA/CTG
V - GTT/GTC/GTA/GTG
598-600—A200 GCC
T - ACT/ACC/ACA/ACG
607-609—A203 GCC
G - GGT/GGC/GGA/GGG
610-612—F204 TTC
Y - TAT/TAC
1228-1230—I410 ATC
V - GTT/GTC/GTA/GTG
1237-1239—W413 TGG
L - TTA/TTG/CTT/CTC/CTA/CTG
1246-1248—Y416 TAC
F - TTT/TTC

### Cell Culture and Assays

All mammalian cells were maintained in DMEM (10% FBSplus relevant antibiotics at 37° C. and 5% CO₂. Selected mutants were incorporated into CHRM4-Tango (Addgene #66251) via Quikchange mutagenesis (Agilent), and DNA obtained via Monarch mini- or midi-prep kits (New England Biolabs). 4 μg of construct DNA was mixed with 12 UL turbofect (Thermo Fisher) in 400 μL optimem (Thermo Fisher), and transfected into T25s of 70% confluent HTLA cells in 4 mL complete medium (HEK293 cell line stably expressing a tTA-dependent luciferase reporter and a β-arrestin2-TEV fusion gene)^{12,13}. After 2 days cells were detached with citric saline and transferred onto white 1/2-area 96 well plates with different concentrations of drugs in 40 μL optimem (Thermo Fisher). Cells were left overnight, and lysed with direct addition of 40 μL GloMax/Glo lysis buffer (1:1; Promega), followed by luminescence counting on a FlexStation 3 (Molecular Devices) with 1 second integration per well.

The GloSensor CAMP assay was used to confirm results of select mutants, with construct DNA cotransfected 1:1 with 22F reporter plasmid (Promega) into T25s of HEK-293T cells with turbofect as above. Cells were detached with citric saline the following day and transferred to white 1/2-area 96 well plates in 100 μL optimem. One day later, medium was removed and cells were washed with 100 μL HBSS (20 mM HEPES, pH 7.4), followed by addition of 40 μL 5% GloSensor reagent in HBSS. After 1 hr incubation, drugs at varying concentrations were added in 10 μL HBSS to each well. Luminescence was counted for 15 min on a FlexStation 3 with 1 second integration per well, followed by addition of 10 μL isoprenaline in HBSS for a final concentration of 200 nM. Plates were read again for 15 min.

For electrophysiological confirmation of Gi dependent Kir3.1/3.2 Gi activation, mutants were inserted into a hM4D(Gi)-plasmid (Addgene #45548). Following transfection as above, Gi dependent Kir3.1/3.2 activation was quantified using the whole cell patch-clamp technique, as described in Weston et. al³.

### REFERENCES FOR METHODS

1. Rosenbaum, D. M., et al. Structure and function of an irreversible agonist-β2 adrenoceptor complex. Nature 469, 236-240 (2011).
2. Shimamura, T., et al. Structure of the human histamine H1 receptor complex with doxepin. Nature 475, 65-70 (2011).
3. Weston, M., et al. Olanzapine: A potent agonist at the hM4D (Gi) DREADD amenable to clinical translation of chemogenetics. Science Advances 5, eaaw1567 (2019).
4. The, P.O.N.E.S. Correction: Targeting Photoreceptors via Intravitreal Delivery Using Novel, Capsid-Mutated AAV Vectors. PloS one 9, e110030 (2014).
5. Young, D., et al. Adenosine kinase, glutamine synthetase and EAAT2 as gene therapy targets for temporal lobe epilepsy. Gene therapy (2014).
6. Masson, N., Ellis, M., Goodbourn, S. & Lee, K. A. Cyclic AMP response element-binding protein and the catalytic subunit of protein kinase A are present in F9 embryonal carcinoma cells but are unable to activate the somatostatin promoter. Molecular and cellular biology 12, 1096-1106 (1992).
7. Berthold, M. R., et al. KNIME: The Konstanz Information Miner. in Studies in Classification, Data Analysis, and Knowledge Organization (GfKL 2007) (Springer, 2007).
8. Pettersen, E. F., et al. UCSF Chimera-A visualization system for exploratory research and analysis. Journal of Computational Chemistry 25, 1605-1612 (2004).
9. Thal, D. M., et al. Crystal structures of the M1 and M4 muscarinic acetylcholine receptors. Nature 531, 335-340 (2016).
10. Hausser, M. Optogenetics: the age of light. Nature methods 11, 1012-1014 (2014).
11. Kumar, S., et al. MEGA X: Molecular Evolutionary Genetics Analysis across computing platforms. Molecular Biology and Evolution 35, 1547-1549 (2018).
12. Barnea, G, et al. The genetic design of signaling cascades to record receptor activation. Proc Natl Acad Sci USA. 105, 64-69 (2008).
13. Kroeze, W. K., et al. PRESTO-Tango as an open-source resource for interrogation of the druggable human GPCRome. Nat Struct Mol Biol 22, 362-369 (2015).

### Example 1: Structural Basis for Effects of Y113 and A203 Mutation

These are the original residues reported in Armbruster et al. PNAS 2007, 104, 5163-5168 The proposed mechanism of action is as follows:

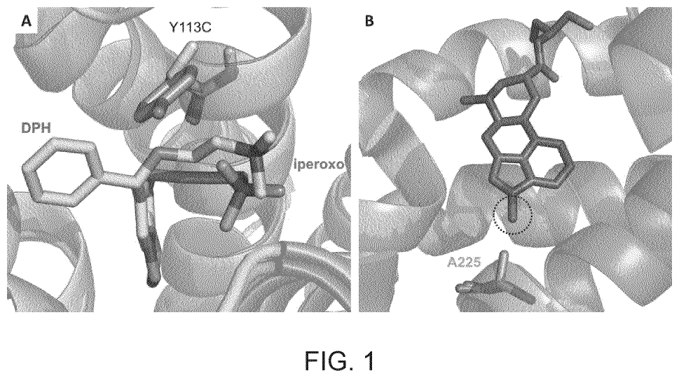

Y113C: Creates space to allow for larger molecules—compared to the endogenous agonist acetylcholine—to bind in an active conformation. In addition, hydrophobic contacts to the endogenous ligand or to other agonists of similar size (such as iperoxo) are lost, thus preventing binding (FIG. 1A).

A203G: Converts antagonists to agonists by removing steric bulk, which prevents movement of helix 5 upon receptor activation. The corresponding A225G mutation in the serotonergic HTR2B receptor converts the antagonist methysergide into a partial agonist, but does not substantially affect potency or efficacy of the agonist methylergonovine (McCorvy et al., Nat. Struct. Mol. Biol. 2018, 25, 787-796, doi.org/10.1038/s41594-018-0116-7)). FIG. 1B highlights how the methyl-group in methysergide would clash with A225 in the active HTR2B conformation, but is compatible with the A225G mutant.

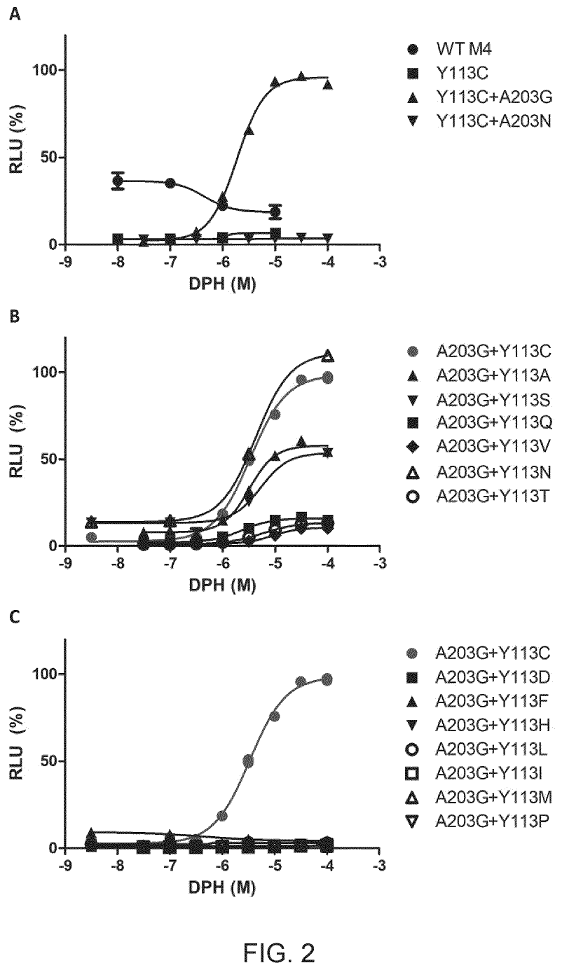

Experimental Results are shown in FIG. 2. It can be concluded that A203G provides for agonist activity.

Y113 tolerates mutations C and N, to a lower level A and V, and to some extent T, Q, and S.

### Example 2: Effect of Mutation at Position 85

The proposed mechanism of action is as follows:

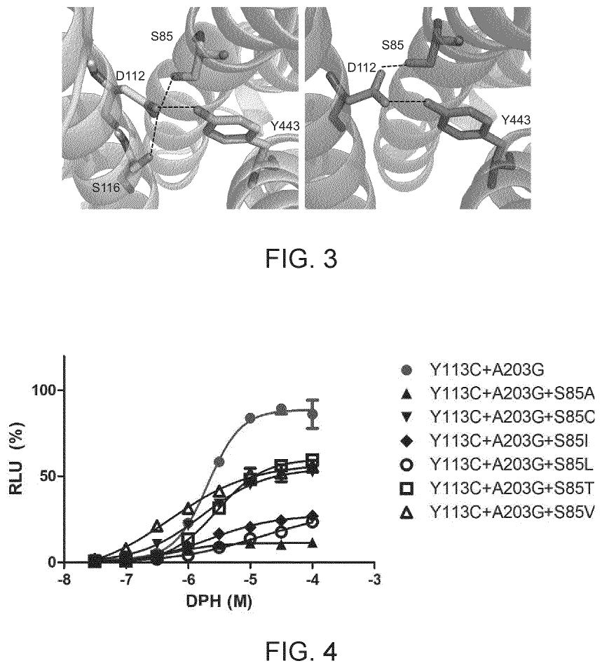

S85V: As shown in FIG. 3, this is part of a hydrogen bond network involving D112, Y443, and S116 in the active (FIG. 3 left), and D112 and Y442 in the inactive (FIG. 3 right), conformation. Upon ligand binding, the carboxy-group of D112 adopts a different rotamer. Introducing hydrophobicity in the S85V mutation is not compatible with the inactive state D112 rotamer, but can form hydrophobic contacts with the Cbeta atom of the D112 active conformation rotamer, thus stabilizing the active conformation.

Experimental Results are shown in FIG. 4. S85V and S85C increase DPH potency (also in combination with Y416F, see below). Both S85V and S85C however decrease efficacy.

### Example 3: Effect of Mutation at Position 416

The proposed mechanism of action is as follows:

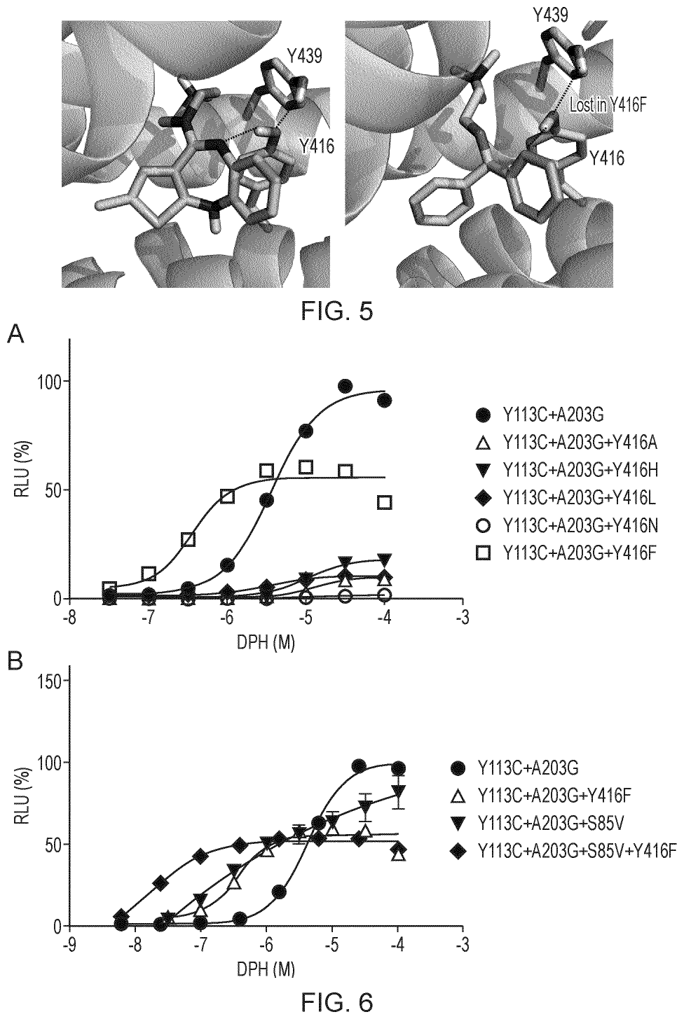

Y416F: As shown in FIG. 5, this removes the polarity of the OH-group, which would otherwise be in close proximity to the hydrophobic phenyl ring of DPH. Interaction with the OH-group was tolerated for olanzapine (which was showed to be a full agonist of hM4D (Gi) in Weston et al 2019, Sci Adv. 2019 Apr. 17; 5 (4): eaaw1567) as the OH-group was potentially involved in a hydrogen bond with the benzodiazepine-nitrogen of olanzapine. A comparison can be made with the crystal structure of the beta2 adrenoceptor in complex with an irreversible agonist (3PDS; Rosenbaum et al., Nature 2011, 469:236-240).

Experimental Results are shown in FIG. 6. Y416F increases the potency of DPH, and Y416F in combination with S85V further increases potency.

### Example 4: Effect of Mutation at Position 120

The proposed mechanism of action is as follows:

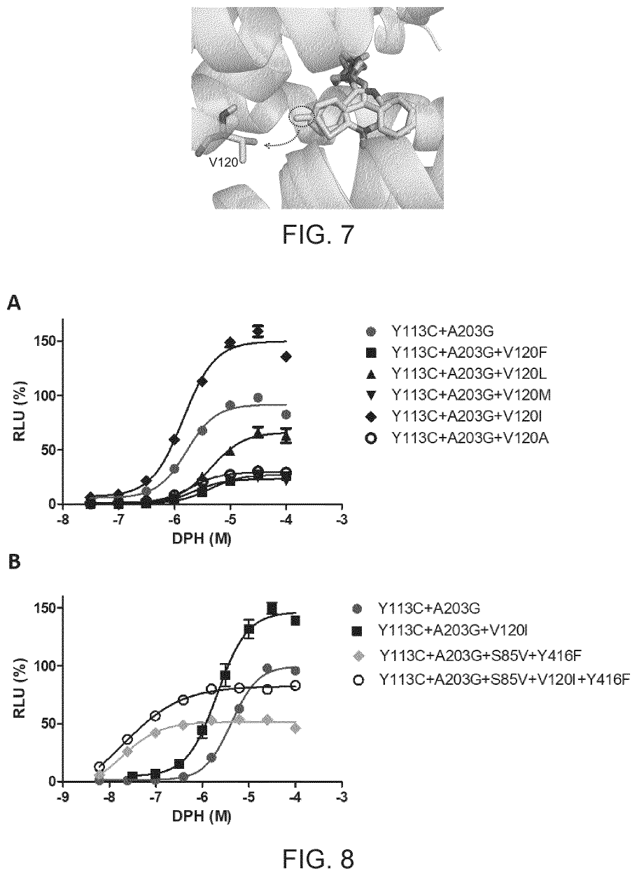

V120I: As shown in FIG. 7, this mutation may ‘fill’ the space of binding site better and thus increase hydrophobic contacts. The additional methyl-group of 11e in comparison to Val compensates for loss of the olanzapine-methyl group. The importance of residue 3.40 and its contact on TM6 is explained in an NMR spectroscopy study of the M2 receptor (Xu et al, Molecular Cell 2019, 75:53-65).

Experimental Results are shown in FIG. 8. The V120I mutation increases the efficacy of DPH.

### Example 5: Effect of Mutation at Position 123

The proposed mechanism of action is as follows:

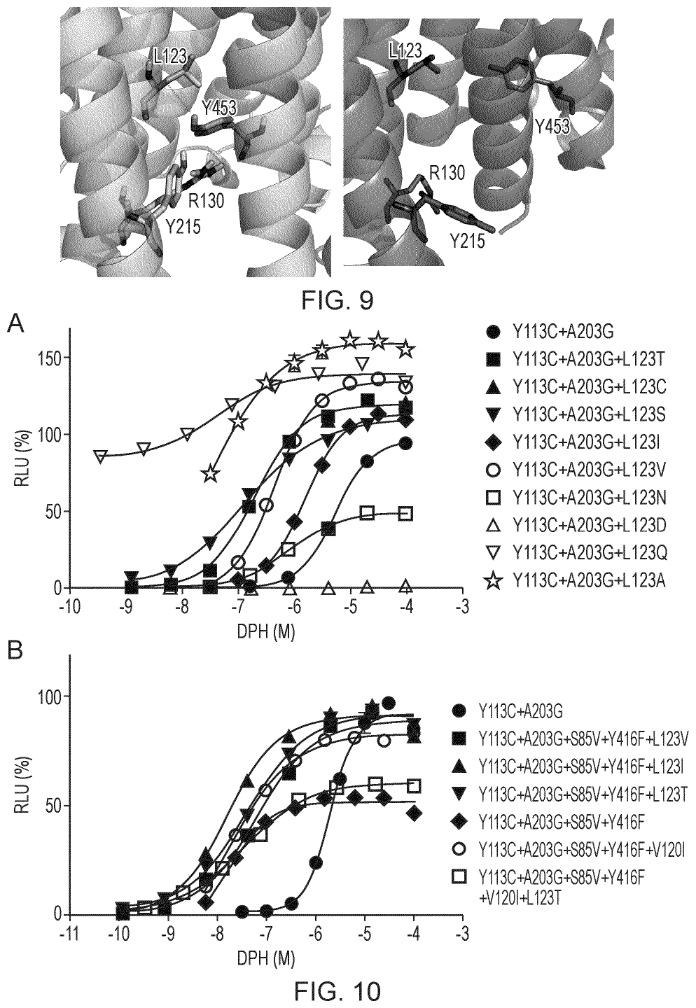

L123T/S: As shown in FIG. 9, this modification may stabilize a network of interactions formed upon receptor activation, but lacking in inactive conformations.

Experimental Results are shown in FIG. 10. L123 mutations increase the potency and/or efficacy of DPH. Some of the other L123 substitutions lead to a constitutively active receptor. FIG. 10B suggests lower efficacy of DPH at Y113C+A203G+S85V+Y416F+V120I+L123T relative to Y113C+A203G+S85V+Y416F+V120I. Note, however that this assay measures β-arrestin recruitment, and lower efficacy is not evident in the assay measuring G_{β/γ}-dependent activation of GIRK (Kir3.1 and Kir3.2) conductance (FIG. 21D). It therefore shows decreased β-arresting recruitment while maintaining G-protein signalling.

### Example 6: Effect of Mutation at Position 128

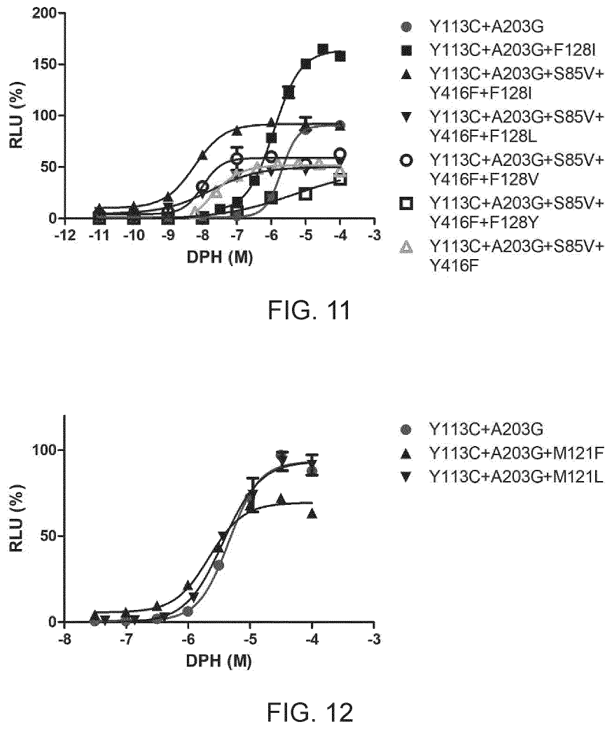

As shown in FIG. 11, mutation of F128 to I, L and V, but not Y, increases the efficacy and/or potency of DPH.

### Example 7: Effect of Mutation at Position 121

As shown in FIG. 12, mutation of M121 to F but not L increases the potency of DPH.

### Example 8: Effect of Mutations at Positions 200 and 204

The proposed mechanism of action is as follows:

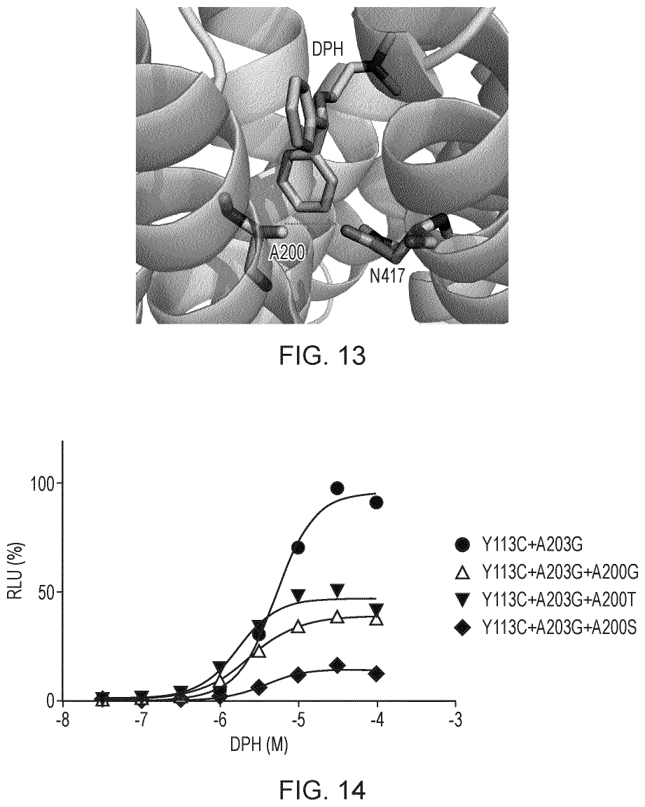

A200T: As shown in FIG. 13, this modification apparently stabilizes the active conformation of the receptor by introducing a hydrogen bond to N417 and potentially introducing new hydrophobic contacts.

Experimental Results are shown in FIG. 14. Mutation of A200 to T, but not G or S increases the potency of DPH, although the effect is small and comes at the cost of decreasing efficacy.

The proposed mechanism of action in relation to position 204 is as follows:

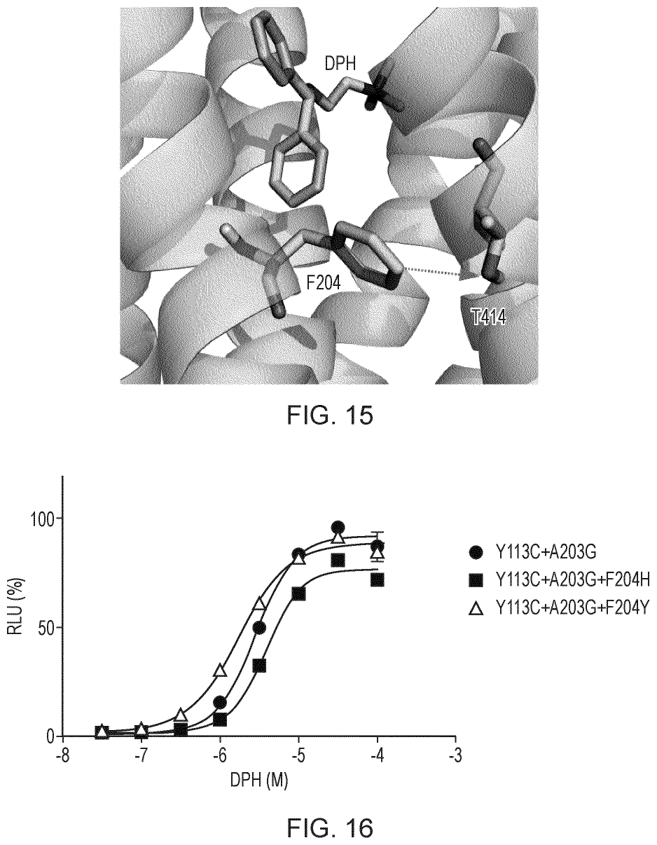

F204Y: As shown in FIG. 15, this modification apparently stabilizes the active conformation of the receptor by introducing hydrogen bond to T414.

Experimental Results are shown in FIG. 16. Mutation of F204 to Y but not H increases the potency of DPH

### Example 9: Effect of Mutation at Position 413

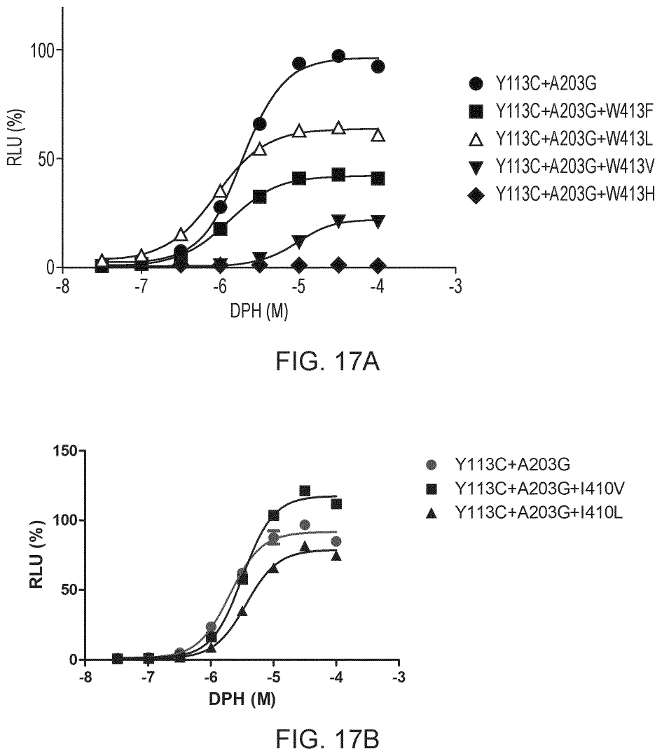

As shown in FIG. 17A, W413L mutation increases the potency of DPH, although at the cost of decreasing efficacy.

As shown in FIG. 17B, I410V mutant leads to an increased efficacy of DPH.

### Example 10: Provision of Alternative Ligands

Alternative ligands were provided by two different search strategies:

Search strategy 1 was based on chemical similarity, and comprised:
a) Approved drugs that were similar to diphenhydramine according to Shape-IT using the Swiss Similarity webserver (www.swisssimilarity.ch; Zoete, V., Daina, A., Bovigny, C., & Michielin, O. SwissSimilarity: A Web Tool for Low to Ultra High Throughput Ligand-Based Virtual Screening., J. Chem. Inf. Model., 2016, 56 (8), 1399-1404.)
b) Compounds that had a similarity of 0.5 to diphenhydramine using DataWarrior and FragFP (Thomas Sander, Joel Freyss, Modest von Korff, Christian Rufener. DataWarrior: An Open-Source Program For Chemistry Aware Data Visualization And Analysis. J Chem Inf Model 2015, 55, 460-473, doi 10.1021/ci500588j).
c) Approved drugs that had a similarity of 0.5 to diphenhydramine using the ChEMBL webserver (www.ebi.ac.uk/chembl; A. Gaulton, L. Bellis, J. Chambers, M. Davies, A. Hersey, Y. Light, S. McGlinchey, R. Akhtar, A. P. Bento, B. Al-Lazikani, D. Michalovich, & J. P. Overington (2012) ‘ChEMBL: A Large-scale Bioactivity Database For Chemical Biology and Drug Discovery’ Nucleic Acids Res. Database Issue, 40 D1100-1107. DOI: 10.1093/nar/gkr777 PMID: 21948594; A. P. Bento, A. Gaulton, A. Hersey, L. J. Bellis, J. Chambers, M. Davies, F. A. Kruger, Y. Light, L. Mak, S. McGlinchey, M. Nowotka, G. Papadatos, R. Santos & J. P. Overington (2014) ‘The ChEMBL bioactivity database: an update’ Nucleic Acids Res. Database Issue, 42 1083-1090. DOI: 10.1093/nar/gkt103 PMID: 24214965

The results are listed in Table 1.

Search strategy 2 was based on functional, and hence structural, similarity, and comprised:
2.) Approved anti-histamines that may be expected to being anti-histamine (e.g. Diphenhydramine) binding GRANPAs.

The results are listed in Table 2.

### Example 11: Further Analysis of GRANPA Mutations with Different Assays

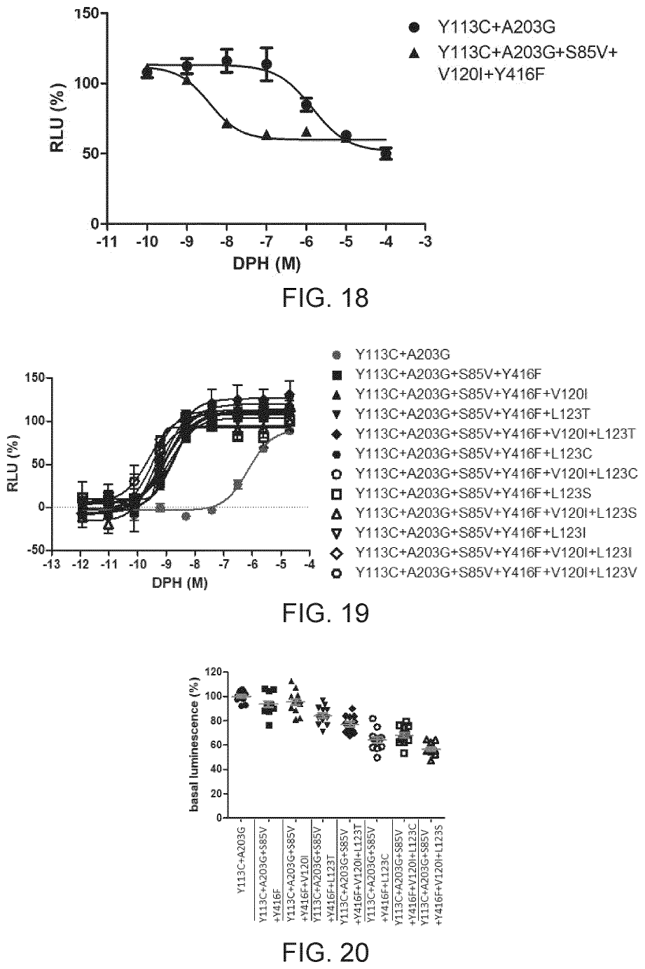

FIG. 18 shows a combination of mutations of the invention measured by the GloSensor assay for the Gi cascade, which was used to verify the ability to inhibit CAMP production.

FIG. 19 shows the results of Gi coupled activity of hM4D (Gi) (Y113C+A203G) and mutants as measured with the GloSensor assay. The addition of S85V+Y416F±V120I±L123 (T/C/S/I/V) increased the potency for activation by DPH. Data was normalised as percentage inhibition of cAMP as measured by light output, relative to control hM4D (Gi) treated with 20 μM DPH. EC50 and span of constructs are as follows:
Y113C+A203G (717 nM; 94%),
Y113C+A203G+S85V+Y416F (1.50 nM; 116%),
Y113C+A203G+S85V+Y416F+V120I (1.80 nM; 111%),
Y113C+A203G+S85V+Y416F+L123T (0.949 nM; 129%),
Y113C+A203G+S85V+Y416F+V120I+L123T (0.956 nM; 129%),
Y113C+A203G+S85V+Y416F+L123C (0.366 nM; 115%),
Y113C+A203G+S85V+Y416F+V120I+L123C (0.252 nM; 101%),
Y113C+A203G+S85V+Y416F+L123S (0.528 nM; 83%),
Y113C+A203G+S85V+Y416F+V120I+L123S (0.321 nM; 110%),
Y113C+A203G+S85V+Y416F+L123| (1.41 nM; 98%)
Y113C+A203G+S85V+Y416F+V120I+L123I (0.880 nM; 119%),
Y113C+A203G+S85V+Y416F+L123V (1.15 nM; 109%).

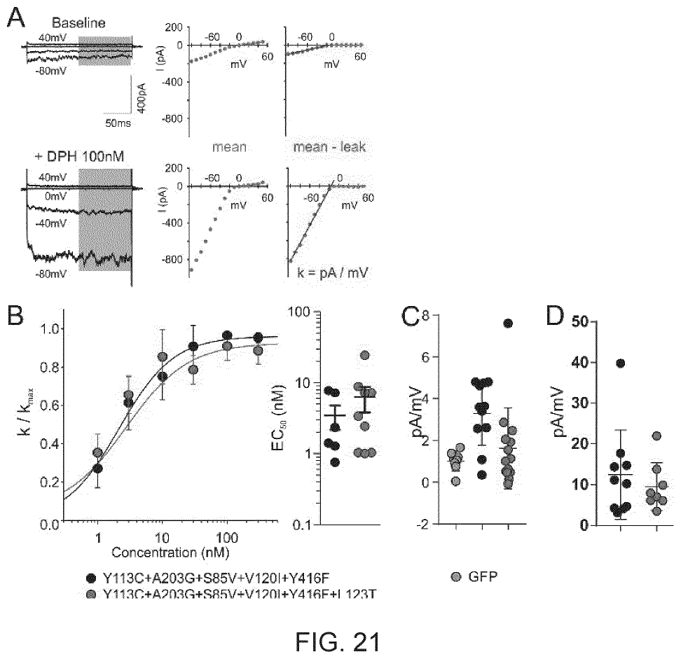

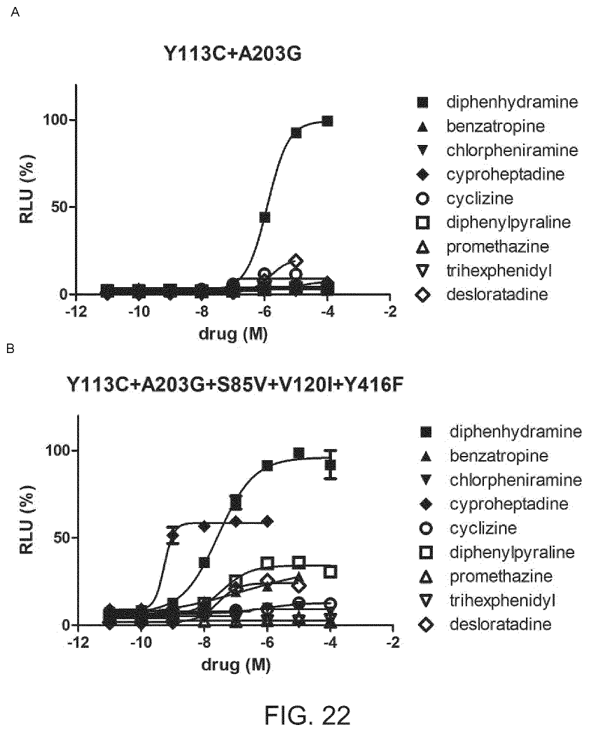

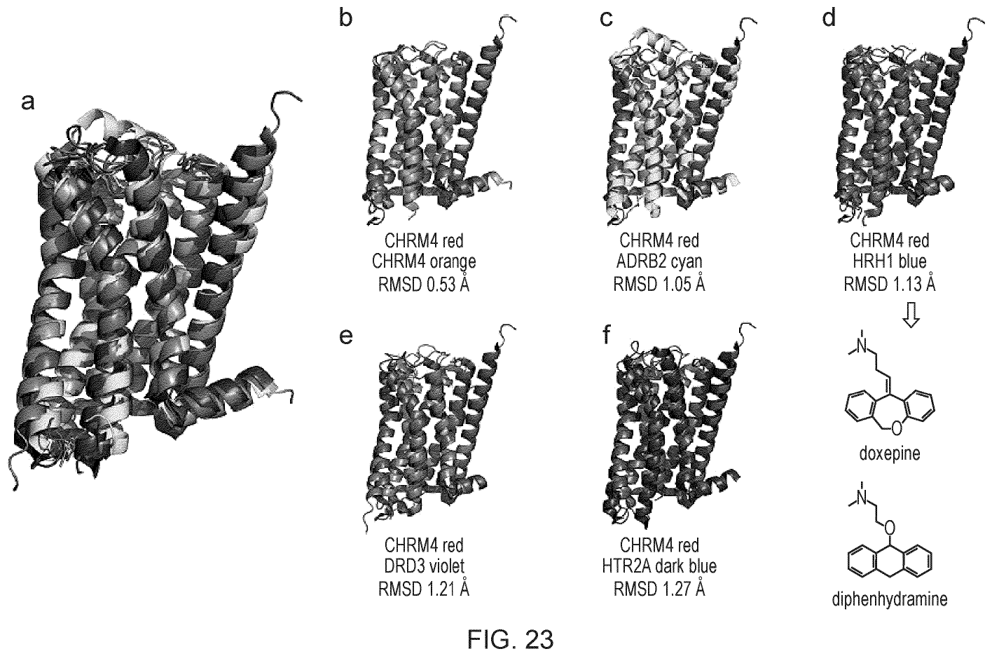

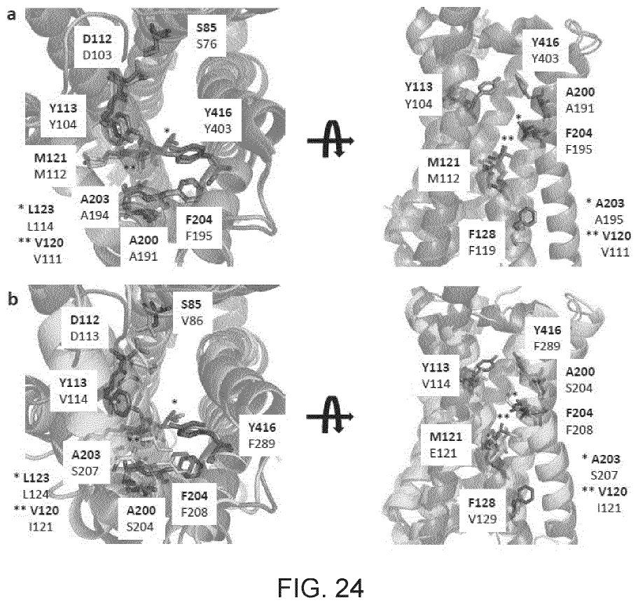

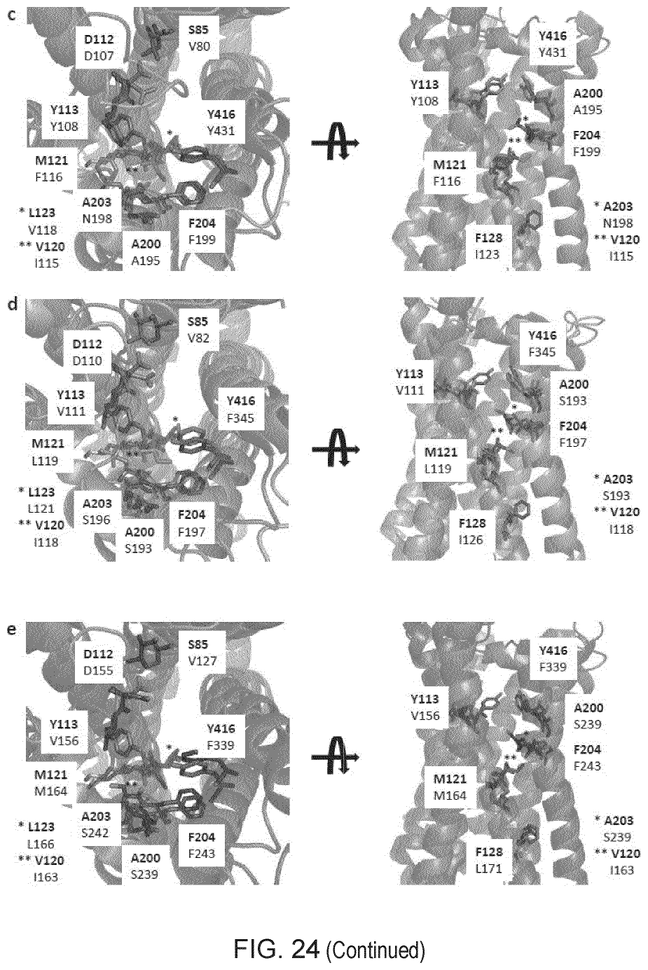

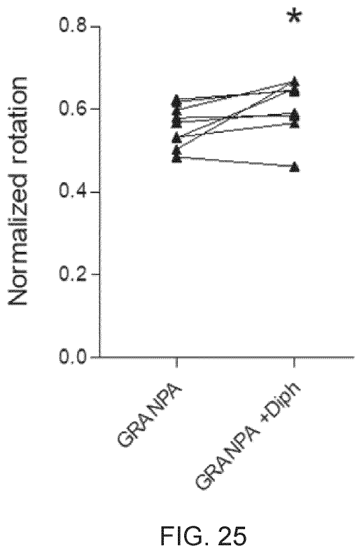

FIG. 20 shows the results of hM4D (Gi) and mutants tested for effects on basal cAMP levels using the GloSensor assay. Data was normalised to hM4D (Gi) (Y113C+A203G). A decrease in basal luminescence without drug treatment demonstrates increase in constitutive activity. Y113C+A203G (100%) was not significantly different to Y113C+A203G+S85V+Y416F (93%) and Y113C+A203G+S85V+Y416F+V120I (95%), while other mutant combinations were (One-way ANOVA, P<0.05).

FIG. 21 shows the results of an electrophysiology assay testing the G_{β/γ}-dependent potentiation of Kir3.1 and Kir3.2 G-protein coupled inward rectifying potassium channels GIRKs).

The GIRK assay confirms that both Y113C+A203G+S85V+V120I+Y416F and Y113C+A203G+S85V+V120I+Y416F+L123T are potently activated by DPH In contrast, Y113C+A203G+S85V+V120I+Y416F+L123T showed less recruitment in the β-arrestin assay data in FIG. 10B. However, although they show similar efficacies in the GIRK assay, Y113C+A203G+S85V+V120I+Y416F, but not Y113C+A203G+S85V+V120I+Y416F+L123T, exhibits some basal activity.

Accordingly Y113C+A203G+S85V+V120I+Y416F+L123T is a preferred GRANPA.

### Example 12: Other Activating Ligands

FIG. 22 confirms other agents, including other antihistamines and antimuscarinic agents, can be used as the activating ligand, and in particular their potency and/or efficacy can be improved by use of the modifications of the invention e.g. S85V, V120I and Y416F.

### Example 13-Summary of Preferred Embodiments and Amino Acid Sequence Correspondence Numbering

The reference mAChR4 numbering is shown, with Ballesteros-Weinstein numbering in brackets

### Original DREADD:

| | |
|---|---|
| Y113 - C/N | (3.33) |
| A203 - G | (5.46) |

Mutations with Substantial Increase in DPH Potency/Efficacy:

| | |
|---|---|
| S85 - V | (2.57) |
| V120 - I | (3.40) |
| L123 - C/I/S/T/V | (3.43) |
| F128 - I/L/V | (3.48) |
| Y416 - F | (6.51) |

### Less Substantial Increase in DPH Potency:

| | |
|---|---|
| M121 - F | (3.41) |
| A200 - T | (5.43) |
| F204 - Y | (5.47) |
| I410 - V | (6.45) |
| W413 - L | (6.48) |

Alignment of closely related GPCR family members shows high structural conservation (FIG. 23) and residues identified in CHRM4 can be readily aligned to corresponding residues in other aminergic GPCRs (FIG. 24).

Corresponding residues in other aminergic GPCRs are shown below. Alignment of FASTA peptide sequences were performed using MEGA X 10.1.8 (Kumar, S. et al., “MEGA X: Molecular Evolutionary Genetics Analysis across computing platforms” Molecular Biology and Evolution 35, 1547-1549 (2018)), with the MUSCLE algorithm (standard settings: Gap Open=−2.9, Gap Extent=0.0, Hydrophobicity Multiplier=1.2, Max Iterations=16, Cluster Method=UPGMA, Min Diag Length=24).

### Example 14—GRANPA Activation with Diphenhydramine In Vivo Alters Behaviour

A recombinant adeno-associated virus, pseudotyped with AAV9 capsid, expressing GRANPA (Y113C+A203G+S85V+V120|+Y416F+L123T) under the hCaMKII promoter (titre: 6.7×10\hat{ }14 vg/mL) was injected in the right substantia nigra (volume: 300 nanolitres) in mice under anaesthesia. After 3 weeks the mice received an intraperitoneal injection of amphetamine (3 mg/kg) to enhance spontaneous locomotion, either alone or together with diphenhydramine (1 mg/kg). The number of whole body rotations to the left and right were then counted using a machine vision tool. Diphenhydramine led to a significant increase in leftward rotations (n=9, p=0.037, Student's paired t-test, FIG. 25).

TABLE 3
S85:

| SEQ ID NO [symbol] | |
|---|---|
| 72 | 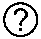 |
| 73 | [symbol] |
| 74 | [symbol] |
| 75 | [symbol] |
| 76 | [symbol] |
| 77 | [symbol] |
| 78 | [symbol] |
| 79 | [symbol] |
| 80 | [symbol] |
| 81 | [symbol] |
| 82 | [symbol] |
| 83 | [symbol] |
| 84 | [symbol] |
| 85 | [symbol] |
| 86 | [symbol] |
| 87 | [symbol] |
| 88 | [symbol] |
| 89 | [symbol] |
| 90 | [symbol] |
| 91 | [symbol] |
| 92 | [symbol] |
| 93 | [symbol] |
| 94 | [symbol] |
| 95 | [symbol] |
| 96 | [symbol] |
| 97 | [symbol] |
| 98 | [symbol] |
| 99 | [symbol] |
| 100 | [symbol] |
| 101 | [symbol] |
| 102 | [symbol] |
| 103 | [symbol] |
| 104 | [symbol] |
| 105 | [symbol] |
| 106 | [symbol] |

[symbol]

indicates data missing or illegible when filed

ACM4 S85 (residue 2.57) corresponds to ACM1 S78, ACM2 S76, ACM3 S121, ACM5 S83, HRH3 C87, HRH4 S68, ADA2B I65.

TABLE 4
V120, M121, L123, F128:

| SEQ ID NO [symbol] | |
|---|---|
| 107 | 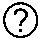 |
| 108 | [symbol] |
| 109 | [symbol] |
| 110 | [symbol] |
| 111 | [symbol] |
| 112 | [symbol] |
| 113 | [symbol] |
| 114 | [symbol] |
| 115 | [symbol] |
| 116 | [symbol] |
| 117 | [symbol] |
| 118 | [symbol] |
| 119 | [symbol] |
| 120 | [symbol] |
| 121 | [symbol] |
| 122 | [symbol] |
| 123 | [symbol] |
| 124 | [symbol] |
| 125 | [symbol] |
| 126 | [symbol] |
| 127 | [symbol] |
| 128 | [symbol] |
| 129 | [symbol] |
| 130 | [symbol] |
| 131 | [symbol] |
| 132 | [symbol] |
| 133 | [symbol] |
| 134 | [symbol] |
| 135 | [symbol] |
| 136 | [symbol] |
| 137 | [symbol] |
| 138 | [symbol] |
| 139 | [symbol] |
| 140 | [symbol] |
| 141 | [symbol] |

[symbol]

indicates data missing or illegible when filed

ACM4 V120 (residue 3.40) corresponds to ACM1 V113, ACM2 V111, ACM3 V156, ACM5 V118, HRH3 A122, HRH4 V102.

ACM4 M121 (residue 3.41) corresponds to ACM1 M114, ACM2 M112, ACM3 M157, ACM5 M119, HRH1 F116, HRH2 L108, HRH3 F123, HRH4 Y103, 5HT1F L112, 5HT1E L111, 5HT1D L127, 5HT1B L138, 5HT5A W130, 5HT7 M171, 5HT1A L125, 5HT2A M164, DRD3 L119, DRD2 L123, DRD4 F124, ADA1B L134, ADA1D L185, ADA1A M115, ADA2B V101, ADA2A V137, ADA2C V140, 5HT6 L115, 5HT2B M144, 5HT2C M143, DRD1B L129, DRD1A L112, 5HT4 F109, ADRB3 E126, ADRB1 E147, ADRB2 E122.

ACM4 L123 (residue 3.43) corresponds to ACM1 L116, ACM2 L114, ACM3 L159, ACM5 L121, HRH1 V118, HRH2 L110, HRH3 I125, HRH4 I105, 5HT1F L114, 5HT1E L113, 5HT1D L129, 5HT1B L140, 5HT5A V132, 5HT7 L173, 5HT1A L127, 5HT2A L166, DRD3 L121, DRD2 L125, DRD4 L126, ADA1B L136, ADA1D L187, ADA1A L117, ADA2B L103, ADA2A L139, ADA2C L142, 5HT6 L117, 5HT2B L146, 5HT2C L145, DRD1B L131, DRD1A L114, 5HT4 L111, ADRB3 L128, ADRB1 L149, ADRB2 L124.

ACM4 F128 (residue 3.48) corresponds to ACM1 F121, ACM2 F119, ACM3 F164, ACM5 F126, HRH1 I123, HRH2 L115, HRH3 Y130, HRH4 Y110, 5HT1F L119, 5HT1E L118, 5HT1D L134, 5HT1B L145, 5HT5A L137, 5HT7 I178, 5HT1A L132, 5HT2A L171, DRD3 I126, DRD2 I130, DRD4 V131, ADA1B 1141, ADA1D V192, ADA1A 1122, ADA2B L108, ADA2A L144, ADA2C L147, 5HT6 L122, 5HT2B V151, 5HT2C L150, DRD1B V136, DRD1A V119, 5HT4 L116, ADRB3 V133, ADRB1 L154, ADRB2 V129.

TABLE 5
A200, F204:

| SEQ ID NO [symbol] | |
|---|---|
| 142 | 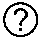 |
| 143 | [symbol] |
| 144 | [symbol] |
| 145 | [symbol] |
| 146 | [symbol] |
| 147 | [symbol] |
| 148 | [symbol] |
| 149 | [symbol] |
| 150 | [symbol] |
| 151 | [symbol] |
| 152 | [symbol] |
| 153 | [symbol] |
| 154 | [symbol] |
| 155 | [symbol] |
| 156 | [symbol] |
| 157 | [symbol] |
| 158 | [symbol] |
| 159 | [symbol] |
| 160 | [symbol] |
| 161 | [symbol] |
| 162 | [symbol] |
| 163 | [symbol] |
| 164 | [symbol] |
| 165 | [symbol] |
| 166 | [symbol] |
| 167 | [symbol] |
| 168 | [symbol] |
| 169 | [symbol] |
| 170 | [symbol] |
| 171 | [symbol] |
| 172 | [symbol] |
| 173 | [symbol] |
| 174 | [symbol] |
| 175 | [symbol] |
| 176 | [symbol] |

[symbol]

indicates data missing or illegible when filed

ACM4 A200 (residue 5.43) corresponds to ACM1 A193, ACM2 A191, ACM3 A236, ACM5 A198, HRH1 A195, HRH2 G187, HRH3 S203, HRH4 S179, 5HT2A S239, DRD3 S193, DRD2 S194, DRD4 S197, ADA1B S208, ADA1D S259, ADA1A A189, ADA2B S177, ADA2A C216, ADA2C C215, 5HT6 S193, 5HT2B S222, 5HT2C S219, DRD1B S230, DRD1A S199, 5HT4 S197, ADRB3 S209, ADRB1 S229, ADRB2 S204.

ACM4 F204 (residue 5.47) corresponds to ACM1 F197, ACM2 F195, ACM3 F240, ACM5 F202, HRH1 F199, HRH2 F191, HRH3 F207, HRH4 F183, 5HT1F F190, 5HT1E F191, 5HT1D F206, 5HT1B F217, 5HT5A 209, 5HT7 248, 5HT1A F204, 5HT2A F243, DRD3 F197, DRD2 F198, DRD4 F201, ADA1B F212, ADA1D F263, ADA1A F193, ADA2B F181, ADA2A F220, ADA2C F219, 5HT6 F197, 5HT2B F226, 5HT2C F223, DRD1B F234, DRD1A F203, 5HT4 F201, ADRB3 F213, ADRB1 F233, ADRB2 F208.

TABLE 6
1410, W413, Y416:

| SEQ ID NO [symbol] | |
|---|---|
| 177 | 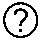 |
| 178 | [symbol] |
| 179 | [symbol] |
| 180 | [symbol] |
| 181 | [symbol] |
| 182 | [symbol] |
| 183 | [symbol] |
| 184 | [symbol] |
| 185 | [symbol] |
| 186 | [symbol] |
| 187 | [symbol] |
| 188 | [symbol] |
| 189 | [symbol] |
| 190 | [symbol] |
| 191 | [symbol] |
| 192 | [symbol] |
| 193 | [symbol] |
| 194 | [symbol] |
| 195 | [symbol] |
| 196 | [symbol] |
| 197 | [symbol] |
| 198 | [symbol] |
| 199 | [symbol] |
| 200 | [symbol] |
| 201 | [symbol] |
| 202 | [symbol] |
| 203 | [symbol] |
| 204 | [symbol] |
| 205 | [symbol] |
| 206 | [symbol] |
| 207 | [symbol] |
| 208 | [symbol] |
| 209 | [symbol] |
| 210 | [symbol] |
| 211 | [symbol] |

[symbol]

indicates data missing or illegible when filed

ACM4 I410 (residue 6.45) corresponds to ACM1 I375, ACM2 I397, ACM3 I501, ACM5 I452, HRH1 I425, HRH2 I244, HRH3 G368, HRH4 A313, 5HT1E I301, 5HT1D I311, 5HT1B 1324, 5HT7 T337, 5HT1A 1355, DRD3 I339, DRD2 I383, DRD4 L356, ADA1B 1304, 5HT6 F278, 5HT2B L334, 5HT2C L321, 5HT4 C269, ADRB3 T302, ADRB1 T334, ADRB2 T283.

ACM4 W413 (residue 6.48) corresponds to ACM1 W378, ACM2 W400, ACM3 W504, ACM5 W455, HRH1 W428, HRH2 W247, HRH3 W371, HRH4 W316, 5HT1F W306, 5HT1E W304, 5HT1D W314, 5HT1B W327, 5HT5A W298, 5HT7 W340, 5HT1A W358, 5HT2A W336, DRD3 W342, DRD2 W386, DRD4 W359, ADA1B W307, ADA1D W361, ADA1A W285, ADA2B W384, ADA2A W402, ADA2C W395, 5HT6 W281, 5HT2B W337, 5HT2C W324, DRD1B W309, DRD1A W285, 5HT4 W272, ADRB3 W305, ADRB1 W337, ADRB2 W286.

ACM4 Y416 (residue 6.51) corresponds to ACM1 Y381, ACM2 Y403, ACM3 Y507, ACM5 Y458, HRH1 Y431, HRH2 Y250, HRH3 Y374, HRH4 Y319.

### Sequence Annex

```
SEQ ID. NO: 1 polypeptide sequence: CHRM4, human muscarinic
receptor type 4
*MANFT PVNGS SGNQS VRLVT SSSHN RYETV EMVFI ATVTG SLSLV TVVGN 50
  ILVML SIKVN RQLQT VNNYF LFSLA CADLI IGAFS MNLYT VYIIK GYWPL 100
  GAVVC DLWLA LDYVV SNASV MNLLI ISFDR YFCVT KPLTY PARRT TKMAG 150
  LMIAA AWVLS FVLWA PAILF WQFVV GKRTV PDNQC FIQFL SNPAV TFGTA 200
  IAAFY LPVVI MTVLY IHISL ASRSR VHKHR PEGPK EKKAK TLAFL KSPLM 250
  KQSVK KPPPG EAARE ELRNG KLEEA PPPAL PPPPR PVADK DTSNE SSSGS 300
  ATQNT KERPA TELST TEATT PAMPA PPLQP RALNP ASRWS KIQIV TKQTG 350
  NECVT AIEIV PATPA GMRPA ANVAR KFASI ARNQV RKKRQ MAARE RKVTR 400
  TIFAI LLAFI LTWTP YNVMV LVNTF CQSCI PDTVW SIGYW LCYVN STINP 450
  ACYAL CNATF KKTFR HLLLC QYRNI GTAR* 479
SEQ ID. NO: 71: Nucleotide sequence: NM_000741
atggccaacttcacacctgtcaatggcagctcgggcaatcagtccgtgcgcctggtcacg 60
tcatcatcccacaatcgctatgagacggtggaaatggtcttcattgccacagtgacaggc 120
tccctgagcctggtgactgtcgtgggcaacatcctggtgatgctgtccatcaaggtcaac 180
aggcagctgcagacagtcaacaactacttcctcttcagcctggcgtgtgctgatctcatc 240
ataggcgccttctccatgaacctctacaccgtgtacatcatcaagggctactggcccctg 300
ggcgccgtggtctgcgacctgtggctggccctggactacgtggtgagcaacgcctccgtc 360
atgaaccttctcatcatcagctttgaccgctacttctgcgtcaccaagcctctcacctac 420
cctgcccggcgcaccaccaagatggcaggcctcatgattgctgctgcctgggtactgtcc 480
ttcgtgctctgggcgcctgccatcttgttctggcagtttgtggtgggtaagcggacggtg 540
cccgacaaccagtgcttcatccagttcctgtccaacccagcagtgacctttggcacagcc 600
attgctgccttctacctgcctgtggtcatcatgacggtgctgtacatccacatctccctg 660
gccagtcgcagccgagtccacaagcaccggcccgagggcccgaaggagaagaaagccaag 720
acgctggccttcctcaagagcccactaatgaagcagagcgtcaagaagcccccgcccggg 780
gaggccgcccgggaggagctgcgcaatggcaagctggaggaggcccccccgccagcgctg 840
ccaccgccaccgcgccccgtggctgataaggacacttccaatgagtccagctcaggcagt 900
gccacccagaacaccaaggaacgcccagccacagagctgtccaccacagaggccaccacg 960
cccgccatgcccgcccctcccctgcagccgcgggccctcaacccagcctccagatggtcc 1020
aagatccagattgtgacgaagcagacaggcaatgagtgtgtgacagccattgagattgtg 1080
cctgccacgccggctggcatgcgccctgcggccaacgtggcccgcaagttcgccagcatc 1140
gctcgcaaccaggtgcgcaagaagcggcagatggcggcccgggagcgcaaagtgacacga 1200
acgatctttgccattctgctagccttcatcctcacctggacgccctacaacgtcatggtc 1260
ctggtgaacaccttctgccagagctgcatccctgacacggtgtggtccattggctactgg 1320
ctctgctacgtcaacagcaccatcaaccctgcctgctatgctctgtgcaacgccaccttt 1380
aaaaagaccttccggcacctgctgctgtgccagtatcggaacatcggcactgccaggtag 1440
```

TABLE 8
GPCR sequences.

| Aa | UniProt | UniProt | | Geninfo | NCBI Protein | NCBI Nucleotide | Nt |
|---|---|---|---|---|---|---|---|
| SEQ ID | Accession | Entry Name | UniProt Protein Name | Identifier | Sequence | Sequence | SEQ ID |
| 1 | P08173 | ACM4_HUMAN | Muscarinic | 52426748 | NP_000732.2 | NM_000741.5 | 36 |
| | | | acetylcholine receptor | | | | |
| | | | M4 | | | | |
| 2 | P11229 | ACM1_HUMAN | Muscarinic | 37622910 | NP_000729.2 | NM_000738.3 | 37 |
| | | | acetylcholine receptor | | | | |
| | | | M1 | | | | |
| 3 | P08172 | ACM2_HUMAN | Muscarinic | 4502817 | NP_000730.1 | NM_000739.3 | 38 |
| | | | acetylcholine receptor | | | | |
| | | | M2 | | | | |
| 4 | P20309 | ACM3_HUMAN | Muscarinic | 4502819 | NP_000731.1 | NM_000740.4 | 39 |
| | | | acetylcholine receptor | | | | |
| | | | M3 | | | | |
| 5 | P08912 | ACM5_HUMAN | Muscarinic | 7108336 | NP_036257.1 | NM_012125.4 | 40 |
| | | | acetylcholine receptor | | | | |
| | | | M5 | | | | |
| 6 | P35367 | HRH1_HUMAN | Histamine H1 receptor | 4504491 | NP_000852.1 | NM_000861.3 | 41 |
| 7 | P25021 | HRH2_HUMAN | Histamine H2 receptor | 197085610 | NP_001124527.1 | NM_001131055.2 | 42 |
| 8 | Q9Y5N1 | HRH3_HUMAN | Histamine H3 receptor | 194018562 | NP_009163.2 | NM_007232.3 | 43 |
| 9 | Q9H3N8 | HRH4_HUMAN | Histamine H4 receptor | 14251205 | NP_067637.2 | NM_021624.4 | 44 |
| 10 | P30939 | 5HT1F_HUMAN | 5-hydroxytryptamine | 10835197 | NP_000857.1 | NM_000866.5 | 45 |
| | | | receptor 1F | | | | |
| 11 | P28566 | 5HT1E_HUMAN | 5-hydroxytryptamine | 4504537 | NP_000856.1 | NM_000865.3 | 46 |
| | | | receptor 1E | | | | |
| 12 | P28221 | 5HT1D_HUMAN | 5-hydroxytryptamine | 4504535 | NP_000855.1 | NM_000864.5 | 47 |
| | | | receptor 1D | | | | |
| 13 | P28222 | 5HT1B_HUMAN | 5-hydroxytryptamine | 4504533 | NP_000854.1 | NM_000863.3 | 48 |
| | | | receptor 1B | | | | |
| 14 | P47898 | 5HT5A_HUMAN | 5-hydroxytryptamine | 13236497 | NP_076917.1 | NM_024012.4 | 49 |
| | | | receptor 5A | | | | |
| 15 | P34969 | 5HT7R_HUMAN | 5-hydroxytryptamine | 10880129 | NP_062873.1 | NM_019859.4 | 50 |
| | | | receptor 7 | | | | |
| 16 | P08908 | 5HT1A_HUMAN | 5-hydroxytryptamine | 55956923 | NP_000515.2 | NM_000524.4 | 51 |
| | | | receptor 1A | | | | |
| 17 | P28223 | 5HT2A_HUMAN | 5-hydroxytryptamine | 10835175 | NP_000612.1 | NM_000621.5 | 52 |
| | | | receptor 2A | | | | |
| 18 | P35462 | DRD3_HUMAN | D(3) dopamine receptor | 89191861 | NP_000787.2 | NM_000796.6 | 53 |
| 19 | P14416 | DRD2_HUMAN | D(2) dopamine receptor | 4503385 | NP_000786.1 | NM_000795.4 | 54 |
| 20 | P21917 | DRD4_HUMAN | D(4) dopamine receptor | 32483397 | NP_000788.2 | NM_000797.4 | 55 |
| 21 | P35368 | ADA1B_HUMAN | Alpha-1B adrenergic | 4501959 | NP_000670.1 | NM_000679.4 | 56 |
| | | | receptor | | | | |
| 22 | P25100 | ADA1D_HUMAN | Alpha-1D adrenergic | 4501957 | NP_000669.1 | NM_000678.4 | 57 |
| | | | receptor | | | | |
| 23 | P35348 | ADA1A_HUMAN | Alpha-1A adrenergic | 111118992 | NP_000671.2 | NM_000680.4 | 58 |
| | | | receptor | | | | |
| 24 | P18089 | ADA2B_HUMAN | Alpha-2B adrenergic | 33598960 | NP_000673.2 | NM_000682.7 | 59 |
| | | | receptor | | | | |
| 25 | P08913 | ADA2A_HUMAN | Alpha-2A adrenergic | 194353970 | NP_000672.3 | NM_000681.4 | 60 |
| | | | receptor | | | | |
| 26 | P18825 | ADA2C_HUMAN | Alpha-2C adrenergic | 71773208 | NP_000674.2 | NM_000683.4 | 61 |
| | | | receptor | | | | |
| 27 | P50406 | 5HT6R_HUMAN | 5-hydroxytryptamine | 4504545 | NP_000862.1 | NM_000871.3 | 62 |
| | | | receptor 6 | | | | |
| 28 | P41595 | 5HT2B_HUMAN | 5-hydroxytryptamine | 222080049 | NP_000858.3 | NM_000867.5 | 63 |
| | | | receptor 2B | | | | |
| 29 | P28335 | 5HT2C_HUMAN | 5-hydroxytryptamine | 4504541 | NP_000859.1 | NM_000868.3 | 64 |
| | | | receptor 2C | | | | |
| 30 | P21918 | DRD5_HUMAN | D(1B) dopamine | 4503391 | NP_000789.1 | NM_000798.5 | 65 |
| | | | receptor | | | | |
| 31 | P21728 | DRD1_HUMAN | D(1A) dopamine | 4503383 | NP_000785.1 | NM_000794.5 | 66 |
| | | | receptor | | | | |
| 32 | Q13639 | 5HT4R_HUMAN | 5-hydroxytryptamine | 11321563 | NP_000861.1 | NM_000870.7 | 67 |
| | | | receptor 4 | | | | |
| 33 | P13945 | ADRB3_HUMAN | Beta-3 adrenergic | 4557267 | NP_000016.1 | NM_000025.3 | 68 |
| | | | receptor | | | | |
| 34 | P08588 | ADRB1_HUMAN | Beta-1 adrenergic | 4557265 | NP_000675.1 | NM_000684.3 | 69 |
| | | | receptor | | | | |
| 35 | P07550 | ADRB2_HUMAN | Beta-2 adrenergic | 1797100823 | NP_000015.2 | NM_000024.6 | 70 |
| | | | receptor | | | | |

### REFERENCES

Berthold, M. R., N. Cebron, F. Dill, T. R. Gabriel, T. Kötter, T. Meinl, P. Ohl, C. Sieb, K. Thiel and B. Wiswedel (2007). KNIME: The Konstanz Information Miner. Studies in Classification, Data Analysis, and Knowledge Organization (GfKL 2007), Springer.
Hausser, M. (2014). “Optogenetics: the age of light.” Nat Methods 11(10): 1012-1014.
Kruse, A. C., A. M. Ring, A. Manglik, J. Hu, K. Hu, K. Eitel, H. Hubner, E. Pardon, C. Valant, P. M. Sexton, A. Christopoulos, C. C. Felder, P. Gmeiner, J. Steyaert, W. I. Weis, K. C. Garcia, J. Wess and B. K. Kobilka (2013). “Activation and allosteric modulation of a muscarinic acetylcholine receptor.” Nature 504 (7478): 101-106.
Masson, N., M. Ellis, S. Goodbourn and K. A. Lee (1992). “Cyclic AMP response element-binding protein and the catalytic subunit of protein kinase A are present in F9 embryonal carcinoma cells but are unable to activate the somatostatin promoter.” Mol Cell Biol 12 (3): 1096-1106.
Pettersen, E. F., T. D. Goddard, C. C. Huang, G. S. Couch, D. M. Greenblatt, E. C. Meng and T. E. Ferrin (2004). “UCSF Chimera-A visualization system for exploratory research and analysis.” Journal of Computational Chemistry 25 (13): 1605-1612.
Rosenbaum, D. M., C. Zhang, J. A. Lyons, R. Holl, D. Aragao, D. H. Arlow, S. G. F. Rasmussen, H.-J. Choi, B. T. DeVree, R. K. Sunahara, P. S. Chae, S. H. Gellman, R. O. Dror, D. E. Shaw, W. I. Weis, M. Caffrey, P. Gmeiner and B. K. Kobilka (2011). “Structure and function of an irreversible agonist-β2 adrenoceptor complex.” Nature 469 (7329): 236-240.
Shimamura, T., M. Shiroishi, S. Weyand, H. Tsujimoto, G. Winter, V. Katritch, R. Abagyan, V. Cherezov, W. Liu, G. W. Han, T. Kobayashi, R. C. Stevens and S. Iwata (2011). “Structure of the human histamine H1 receptor complex with doxepin.” Nature 475 (7354): 65-70.
Thal, D. M., B. Sun, D. Feng, V. Nawaratne, K. Leach, C. C. Felder, M. G. Bures, D. A. Evans, W. I. Weis, P. Bachhawat, T. S. Kobilka, P. M. Sexton, B. K. Kobilka and A. Christopoulos (2016). “Crystal structures of the M1 and M4 muscarinic acetylcholine receptors.” Nature 531 (7594): 335-340.
The, P. O. N. E. S. (2014). “Correction: Targeting Photoreceptors via Intravitreal Delivery Using Novel, Capsid-Mutated AAV Vectors.” PLOS One 9 (9): e110030.
Weston, M., T. Kaserer, A. Wu, A. Mouravlev, J. C. Carpenter, A. Snowball, S. Knauss, M. von Schimmelmann, M. J. During, G. Lignani, S. Schorge, D. Young, D. M. Kullmann and A. Lieb (2019). “Olanzapine: A potent agonist at the hM4D (Gi) DREADD amenable to clinical translation of chemogenetics.” Science Advances 5 (4): eaaw1567.
Young, D., D. M. Fong, P. A. Lawlor, A. Wu, A. Mouravlev, M. McRae, M. Glass, M. Dragunow and M. J. During (2014). “Adenosine kinase, glutamine synthetase and EAAT2 as gene therapy targets for temporal lobe epilepsy.” Gene Ther.

The invention claimed is:

1. A modified G-protein coupled receptor (GPCR) wherein the modified GPCR has:
(i) a decreased responsiveness to an endogenous activating ligand, and
(ii) a retained or enhanced responsiveness to an exogenous agonist;
compared to a parent which is the native GPCR,
wherein the modified GPCR comprises one, more preferably two, three or four, modifications compared to the parent GPCR at positions selected from:
(i) 85
(ii) 416
(iii) 120
(iv) 123
plus optionally one or more modifications compared to the parent GPCR at positions selected from:
(v) 128
(vi) 121
(vii) 200
(viii) 204
(viii) 410
(vix) 413
wherein the amino acid positions of the modified GPCR are numbered by correspondence with the amino acid sequence of SEQ ID NO:1.
***

2. A modified GPCR as claimed in claim 1, wherein the exogenous agonist is selected from the group consisting of:
(i) diphenhydramine or an analog thereof;
(ii) an exogenous agonist selected from Table 1 or Table 2; and
(iii) cyproheptadine, diphenylpyraline, desloratadine, and benzatropine.

3. (canceled)

4. (canceled)
***

5. A modified GPCR as claimed in claim 1 wherein the modified GPCR comprises the following residues at the following positions:
(a) 113C or 113N, and
(b) 203G.

6. A modified GPCR as claimed in claim 5 wherein the modified GPCR comprises one or more of the following substitutions at the following positions:
(a) Y113C or Y113N, and
(b) A203G.

7. A modified GPCR as claimed in claim 1 wherein the modified GPCR comprises one or more of the following residues at the following positions:
(i) 85V or 85C
(ii) 120I.
(iii) 416F
(iv) 123C, 123I, 123T or 123S.

8. A modified GPCR as claimed in claim 7 wherein the modified GPCR comprises one or more of the following substitutions at the following positions:
(i) S85V or S85C, most preferably S85V
(ii) V120I
(iii) Y416F
(iv) L123C, L123I, L123T, or L123S.

9. (canceled)

10. A modified GPCR as claimed in claim 1 wherein the modified GPCR comprises one or more of the following residues at the following positions:
(v) 128I, 128L or 128V
(vi) 121F
(vii) 200T
(viii) 204Y (viii) 410V
(ix) 413L.

11. A modified GPCR as claimed in claim 1 wherein the modified GPCR comprises one or more of the following substitutions at the following positions:
(v) F128I, F128L or F128V
(vi) M121F
(vii) A200T
(viii) F204Y
(viii) I410V
(ix) W413L.

12. (canceled)

13. A modified GPCR as claimed in claim 1 wherein the modified GPCR comprises Y113C+A203G+S85V+Y416F, optionally Y113C+A203G+S85V+L123T+V120I+Y416F.

14. (canceled)
***

15. A modified GPCR as claimed in claim 1 wherein the GPCR is a Gi-coupled GPCR.

16. A modified GPCR as claimed in claim 1 wherein (i) the GPCR is coupled via a G-protein to an ion channel, wherein the ion channel is optionally inwardly rectifying and/or wherein the ion channel is optionally a potassium channel, which is preferably a protein-coupled inwardly-rectifying potassium channel, and/or (ii) the GPCR is a Gq-coupled or Gs coupled GPCR.

17. A modified GPCR as claimed in claim 1 wherein the GPCR is selected from a cholinergic muscarinic receptor (CHRM); a histamine receptor (HRH); a 5-Hydroxytryptamine (serotonin) receptor (HTR); a dopamine receptor (DRD); an alpha adrenergic receptor (ADRA); a beta adrenergic receptor (β1-4 adrenoceptor) (ADRB).

18. A modified GPCR as claimed in claim 1 wherein the GPCR is selected from: CHRM4, CHRM3, CHRM1, CHRM2, CHRM5, HRH1, HRH2, HRH3, HRH4, 5HTR-1A, 5HTR-1B, 5HTR-1D, 5HTR-1E, 5HTR-1F, 5HTR-2A, 5HTR-2B, 5HTR-2C, 5HTR-4, 5HTR-5A, 5HTR-6, 5HTR-7, DRD-1, DRD-2, DRD-3, DRD-4, DRD-5, ADRA-1A, ADRA-1B, ADRA-1D, ADRA-2A, ADRA-2B, ADRA-2C, ADRB-1, ADRB-2, ADRB-3.

19. A modified GPCR as claimed in claim 1 wherein the GPCR is selected from a GPCR identified in Table 3.

20. A modified GPCR as claimed in claim 1 wherein the modified GPCR has at least 70% sequence identity with its native parent GPCR of any one of SEQ ID 1-35 of Table 8.

21. A modified GPCR as claimed in claim 1 wherein the modified GPCR comprises a sequence shown in any one of Tables 3-6 comprising said modifications.
***

22. A polynucleotide comprising a nucleic acid sequence encoding the modified GPCR of claim 1.

23. A polynucleotide as claimed in claim 22 wherein the nucleic acid has at least 70% sequence identity with its native parent GPCR of any one of SEQ ID 36-70 of Table 8.

24. An expression vector comprising the polynucleotide of claim 22, which is optionally a viral vector.
***

25. (canceled)

26. A vector as claimed in claim 24 wherein the vector is an adenovirus vector and/or an adeno-associated vector (AAV), which is optionally selected from the group consisting of: AAV1, AAV2, AAV3, AAV4, AAV5, AAV6, AAV7, AAV8, AAV9, AAV10, AAV11, AAV 12, and hybrids thereof.

27. A vector as claimed in claim 24 wherein the vector is a herpes virus vector, a retrovirus vector, or a lentivirus vector.

28. A vector as claimed in claim 24 wherein the nucleic acid encoding the modified GPCR is operably linked to a tissue or cell specific promoter.

29. A vector as claimed in claim 28 wherein the promoter is a neuronal cell type-specific promoter which is optionally the CaMk2A promoter.

30. (canceled)

31. (canceled)

32. (canceled)

33. (canceled)

34. (canceled)

35. A process for producing a modified G-protein coupled receptor (GPCR) having modified responsiveness to an exogenous agonist, which process comprises modifying a parent GPCR with two, three or four, modifications compared to the parent GPCR at positions selected from:
(i) 85
(ii) 416
(iii) 120
(iv) 123
plus optionally one or more modifications compared to the parent GPCR at positions selected from:
(v) 128
(vi) 121
(vii) 200
(viii) 204
(viii) 410
(ix) 413
wherein the amino acid positions of the modified GPCR are numbered by correspondence with the amino acid sequence of SEQ ID NO:1.

36. A process as claimed in claim 35 wherein the potency and/or efficacy of the exogenous agonist is increased in the modified GPCR compared to the parent GPCR.

37. (canceled)

38. (canceled)

39. (canceled)

40. (canceled)

41. (canceled)

42. A process as claimed in claim 36, wherein said modification(s) or further modification(s) are performed on a polynucleotide comprising a nucleic acid sequence encoding the parent GPCR.

43. (canceled)

44. A method of selectively modifying G-protein activation, or activating a G-protein, in a cell of a subject or organism, the method comprising the steps of:
(i) expressing in the cell a modified GPCR as defined in claim 1; and
(ii) administering to the subject or organism an agonist to the expressed modified GPCR.

45. A method as-claimed in claim 44 wherein the subject is selected from the list consisting of: a human subject, a non-human mammal, a bird, a fish, a reptile and an amphibian.

46. (canceled)

47. A method as-claimed in claim 44, wherein the cell is a non-excitable cell, which is optionally a hepatocyte.

48. A method as-claimed in claim 44, wherein the cell is an excitable cell selected from the list consisting of: a neuron from the CNS or PNS; a muscle cell; an endocrine cell, and wherein activation of the G-protein alters the excitability of the excitable cell.

49. (canceled)

50. (canceled)

51. (canceled)

52. (canceled)

53. A method of treating a disease or disorder in a subject, the method comprising the steps of:
(a) administering to the subject an effective amount of a polynucleotide as defined in claim 22 or an expression vector comprising the polynucleotide of claim 22 encoding a modified GPCR;
(b) expressing the modified GPCR of step (a) in a target cell or organ of the subject; and
(c) administering to the subject exogenous agonist to the expressed modified GPCR.

54. A method as claimed in claim 53 wherein said polynucleotide or vector is administered to the subject by direct injection and/or the exogenous agonist is selected from the group consisting of:
(i) diphenhydramine or an analog thereof;
(ii) an exogenous agonist selected from Table 1 or Table 2; and
(iii) cyproheptadine, diphenylpyraline, desloratadine, and benzatropine.

55. A method as claimed in claim 53 wherein the disease or disorder is a disease or disorder of the nervous system, and wherein the modified GPCR is expressed in the CNS or PNS.

56. A method as claimed in claim 55 wherein the disease or disorder of the nervous system is selected from the group consisting of:
(i) a neurological circuit disorder;
(ii) a neuropsychiatric disorder, a neurodegenerative disease, chronic pain, cerebrovascular accident, stroke; and
(iii) a seizure disorder.

57. (canceled)

58. (canceled)

59. (canceled)

60. A method as claimed in claim 56 wherein the disorder is a seizure disorder and the modified GPCR is expressed in neurons of a seizure focus in brain of the patient, and whereby activation of said modified GPCR reversibly alters the excitability of the neurons in the seizure focus.

61. A method as claimed in claim 60 wherein activation of said modified receptor (i) reversibly inhibits the excitability of and neurotransmission by excitatory neurons in the seizure focus, or (ii) reversibly excites inhibitory neurons in the seizure focus.

62. A method as claimed in claim 56 wherein:
(i) the seizure disorder is epilepsy, which is optionally focal epilepsy or generalized epilepsy, and/or
(ii) the subject has been diagnosed as having drug-resistant or medically-refractory epilepsy, and/or
(iii) wherein the subject is under an existing treatment with anti-epileptic drugs, wherein the method has the purpose of permitting the existing treatment to be discontinued or the drug regime to be reduced, and/or
(iv) the subject has been diagnosed as having epilepsia partialis continua, and/or
(v) the exogenous ligand is administered to the subject prior to the patient having an epileptic seizure, and/or
(vi) the exogenous ligand is administered to the subject during an epileptic seizure, and/or
(vii) the exogenous ligand is administered to the subject after having an epileptic seizure, and/or
(viii) the exogenous ligand is administered to the subject within 30 minutes before or 24 hours after the human has an epileptic seizure, and/or
(ix) the exogenous ligand is administered to the subject automatically either (i) by a device that is either coupled to an automated seizure detection mechanism, or (ii) in response to a predicted seizure by EEG analysis, and/or
(x) the exogenous ligand is administered in a combination therapy with one or more other agents for treating the seizure disorder.

63. (canceled)

64. A method as claimed in claim 53 where the agonist is administered by a route selected from the list consisting of: oral; parenteral by intravenous or intra-muscular route; sub-cutaneous injection; sub-lingual, buccal, intranasal, rectal; via an inhaler or nebuliser; topical to the eye or skin; otic, intrathecal, intravaginal, intravesicular.

65. (canceled)

66. (canceled)

67. (canceled)

68. (canceled)


SEQUENCE LISTING (212 sequences)

SEQ ID NO: 1 - protein, 479 aa, Homo sapiens
MANFTPVNGSSGNQSVRLVTSSSHNRYETVEMVFIATVTGSLSLVTVVGNILVMLSIKVNRQLQTVNNYFLFSLACADLIIGAFSMNLYTVYIIKGYWPLGAVVCDLWLALDYVVSNASVMNLLIISFDRYFCVTKPLTYPARRTTKMAGLMIAAAWVLSFVLWAPAILFWQFVVGKRTVPDNQCFIQFLSNPAVTFGTAIAAFYLPVVIMTVLYIHISLASRSRVHKHRPEGPKEKKAKTLAFLKSPLMKQSVKKPPPGEAAREELRNGKLEEAPPPALPPPPRPVADKDTSNESSSGSATQNTKERPATELSTTEATTPAMPAPPLQPRALNPASRWSKIQIVTKQTGNECVTAIEIVPATPAGMRPAANVARKFASIARNQVRKKRQMAARERKVTRTIFAILLAFILTWTPYNVMVLVNTFCQSCIPDTVWSIGYWLCYVNSTINPACYALCNATFKKTFRHLLLCQYRNIGTAR

SEQ ID NO: 2 - protein, 460 aa, Homo sapiens
MNTSAPPAVSPNITVLAPGKGPWQVAFIGITTGLLSLATVTGNLLVLISFKVNTELKTVNNYFLLSLACADLIIGTFSMNLYTTYLLMGHWALGTLACDLWLALDYVASNASVMNLLLISFDRYFSVTRPLSYRAKRTPRRAALMIGLAWLVSFVLWAPAILFWQYLVGERTVLAGQCYIQFLSQPIITFGTAMAAFYLPVTVMCTLYWRIYRETENRARELAALQGSETPGKGGGSSSSSERSQPGAEGSPETPPGRCCRCCRAPRLLQAYSWKEEEEEDEGSMESLTSSEGEEPGSEVVIKMPMVDPEAQAPTKQPPRSSPNTVKRPTKKGRDRAGKGQKPRGKEQLAKRKTFSLVKEKKAARTLSAILLAFILTWTPYNIMVLVSTFCKDCVPETLWELGYWLCYVNSTINPMCYALCNKAFRDTFRLLLLCRWDKRRWRKIPKRPGSVHRTPSRQC

SEQ ID NO: 3 - protein, 466 aa, Homo sapiens
MNNSTNSSNNSLALTSPYKTFEVVFIVLVAGSLSLVTIIGNILVMVSIKVNRHLQTVNNYFLFSLACADLIIGVFSMNLYTLYTVIGYWPLGPVVCDLWLALDYVVSNASVMNLLIISFDRYFCVTKPLTYPVKRTTKMAGMMIAAAWVLSFILWAPAILFWQFIVGVRTVEDGECYIQFFSNAAVTFGTAIAAFYLPVIIMTVLYWHISRASKSRIKKDKKEPVANQDPVSPSLVQGRIVKPNNNNMPSSDDGLEHNKIQNGKAPRDPVTENCVQGEEKESSNDSTSVSAVASNMRDDEITQDENTVSTSLGHSKDENSKQTCIRIGTKTPKSDSCTPTNTTVEVVGSSGQNGDEKQNIVARKIVKMTKQPAKKKPPPSREKKVTRTILAILLAFIITWAPYNVMVLINTFCAPCIPNTVWTIGYWLCYINSTINPACYALCNATFKKTFKHLLMCHYKNIGATR

SEQ ID NO: 4 - protein, 590 aa, Homo sapiens
MTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 5 - protein, 532 aa, Homo sapiens
MEGDSYHNATTVNGTPVNHQPLERHRLWEVITIAAVTAVVSLITIVGNVLVMISFKVNSQLKTVNNYYLLSLACADLIIGIFSMNLYTTYILMGRWALGSLACDLWLALDYVASNASVMNLLVISFDRYFSITRPLTYRAKRTPKRAGIMIGLAWLISFILWAPAILCWQYLVGKRTVPLDECQIQFLSEPTITFGTAIAAFYIPVSVMTILYCRIYRETEKRTKDLADLQGSDSVTKAEKRKPAHRALFRSCLRCPRPTLAQRERNQASWSSSRRSTSTTGKPSQATGPSANWAKAEQLTTCSSYPSSEDEDKPATDPVLQVVYKSQGKESPGEEFSAEETEETFVKAETEKSDYDTPNYLLSPAAAHRPKSQKCVAYKFRLVVKADGNQETNNGCHKVKIMPCPFPVAKEPSTKGLNPNPSHQMTKRKRVVLVKERKAAQTLSAILLAFIITWTPYNIMVLVSTFCDKCVPVTLWHLGYWLCYVNSTVNPICYALCNRTFRKTFKMLLLCRWKKKKVEEKLYWQGNSKLP

SEQ ID NO: 6 - protein, 487 aa, Homo sapiens
MSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPLRYLKYRTKTRASATILGAWFLSFLWVIPILGWNHFMQQTSVRREDKCETDFYDVTWFKVMTAIINFYLPTLLMLWFYAKIYKAVRQHCQHRELINRSLPSFSEIKLRPENPKGDAKKPGKESPWEVLKRKPKDAGGGSVLKSPSQTPKEMKSPVVFSQEDDREVDKLYCFPLDIVHMQAAAEGSSRDYVAVNRSHGQLKTDEQGLNTHGASEISEDQMLGDSQSFSRTDSDTTTETAPGKGKLRSGSNTGLDYIKFTWKRLRSHSRQYVSGLHMNRERKAAKQLGFIMAAFILCWIPYFIFFMVIAFCKNCCNEHLHMFTIWLGYINSTLNPLIYPLCNENFKKTFKRILHIRS

SEQ ID NO: 7 - protein, 397 aa, Homo sapiens
MAPNGTASSFCLDSTACKITITVVLAVLILITVAGNVVVCLAVGLNRRLRNLTNCFIVSLAITDLLLGLLVLPFSAIYQLSCKWSFGKVFCNIYTSLDVMLCTASILNLFMISLDRYCAVMDPLRYPVLVTPVRVAISLVLIWVISITLSFLSIHLGWNSRNETSKGNHTTSKCKVQVNEVYGLVDGLVTFYLPLLIMCITYYRIFKVARDQAKRINHISSWKAATIREHKATVTLAAVMGAFIICWFPYFTAFVYRGLRGDDAINEVLEAIVLWLGYANSALNPILYAALNRDFRTGYQQLFCCRLANRNSHKTSLRSNASQLSRTQSREPRQQEEKPLKLQVWSGTEVTAPQGATDRPWLCLPECWSVELTHSFIHLFIHSFANIHPIPTTCQEL

SEQ ID NO: 8 - protein, 445 aa, Homo sapiens
MERAPPDGPLNASGALAGEAAAAGGARGFSAAWTAVLAALMALLIVATVLGNALVMLAFVADSSLRTQNNFFLLNLAISDFLVGAFCIPLYVPYVLTGRWTFGRGLCKLWLVVDYLLCTSSAFNIVLISYDRFLSVTRAVSYRAQQGDTRRAVRKMLLVWVLAFLLYGPAILSWEYLSGGSSIPEGHCYAEFFYNWYFLITASTLEFFTPFLSVTFFNLSIYLNIQRRTRLRLDGAREAAGPEPPPEAQPSPPPPPGCWGCWQKGHGEAMPLHRYGVGEAAVGAEAGEATLGGGGGGGSVASPTSSSGSSSRGTERPRSLKRGSKPSASSASLEKRMKMVSQSFTQRFRLSRDRKVAKSLAVIVSIFGLCWAPYTLLMIIRAACHGHCVPDYWYETSFWLLWANSAVNPVLYPLCHHSFRRAFTKLLCPQKLKIQPHSSLEHCWK

SEQ ID NO: 9 - protein, 390 aa, Homo sapiens
MPDTNSTINLSLSTRVTLAFFMSLVAFAIMLGNALVILAFVVDKNLRHRSSYFFLNLAISDFFVGVISIPLYIPHTLFEWDFGKEICVFWLTTDYLLCTASVYNIVLISYDRYLSVSNAVSYRTQHTGVLKIVTLMVAVWVLAFLVNGPMILVSESWKDEGSECEPGFFSEWYILAITSFLEFVIPVILVAYFNMNIYWSLWKRDHLSRCQSHPGLTAVSSNICGHSFRGRLSSRRSLSASTEVPASFHSERQRRKSSLMFSSRTKMNSNTIASKMGSFSQSDSVALHQREHVELLRARRLAKSLAILLGVFAVCWAPYSLFTIVLSFYSSATGPKSVWYRIAFWLQWFNSFVNPLLYPLCHKRFQKAFLKIFCIKKQPLPSQHSRSVSS

SEQ ID NO: 10 - protein, 366 aa, Homo sapiens
MDFLNSSDQNLTSEELLNRMPSKILVSLTLSGLALMTTTINSLVIAAIIVTRKLHHPANYLICSLAVTDFLVAVLVMPFSIVYIVRESWIMGQVVCDIWLSVDITCCTCSILHLSAIALDRYRAITDAVEYARKRTPKHAGIMITIVWIISVFISMPPLFWRHQGTSRDDECIIKHDHIVSTIYSTFGAFYIPLALILILYYKIYRAAKTLYHKRQASRIAKEEVNGQVLLESGEKSTKSVSTSYVLEKSLSDPSTDFDKIHSTVRSLRSEFKHEKSWRRQKISGTRERKAATTLGLILGAFVICWLPFFVKELVVNVCDKCKISEEMSNFLAWLGYLNSLINPLIYTIFNEDFKKAFQKLVRCRC

SEQ ID NO: 11 - protein, 365 aa, Homo sapiens
MNITNCTTEASMAIRPKTITEKMLICMTLVVITTLTTLLNLAVIMAIGTTKKLHQPANYLICSLAVTDLLVAVLVMPLSIIYIVMDRWKLGYFLCEVWLSVDMTCCTCSILHLCVIALDRYWAITNAIEYARKRTAKRAALMILTVWTISIFISMPPLFWRSHRRLSPPPSQCTIQHDHVIYTIYSTLGAFYIPLTLILILYYRIYHAAKSLYQKRGSSRHLSNRSTDSQNSFASCKLTQTFCVSDFSTSDPTTEFEKFHASIRIPPFDNDLDHPGERQQISSTRERKAARILGLILGAFILSWLPFFIKELIVGLSIYTVSSEVADFLTWLGYVNSLINPLLYTSFNEDFKLAFKKLIRCREHT

SEQ ID NO: 12 - protein, 377 aa, Homo sapiens
MSPLNQSAEGLPQEASNRSLNATETSEAWDPRTLQALKISLAVVLSVITLATVLSNAFVLTTILLTRKLHTPANYLIGSLATTDLLVSILVMPISIAYTITHTWNFGQILCDIWLSSDITCCTASILHLCVIALDRYWAITDALEYSKRRTAGHAATMIAIVWAISICISIPPLFWRQAKAQEEMSDCLVNTSQISYTIYSTCGAFYIPSVLLIILYGRIYRAARNRILNPPSLYGKRFTTAHLITGSAGSSLCSLNSSLHEGHSHSAGSPLFFNHVKIKLADSALERKRISAARERKATKILGIILGAFIICWLPFFVVSLVLPICRDSCWIHPALFDFFTWLGYLNSLINPIIYTVFNEEFRQAFQKIVPFRKAS

SEQ ID NO: 13 - protein, 390 aa, Homo sapiens
MEEPGAQCAPPPPAGSETWVPQANLSSAPSQNCSAKDYIYQDSISLPWKVLLVMLLALITLATTLSNAFVIATVYRTRKLHTPANYLIASLAVTDLLVSILVMPISTMYTVTGRWTLGQVVCDFWLSSDITCCTASILHLCVIALDRYWAITDAVEYSAKRTPKRAAVMIALVWVFSISISLPPFFWRQAKAEEEVSECVVNTDHILYTVYSTVGAFYFPTLLLIALYGRIYVEARSRILKQTPNRTGKRLTRAQLITDSPGSTSSVTSINSRVPDVPSESGSPVYVNQVKVRVSDALLEKKKLMAARERKATKTLGIILGAFIVCWLPFFIISLVMPICKDACWFHLAIFDFFTWLGYLNSLINPIIYTMSNEDFKQAFHKLIRFKCTS

SEQ ID NO: 14 - protein, 357 aa, Homo sapiens
MDLPVNLTSFSLSTPSPLETNHSLGKDDLRPSSPLLSVFGVLILTLLGFLVAATFAWNLLVLATILRVRTFHRVPHNLVASMAVSDVLVAALVMPLSLVHELSGRRWQLGRRLCQLWIACDVLCCTASIWNVTAIALDRYWSITRHMEYTLRTRKCVSNVMIALTWALSAVISLAPLLFGWGETYSEGSEECQVSREPSYAVFSTVGAFYLPLCVVLFVYWKIYKAAKFRVGSRKTNSVSPISEAVEVKDSAKQPQMVFTVRHATVTFQPEGDTWREQKEQRAALMVGILIGVFVLCWIPFFLTELISPLCSCDIPAIWKSIFLWLGYSNSFFNPLIYTAFNKNYNSAFKNFFSRQH

SEQ ID NO: 15 - protein, 479 aa, Homo sapiens
MMDVNSSGRPDLYGHLRSFLLPEVGRGLPDLSPDGGADPVAGSWAPHLLSEVTASPAPTWDAPPDNASGCGEQINYGRVEKVVIGSILTLITLLTIAGNCLVVISVCFVKKLRQPSNYLIVSLALADLSVAVAVMPFVSVTDLIGGKWIFGHFFCNVFIAMDVMCCTASIMTLCVISIDRYLGITRPLTYPVRQNGKCMAKMILSVWLLSASITLPPLFGWAQNVNDDKVCLISQDFGYTIYSTAVAFYIPMSVMLFMYYQIYKAARKSAAKHKFPGFPRVEPDSVIALNGIVKLQKEVEECANLSRLLKHERKNISIFKREQKAATTLGIIVGAFTVCWLPFFLLSTARPFICGTSCSCIPLWVERTFLWLGYANSLINPFIYAFFNRDLRTTYRSLLQCQYRNINRKLSAAGMHEALKLAERPERPEFVLRACTRRVLLRPEKRPPVSVWVLQSPDHHNWLADKMLTTVEKKVMIHD

SEQ ID NO: 16 - protein, 422 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 17 - protein, 471 aa, Homo sapiens
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 18 - protein, 400 aa, Homo sapiens
MASLSQLSGHLNYTCGAENSTGASQARPHAYYALSYCALILAIVFGNGLVCMAVLKERALQTTTNYLVVSLAVADLLVATLVMPWVVYLEVTGGVWNFSRICCDVFVTLDVMMCTASILNLCAISIDRYTAVVMPVHYQHGTGQSSCRRVALMITAVWVLAFAVSCPLLFGFNTTGDPTVCSISNPDFVIYSSVVSFYLPFGVTVLVYARIYVVLKQRRRKRILTRQNSQCNSVRPGFPQQTLSPDPAHLELKRYYSICQDTALGGPGFQERGGELKREEKTRNSLSPTIAPKLSLEVRKLSNGRLSTSLKLGPLQPRGVPLREKKATQMVAIVLGAFIVCWLPFFLTHVLNTHCQTCHVSPELYSATTWLGYVNSALNPVIYTTFNIEFRKAFLKILSC

SEQ ID NO: 19 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 20 - protein, 419 aa, Homo sapiens
MGNRSTADADGLLAGRGPAAGASAGASAGLAGQGAAALVGGVLLIGAVLAGNSLVCVSVATERALQTPTNSFIVSLAAADLLLALLVLPLFVYSEVQGGAWLLSPRLCDALMAMDVMLCTASIFNLCAISVDRFVAVAVPLRYNRQGGSRRQLLLIGATWLLSAAVAAPVLCGLNDVRGRDPAVCRLEDRDYVVYSSVCSFFLPCPLMLLLYWATFRGLQRWEVARRAKLHGRAPRRPSGPGPPSPTPPAPRLPQDPCGPDCAPPAPGLPRGPCGPDCAPAAPSLPQDPCGPDCAPPAPGLPPDPCGSNCAPPDAVRAAALPPQTPPQTRRRRRAKITGRERKAMRVLPVVVGAFLLCWTPFFVVHITQALCPACSVPPRLVSAVTWLGYVNSALNPVIYTVFNAEFRNVFRKALRACC

SEQ ID NO: 21 - protein, 520 aa, Homo sapiens
MNPDLDTGHNTSAPAHWGELKNANFTGPNQTSSNSTLPQLDITRAISVGLVLGAFILFAIVGNILVILSVACNRHLRTPTNYFIVNLAMADLLLSFTVLPFSAALEVLGYWVLGRIFCDIWAAVDVLCCTASILSLCAISIDRYIGVRYSLQYPTLVTRRKAILALLSVWVLSTVISIGPLLGWKEPAPNDDKECGVTEEPFYALFSSLGSFYIPLAVILVMYCRVYIVAKRTTKNLEAGVMKEMSNSKELTLRIHSKNFHEDTLSSTKAKGHNPRSSIAVKLFKFSREKKAAKTLGIVVGMFILCWLPFFIALPLGSLFSTLKPPDAVFKVVFWLGYFNSCLNPIIYPCSSKEFKRAFVRILGCQCRGRGRRRRRRRRRLGGCAYTYRPWTRGGSLERSQSRKDSLDDSGSCLSGSQRTLPSASPSPGYLGRGAPPPVELCAFPEWKAPGALLSLPAPEPPGRRGRHDSGPLFTFKLLTEPESPGTDGGASNGGCEAAADVANGQPGFKSNMPLAPGQF

SEQ ID NO: 22 - protein, 572 aa, Homo sapiens
MTFRDLLSVSFEGPRPDSSAGGSSAGGGGGSAGGAAPSEGPAVGGVPGGAGGGGGVVGAGSGEDNRSSAGEPGSAGAGGDVNGTAAVGGLVVSAQGVGVGVFLAAFILMAVAGNLLVILSVACNRHLQTVTNYFIVNLAVADLLLSATVLPFSATMEVLGFWAFGRAFCDVWAAVDVLCCTASILSLCTISVDRYVGVRHSLKYPAIMTERKAAAILALLWVVALVVSVGPLLGWKEPVPPDERFCGITEEAGYAVFSSVCSFYLPMAVIVVMYCRVYVVARSTTRSLEAGVKRERGKASEVVLRIHCRGAATGADGAHGMRSAKGHTFRSSLSVRLLKFSREKKAAKTLAIVVGVFVLCWFPFFFVLPLGSLFPQLKPSEGVFKVIFWLGYFNSCVNPLIYPCSSREFKRAFLRLLRCQCRRRRRRRPLWRVYGHHWRASTSGLRQDCAPSSGDAPPGAPLALTALPDPDPEPPGTPEMQAPVASRRKPPSAFREWRLLGPFRRPTTQLRAKVSSLSHKIRAGGAQRAEAACAQRSEVEAVSLGVPHEVAEGATCQAYELADYSNLRETDI

SEQ ID NO: 23 - protein, 466 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLCRKQSSKHALGYTLHPPSQAVEGQHKDMVRIPVGSRETFYRISKTDGVCEWKFFSSMPRGSARITVSKDQSSCTTARVRSKSFLQVCCCVGPSTPSLDKNHQVPTIKVHTISLSENGEEV

SEQ ID NO: 24 - protein, 450 aa, Homo sapiens
MDHQDPYSVQATAAIAAAITFLILFTIFGNALVILAVLTSRSLRAPQNLFLVSLAAADILVATLIIPFSLANELLGYWYFRRTWCEVYLALDVLFCTSSIVHLCAISLDRYWAVSRALEYNSKRTPRRIKCIILTVWLIAAVISLPPLIYKGDQGPQPRGRPQCKLNQEAWYILASSIGSFFAPCLIMILVYLRIYLIAKRSNRRGPRAKGGPGQGESKQPRPDHGGALASAKLPALASVASAREVNGHSKSTGEKEEGETPEDTGTRALPPSWAALPNSGQGQKEGVCGASPEDEAEEEEEEEEEEEECEPQAVPVSPASACSPPLQQPQGSRVLATLRGQVLLGRGVGAIGGQWWRRRAQLTREKRFTFVLAVVIGVFVLCWFPFFFSYSLGAICPKHCKVPHGLFQFFFWIGYCNSSLNPVIYTIFNQDFRRAFRRILCRPWTQTAW

SEQ ID NO: 25 - protein, 465 aa, Homo sapiens
MFRQEQPLAEGSFAPMGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGTERRPNGLGPERSAGPGGAEAEPLPTQLNGAPGEPAPAGPRDTDALDLEESSSSDHAERPPGPRRPERGPRGKGKARASQVKPGDSLPRRGPGATGIGTPAAGPGEERVGAAKASRWRGRQNREKRFTFVLAVVIGVFVVCWFPFFFTYTLTAVGCSVPRTLFKFFFWFGYCNSSLNPVIYTIFNHDFRRAFKKILCRGDRKRIV

SEQ ID NO: 26 - protein, 462 aa, Homo sapiens
MASPALAAALAVAAAAGPNASGAGERGSGGVANASGASWGPPRGQYSAGAVAGLAAVVGFLIVFTVVGNVLVVIAVLTSRALRAPQNLFLVSLASADILVATLVMPFSLANELMAYWYFGQVWCGVYLALDVLFCTSSIVHLCAISLDRYWSVTQAVEYNLKRTPRRVKATIVAVWLISAVISFPPLVSLYRQPDGAAYPQCGLNDETWYILSSCIGSFFAPCLIMGLVYARIYRVAKLRTRTLSEKRAPVGPDGASPTTENGLGAAAGAGENGHCAPPPADVEPDESSAAAERRRRRGALRRGGRRRAGAEGGAGGADGQGAGPGAAESGALTASRSPGPGGRLSRASSRSVEFFLSRRRRARSSVCRRKVAQAREKRFTFVLAVVMGVFVLCWFPFFFSYSLYGICREACQVPGPLFKFFFWIGYCNSSLNPVIYTVFNQDFRRSFKHILFRRRRRGFRQ

SEQ ID NO: 27 - protein, 440 aa, Homo sapiens
MVPEPGPTANSTPAWGAGPPSAPGGSGWVAAALCVVIALTAAANSLLIALICTQPALRNTSNFFLVSLFTSDLMVGLVVMPPAMLNALYGRWVLARGLCLLWTAFDVMCCSASILNLCLISLDRYLLILSPLRYKLRMTPLRALALVLGAWSLAALASFLPLLLGWHELGHARPPVPGQCRLLASLPFVLVASGLTFFLPSGAICFTYCRILLAARKQAVQVASLTTGMASQASETLQVPRTPRPGVESADSRRLATKHSRKALKASLTLGILLGMFFVTWLPFFVANIVQAVCDCISPGLFDVLTWLGYCNSTMNPIIYPLFMRDFKRALGRFLPCPRCPRERQASLASPSLRTSHSGPRPGLSLQQVLPLPLPPDSDSDSDAGSGGSSGLRLTAQLLLPGEATQDPPLPTRAAAAVNFFNIDPAEPELRPHPLGIPTN

SEQ ID NO: 28 - protein, 481 aa, Homo sapiens
MALSYRVSELQSTIPEHILQSTFVHVISSNWSGLQTESIPEEMKQIVEEQGNKLHWAALLILMVIIPTIGGNTLVILAVSLEKKLQYATNYFLMSLAVADLLVGLFVMPIALLTIMFEAMWPLPLVLCPAWLFLDVLFSTASIMHLCAISVDRYIAIKKPIQANQYNSRATAFIKITVVWLISIGIAIPVPIKGIETDVDNPNNITCVLTKERFGDFMLFGSLAAFFTPLAIMIVTYFLTIHALQKKAYLVKNKPPQRLTWLTVSTVFQRDETPCSSPEKVAMLDGSRKDKALPNSGDETLMRRTSTIGKKSVQTISNEQRASKVLGIVFFLFLLMWCPFFITNITLVLCDSCNQTTLQMLLEIFVWIGYVSSGVNPLVYTLFNKTFRDAFGRYITCNYRATKSVKTLRKRSSKIYFRNPMAENSKFFKKHGIRNGINPAMYQSPMRLRSSTIQSSSIILLDTLLLTENEGDKTEEQVSYV

SEQ ID NO: 29 - protein, 458 aa, Homo sapiens
MVNLRNAVHSFLVHLIGLLVWQSDISVSPVAAIVTDIFNTSDGGRFKFPDGVQNWPALSIVIIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDEEKVFVNNTTCVLNDPNFVLIGSFVAFFIPLTIMVITYCLTIYVLRRQALMLLHGHTEEPPGLSLDFLKCCKRNTAEEENSANPNQDQNARRRKKKERRPRGTMQAINNERKASKVLGIVFFVFLIMWCPFFITNILSVLCEKSCNQKLMEKLLNVFVWIGYVCSGINPLVYTLFNKIYRRAFSNYLRCNYKVEKKPPVRQIPRVAATALSGRELNVNIYRHTNEPVIEKASDNEPGIEMQVENLELPVNPSSVVSERISSV

SEQ ID NO: 30 - protein, 477 aa, Homo sapiens
MLPPGSNGTAYPGQFALYQQLAQGNAVGGSAGAPPLGPSQVVTACLLTLLIIWTLLGNVLVCAAIVRSRHLRANMTNVFIVSLAVSDLFVALLVMPWKAVAEVAGYWPFGAFCDVWVAFDIMCSTASILNLCVISVDRYWAISRPFRYKRKMTQRMALVMVGLAWTLSILISFIPVQLNWHRDQAASWGGLDLPNNLANWTPWEEDFWEPDVNAENCDSSLNRTYAISSSLISFYIPVAIMIVTYTRIYRIAQVQIRRISSLERAAEHAQSCRSSAACAPDTSLRASIKKETKVLKTLSVIMGVFVCCWLPFFILNCMVPFCSGHPEGPPAGFPCVSETTFDVFVWFGWANSSLNPVIYAFNADFQKVFAQLLGCSHFCSRTPVETVNISNELISYNQDIVFHKEIAAAYIHMMPNAVTPGNREVDNDEEEGPFDRMFQIYQTSPDGDPVAESVWELDCEGEISLDKITPFTPNGFH

SEQ ID NO: 31 - protein, 446 aa, Homo sapiens
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 32 - protein, 388 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 33 - protein, 408 aa, Homo sapiens
MAPWPHENSSLAPWPDLPTLAPNTANTSGLPGVPWEAALAGALLALAVLATVGGNLLVIVAIAWTPRLQTMTNVFVTSLAAADLVMGLLVVPPAATLALTGHWPLGATGCELWTSVDVLCVTASIETLCALAVDRYLAVTNPLRYGALVTKRCARTAVVLVWVVSAAVSFAPIMSQWWRVGADAEAQRCHSNPRCCAFASNMPYVLLSSSVSFYLPLLVMLFVYARVFVVATRQLRLLRGELGRFPPEESPPAPSRSLAPAPVGTCAPPEGVPACGRRPARLLPLREHRALCTLGLIMGTFTLCWLPFFLANVLRALGGPSLVPGPAFLALNWLGYANSAFNPLIYCRSPDFRSAFRRLLCRCGRRLPPEPCAAARPALFPSGVPAARSSPAQPRLCQRLDGASWGVS

SEQ ID NO: 34 - protein, 477 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRPSRLVALREQKALKTLGIIMGVFTLCWLPFFLANVVKAFHRELVPDRLFVFFNWLGYANSAFNPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKV

SEQ ID NO: 35 - protein, 413 aa, Homo sapiens [sequence omitted]

SEQ ID NO: 36 - RNA, 3050 nt, Homo sapiens
  mRNA (1)..(3050): mRNA
gccacagcgccgggagcugcgggcggagcaggggccgccccucgacaccccguccccgcccccggcccucggcccggccgcccgccccgccggcccgcggggccgcgcguccgucccgccgucugucuggcgcgaggggagcugggcagaggcggcggguaaguggcuccgcggcggcgcgggggcggggggcucgccgggaagagcaggaagcagguccccgggacgcucugaggcuccccagagacacggccuacccaccccuggcaaccaacauggccaacuucacaccugucaauggcagcucgggcaaucaguccgugcgccuggucacgucaucaucccacaaucgcuaugagacgguggaaauggucuucauugccacagugacaggcucccugagccuggugacugucgugggcaacauccuggugaugcuguccaucaaggucaacaggcagcugcagacagucaacaacuacuuccucuucagccuggcgugugcugaucucaucauaggcgccuucuccaugaaccucuacaccguguacaucaucaagggcuacuggccccugggcgccguggucugcgaccuguggcuggcccuggacuacguggugagcaacgccuccgucaugaaccuucucaucaucagcuuugaccgcuacuucugcgucaccaagccucucaccuacccugcccggcgcaccaccaagauggcaggccucaugauugcugcugccuggguacuguccuucgugcucugggcgccugccaucuuguucuggcaguuugugguggguaagcggacggugcccgacaaccagugcuucauccaguuccuguccaacccagcagugaccuuuggcacagccauugcugccuucuaccugccuguggucaucaugacggugcuguacauccacaucucccuggccagucgcagccgaguccacaagcaccggcccgagggcccgaaggagaagaaagccaagacgcuggccuuccucaagagcccacuaaugaagcagagcgucaagaagcccccgcccggggaggccgcccgggaggagcugcgcaauggcaagcuggaggaggcccccccgccagcgcugccaccgccaccgcgccccguggcugauaaggacacuuccaaugaguccagcucaggcagugccacccagaacaccaaggaacgcccagccacagagcuguccaccacagaggccaccacgcccgccaugcccgccccuccccugcagccgcgggcccucaacccagccuccagaugguccaagauccagauugugacgaagcagacaggcaaugagugugugacagccauugagauugugccugccacgccggcuggcaugcgcccugcggccaacguggcccgcaaguucgccagcaucgcucgcaaccaggugcgcaagaagcggcagauggcggcccgggagcgcaaagugacacgaacgaucuuugccauucugcuagccuucauccucaccuggacgcccuacaacgucaugguccuggugaacaccuucugccagagcugcaucccugacacggugugguccauuggcuacuggcucugcuacgucaacagcaccaucaacccugccugcuaugcucugugcaacgccaccuuuaaaaagaccuuccggcaccugcugcugugccaguaucggaacaucggcacugccagguaggcaggcaggagugcccuaggaggugcugguguugcgugcgugugcuggggggaccacacggcucacuugcuguggggaagaguugcaggcaccauucugcguucacguuugcugaggaggaaguucagaagaggcucuguggcugcauucagagaccagaucucugcucaggcugaggaggcucaccccagggagugucugaacuggggcugccuggcccaccucuguggcccugcuucagcgagcugcggggcacuggccugggugggcaccugccccacugugaccaaccaucagcagugcuggaagaauggacaucuggaugggggccgaagcccagggcccccucaggaagaacaaaggggccagugcuuggaaaaggaggcuugggaagggaguagaaggguggggucccccauucugcuguaaucggugcuuucugcccccuugcaccccgcauguaggcucagcccccucuccccaaaucccacuccaggggcagaaaucuccucuggcugcagcuacuuagccagguucuggguggguaugauggaggccaggucccccccgccccagcagcacugcccuugucacauuaagauggggcagcugggaggucagagguucugucucaggaacugaggucuggccuucugcgaucccaggcugagccgugagugguccacguauucagaagacagcccccugcagccccggccuuagaaggcugucccaggucacacaucgagucagggugaggcugugguggauggcauucccaucugcccucuucaaaccucucacccccuggcuucugcaggguggaggccugggucacacaggcaagaggaaaggggucaaccugguggcucacucggguuuccaugacagugggaaggggggaggugggcagugagguaggcacgcagcccucgcaucugcucucccccauggguagaaccuuccugauuggcuguggguuguaucaaaagaccuuugacugggugaggcugagauggguguggugguuccagugagucagccagguucccuuccccagcccuggucccugagggagaugaggaccgaaaggcuuaggugacuugauggggagcacccuuucaggggcccuggaagaaauguuuguaauuguucugcuuuuuccaugagcucuggagagcacccaccaccaagcccccugaaacccaccagucccuacagccuggucuuucuccccaccuucccacucccaaacacauuuccacagcaaacuccuugggaaggggcuuuuauauauuaaaaaagaagagcuuuuauuggggggaaaaagaaaaa

SEQ ID NO: 37 - RNA, 2646 nt, Homo sapiens
  mRNA (1)..(2646): mRNA
augccaaucacuggcugugccucucccgcugccagcacagggcgcagcucccccugggagccagguguuugggucccuggagacgccgcaggcccccagggaggcaguggggcugaggacccuacagaccccucuucagccccguggugaugacuuuccccugaggaagcccuguagcgugccuggaggaaggggcucuccaaccccagccccaccuagccaccaugaacacuucagccccaccugcugucagccccaacaucaccguccuggcaccaggaaagggucccuggcaaguggccuucauugggaucaccacgggccuccugucgcuagccacagugacaggcaaccugcugguacucaucucuuucaaggucaacacggagcucaagacagucaauaacuacuuccugcugagccuggccugugcugaccucaucaucgguaccuucuccaugaaccucuauaccacguaccugcucaugggccacugggcucugggcacgcuggcuugugaccucuggcuggcccuggacuauguggccagcaaugccuccgucaugaaucugcugcucaucagcuuugaccgcuacuucuccgugacucggccccugagcuaccgugccaagcgcacaccccgccgggcagcucugaugaucggccuggccuggcugguuuccuuugugcucugggccccagccauccucuucuggcaguaccugguaggggagcggacagugcuagcugggcagugcuacauccaguuccucucccagcccaucaucaccuuuggcacagccauggcugccuucuaccucccugucacagucaugugcacgcucuacuggcgcaucuaccgggagacagagaaccgagcacgggagcuggcagcccuucagggcuccgagacgccaggcaaaggggguggcagcagcagcagcucagagaggucucagccaggggcugagggcucaccagagacuccuccaggccgcugcugucgcugcugccgggcccccaggcugcugcaggccuacagcuggaaggaagaagaggaagaggacgaaggcuccauggagucccucacauccucagagggagaggagccuggcuccgaaguggugaucaagaugccaaugguggaccccgaggcacaggcccccaccaagcagcccccacggagcuccccaaauacagucaagaggccgacuaagaaagggcgugaucgagcuggcaagggccagaagccccguggaaaggagcagcuggccaagcggaagaccuucucgcuggucaaggagaagaaggcggcucggacccugagugccauccuccuggccuucauccucaccuggacaccguacaacaucauggugcugguguccaccuucugcaaggacuguguucccgagacccugugggagcugggcuacuggcugugcuacgucaacagcaccaucaaccccaugugcuacgcacucugcaacaaagccuuccgggacaccuuucgccugcugcugcuuugccgcugggacaagagacgcuggcgcaagauccccaagcgcccuggcuccgugcaccgcacucccucccgccaaugcugauaguccccucuccugcaucccuccaccccaguccccgggaaaggccggugggaagagggcaggggcugcauccucagccccagggcccugcucaggccucaccuggcuucccaggacccugggucaccuuccugggcagcccagagagacccugccaacuuuccagacuucgcuauucccaggcagggagggaaacccggggaacugguuuuucuguucccugcugggugggaaugcgcucuucaccaggaagaaggcccgggaggaggauccgggcuuuggacuccuuguuugccuuuaggcaggaagucaggagccagcagggcgggccaggagaaagaaggcuuaacauuaaguauuccuuggcccagcagcggcccagauugcggugugagauggugcccccuggggggcacagccagaaacugaacuggccgcugggagaaaagccagaugacagggagcuggggaauccccucgcuucauaggcagagcccgcccaccugggcccuaggcauacucuccaggauuguccacaaauguccucagagggucccuaggugggucaacuccaaggcaaauguccaagcaucagcaagacaaugacacuggaaggguccggcuuggcuagucacauaucaagucccgaggcagcaacaggaccaggagccagguguccugacuguccuacaauaucauuuuccugggagugggagucaagugugccugcuauccagccgcaaauccauacccccugccccagagaagccucagucccucccuccuggcucacagccaccaccuggauggaucugcuccaugcagaucuagccaggccucccgcaugcugccugccuccggcccugccccacacaggccuggcccagccagcagguucucuccugugagcuccccaauccaacccaugcauggccucccagccacccggaucuccaggcccagccuggccccaaauguucuuuccuuucauccucagcaagugcugagucugugaauaaagccacauaaccagcgggca

SEQ ID NO: 38 - RNA, 5719 nt, Homo sapiens
  mRNA (1)..(5719): mRNA
acagacuagccaaguggcugagacgaguguggggugcgugacucugccugcgcgcgcgccagccccgcagcucucgccagagccuugggaucaagggagaaagagaaccggcagcuggccucggacucuaagcggugcgcagcuccagcccgagcggaucggcccugaacccacaaaggacuccucgcuccuucaagccuccaccaccucgcagccggggaggcaacuggagcgaaaccagcgacagguuguuagcgacaugaguacagggucuggaucuuacccauucaagaguguauucucagcacccaaugccuggcauauaguuuaaauguuuauuugcuacuuggcuacugauuagagaacgcaaaaugaauaacucaacaaacuccucuaacaauagccuggcucuuacaaguccuuauaagacauuugaagugguguuuauuguccugguggcuggaucccucaguuuggugaccauuaucgggaacauccuagucaugguuuccauuaaagucaaccgccaccuccagaccgucaacaauuacuuuuuauucagcuuggccugugcugaccuuaucauagguguuuucuccaugaacuuguacacccucuacacugugauugguuacuggccuuugggaccuguggugugugaccuuuggcuagcccuggacuauguggucagcaaugccucaguuaugaaucugcucaucaucagcuuugacagguacuucugugucacaaaaccucugaccuacccagucaagcggaccacaaaaauggcagguaugaugauugcagcugccuggguccucucuuucauccucugggcuccagccauucucuucuggcaguucauuguaggggugagaacuguggaggauggggagugcuacauucaguuuuuuuccaaugcugcugucaccuuugguacggcuauugcagccuucuauuugccagugaucaucaugacugugcuauauuggcacauaucccgagccagcaagagcaggauaaagaaggacaagaaggagccuguugccaaccaagaccccguuucuccaagucugguacaaggaaggauagugaagccaaacaauaacaacaugcccagcagugacgauggccuggagcacaacaaaauccagaauggcaaagcccccagggauccugugacugaaaacuguguucagggagaggagaaggagagcuccaaugacuccaccucagucagugcuguugccucuaauaugagagaugaugaaauaacccaggaugaaaacacaguuuccacuucccugggccauuccaaagaugagaacucuaagcaaacaugcaucagaauuggcaccaagaccccaaaaagugacucauguaccccaacuaauaccaccguggagguaguggggucuucaggucagaauggagaugaaaagcagaauauuguagcccgcaagauugugaagaugacuaagcagccugcaaaaaagaagccuccuccuucccgggaaaagaaagucaccaggacaaucuuggcuauucuguuggcuuucaucaucacuugggccccauacaaugucauggugcucauuaacaccuuuugugcaccuugcauccccaacacuguguggacaauugguuacuggcuuuguuacaucaacagcacuaucaacccugccugcuaugcacuuugcaaugccaccuucaagaagaccuuuaaacaccuucucaugugucauuauaagaacauaggcgcuacaagguaaaauaucuuugaaaaagauagaaggugggcaaggggagcuugagaagaauaaaagggauaaacgagcuccuaguuuuaaaaucucugccauugcacuuuauagucugauuacaaaacgugcaauucaggagcccagcagugacacacuuaucacgccuaggcuccaguuugcaaaaauugcaccuuauaaacugucaguauuaggagcaaugagacaaugaaagaaacauguugggaucguggauuuaagaaacuauacacuguuucucauaaucucuugaagaagggcuucugauucuacaauuuuaucagucucugcacaagaggaauaaccuuguuccuuuuuuguuacuuuuguuguuguuguucucauguguccuuaagagaaggaaaugccacaguuacaagguaaacauggagacuuaaacauaaagaaauaggcacuauacaauggggacauaaaaaaagaaaaucaaagaaggaugcagaaauugucuccggaguguuaagcauauuuuauucuuuuguuacgguccuauuuagaggauuggaauguaauaaaugcuuauuuuuugccuuucuuuuucccaccaugaagagaaagcaaacaaacagaaaccccaacuaggucacaccauuuuucuucuuguuaugccacuauuuuguacaucccuguucuauauuguuuauagggaaaaccuacaggacuuucacuaaagcuagccagagacagccaugaaaauggacguuucaccccuccucacuguguccuuugcauucguuggggauaucaaggucuauaaggauuuagugcacuuauaauccuauaugcgaaauguuuaaaccaaucugauuuucuccuaaauagccuuuuauaucaacagaauauccuaccuaaaacauaaaccuguuuaagaaaccaucgucacuguaaaguugagguuucaucaaauuaaauaauggacuaucuaguuggaccaaguuuauuaaagucaggugaauuuuaagauuaaaaaauagaaguuuaggacuuucuauuucaaccugaauuugccugacccauguaugaaauguuaacuuauagaaaaugaacuccugacuuucucagcagaaaggaaugcuguuuuauuuauuuggaauccuaaacaggauuaacauacucacauagcaccagugauuucugggccagucccuagagaauagaaccuucuauccaaugccugcuaaaggaugauauucaaauuauaaccaagugaggaugucugcaauuauccuaugaccccaugaaaggggaaaaaacgauauuuuaaaaacuauuacugaaacaauuacauauuccauauuccucaaauugucaauucucccuaacauuauuuaacuucaaaauucucuuaucaauagaauuauaguaaucuuugugacagaaauguaguggcuaacuaaauagaggccaaacgagcuuacagaaucugacagaaggagguagguggcuguugccaugccacaucucaccaucuaauucuagaccauaauaaucaugucaggaacuauuaucuaagugguggcauuauauagaaucuugccacguagagauuuuuuauuuggucgucuaaauaaauauaggauuaggauuauacggauucacauacaacuaguuauuuuuauccuuuauauuuuucuuacaguauuugcuguaguuuuccaaugagauuuucuaaaaauggcucaaucaauggaaaucaaauuauaaacgcauuugcaaauuucauauuuuggauuuuagauuuaugauaucuuguucaguaauaauaauguuaguuacaugguaaauuuaucaauauuagaccccagcacuuaaaagaauuauuuuguaugauuagccucauagaugccaccucaacauuuuggagaguuaaggaggaaaaaaagcaugagggaaaaaaaaaaccaaaaaacuuauaaguuaguaagaagcaaauugccagcaguauuuauauuugacuuauccuucguaaaaacaauuuccuguuuaaauaacuucagucauuacccccccaaaguaaucuuccauauuuaaagcauucauuuauagagauucccuuaccuacaguuacuucaacaugcacuuuuacauuuuaaauucauauuuauuuucauauuguuucuucaaaugaaggaaucaguugaacggaaauaacgucaagccagugggcuccucgaguuucugaaaaccagagaauugacuagccaaguauuucccuuuuuaauguauuguauuauuauuauguagauuuggcuucaguugggaagaacccuuuggcauccaggauucaggauucagggucuuuuuuuguuugcucuaauaauccaacgcaccaugaaucaagcuuuuagucucugcugccauuguauccuuccuuuuccucccucuguuauugauccuugccacagccacugcaaccucauugaaagagaaacugucacuuuaaagaaagaaauugaagauaggcugacaccauggaaacuacgucaaacagauaagaaaaauggaaggaauacugaaaaugauagaugauuggagagauauggagauuauauucaaggauuuuuauuaaccacugcuggauuugucuauuucaguggcucucaaacagagacaauuccccucuccccgaaaggggacacuuagcaauguguaaagacauuuuugguugccacacugggaggaaugcuauugguaucuagugcauagaggcugggaaugcugcuaaauagccuauaaugcucaggacauuuucccccaaccccuuuaaacaaauauuauuuggcucaaaaugucaauaguuccaagauugagaaauucuugucuacuuuaagggauuucuaaagcuugccucuuauuagugggaaaauaacccugcauuauuuuuagaauaucaaugcaaauuccucaccuguuagaaaaagguaauacucaccaaccauguaucagaaaaacuuuucuccugagucugguguaugugucagucacagauugggaacucaggccacaucuacauaucaaaaaucaccagacuaugcauaucgcacaucuuuuuuccagcaucuuaauguucaauuaugauugccacagaucuagucaauugagguugcaguaguuggccaucaaaauauuugcaguugauaugaucaguuucuacuggagcugcaauuuuuccuuucaaaaaauauuaagagccucuuucagaaacaauaucauuuaucuucuuugcauuuacuggaaagguguaucuguaaggucuuuguuuuuucucuccucauagcaauaauguagcuaucccagagugauauuauaagaagaggagauuaagaauauggaaaaagucuguucauuucuucgaaacaagaaaugauuuuccuuuuaccaguucaucauuaauuuaacgccagcucccaaguauuagggcagaguaaucaauaugagaagccacaacggguaagcuuucugauuugaaauguacaagcuggauguccaacauacuccugcucuuuguucauuugcuuuuuugauccugaagaucugacauugggagagaugggaauucaaaaugauggcaacaauuacucagcaaugcagucuguuuuuauuuucuccuacuuaguucauugacaguuuccuccucgccccaguuugauaauaucccuuucaacaaccauaguaccgguguugcaaauaguuuaggaaagcacccaaagcuggaugaaauugugcaaaguguauucucagcauguauauucaauaccaugccucuuaaaaaugcuucucuuaaagggugcacgcuguagcaugaaaugugugcauauuuaauguaucuuccuggaauaugcuaugugcuaaauauauauauauucuauaaauaaaauaauaaaauguacaauagaaauaca

SEQ ID NO: 39 - RNA, 8778 nt, Homo sapiens
  mRNA (1)..(8778): mRNA
gggcaauggcgggcgccccucccccagccucguugccgccuugcaguuugaucucagacugcugugcuagcaaucagcgagacuccgugggcguaggacccuccgagccaggugugggauauagucucguggugcgccguuucuuaagcuggucugaaaagcgcaauauucgggugggagugacccgauuuuccaggugcuuccgucaccccuuucuuugacucggaaagggaacucccugaccccuugcgcuucccaggugaggcaaugccucgcccugcuucggcucgcgcacggugcgcgcacacacuggccugcgcccacugucuagcacucccuagugagaugaacccgguaccucagauggaaaugcagaaaucacccgucuucugcgucgcucacgcugggagcuguagaccggagcuguuccuauucggccaucuuggcuccuaguauuuuuaauuaagcacuuguguucugauuaguggccaaauaaaugacagucagaacuucagcuaagguacaauaagguuuugcuguggcguggcaccuggucucuuucuagaaggaaaguucaacauacagcacaauucuggacacauuguauugguuugaugcuccuaccuggaacagguggagcuggucucuugggcagccugacaucugguccacuccucuguccuaugccgggaucaucaugaccguagagauuaugucacuguuuugcauccuuguuacauaacucaguuccugguagauuacuaugucagagagucacaaugaccuugcacaauaacaguacaaccucgccuuuguuuccaaacaucagcuccuccuggauacacagccccuccgaugcagggcugcccccgggaaccgucacucauuucggcagcuacaauguuucucgagcagcuggcaauuucuccucuccagacgguaccaccgaugacccucugggaggucauaccgucuggcaaguggucuucaucgcuuucuuaacgggcauccuggccuuggugaccaucaucggcaacauccugguaauugugucauuuaaggucaacaagcagcugaagacggucaacaacuacuuccucuuaagccuggccugugccgaucugauuaucggggucauuucaaugaaucuguuuacgaccuacaucaucaugaaucgaugggccuuagggaacuuggccugugaccucuggcuugccauugacuacguagccagcaaugccucuguuaugaaucuucuggucaucagcuuugacagauacuuuuccaucacgaggccgcucacguaccgagccaaacgaacaacaaagagagccggugugaugaucggucuggcuugggucaucuccuuuguccuuugggcuccugccaucuuguucuggcaauacuuuguuggaaagagaacugugccuccgggagagugcuucauucaguuccucagugagcccaccauuacuuuuggcacagccaucgcugcuuuuuauaugccugucaccauuaugacuauuuuauacuggaggaucuauaaggaaacugaaaagcguaccaaagagcuugcuggccugcaagccucugggacagaggcagagacagaaaacuuuguccaccccacgggcaguucucgaagcugcagcaguuacgaacuucaacagcaaagcaugaaacgcuccaacaggaggaaguauggccgcugccacuucugguucacaaccaagagcuggaaacccagcuccgagcagauggaccaagaccacagcagcagugacaguuggaacaacaaugaugcugcugccucccuggagaacuccgccuccuccgacgaggaggacauuggcuccgagacgagagccaucuacuccaucgugcucaagcuuccgggucacagcaccauccucaacuccaccaaguuacccucaucggacaaccugcaggugccugaggaggagcuggggaugguggacuuggagaggaaagccgacaagcugcaggcccagaagagcguggacgauggaggcaguuuuccaaaaagcuucuccaagcuucccauccagcuagagucagccguggacacagcuaagacuucugacgucaacuccucaguggguaagagcacggccacucuaccucuguccuucaaggaagccacucuggccaagagguuugcucugaagaccagaagucagaucacuaagcggaaaaggaugucccuggucaaggagaagaaagcggcccagacccucagugcgaucuugcuugccuucaucaucacuuggaccccauacaacaucaugguucuggugaacaccuuuugugacagcugcauacccaaaaccuuuuggaaucugggcuacuggcugugcuacaucaacagcaccgugaaccccgugugcuaugcucugugcaacaaaacauucagaaccacuuucaagaugcugcugcugugccagugugacaaaaaaaagaggcgcaagcagcaguaccagcagagacagucggucauuuuucacaagcgcgcacccgagcaggccuuguagaaugagguuguaucaauagcagugacaaaacgcacacaucaacccacagaccuuaggaggaggaaggcgagggcggggugacuucuggugaugauaaaaaugguuuuaucacccagaugugaaagaagcugccuguuuacugauccauugaauaaacccauuuuaauagaaaaagucaauaccaauucagcaaaaagaaaaaaaaaacauacuacugaauauaaagaaauuuauucugaaauagacuuuacguguuuuuuucuuaaagaggagaaaaauauugcuugacggcaauuauauacccaaagugauuugccuggguccuuuaauucccauuagcuuuggaaucucagaugagcauagcugacccaguucccacauucuucccaaggaucccaaaagugggaauccagaccccaaguggaacacugcaggcuuacgaaucugugguuccaaaauuauuucauacguugcaaagcugaaucuucuugucccaauagagcuuccugucuuuucuuugguguguuguuaaacucuauuuguggacuugauucuugauucuugcaaaguacuguuuugugcaguucaaguuucguacaaauaaaaauacuuaaguauauauauaugugugaguucugcacgcacacacauaguguauauaauaucaugggaaacacugaacuggcaaauuauuccugcaacauacgcuuucaguacuuugguaacugaaguucucuaggauccuaaugcaacauuaacgugaaauaagcccaguguaauguuuuugaaaccagggcuguuuuccacagagagcagccaggccuucccagcaggucugugcagagcggacaggcucgugagucagcugagcgccguggcuucgccagacuugguguuaagcaaccuccuuuguugaugucucaacagagcuaaaucggggccccucugagcucaaagaaugaaccacauccacacguuugaauuuaaucaucuaaaucugaauguuucagaacaaaauuucugcuaucuaaacugcuugaaacucaauaauagugucacguuugaaugucauacacagcaauauauauauauguguauauauauauauauggcaaagcaaaaaaaaaaacaugguaagagagaaugaaggagaacauuguguuugauucuugcugaauggcaccuucucaaagaaaauagggcuugcaccuuuguuaaucagcuguggccagugcuuucugguguucauuguguaaccuucacccaggaauaggugagguuuuaggaaguuacauguccucugaagaaagaauuacacucugaaaaguaaugcuucaaauugauuuccuuaccuuuugggaaaaaaaaaaaauuguuuuuuugcauucucccuugaauugaccaaaauguuaacuguuucauuuggggaggggauggggugcugccaucauugucguuguuguugcugcuguagcuguugggguuucuuuuccuguugccggggcuguuuggggagagggaggggagggaggugggagggccgcggagauaucuucccuuuguacagggcauucguguugugaacccagagcuggguagaagcugcuuuuguauucagugugaggugguguuuacagacgacuuugacaacaguagaaguguacucaguggugucuguguaucugaacuauuuaauuucguguuauguuuauaugcagaaauauuuauggauacuacaccaaguguuuauuuaauguugauaaauaugacucuucagucgucagccaugguguccuuucaaagugauucuuuaagguccacuugagcaaugaauagaguauauuggagcuuuccuguggcuaagaagaagaaacaugucauccuguuggcaucaccaagcaccuaacucuuucuagguaauaaaaagucaacggucacuucaacguaaauguuuaaaaccacucuuuaguuaaaucugaugggaacugaacaaagcuccaccauugugucugaaaucauuuuacacagggaaaaaaaaaaacaaucagcauaauuguaagaaugauuuuuuugggcccacauucaaccaaagaacaagaaagaaacagacgauuuucuaauauguacacagcaaacuacagaaugucauauuauccaccugugaaacaucuaguaagcaauugcaucuauauuuuauuucucaaguuggccuggcacuaauaaauaugugaaagcuuauucaagugucccaguacuucagcacuucccauccuuuuggaaacuccuaucuugccucccucccaaaagggcuguuucuuuuauuaaaaaaaaaaaagaaauagaauuaaauuugagaaaaggagaaacccugaaauuuaacagaacuuugagagucacuuccaaucuccuuaaacaugauuaucuccuuauccuuguccuuuugugaugcuaauaauagcagaaaaggaacaaauccaagacucuaggucucccuuuuuuguuuuacugguuuuauuauagaauuguuuuuaagccucuuauggaugaaagcugcauuuaaauacuaucuacugauuuuauagaaagguauguccauuucuugggucacuuucagagaaagaaaacaagcaaaauagguucuuuugaauaugaauaauuuaaaagcauaauugaagauguagugaguauuuauaauuggaccuuaguuacacgugcauuucucaaagggcuugucauuacaggagauauucagcacuuaccuaagaucaaaaugucuucaucccaaauugggaaucgaauuauuucaugccauaugcaugagguuugcagacauuucaagauuagccacuuugcuugaaagaagaaaaugaucaaaccuaaaauaaacuacacauggagaaucucucucucucucucugccucucucucucucuuucucccucuucuuccuucucucucucucacaagcacacacacacacacucaaucuccauucuccaggcauugccagaugguuaggagaagugagcucucucugguccccuagaucccucggcucugcucuccuuugcccaucagcucccuguggccucuaugcuaguccucccuauucuccuagucacccuuugccucuuaagcccagccuugacucucuacauauuuuucauuucucuuuacccuuuaguguacaauaguuguagacucugacggaaugaaaacaaacaagaagccuaagcguuagacccugcuguggcuaaagaucuagaaggcauaucaguuuucucugucaggcuuccuacguugcccucgugcagggcucauugcucugcagagcgcuggaagccccuucauguaucgagagagaagcuggagcacagagcucugaugcacaguuggcuguuucagcucugacucagggaggaaagaaggaugccaaggcucugucacagcggagaagucuaauaccagaguggcacagagaaggauuccagggaucagucaggguguccuguaccuaucugaguagagaugauucaaggauggggacauauuccaggaaauugcacaaucuuccccuggcagcuucagaggacgagugaaaauagugcaguauagucaaagaagcaaugaauuuaggaugaggaaccugaaguuucuggaccaguuuggucacugcuuaguuacauguccuuaggaaugcuugucacuuaaccuaaacuuucuggguuuuauccucauugcucaaacuaagcaacaacaccgaagcuucucuaauuugccuucugaugccaaaucggucaaaacugaggucagcagcuaccaugacgaaaguaaaaugguauauugggugaauguuugaaaauuucagauuuuauauucuguaaaguuauuuacuuacucuauuucaacaccacaaaaucuugaaauuuggaagguaaccaaggaagcuuugaaauuuguuggcaaacccggaguaguaugguuucaaauaccacuccaacccagcaccugaggucgggcaaacucacaaaauagaguuucuagguagcagguguuugcuuugguuuugcaauagcauuucucauaacagaacugaaacuguauagauuacaacacuccaaaguuauuuuuuuuuaaccuaaagaacccuacuucuagcaccuccucugaaagaauauuuugcuccaauaccacgugcaacaaacuuauuuagacaguaucaacucuagcccuuaauagguucaaggacaaguuuacugcuuccuuucuacacgcuuaaguuacacucucuucuccuguucugaugacucuaaaucuucagccucucccagcauuaaugugugagucaacauugcagaccugagaaagguuucuagaaugguaacccaauguucuuuagaacuggagaauuccauuuuccuuuucuggcuuuaguguuaaugucugggaagggaggcucaugauaagaaaauaaaaaugagaaaagaagaucuaguucauaaaaugacuauaccccacguaaguuuguaacugcacaguguuuuguuguacggcucagaaaugagcaaguguuaguauuauuaggcacacaucacguucacagcuucgcgcagagacggauaguguauuuggauauauggaagucauccaaaugcguugcuagaauaucaggccuauguuuguucugguagaaccacagagacucacacaaaguucugacccuaccacucuggucugggcugagaccaccccaaaaaaucaucuagagaaucaaaggggcucacaucucaguugcauucuauuuaaaagauaaaggggccauacucuccacugcaaagaagcauuaguaaaacuucccgcacccaaauuauaauccggcaacccuagcuucagcuaaaaagauuauccccagaaucuaaaagcuuacuauuguucuuucuuggaguccaaaaugcuuagcaguucugcaguucacauacaaaacucaaaacucuuuuuauccuguauucugugaguuccuugaugauaguaauugggucuuauucaucacucuguauccuguggcucacacagaaucuggcaaauugcauauauucaauaaauguugaauaucuaauaaccuccuuauuaauauaacaaauauuaucaacauucugugcacaucaaugucccaugcugcuacuguagucaggaguuauuuggcugggaaguggaaaguguucacaaacaugaugcuuaucuaauaaaauaucacugagcaauaaggagaaauauuuuaaauagauuugaaguugugaacaaauaauuuagaguccaaagaggaaaaagaaaauuaacucuguuuuuuaucccuagaacucagaaacuuuacuggauuggucaacaaagacaaacuuuuuauuguauaaaacaguagaauucauggaagggauaauucuuuuggaacaggcuucucgguuacuugccuuaguaaaagaaguaucuggagggccuggagaguucauaaacauaguuuucugugcaagugaagaucuguaguuuggcuuaaaaauccuugcauguaguuuugcuuggcguggaaaguuauuaggagacaauccuuuauccuguucugcaagcaaucaaacacaugaucucccuucuagccauuccucauccaguacaugggcauuuccagccuccuacauguaacacuucaaacuuccucugggcgucugcuuagagcuaugugauggccacaguuugggcaaagcccuucuuucuuuuaaauauaggaacuguuaccaaguuuggugcaggcuugaggaauaauuacauaaaaaugggcuuccacgauggugaaucagcaugcugauuucucuuggcaggaagcucucagggaaaugucugugaaucauuuaaagaugcucucugguggauagagaaaccgcaguccaaaaacagauggguguauauuugcuuuguaagcaaagggcauguaucauacucgcaucuaaaacaguguccacaucuuugccaaaggcacugaauuuuaccuuuuuuuauuaccucguggccagcacuuccucaguaggcauuucaggugugucacaaaccauguucauuuuuaaucuugaaaaagucaaucgucuguagucuguacuauaaaaugaugccugcaacuuuguggugaauauuauccccaaguuaacagugaaauaaguaaauaaaacuauuauugaacaaa

SEQ ID NO: 40 - RNA, 5802 nt, Homo sapiens
  mRNA (1)..(5802): mRNA
auucaugccaaguacgcucaccguccagagacaugauaaagccguacuuccuucuggauuuuuaccucacuauucaaaaaacaggguaccagaagcauggguuacaaaagcaugaggaggacagaagaaaugccuucaucuucuacagaaaggaagcugauggacugaaaaguucguauggagaaaggaaagcaagagugcaagaacauccaugcuucuaguccagauaauguuuuuuaaagcagcuugauuuggaagcguguguaauucugaaauacgaacaagcagaacuuuuccagggggucaaaugcauauaggaacaaccaggauuucucauugcugaagcauucaaugaaucuuuaauaaguucucaacacucagauuguuugugaucagaucaacugcuguuaccagauaaaggcaaaacucacccuucugguucuauaauccuauccaaauggagucuucaucuaccgcauacuauccuaacuaaugcuacaggauuaucaggaauacagauauaaagacacugcaagaaauaaaacacuggcaaaauaaaacaugaaaggaaaguagagaaggcauuuugggucucgaagcuuacuauuuccagccagaguucaaauuacugagcuucaaacaaaccacugccaggaaccuaggaaaucauucucagccuugcaacuuaaaauaugguacauggaucaguagcaucaagaauacucaggagcuuuucagaaaugugcaauugugaaauauggccaagauggcggauuagaagcagcggcagcccgcagcucucaugcaaaggaaugaaaggugugagugaauacagcgccuucaacugaaauauccagguucucgcuuugggacuaauugggcaaacaacuugacccacagaaaacaaagaaaagcaggguggggugacggcccaccugggagcagcacagagccaaaggaacccccaccuccagccaagggaagcgaugcuggccaagaagagcugaaauagaaaacagccuagaaccuaacacuauuuacuguaaaauuuuugcaccaggauggaaggggauucuuaccacaaugcaaccaccgucaauggcaccccaguaaaucaccagccuuuggaacgccacagguugugggaagucaucaccauugcagcugugacugcugugguaagccugaucaccauugugggcaaugucuuggucaugaucuccuucaaagucaacagccagcucaagacaguuaacaacuauuaccugcucagcuuagccugugcagaucucaucauuggaaucuucuccaugaaccucuacaccaccuacauccucaugggacgcugggcucucgggagucuggcuugugaccuuuggcuugcacuggacuacguggccagcaacgcuucugucaugaaccuucuggugaucaguuuugaccguuacuuuuccaucacaagacccuugacauaucgggccaagcguacuccgaaaagggcuggcaucaugauuggcuuggccuggcugaucuccuucauccucugggccccagcaauccucugcuggcaguacuugguugggaagcggacaguuccacuggaugagugccagauccaguuucucucugagcccaccaucacuuuuggcacugccauugcugccuucuacaucccuguuucugucaugaccauccucuacugucgaaucuaccgggaaacagagaagcgaaccaaggaccuggcugaccuccaggguucugacucugugaccaaagcugagaagagaaagccagcucauagggcucuguucagauccugcuugcgcuguccucgacccacccuggcccagcgggaaaggaaccaggccuccuggucauccucccgcaggagcaccuccaccacugggaagccaucccaagccacuggcccaagcgccaauugggccaaagcugagcagcucaccaccuguagcagcuacccuuccucagaggaugaggacaagcccgccacugacccuguccuccaaguggucuacaagagucaggguaaggaaagcccaggggaagaauucagugcugaagagacugaggaaacuuuugugaaagcugaaacugaaaaaagugacuaugacaccccaaacuaccuucugucuccagcagcugcucauagacccaagagucagaaauguguggccuauaaguuccgauuggugguaaaagcugacgggaaccaggagaccaacaauggcugucacaaggugaaaaucaugcccugccccuucccaguggccaaggaaccuucaacgaaaggccucaaucccaaccccagccaucaaaugaccaaacgaaagagagugguccuagucaaagagaggaaagcagcccagacacugagugccauucuccuggccuucaucaucacauggaccccguauaacaucaugguccugguuucuaccuucugugacaagugugucccagucacccuguggcacuugggcuauugguugugcuaugucaauagcacugucaaccccaucugcuaugcccucugcaacagaaccuucaggaagaccuuuaagaugcugcuucucugccgauggaaaaagaaaaaaguggaagagaaguuguacuggcaggggaacagcaagcuacccugaaaagucaacaacuccucucgaaagaacaaugaccacagucaacauccucugaggaugagcaagcugauucugguuuguauauuuucaaaaagaagacaucucauuuugaguccuugaagauuuuuguaaaggcucaaguuugguugccaaauggaaggggccauagcugcagcaauugcugacauauuaaaugacucuugccuaugaccaaggccauuugaugccaggggaguuugccaaugaaguaaagggauaggcucauggcccuucacaagaggaagcacacuggguaacaaugaacagugacucagggaacuuaugccccuucuguaggaaacagcagagaccagguggaaaccuuuuccuguggaaaccugucauagaauuuugugcaauauguaugugucuaugaagcugucuugugccagugagaaccagcaggagaauguacaacagugucacuuguuaacgagacugauauucaacaggcuucuaagaauauguaucuucauaaacugaucacuaucuauuaugcagcuauuauguggucuauacuauacugugguuuguuuuccuguccccacaucugagugaaggucuugcucuucccuuucauauccaaugucaauuccuuguacucacugaacccaugcugaucuccagggaaccauccuuccucucagaauccagagucuggaaggacugaaaccuggucauacccaguccuuucaaggggccucuuuucuacuaauaaagauggaucaagucuccacagucaguuauuuuaguuuuugccaguggaaauuuuuucuuuuaucuaaacagauauugugcauucuauuuugugcccauuugaacccauuuauuuaugcucuguucuuaggaagacuaauuguguaagagcccuucacuggcugaagauuuucuccaggcuguugaauuaagguucuuauauuguuuucagagaucuugcuuucuaaucucuucacagggcaaaccugagcaaaauguggaaaacagauuugcuuuugaauuucuuccagagcccuuuugcagccuccaauuucccuuguucagauccccaguucacaccaagacgcaagcaggucagcugugcugccuccaagcaggggguuguggggugagguggggguagaaagucuuuuuuuauagguccuucaaaucagggccuaaaaaugacuucuuuauuccucuccaaauucggucacaaaugagaaggccagaaaagcacacuugagggugcgaugaagcugaaaaugaucugaaagcuucauuuuuaaaaacaaaguguaggucauaguaauacauaaaaauaaaacaguugacaugcaugacagcaugaggauuauuaaaaaccacacuuaauugaaaucucuaagugcguauaaacaaacggaaucaaaggaccuuuguggacaaaaguccacaaagaagcaguagugugguacacagcaaggauuuuaaagaugaagcacuccugucagacauuauuuaacucguugguuacaucugcagcuggacuuaaaaaucaccagacuucaugcgucuuaguguaggccggguccuuuucagucucauuugcuaagaggauggaaaauuagcugguuuaguuguccugggaauuagauauguaccugaguaugggagagcuucagccuaccacugaagagcaucaucagccucucacaggaugucccacggaggcuucaggguaguagaaucucaggcaggacgggacuggucccuuauaaagacuucacguugacaugaaacaucuuguaagcagugcaucugcugagccuuuguaaagggcagguugacaccugcaaagaauucggugggaacaguaauuggcucaguucuccagcacagaggcuuucgggccuccgugaugccaacugggugugacuucaugagcaaaccugcucuaccagcgccagauaaacaguugagucuucguuuaaggcuuugauuccugauaaagaugaaaaccccuucaugagaauaggcuuuuaagcccauguugggcauucaccauucgcaguugagagugaaacaagcuaauuuaaacaaaagaacacaaaucgggaaaggaugccauggagggcacuccucgcacacuccgcacuugcuuaggacuuggccuuuuuuguucaucuuuugugaaauugauuaagaaguguauugcugggugugguggcugacaccuguaaucccagcacuuugggaggcugaggcaagaggacugcuugacccuaggggcucaagaccagccuggcaacauagggagacccugucucuacaaaaaaaauuuuaaauaccugggcauuagcagggcaucguagugugugccuguagacccagcuacuucagagguugaagcaggaggaucgcaugagcccaggaguuagagguugcagugagcuaugaucacacuacugcauuccagucuaagugacagagcaagacccugccucaaaaaaaaaaagagagagagagaggucaaugcuucuuuuuuuaucauugguuuuggaaagcagccaguuuugcagacuaccuaugccucuugacauagacaaaaaauaucccaaacuacuagggacaacuagacuucuauagaauagcggcuuuagugggguccucagaaugaauagaccuaauuguaaacuacagggaccugaugggugggugggaggcgcauggccugacuugagaucuuaggccuucuaugcccccagucuuccaggcuuugucuauaguaaaccauguaugacuuuggcuaguuaccuacuggaugcuguuacuuugcuaucauuguaauuuguuuccccuuggcugugaauuugaugacuugucagggaaucuguuuuccauucaauuuuugguugaacaaagcauuuugucaauauucucagcuuuugcuucgcuccuuuggugaauguccuuugaagacaauacugaugccagcucuuuguaauugugaaaucuguacccaaaccucuggauuagaaucuccaguugucuacuguaaauacuggaauuacagcaaaggauauggggacugggcugcuuuucuguauuguauaagcacuauucuagauauuaaagaaauuuaaccgca

SEQ ID NO: 41 - RNA, 4427 nt, Homo sapiens
  mRNA (1)..(4427): mRNA
aguauuggaguguuacagggagacauacaggauuuaagaagcccaucauggagaagaccuucaauuacagagauaaaaaguuuuucuugugaacaaguuacacuagauggaagauaacagacugaggagugagcugcuucugacucgauuaaaaagggagugagccauaacugguggcugcucuugcgccaaugagccuccccaauuccuccugccucuuagaagacaagaugugugagggcaacaagaccacuauggccagcccccagcugaugccccugguggugguccugagcacuaucugcuuggucacaguagggcucaaccugcuggugcuguaugccguacggagugagcggaagcuccacacuguggggaaccuguacaucgucagccucucgguggcggacuugaucgugggugccgucgucaugccuaugaacauccucuaccugcucauguccaaguggucacugggccguccucucugccucuuuuggcuuuccauggacuauguggccagcacagcguccauuuucagugucuucauccugugcauugaucgcuaccgcucuguccagcagccccucagguaccuuaaguaucguaccaagacccgagccucggccaccauucugggggccugguuucucucuuuucuguggguuauucccauucuaggcuggaaucacuucaugcagcagaccucggugcgccgagaggacaagugugagacagacuucuaugaugucaccugguucaaggucaugacugccaucaucaacuucuaccugcccaccuugcucaugcucugguucuaugccaagaucuacaaggccguacgacaacacugccagcaccgggagcucaucaauaggucccucccuuccuucucagaaauuaagcugaggccagagaaccccaagggggaugccaagaaaccagggaaggagucucccugggagguucugaaaaggaagccaaaagaugcuggugguggaucugucuugaagucaccaucccaaacccccaaggagaugaaauccccaguugucuucagccaagaggaugauagagaaguagacaaacucuacugcuuuccacuugauauugugcacaugcaggcugcggcagaggggaguagcagggacuauguagccgucaaccggagccauggccagcucaagacagaugagcagggccugaacacacauggggccagcgagauaucagaggaucagauguuaggugauagccaauccuucucucgaacggacucagauaccaccacagagacagcaccaggcaaaggcaaauugaggagugggucuaacacaggccuggauuacaucaaguuuacuuggaagaggcuccgcucgcauucaagacaguauguaucuggguugcacaugaaccgcgaaaggaaggccgccaaacaguuggguuuuaucauggcagccuucauccucugcuggaucccuuauuucaucuucuucauggucauugccuucugcaagaacuguugcaaugaacauuugcacauguucaccaucuggcugggcuacaucaacuccacacugaacccccucaucuaccccuugugcaaugagaacuucaagaagacauucaagagaauucugcauauucgcuccuaagggaggcucugaggggaugcaacaaaaugauccuuaugauguccaacaaggaaauagaggacgaaggccuguguguugccaggcaggcaccugggcuuucuggaauccaaaccacagucuuaggggcuugguaguuuggaaaguucuuaggcaccauagaagaacagcagauggcggugaucagcagagagauugaacuuugaggaggaagcagaaucuuugcaagaaagucagaccuguuucuuguaacuggguucaaaaagaaaaaaauaauaaaaauaaaagagagagagaaucagaccuggguggaacucuccugcuccucaggaacuaugggagccucagacucauuguaauucaagcuuuccgagucaagugauugacaacugaagagacacguggcuaggguuccacuggagaauugaaaaggacucuugagcccuccuggaauggagcuguauaacugugcagagacuuuauccaugccaauaguugcuguccccuuccaggggucaccuugagaggcaugacagcuguuccacaggggcuaucccuucucagaaaacuucucuucugagccucuuuaacagcuuucuccagaaccagugucugaaccacccuggaaauucugccuuauuauuucuuacucaaacauguuuagaguggauagaaaauuaugcagcuugcacacccaucgucuuuaaccccaaauuuccuuuggcuauuaaaaaagugguggcaaaagacauccucaaaagaaagagaaaugaaauauuuuugaaugguugcacguuaaaaauuaaaagaaggaaugggggcagaaugccauauuuuugagggcuguacuagguuuaucucauuuaagccccacaacaccccacaggaggguaauuuucuaacucuaguuugcagaggagcaaauugagguucagcaaggugagagagguacccaaggucacauagcuaguuaugugagaaaguuagaguacagauccucugggguuucagcuuauuguagcauauuuucuccgaaaggcaaaaaugugcccuuuuggccgggcaugguagcucaagccuauaaucccagcauguugagaggcugaggugggcagaucauuugaggccaggaguucaagaccagucuggccaauauggagaaaccuugucucuacuaaaaacacaaaaauuaucugggcaugguggggcaugccuguagucccacuuacuugggaggccgaggcacgagaauugcuugaacccgggagguggagguugccgugagccaagaucacgccacugcacuccagccugggcaacagagcaagacucugucucaaaaaaaaaaauacaauauuuuaacaaugugcccucuuaagugugcacagauacacauacacgguauucccaagagugguggcagcucaaaaugauauguuugaguagacgaacagcugacauggaguucccgugcaccuacggaaggggacgcuuugaaggaaccaagugcauuuuuaucugugaguucuguuguguuugucaaaaagucauuguaaucuuucauagccauaccugguaagcaaaaacuaguaaagacauaggaacauguaguuuuacuugguguuuauguugcaaucugguugugauuuauauuuuaaagcuuggugcuaaaccacaauauguauagcauauggagugccuguacaagcugauguuuuguauuuuguguuccucuuugcaugaucugucaaagugagauauuuuuaccugccuaaaauaugauguuuaaaagcauacucuaugugauuuauuuauuucuaccuuucugagucucuuggacuaagaagauguuuugaaauguaccaucaaauguuaacagaguuugauaugggcuuucucuuugguuucucaucacauuuguaaaugucuuuucaaaaggauuuacuuuuuguaaaaagcuucauucucacucugcuuugcaucccccaaacuucuuguucaaaacggggggaguuuaggagacuuuaaucccgguuucagaagcugcagcuggucuguuuccaggucagaaaccauuguucagaagaccucccugugagagaguugcuccucagggucccucaggaccaaagaacacucgaaaagagcacuucacacagacaaguggcuaaguguccauuauuuaccuugaacaaucaaggcaacuaguggagagaacugauugugagcucugccucugggucagagagaccuggauuugagucugacaagaacaagaaauggucaauaaauauaaauuaccagcgucuaaggaacaaggucuaugcauuauuguauacagugucucuagugcuuguauagugucugguauacagagggcacuccuaugcauuuuuaaaacaugcugagcacauaccaugugccaggcuuuguguuuuaucuaauguuaucuaaugguauuggugccauuauguaauguugccuuuacaacaaccucaugagggagauuuccaucuuuacaaauaggcaaacugaggcccagagagauugaggaacugcuccgaggucugauucuggaaugugcuuccuuuccacuuuaucaaucugcucuucguacuccugucugaacgauggaaauuaauuuuugaauguauaaaagacaacagacuaugauacagaaaugucagccccagcccacuaagaaagccccagcccaucaguggcuaauggcuuuaauaaauuggucauuuggcuaaaaaaaaaaaaaaaaaaaaa

SEQ ID NO: 42 - RNA, 2520 nt, Homo sapiens
  mRNA (1)..(2520): mRNA
gcaucccugcccggagcgcagccggcgcgggcgcgggaccgaggcgaaccgggugcggagccaauacccgcggcagcagcaccagcuccugcccuccacugacuccagagagggagauccccaguacuugacuccaucacgcagaugggagcaggcaccagcuauggagagggauacagcugcgucuccacaugacccauccugcaugacaccaaagccaccgccagacagugccucggauucuaugcaaaaccugggaagcggagaccuaccccagccccgggaggaagcuagcucuucaggggaccgucugaggacuggaguuugauccaugaaccuggcuucgaggccuugcuuuucucucuucuucauucauauucauucccaacaccuuagaagguguugcuuaauuuauuucuagaaaagcagcccagagucagucauugaagccuuccccacccccuggccaaaaaaaaaaaaaaaaaaaaacuggacacauuuuggaucuguugggagcuuggaguccagugguuggcauaguugucacauugggagcagagaagaagcaaccaggggcccugaucaggggacugagccguagagucccaggauggcacccaauggcacagccucuuccuuuugccuggacucuaccgcaugcaagaucaccaucaccgugguccuugcgguccucauccucaucaccguugcuggcaauguggucgucugucuggccgugggcuugaaccgccggcuccgcaaccugaccaauuguuucaucguguccuuggcuaucacugaccugcuccucggccuccuggugcugcccuucucugccaucuaccagcuguccugcaaguggagcuuuggcaaggucuucugcaauaucuacaccagccuggaugugaugcucugcacagccuccauucuuaaccucuucaugaucagccucgaccgguacugcgcugucauggacccacugcgguacccugugcuggucaccccaguucgggucgccaucucucuggucuuaauuugggucaucuccauuacccuguccuuucugucuauccaccugggguggaacagcaggaacgagaccagcaagggcaaucauaccaccucuaagugcaaaguccaggucaaugaaguguacgggcugguggaugggcuggucaccuucuaccucccgcuacugaucaugugcaucaccuacuaccgcaucuucaaggucgcccgggaucaggccaagaggaucaaucacauuagcuccuggaaggcagccaccaucagggagcacaaagccacagugacacuggccgccgucaugggggccuucaucaucugcugguuucccuacuucaccgcguuuguguaccgugggcugagaggggaugaugccaucaaugagguguuagaagccaucguucuguggcugggcuaugccaacucagcccugaaccccauccuguaugcugcgcugaacagagacuuccgcaccggguaccaacagcucuucugcugcaggcuggccaaccgcaacucccacaaaacuucucugagguccaacgccucucagcuguccaggacccaaagccgagaacccaggcaacaggaagagaaaccccugaagcuccagguguggagugggacagaagucacggccccccagggagccacagacagaccauggcuuugccuuccagaaugcuggucuguggaacugacccauucauucauucauuuguucauucauucauuugcaaacauucauccaauucccaccacaugccaggaauuaugaugaauacuggggauguagaaaugagauuuuuuggguuuauggugaugaaguuacagaaaaucaauggcacagcccuggucucagcucugccgucacucaacuuguaucuugagcaaguuccuccccaucucaggccucaguuuccucaucgcauagagaguuggucuucccucaccgggcuuccuacuugcccuccagaguugccugaagcucacaugagcgaagggacaugaacaugcucugccaucucccaauaggacauaaguccacgcuggagcgcugagcaucagacucaagaaaagcuugcguggauauugcagacccaccaacaagggcuccagguucuggaguuguggaggcaaaacugccuagugagaacccuacauauucccucugguccuugcagcugccacccccauucccagggguggagugucccuccauagacuuggauccugggcccucugcuccuucaccacuguccaucugaagcaagccugguugcuccggcacugaacuguggccucgggcccuggacccugccccucaaauccacccuccaagugcacaccuggaaacuuccgcuuccaguccugauggguaaaacgcccaccuaccauauccuugcgcaaucucuuauucaggaggcagcuggagugcaaagcugacuuaguucacaaaguauaaaaaaauacagggaugugguucagcuagaa

SEQ ID NO: 43 - RNA, 2692 nt, Homo sapiens
  mRNA (1)..(2692): mRNA
gcacucggcugcgcguugcgcuccggcugcacggucgcaccggcagcggcucaggcuccggcuccucucccgcugcagcagccgcgcugccggccccacugggcucggauccggccccggcccccucggcaccgccugcucuggccccggccccggccccgcggaccaugcgcugggcgcccccaggggaacccgacccggccaagggcccgcaaagacgaggcucccgggccggggccccucccggccgcccagcucucggccggcgcccugccccgcgucccggagccgcgugagccugcggggccauggagcgcgcgccgcccgacgggccgcugaacgcuucgggggcgcuggcgggcgaggcggcggcggcgggcggggcgcgcggcuucucggcagccuggaccgcggugcuggccgcgcucauggcgcugcucaucguggccacggugcugggcaacgcgcuggucaugcucgccuucguggccgacucgagccuccgcacccagaacaacuucuuccugcucaaccucgccaucuccgacuuccucgucggcgccuucugcaucccacuguauguacccuacgugcugacaggccgcuggaccuucggccggggccucugcaagcuguggcugguaguggacuaccugcugugcaccuccucugccuucaacaucgugcucaucagcuacgaccgcuuccugucggucacccgagcggucucauaccgggcccagcagggugacacgcggcgggcagugcggaagaugcugcuggugugggugcuggccuuccugcuguacggaccagccauccugagcugggaguaccuguccgggggcagcuccauccccgagggccacugcuaugccgaguucuucuacaacugguacuuccucaucacggcuuccacccuggaguucuuuacgcccuuccucagcgucaccuucuuuaaccucagcaucuaccugaacauccagaggcgcacccgccuccggcuggauggggcucgagaggcagccggccccgagcccccucccgaggcccagcccucaccacccccaccgccuggcugcuggggcugcuggcagaaggggcacggggaggccaugccgcugcacagguauggggugggugaggcggccguaggcgcugaggccggggaggcgacccucggggguggcggugggggcggcuccguggcuucacccaccuccagcuccggcagcuccucgaggggcacugagaggccgcgcucacucaagaggggcuccaagccgucggcguccucggccucgcuggagaagcgcaugaagauggugucccagagcuucacccagcgcuuucggcugucucgggacaggaaaguggccaagucgcuggccgucaucgugagcaucuuugggcucugcugggccccauacacgcugcugaugaucauccgggccgccugccauggccacugcgucccugacuacugguacgaaaccuccuucuggcuccugugggccaacucggcugucaacccuguccucuacccucugugccaccacagcuuccgccgggccuucaccaagcugcucugcccccagaagcucaaaauccagccccacagcucccuggagcacugcuggaagugaguggcccaccagagccucccucagccacgccucucucagcccaggucuccugggcaucuggcccugcugcccccuacccggcucguucccccaggggugagccccgccgugucuguggcccucucuuaaugccacggcagccacccugccauggaggcgccuuccuggguuggccagagggccccucacuggcuggacuggaggcuggguggccggcccugccccccacauucuggcuccaccgggagggacagucuggaggucccagacaugcugcccacccccugcuggugcccacccuucgcaguuacugguugguguucuucccaaagcaagcaccugggugugcuccaggcuuccugcccuagcaguuugccucugcacgugcacacaccugcacaccccugcacacaccugcacaccgucccucuccccggacaagcccaggacacugccuuugcugccuucugucucuugcauaagccucaggccuggcccuuucaccccucuucccaccaacucucucugcccccaaaagugucaaggggcccuaggaaccucgaagcuguucucugcuuuuccauucuggguguuuucagaaagaugaagaagaaaacaugucugugaacuugauguuccugggauguuuaaucaagagagacaaaauugcugaggagcucagggcuggauuggcaggugugggcucccacgcccuccucccuccgcuaaggcuuccggcugagcugugccagcugcuucugcccaccccgccucugggcucacaccagcccugguggccaagccugccccggccacucuguuugcucacccaggaccucuggggguuguugggaggagggggcccggcugggcccgagggucccaaggcgugcaggggcgguccagaggaggugcccgggcaggggccgcuucgccaugugcugugcacccgugccacgcgcucugcaugcuccucugccugugcccgcugcgcugcccugcaaaccgugaggucacaauaaaguguauuuuuuuauuggugcuga

SEQ ID NO: 44 - RNA, 3664 nt, Homo sapiens
  mRNA (1)..(3664): mRNA
acacauuuuagguaugugauuagaaaacauacuugucagaauugucuggcuggauuaauuugcuaauuugaccuucuucaucauuugaugugaugccagauacuaauagcacaaucaauuuaucacuaagcacucguguuacuuuagcauuuuuuauguccuuaguagcuuuugcuauaaugcuaggaaaugcuuuggucauuuuagcuuuugugguggacaaaaaccuuagacaucgaaguaguuauuuuuuucuuaacuuggccaucucugacuucuuugugggugugaucuccauuccuuuguacaucccucacacgcuguucgaaugggauuuuggaaaggaaaucuguguauuuuggcucacuacugacuaucuguuauguacagcaucuguauauaacauuguccucaucagcuaugaucgauaccugucagucucaaaugcugugucuuauagaacucaacauacuggggucuugaagauuguuacucugaugguggccguuugggugcuggccuucuuagugaaugggccaaugauucuaguuucagagucuuggaaggaugaagguagugaaugugaaccuggauuuuuuucggaaugguacauccuugccaucacaucauucuuggaauucgugaucccagucaucuuagucgcuuauuucaacaugaauauuuauuggagccuguggaagcgugaucaucucaguaggugccaaagccauccuggacugacugcugucucuuccaacaucuguggacacucauucagagguagacuaucuucaaggagaucucuuucugcaucgacagaaguuccugcauccuuucauucagagagacagaggagaaagaguagucucauguuuuccucaagaaccaagaugaauagcaauacaauugcuuccaaaauggguuccuucucccaaucagauucuguagcucuucaccaaagggaacauguugaacugcuuagagccaggagauuagccaagucacuggccauucucuuagggguuuuugcuguuugcugggcuccauauucucuguucacaauuguccuuucauuuuauuccucagcaacagguccuaaaucaguuugguauagaauugcauuuuggcuucagugguucaauuccuuugucaauccucuuuuguauccauugugucacaagcgcuuucaaaaggcuuucuugaaaauauuuuguauaaaaaagcaaccucuaccaucacaacacagucggucaguaucuucuuaaagacaauuuucucaccucuguaaauuuuagucucaaucucaccuaaaugaaucaggucugcccuuuaucuugcccuuuucauucuaccaacagaucugcacuuugaagucaaugguaaauuacuccagugaauaauagcaguauaauaugacuugauaauauuuuuguaaacuuguagucauaauaguacuauauucuucuuaguccucaccucuuccuugucuuuuagaucuuaauuucaugcugauuacaaaaauccaguuuuguuuucuuucuauguuccaugcauaauacagucuuaagugaauuucucuuuuuuaauuuuaucguaauagaaacuuauccaguuugaaaaucauucccuaaagcaugcaauaggaaaaagaaccuccuggcugggacugcccaacucuguucugaucagugggugggugagguaggguuugaguuggcaagagcagggaacgggcaugugcccaggugagcuccuguguguguccagauuuuauauuccuaaucccaguaaggaagaaagcguagugugggagaggagagagcugaugacugcaguucucaaagguccucagugaaguuauuuuggaggcccugguggucacaggaucagaaggcaagggauaggcaguggucaccaaugguugaaaguauggcuugucccauuucuuccuguucucuuuuucuagcuuccacaucagcuuccuuuuuugagaacauauagaagaagaaggcuaagagauggugaagagacugcaugauuaaacuagauagaccugguauacagucacugaacuaguagaugucaauaauuauuauuuuuaaaaauuuuuauuuguuggccgggcaugguggcucacgccugaaaucccagcacuuugggaggccaaggugggcggaucaugaggucaggagaucgagaccauccuggccaacauggugaaaccccaucuguacuaaaauacaaacaaguagcugguuguggcgccgcaugccuguagucccagcuacucgggaggcugaggcaggggaauugcuugaacccgggaggcggaguuugccagccuggcaacagaacaagacucugucuaaaaagaaaaaaaaauuuuuuuguuuaagacagcaucuugcucugucucccaggcuggagcguaguaaugcaaucauagcucacugcagccuggaacuccuuggcucaagcaauccugcugccuuggccucccaaguauguaggacuacagguacucgccaccacaccuggauaauuaaaaaauuauuucuguagagaugaagucucacuguguugcccagccugggugucaauaauuauuuuuuaaaaaaaauuuuuaaaaagguuuuuugagacagauucuugcucugucacccaggcuggagugcaguagcaugaucagggaucacugcaaccucugccuccuggguucaagcgauucuugugccuaagccaccugagcagcugggauuacaggugcaugccaccaugccuggcuaauuuugguauuuuuaguagagaugagguuuugccauuuuggucaggcuggaauuuuuuuuuuuuuaauuuugauaagacaggguauugccguguuggccagacuggucucaaacuccugggcugaaacaauccucccgccuuggccucccaaagugcugggauuauaggcacaagacaccacaauaauuauugccuguaugucaauuauuauuuuaaaauauuguuguauuuacuuaaugucuuuaaugcauuugcccaauauuuuacauuguacugcucagagguauuccuuuauuaugugguuagcauagguuauacuuugcugacgauucacauuuuauuaguuugguuauguuuuguccuuuuaaaacauuuucuuuugagaugggggucuugcucuguugcccacgcaggagugcaguggcaugcucucagcucacugcagcccugacugccuaggcuccagcaaucuucuuacgucagccuccagaguagcugggaccgcaggcacuugccaccacgccccacuaaaaauuuuuuaaauuguugccuuucuugaaguguucucugccugucuuugucacaaaauuucauuuuucucauaguuaauuucaucucuccgguaagauuuuauugguguuucuuuuauaacuuugcaguucuuacaccguuuggugauuuucauguuucuuagaaacuuuaaaccuuuaacuucaaacauuaaaauacaagucuuuuaaguacaugagugcuuagaaauguacauaauguuuauauacacuuaugccuuacauuaaaguccaauaugagaaauacauguuuaacauucaauaauaauuuuaaaaauuugagaaauaaacucucauaaaugcacaca

SEQ ID NO: 45 - RNA, 3248 nt, Homo sapiens
  mRNA (1)..(3248): mRNA
gagaccagacaggaucucuucaagagcagagucaagagccuggagggcguuucaagagccguggagugcagugaauuagagaaccaaaucagaagcagaagugggcccuagugaagguauccauuuuucagcuauauuaaucuuuuaaaacaaagaaaauggauuucuuaaauucaucugaucaaaacuugaccucagaggaacuguuaaacagaaugccauccaaaauucuggugucccucacucugucugggcuggcacugaugacaacaacuaucaacucccuugugaucgcugcaauuauugugacccggaagcugcaccauccagccaauuauuuaauuuguucccuugcagucacagauuuucuuguggcuguccuggugaugcccuucagcauuguguauauugugagagagagcuggauuauggggcaaguggucugugacauuuggcugaguguugacauuaccugcugcacgugcuccaucuugcaucucucagcuauagcuuuggaucgguaucgagcaaucacagaugcuguugaguaugccaggaaaaggacuccaaagcaugcuggcauuaugauuacaauaguuuggauuauaucuguuuuuaucucuaugccuccucuauucuggaggcaccaaggaacuagcagagaugaugaaugcaucaucaagcacgaccacauuguuuccaccauuuacucaacauuuggagcuuucuacaucccacuggcauugauuuugauccuuuacuacaaaauauauagagcagcaaagacauuauaccacaagagacaagcaaguaggauugcaaaggaggaggugaauggccaaguccuuuuggagaguggugagaaaagcacuaaaucaguuuccacauccuauguacuagaaaagucuuuaucugacccaucaacagacuuugauaaaauucauagcacagugagaagucucaggucugaauucaagcaugagaaaucuuggagaaggcaaaagaucucagguacaagagaacggaaagcagccacuacccugggauuaaucuugggugcauuuguaauauguuggcuuccuuuuuuuguaaaagaauuaguuguuaaugucugugacaaauguaaaauuucugaagaaauguccaauuuuuuggcauggcuuggguaucucaauucccuuauaaauccacugauuuacacaaucuuuaaugaagacuucaagaaagcauuccaaaagcuugugcgaugucgauguuaguuuuaaaaauguuuauuauugaaggauggggguuuuugaggggaggaauaacuagaugaaugccaaauaauaaaacacuuaagcuuuuagagggaaauacaugaaaacugcuaaauugauaaggcuauaauuuauauuuuaauagcaaugugaauauaaaaguuauugaucaccacuauucuaggguauucaaaauuagaaaauaauuuauguagguuauaaacaauauuuugccuaugcaauguuccuaaaaagcuaacuggaaaaaauauauauauauacaauaguaaugacagugauuuuccauguuuacaucuuacaauaauuauagaggaguuuucaauuaacaacauaccucccuccauaauuucuaucuaguccuacuguuuuauaauauaauucuauuauuuuguauaagaaaauauaauucaccacaaaguacacauuucuccugucuccauucuugucccuucuccaucuuacccugagcaaaagggagggggaaggagcaacucuuacuuagugugacugcagaaaauguaaaaaagucagagcuugaaauuguccuuguguuuagguagcaaagaguaaucauaaaguggguuagguacagcaaaauaauucaauacugaacuugugcauauuuaauguauuauguuugauauaaacuucuagaauaauauuaucuuauccuuauauuaucagcauuugaaaucaaagccuuaccuuaguaacaauaacucaacaaaggggcaugggugacguuguuauaauugcucucuguuuacuuaggaaucaaauuuaaagcacuaaauaucauagauuacucaccacaguuauacucuuaacaucuuuuuaaaaguauaaaauguauuucauuucuguuaauaagcucaguuauuaacuaaauugucugugguauuuugcacuucaguauuuuccauaccuuugauuucaacauuauuucccguuuacuagacagaauauaauggaaauuuuuagaacuuucccuuuacuuuaaaauauuucuacuuuuccaauugguagaaaugaacaggaaaaaagacucauauuacacuaauuaauaaauguacacacauauauauuuuuguaauuagucacucuacuaguucagugagccaaagcaauuccauaaguuuuucuuccauauuuauaaaaggauuugaaaacaauagaaccaugaguuugagaggauuuuauuuuauuugaaccagcaaggucauagacuaccuuugggaagaccuaucaucaaccaagaucguuugaaaaacaauauauaccuuuuuuuaaguuguacauuuaugugcauuguaaauauuccuuccugauggugugguuaagaguacauuacaucaauuuuauguauauauagaucugugacaaauuacucucaaucuaacaaacauuuacagaguucccauuuuauacaaggcauugagacauauacuagauagaagauauacgaaauaauaaaauacaguuuuuuuuccugaauauguuuacagucuaaaaggagaaacaagaaaaaauaaaacuauaauagaaaagagcaaacuaaaaucuggcauggaaguuguuuacacaguuaugauggugcagaggaaaaacugaucgcaucuuguuuagaaaaaucagaaauauuucacaaagaauguaucuagaacacaugucaaauacacagcacuggggcaagaaaaauggaagagcacacuccagguugugacaacaucaggauacaaacugcaccaugcaaguauuugguggggcauguucauaaauagcaaugaguuuggcaacuauuuagagcaauagaggaaaaggauauuaaaagaaaaauugggcauuuuuguggcugacuuugaacgucagaaaaaagucuacagucaauuuuauugcugacgauggguaaucacugauacuuuuagcaaaaauauuacaaguuuagagcaaaucuaaacaaaaaagaaa

SEQ ID NO: 46 - RNA, 1826 nt, Homo sapiens
  mRNA (1)..(1826): mRNA
aguccaggcgcccagacucggcgaagcgcagcgccugccaccagcggugccucgggcugcccggcgcgccgcgcucccagguucugucucgccgcaccccggcgggcacuggcgcgggcaaggacgcuggcggggagaacgcccugggcucaacguacuccagaaucgaauguugagagaagcagugcucugauccagcucaggagaaaaaggagcggguuccgagugagacuucuggagccagcuggacgugccgguuugcccagugcggcgcggcugcacgcaccguccacaaggaaccuucacucagaagaaaugcuguggcccuucccuuuaccaacagaaaauggaacacaagagaccacauagcugaacaaauuauagccuccuuacaagugagaaaccuucgaggcuacauaguuuucagccaaaggaaaauaaccaacagcuucuccacaguguagacugaaacaagggaaacaugaacaucacaaacuguaccacagaggccagcauggcuauaagacccaagaccaucacugagaagaugcucauuugcaugacucugguggucaucaccacccucaccacguugcugaacuuggcugugaucauggcuauuggcaccaccaagaagcuccaccagccugccaacuaccuaaucuguucucuggccgugacggaccuccugguggcagugcucgucaugccccugagcaucaucuacauugucauggaucgcuggaagcuuggguacuuccucugugagguguggcugaguguggacaugaccugcugcaccugcuccauccuccaccucugugucauugcccuggacagguacugggccaucaccaaugcuauugaauacgccaggaagaggacggccaagagggccgcgcugaugauccuuaccgucuggaccaucuccauuuucaucuccaugcccccucuguucuggagaagccaccgccgccuaagcccucccccuagucagugcaccauccagcacgaccauguuaucuacaccauuuacuccacgcugggugcguuuuauauccccuugacuuugauacugauucucuauuaccggauuuaccacgcggccaagagccuuuaccagaaaaggggaucaagucggcacuuaagcaacagaagcacagauagccagaauucuuuugcaaguuguaaacuuacacagacuuucugugugucugacuucuccaccucagacccuaccacagaguuugaaaaguuccaugccuccaucaggaucccccccuucgacaaugaucuagaucacccaggagaacgucagcagaucucuagcaccagggaacggaaggcagcacgcauccuggggcugauucugggugcauucauuuuauccuggcugccauuuuucaucaaagaguugauugugggucugagcaucuacaccguguccucggaaguggccgacuuucugacguggcucgguuaugugaauucucugaucaacccucugcucuauacgaguuuuaaugaagacuuuaagcuggcuuuuaaaaagcucauuagaugccgagagcauacuuagacuguaaaaagcuaaaaggcacgacuuuuuccagagccucaugaguggauggggguaaggggugcaacuuauuaauucuugaacauacuugguucaggagaguuuguaaguauguguggucuuguuuccuuguuuguuuguuuguuuuguucuguuuuguuugaggauuguuauuuggcgugcuguuuucuaccucuggucuuaucugugauacauaauuucaaauaaacauuaucauacaaaaaca

SEQ ID NO: 47 - RNA, 3319 nt, Homo sapiens
  mRNA (1)..(3319): mRNA
ggagaaggagccgcagggcucugggagaggcggaggcaggugcgggaucgcgagggcgcgagccggaaagggccccggggcggcggggcgggcggcccccgaguucggcgggggucgcggcggagacaaggcggccgcgacggggagacgccgcgcgaggaaguggaucgggcggguacgccgggugugcgcaccugcccgccgggcagcggaucuucugaaugugucccugcucugugagcccacagugaucuguuuguuuugccuggucccagcuuaugcauucuucgagucuugguaaccaggauguuuugucuguucaggcucuaaaacgaucagugggauaggaaccgaaguuaaguagcucaggaugcgucaagaugaaccugggaaaaccgcgagcugcugugcugacuggacugcuucucugaaucggcuggaacuucuuuuugugucuaguuuuaaaagcuccucagaagcaacccuaaauccagaaucguuucaauuacugugaggggaauucugaaguucucauuaaacacacgaaugucucaagcuuugaaaagagccagaugccaaaggcaagaaucccagggcauugcccaccugggugugauccccugauaauugucuaacaaauucagcauguguccccugggcaugaccaucugcugggaaaaguuccaggacccugagacaucuugggauuccugugguuuaggaaagaccuuuaacuaccagcugguaguugucucagcauucuucaaauaguccggucuuguuuaauauuauuauuauuauuguuauuuaauuuuauuuuauugcaacuguacuuagagaauagucuggucuugagaccuuuucacuguggucuguucugguguacggcucccaccagugugaagcagaaggaugacuuugcucuguugucaggacaaccuugaaggaaggagccaaauguguggaggucugugggaagagagagccaccuagcauguccccacugaaccagucagcagaaggccuuccccaggaggccuccaacagaucccugaaugccacagaaaccucagaggcuugggaucccaggacccuccaggcgcucaagaucucccuugccgugguccuuuccgucaucacacuggccacaguccucuccaaugccuuuguacucaccaccaucuuacucaccaggaagcuccacaccccugccaacuaccugauuggcucccuggccaccaccgaccucuugguuuccaucuugguaaugcccaucagcaucgccuauaccaucacccacaccuggaacuuuggccaaaucuugugugacaucuggcuguccucugacaucacgugcugcacagccuccauccugcaucucugugucauugcucuggacagguacugggcaaucacagaugcccuggaauacaguaaacgcaggacggcuggccacgcggccaccaugaucgccauugucugggccaucuccaucugcaucuccauccccccgcucuucuggcggcaggccaaggcccaggaggagaugucggacugucuggugaacaccucucagaucuccuacaccaucuacuccaccuguggggccuucuacauucccucgguguugcucaucauccuauauggccggaucuaccgggcugcccggaaccgcauccugaauccacccucacucuaugggaagcgcuucaccacggcccaccucaucacaggcucugccggguccucgcucugcucgcucaacuccagccuccaugaggggcacucgcacucggcuggcuccccucucuuuuucaaccacgugaaaaucaagcuugcugacagugcccuggaacgcaagaggauuucugcugcucgagaaaggaaagccacuaaaauccugggcaucauucugggggccuuuaucaucugcuggcugcccuucuucguggugucucugguccuccccaucugccgggacuccugcuggauccacccggcgcucuuugacuucuucaccuggcuaggcuauuuaaacucccucaucaauccaauaaucuacacuguguuuaaugaagaguuucggcaagcuuuucagaaaauugucccuuuccggaaggccuccuagucuuauucggugaugacucuuguuaucuuuuguguccuguaaccucaucgggauugucuuuuuuuuuuuuaauuauuuucugagacuuggauuaauucaugguaucuugagucuugguucaaucaacagaauuguucuuuuuccuguuguuuucuuaacuucugaaagagcugugggugguggggggcggggggcaggacugaagauuucacuacuuagcauaauauuuucuuucuuuuuuuuuuuuuuugagacggagucuugcccucucgccaggcuggaguguaguggcacaaucucagcucacugcaaccuccgcaucccggguucaagagauucugccucagccuccagaguagcugggacuacaaacgugugucaccaggcccagcuaauuuuuguauuuuuaauaaagacgggguuucaucauguuggccaggguggucucgaucucuugaccucgugauccgcccgccucagcaucccaaagugcugggauuacaggcaugagccacugugccuggccaacauaauguuuucacaguuccucuuuguuggaagaagugugugaccuacaagauuccugguugauacaguucaggugagaaacuguuugauuauagaaaagaccccagacucagagggaaggcucucuggucuaccugagaucuauauggagcuuaaaggagggugaauaagauuaaugaccugaauugaaagagaauuuggagccaggaugggucuccauucuguguggcaccuugauuggaaaauaauauccaugcguuucuuccacugagucuuucuuagauuaaaucucucccaguuguccgugaagagaaagacaguauuugcagugccgaugauauagagugacuuggagagagauucagaaguuagggguagcggggguaggaugguugggaccagauugacuacccuguacccuuccuucuuccccaauaugccugcgccaucuucacauucugaaguugaaacccaugaucaccccuuugagguagauugaccggcuuuagacgcccaccccccacccaccgcaagcuuugagugggacaccauucugaauuuuuuuucugaauuuuuuuuuaacaucucuuucucauuagagaucugaaaacucauuagagauguugagucuuaucaaauaaaaagcaugagacaucauggcca

SEQ ID NO: 48 - RNA, 2568 nt, Homo sapiens
  mRNA (1)..(2568): mRNA
aaaccugcgaucgccacgguccuuccgcccucuccuucguccgcuccaugcccaagagcugcgcuccggagcuggggcgaggagagccauggaggaaccgggugcucagugcgcuccaccgccgcccgcgggcuccgagaccuggguuccucaagccaacuuauccucugcucccucccaaaacugcagcgccaaggacuacauuuaccaggacuccaucucccuacccuggaaaguacugcugguuaugcuauuggcgcucaucaccuuggccaccacgcucuccaaugccuuugugauugccacaguguaccggacccggaaacugcacaccccggcuaacuaccugaucgccucucuggcggucaccgaccugcuuguguccauccuggugaugcccaucagcaccauguacacugucaccggccgcuggacacugggccagguggucugugacuucuggcugucgucggacaucacuuguugcacugccuccauccugcaccucugugucaucgcccuggaccgcuacugggccaucacggacgccguggaguacucagcuaaaaggacucccaagagggcggcggucaugaucgcgcuggugugggucuucuccaucucuaucucgcugccgcccuucuucuggcgucaggcuaaggccgaagaggaggugucggaaugcguggugaacaccgaccacauccucuacacggucuacuccacggugggugcuuucuacuuccccacccugcuccucaucgcccucuauggccgcaucuacguagaagcccgcucccggauuuugaaacagacgcccaacaggaccggcaagcgcuugacccgagcccagcugauaaccgacucccccggguccacguccucggucaccucuauuaacucgcggguucccgacgugcccagcgaauccggaucuccuguguaugugaaccaagucaaagugcgagucuccgacgcccugcuggaaaagaagaaacucauggccgcuagggagcgcaaagccaccaagacccuagggaucauuuugggagccuuuauuguguguuggcuacccuucuucaucaucucccuagugaugccuaucugcaaagaugccugcugguuccaccuagccaucuuugacuucuucacauggcugggcuaucucaacucccucaucaaccccauaaucuauaccauguccaaugaggacuuuaaacaagcauuccauaaacugauacguuuuaagugcacaaguugacuugccguuugcaguggggucgccuaagcgaccuuuggggaccaaguugugucugguuccacagguaggucgaaucuucuuucgcgguuucugggucccagcgaggcucucucuccugggcaagggcaauggauccugagaagccagaauaguccugagagagagcucugaaaggagaaguguugaaacuaaauguagagcuucccugcccaggaggaggcucacuuccuccccucaagccccgggcucagcacugacccugcggcagccaaucccaaaggggguugcaacuuuuaaaaauugauaauggaagggaaucccugcccugcuuugguaucguggauaaugcccacuagaagcaguguacuuguaauuguugucugaagccugucugagacagaucuacauacagccuggcaguacuugaacuagacgcuuaaugcccuguguuuuuggggggagaacuuuguguuacagcuuaauuuaagaacaguuacuuuggcaucauucagucuucacuuuuugucuauuuaaacuugguuggagaaacuuguggauuuggugcuucaaacccuauguguggcuuggauggcgcagagaaaccuugaagaguuaacagcaaaauucugaugcugagaucucuauuuuuauuauacuugaaacuauaugggggugggugggugggaaugggagaugaggaguguuaaacugagaaucaacaccuaugauuguuuguuuucugcagauuuacaauuuuguaauuccuguuuagcgauugucaagccacaacucuaacaaacaaaccauuaugugugcuagugccgaagucugcagacugcuuuauuuuuucucuuaauuucauguaccugucacuuuacacauuuaaauccccauaaaugaaggguaugaugggugacucagcccacacugcugcuauauuucuuacuaaugcaauugguaaaaccgauuaguauuggaaauauacuguuucuuaacaagaaaagugucuuuauuucuuauccaauuuagugagaugugaaggagacugaugacauggggauaguucuuacacaauugaggaauggggugggggcaauaggaggauguauauuuugacuuguaaaaaaaucuuaaaaugcaugaaacuuuuaucugauagucauuugcacucuccuucccaucugugauuccuugugugcuaacauauaaagaaaccaagagaacuaucuuccuucuccagaaaccuuaaaaauacaguuaagggcccuaaaaacgauauugaaaagaaaauaaacuuguuucuuuuu

SEQ ID NO: 49 - RNA, 4555 nt, Homo sapiens
  mRNA (1)..(4555): mRNA
agcauucgcucuccggagcugcagccuccgaagggguggcgggggcaacagggacagaaggcaggucccagaaagcagguucccccaaaacuggcuucccuagcacggaguuaaggcugcagccggcugccuagagagaagggugggcagggagacaacgcggugagagcgacugcucugacgcaccagccccucuccugcacccacacgcugcuggccgcccagcuucuccccgaucugggagaugcucggcucugggcggccaaacagccuuuccaccaaggcgaggagcccugggcuccugacagcuuaggcgggcccuggcugcgacacgcagccccucgccucugcuuccuuagccucugagggcuucagaccugccgcgcuugcagccacaccaucuccaacggaugcucacuagcaggaaauuggggccaaauucacaggcacuuuccagaaacucccccacuggccagagguugcaaacauccggauuggcucugggcacaguggccgccuuaaguccuccugaacaccccuucugcaaguaccccagggcggucuccugacccagagauggauuuaccugugaaccuaaccuccuuuucccucuccacccccuccccuuuggagaccaaccacagccucggcaaagacgaccugcgccccagcucgccccugcucucggucuucggagugcuuauucucaccuugcugggcuuucugguggcggcgacguucgccuggaaccugcuggugcuggcgaccauccuccguguacgcaccuuccaccgcgugccccacaaccugguggcauccauggccgucucggauguccugguggccgcgcuggucaugccgcugagccuggugcacgagcuguccgggcgccgcuggcagcuaggucggaggcugugccagcuuuggaucgcgugcgacgugcuuugcugcacggccagcaucuggaacgugacggccauagcccuggaccgcuacugguccaucacgcgccacauggaauacacgcuccgcacccgcaagugcgucuccaacgucaugaucgcgcucaccugggcacucuccgcugucaucucucuggccccgcugcuuuuuggcuggggagagacguacucugagggcagcgaggagugccagguaagccgcgagccuuccuacgccguguucuccaccguaggcgccuucuaccugccgcucuguguggugcucuucguguacuggaagaucuacaaggcugccaaguuccgcgugggcuccaggaagaccaauagcgucucacccauauccgaagcuguggaggugaaggacucugccaaacagccccagaugguguucacgguccgccacgccaccgucaccuuccagccagaaggggacacguggcgggagcagaaggagcagcgggccgcccucauggugggcauccucauuggcguguucgugcucugcuggauccccuucuuucucaccgagcucaucaguccccucugcuccugugacauccccgccaucuggaaaagcaucuuccuguggcuuggcuacuccaacuccuucuuuaacccccugaucuauacggcuuucaacaagaacuacaacagcgccuucaagaacuucuuuucuaggcaacacugagggagaggaccaggauugaaaaaaguuucuucccauaauucaguggaauucccaguucauccauuucccauccccacccaacagccauguggacgggaugaauccucaccauucuccagggucaucuucagaacugcacuggccucuuuuccaccuccucaguaggaauaugacuccucauagaguuacggugacaugauguaucuaacuuaucuacuaucccuuucucugugcugacagucauggucuuugcccgcaaaguguccuuuccuccccaaauucacucuggcauggugaucgacauugucuuaaagaagucaaggcaaauaagaaggaggagguaaaacaauaugggcagguugagauugaauagaaaagaaugaacaagucaugauguagaccucugaggguaggagaccauaguuccaggcuuucuggcacaguccccauuucaaauauucucugugguccuaguacauauauccuaauacugaugggguaugacccaagaguugaccacaaaacacugucuccaaauauauuaaaguauauaguccuauccaugagagcagggaaucuuagcuuuaagguuuuguuuguggcaaccacacacgaacauacacacacacacacacacaacuaugaaagaggcagguguucuagcuagcuaguuuacuccaaaaugcaauccguugacaugaaacaggcacugaggguaacuacuuccuuucauuuugaagcaacaguuuauaaacaggaaagucuacugguuucauagaaucugucagauuuguuuauuugcuacugguuucacgcgcguggacuauauuccaguguuucuagucaaagaccuuuauccucaaaucaugagauuagaguaaaacaacugcuguaagguugagcauuucaugaccaaugacuaugaaggcacauucaccaaacacaggcacuuuaagggcauccuugauuaauuugcuaauugcuauuuuuuguugguaugugaauuccugcuaggaggccgaggcuaguaggcaaugaaaaagacagaacaaugguaaaaugcagccccaguccucuacaaauugacaguauaauuuaugaggugaggcaguuaagugaugaugcagcauuuggaugaucauguaaaaccagugucauuacacaaaacuuaaaaaaaaauccuaggcaauaaaacugccuguggucugcuaugucucugacucuucaaaugcaugaauuaaagaaugauauaauaaucucugaauuauuguaauucauguaguauauuguauauaauaaauuauaucaauuaagaguucaaacauaggaaauuugugauaauuuauauguggucuuggcuugaaguuuagucuugucuuuauugcaaagacuagcuuuaucaaaagaaugugagcagauaaagaaauguuagacaccugugaagucacaauuaauaugcuaaaugcagaucuuuaugcauucaagcagauucauugagaaauuuagugauaagaauugaagaauuuauuuagaaacguccuuagauuuuucaucuguauauaucacuugaagugcuaaacccacaaguauuuaaaauacagacucuucacuuacucaaguuacuuuuccccauuauuacucgaaaucagagagaauaacaaaugcuauuguuuucuaguguuuugaauaagggaaguguuugaauaagggaagggaaguguuugaaaaaccuuauuaaguaccccauggcagagaagaaaaauucagacaugaggagucuggagagguaacacuagaaucaaggguaaacgcuuaagucuauucuaccugaaaauccaugugaaaacuuuuaauccauaaaucgugagugucuuacuguuuaaaaaaguucauacucuucaguaaagcgaagcuugaggaagaugugccacauuuuucugguggcuucaaacacuccccaacucuccaaggauucuguauaccccaguuggagaacgauguuauuauaauauuaauucaaucuccagcugaggguuuuauuaauguuuucauaagauggauaagccucauuauucuuaucacucaauauguauuuaacaccucucaggugucacuuaaauuauacauuauauacaacauacuccacauacacuaucuagagcaagaaacauuccacauaagagaaggaagaaguggcaauuaaugaauuauuuugcuuccauuucaauaguaaaaguuguuuauacccugguaagaaauguagacauucauugacuuugagucucuacuaugcaaaucaaauauuugugaaacacucucauuuaugcuugacugcuaauauuuuaucagguuagaaauuuauaugaaucauaaacagaaguaauugcucaaaauaacuaauuguguucccgugccagauugcaacaaccaccuuuauuagauggugcaccuagagaaggcuggaggguuuccugcaagcccucucuguagggugacugaggugaaccaauccaaagcccuccuuucagcucuuguagaauaugucgaacaaaacgcacaguucugcccuccugccuaaagacaaaguacaaaggagauaauccucuccuuaugagugaucuuagcccucuugaggucacuaucaguuuuauauuuucauugugcgguagugaaagacaaguggaagaugucccugagucuguauugacaucagaccauauuguagggauuaauucuugauaaaaaaaaagcuuguuauuucucucuucuuuucaugaggagcauuuuugcuuccguguuugucuguuggauuugcaguucccugcucacagccaaagaggcuguagagacaacacuuugccauauuucuauuaaagagaaaauaaagauugaguccacagcucaa

SEQ ID NO: 50 - RNA, 3467 nt, Homo sapiens
  mRNA (1)..(3467): mRNA
gcacuccgcaacuucugccgcugccgcccgggcgcucucggcgcagccgagcugcugcugcgaacuguggagcugccagcggagcccagcccgagcucccucgacuccgcgcgucccaggcguccgucgggggccgaagcgccucccgcagggaguccccgugaggccgcgcugccgcguuacccgcagcggccggggguccggggagugcggggcuccgagacagagccaggcgccccccagcggucggcgcgggccccauggcugggccgggcggagcggaaccggugaggugaagccccggggccggcagccggaggcgcguggccggggcgccggcuccaugggcagcggcacacggcggcgcgaugauggacguuaacagcagcggccgcccggaccucuacgggcaccuccgcucuuuccuucugccagaaguggggcgcgggcugcccgacuugagccccgacgguggcgccgacccggucgcgggcuccugggcgccgcaccugcugagcgaggugacagccagcccggcgcccaccugggacgcgcccccggacaaugccuccggcuguggggaacagaucaacuacggcagagucgagaaaguugugaucggcuccauccugacgcucaucacgcugcugacgaucgcgggcaacugccugguggugaucuccgugugcuucgucaagaagcuccgccagcccuccaacuaccugaucgugucccuggcgcuggccgaccucucgguggcuguggcggucaugcccuucgucagcgucaccgaccucaucgggggcaaguggaucuuuggacacuuuuucuguaaugucuucaucgccauggacgucaugugcugcacggccucgaucaugacccugugcgugaucagcauugacagguaccuugggaucacaaggccccucacauacccugugaggcagaaugggaaaugcauggcgaagaugauucucuccgucuggcuucucuccgccuccaucaccuuaccuccacucuuuggaugggcucagaauguaaaugaugauaaggugugcuugaucagccaggacuuuggcuauacgauuuacucuaccgcaguggcauuuuauauccccauguccgucaugcuuuucauguacuaccagauuuacaaggcugccaggaagagugcugccaaacacaaguuuccuggcuucccucgaguggagccagacagcgucaucgcccugaauggcauagugaagcuccagaaggagguggaagagugugcaaaccuuucgagacuccucaagcaugaaaggaaaaacaucuccaucuuuaagcgagaacagaaagcagccaccacccuggggaucaucgucggggccuuuaccgugugcuggcugccauuuuuccuccucucgacagccagacccuucaucuguggcacuuccugcagcugcaucccacuguggguggagaggacauuucuguggcuaggcuaugcaaacucucucauuaacccuuuuauauaugccuucuucaaccgggaccugaggaccaccuaucgcagccugcuccagugccaguaccggaauaucaaccggaagcucucagcugcaggcaugcaugaagcccugaagcuugcugagaggccagagagaccugaguuugugcugagggccugcacaaggagggugcuguugagaccagaaaagaggccaccgguaucugugugggugcuacaaucuccagaccaucacaauugguuagcagacaaaaugcugacuacuguagaaaaaaaggucaugauucaugauugaaagcagaacaauggagaugaaauaaacaaggcaaaauagagguggaaacagaaggaagucauuugcugagucugcagaauggaaugcagcuucuguccuuucuugggaugucuaaaacaugacaaacagggugaucuguuguacacacuaucuuaugagggagauggugacuucuccuuuuuucuguggaucagugcuauuguguguucucaguuuaagauagcagaucaucucagcaguaagcacaccaacagaacugaguuccagaaaggaagcaguuucuggugcuuugcauauguccaaauccaugcaagugggagaaaguuccagugcacacuucccaugcuucugagucuaggcguuguggugaauaugcaggaaucauucaugagacagaauguauuuuguuguaugacagaaggcuuuuccaagcaaacugcggugagcguaguguugaauauguugcauguccauguuagaaaacagaauccagucaucagcuaauacaaaugauuucccagagcaguguuuguuucaagcuucugucaaauaguuuggcuuuucugcagggucucugugauuuccccaccuauguacuuuucuguguuaacucacuuauuaugguauaacucaggaacaggucucaggauggagagaagcauacaacaugagcaaagcuuuuucucgugcucugaagggucuugagaggggagugggaugccuucugugaggauuuuuaucgcagugaagcucaagugguacccaacugcguuguaauuaacaugauuauuggcauguuuuugacucucauuuucuacaaggcugcagugggcccacccaugggcuggcccauaacuguauguuauguggaagaagugugggaaugauggguuuuccaugaaaugugaugcuucuggagauggggcauaaacauguucauucuuguugagcugcauuguguuaaaccaaauugugugaagcgaggucaccuggaccacucuugggaaaugcuucagaacaccagucuguuccuucuagagaauuuguuuuuguguucuauguaucuuaaaucaaaugugugccuaagcaauucuguccucugauucccaccccuacccaccaaaaaaugguagacaugaaaggcaggugaggaaacagaaaaggucucuuugcagauauauauuuuuuaaauggcacuauguaguaauuaagagcuaaggagacagaaagacaguagaugagaauagaagaguaccuguaccuacaaggggaaaaaugaaugcaauucuuuauuugauaagagugaacugaaagagaauauuauuuuccuacaauaaagcaagcuguccucucacaggauaaaguggaauucugacauggauuauggacuucuucuaacucccauugacauugcuucuuaccagacauggggaaaagcuaccuuuccugaaaggggaggagggcacuguccuuucagacaaagcuuguacaaauuccugaaccgcaaauuugcugaagugaccguauauauuauauucauaauuuaaaugauuauuuauggucagauacauauuguuaaacuugaaaauguuuuauuacauuacaucaaauaaaguuuauuuguagcugaaauaaagcaacguaaguaacaauuuuuaa

SEQ ID NO: 51 - RNA, 4572 nt, Homo sapiens
  mRNA (1)..(4572): mRNA
guugacaaaaagagacucgaaugcaaagacgcugagcuagagggagaggagggcggggacccagaggaaagaggcacuccucgggguuggggaaguauuaggaggggaggguuagagugggagggaaggagccuggcuuucgaagcgacucacagagggauaaauaaagggaagugaggaggaagagggagacugaaagggaaggcagguggggagaagggggacgaaagaggcagaagagagagaagagaggaggagagagggggagagagggaaggaaggaaauagggagaggagggucacagagugaccguggaggauggggcuucucgguucuagauauuucugggauuggagacuguuugcuaguggggagacuccagcuccggcagccaguucgggagcggcaaaguaaaauggacagcgacagacagacguuccagccaccucuccgccgccgggagauccuggagcugcuuucaggccaacuccaguuucccagcuggagcuucugaacgcgcuggacugcgagagcccagggagcgccugaaagcugcuccucggagauacccuucgccgaagcaguaagaacuuccugcuugggucucugcauucccuuccuccgaaacuucccaggagaagggcggaagaccccaggggaaggggcgaggcgaaucuucgcgcugcuuuuucuucccucccccuucccgcgccgggcgcgcaggcauggaugugcucagcccuggucagggcaacaacaccacaucaccaccggcucccuuugagaccggcggcaacacuacugguaucuccgacgugaccgucagcuaccaagugaucaccucucugcugcugggcacgcucaucuucugcgcggugcugggcaaugcgugcgugguggcugccaucgccuuggagcgcucccugcagaacguggccaauuaucuuauuggcucuuuggcggucaccgaccucauggugucgguguuggugcugcccauggccgcgcuguaucaggugcucaacaaguggacacugggccagguaaccugcgaccuguucaucgcccucgacgugcugugcugcaccucauccaucuugcaccugugcgccaucgcgcuggacagguacugggccaucacggaccccaucgacuacgugaacaagaggacgccccggcgcgccgcugcgcucaucucgcucacuuggcuuauuggcuuccucaucucuaucccgcccaugcugggcuggcgcaccccggaagaccgcucggaccccgacgcaugcaccauuagcaaggaucauggcuacacuaucuauuccaccuuuggagcuuucuacaucccgcugcugcucaugcugguucucuaugggcgcauauuccgagcugcgcgcuuccgcauccgcaagacggucaaaaagguggagaagaccggagcggacacccgccauggagcaucucccgccccgcagcccaagaagagugugaauggagagucggggagcaggaacuggaggcugggcguggagagcaaggcugggggugcucugugcgccaauggcgcggugaggcaaggugacgauggcgccgcccuggaggugaucgaggugcaccgagugggcaacuccaaagagcacuugccucugcccagcgaggcugguccuaccccuugugcccccgccucuuucgagaggaaaaaugagcgcaacgccgaggcgaagcgcaagauggcccuggcccgagagaggaagacagugaagacgcugggcaucaucaugggcaccuucauccucugcuggcugcccuucuucaucguggcucuuguucugcccuucugcgagagcagcugccacaugcccacccuguugggcgccauaaucaauuggcugggcuacuccaacucucugcuuaaccccgucauuuacgcauacuucaacaaggacuuucaaaacgcguuuaagaagaucauuaaguguaaguucugccgccagugaugacggaggaguagccggccagucgaggcuacaggauccgucccauucacuaugcuucccccaacccuagggaaucaacacuuaagauaauucgccacuucuccucuuucucucugcuccgcucacggcuugcagaccugguccccuccccacuuccugcuccacggcagggcccuuugugcaaaggagacccagcggaggagcguugagagcccaggaaauucagagaguuugugagaagcgacauuggcucagacuucgccuguaucaucaguuuugaucccaguaauugccucuucucucuucuaucuccuaaaucuuugcgguggauguuuaaugcuuaguucaaggcagaaaauccaaaaaauaaauaaacuguacacacagccgccgcgcccacuaaggggcuccaccccaucacccaacuucuagaacuuggacuugagguuugggaauuugcuccaucucucccgcccuguggcccuugaaauauucaccuuuuucuccccauuuuuaucuuuucuuuuuuucauugcuucccucucugcccucgcccauccuuaugcacgaaucugcugguccggagaggcggaagaggcugcuuuuccccgugccagcggugcccccgggauuccccgucuucucgcagacuugagauuaacucucucgugcagugcacaaccccuauuuccuuuguuuccuuggacucaaagcaaguggcuagaagguaucuuuuagaguggacuuguaaguacaaggccgggccagugggggagagcaaggaggaggagggugugcagaccacuuuguuuaugcuugccuuggugucagcacucaccaaagaaacugacaaucauacaguggagagagcggacgcuguuuguacagguagcuggcgaguguguacgaccugacaucgccacuuacuuacugcgggaggcgccagccggccgcuguuugcgcucuggggaagcugagaggccuggaauccgcgcgccuccccagccaucugugcucucgcagcgcgcuaggcgcucccggcugcugacuuacaggaucuggguuccuccgccuacgcucuggggcgcccugaagcgcggguagacaccugagguuuuugccuuagccaguggcgcaagaugccgcggaacucuugggauuggagagaccaagaaggcuuuaaaggggcgacacgcaggggcggcaaagaggaaagguuccccagggcgcgcgugcccaaggcuccagcuggcucccccaguaaaaccugaaagagcaauuuuauccuuacgauguagaauuuuuccuccuaauucacgcucuuguccuuuuuggaucggggaauaauucuguuuagcgugucccaacacucgcugcauuugcuuuacaggacauccaagacacaaaguuagcagaacuuuguuuuccaaauaggaauucugggcuuucaauggaaugcccagauaguccauagguuagcacuaucugugacaacaggcagauaguuuaaauagcaggguccuaaaggacuaguuaggagguggucuuccuggggaggcugagucuuuuaugggauuaauugcucccuagcgugaccauucugcuguaccaagggucugcggaaaccaccuuuuugcccuuugaauugugcagcauuccaggcuuucucuccuucaguuauucuugaagccuugccacuccugccgagaaacugguuaaaaucuuugcugccccagaaagggaaguuauuuagguacucuggauguaaauuuccaacuacaugauggagaucuagagguuugucauuguuugaucuagccuccaauuuucuuaugaauaauggucuuguuuuccagaaaauucacaaaguaauugaucccuacucuuuucucucuucagcuguaccuuucgaauguuuucauuugcuguuuugagaaccguauuacauuuacagauugcaaaaauugccagugaaaaguucauuuguuuauaauuaagaaagugaaaggacagcucugucaguugaagccauuuuauuguuugucagacugaacaaagcuuuccuuuggugucugcuucuguauucuaaacuggaaguagacaucauaucccagcuaaacagggaaaguuuuagaacuuugggaauaguuugucuaugcauguauaaugcugagaagggucugcagugaagaagauaagauagacuucuccagcacaacuauuacucauuugauaauucaguuucauuuuugcucacauaaucuaaguaaagccauaucagaaaccaaguuuuucuguauguuacacuaaauuuuaagaacaaagucuauauuuaaaauaauccaaaccccaaauuugcucauccaaauuaugucuuggccagcaaguuuuacauuagaaguuuugagacuuccauuuauuugugucuuuuacagaauugugugacaaugacuuuuggacauuuguucuuucggcuuuggaauauuuacacaagaauggagaaggaacauccaacuggagcauaauugucaaggcaauagagaauuuagagaaauaaaauagcugcauaucucaaaa

SEQ ID NO: 52 - RNA, 5415 nt, Homo sapiens
  mRNA (1)..(5415): mRNA
agagagcuacuccauucccguggaaaccaggagucccuuggugcagacagcucuuccuacuuucccaugcaguucuuuugugcgacuuugaggggcucgugaaugauuucuaaaugugugccugcugaggcgagccgcacagggagggaggaacccagccgagccgugccagaggaagccaacaggauccuagcagugcgggagcuggcucagcucuugcaugcaguuuuugaagucagcaaaacagaaaccaaauuacuaucauauuaugcugguggaagaucaagaagaggggacucuacaccaguuuaauuacugugagagaugcagcgagucacagaauaacaaauguaucucauguguggacccugaagacaaaugacauuuaucuucccgagcgcucaaaaaaaacccugcaaccucuaugcuaaaaguucauucugcuuuuuuguccucgguuuggugagaaaauaauaaaaccaaacaguggacucuccuaaaauugugaaugaagaaaacuuacagccaccacaguucaguucuuuaacuaucauuguaauaauggaagacaaaaauccagccccgggagaacagcauguacaccagccucaguguuacagaguguggguacaucaaggugaauggugagcagaaacuauaaccuguuaguccuucuacaccucaucugcuacaaguucuggcuuagacauggauauucuuugugaagaaaauacuucuuugagcucaacuacgaacucccuaaugcaauuaaaugaugacaccaggcucuacaguaaugacuuuaacuccggagaagcuaacacuucugaugcauuuaacuggacagucgacucugaaaaucgaaccaaccuuuccugugaagggugccucucaccgucgugucucuccuuacuucaucuccaggaaaaaaacuggucugcuuuacugacagccguagugauuauucuaacuauugcuggaaacauacucgucaucauggcagugucccuagagaaaaagcugcagaaugccaccaacuauuuccugaugucacuugccauagcugauaugcugcuggguuuccuugucaugcccguguccauguuaaccauccuguauggguaccgguggccucugccgagcaagcuuugugcagucuggauuuaccuggacgugcucuucuccacggccuccaucaugcaccucugcgccaucucgcuggaccgcuacgucgccauccagaaucccauccaccacagccgcuucaacuccagaacuaaggcauuucugaaaaucauugcuguuuggaccauaucaguagguauauccaugccaauaccagucuuugggcuacaggacgauucgaaggucuuuaaggaggggaguugcuuacucgccgaugauaacuuuguccugaucggcucuuuugugucauuuuucauucccuuaaccaucauggugaucaccuacuuucuaacuaucaagucacuccagaaagaagcuacuuuguguguaagugaucuuggcacacgggccaaauuagcuucuuucagcuuccucccucagaguucuuugucuucagaaaagcucuuccagcggucgauccauagggagccaggguccuacacaggcaggaggacuaugcaguccaucagcaaugagcaaaaggcaugcaaggugcugggcaucgucuucuuccuguuuguggugauguggugcccuuucuucaucacaaacaucauggccgucaucugcaaagaguccugcaaugaggaugucauuggggcccugcucaauguguuuguuuggaucgguuaucucucuucagcagucaacccacuagucuacacacuguucaacaagaccuauaggucagccuuuucacgguauauucagugucaguacaaggaaaacaaaaaaccauugcaguuaauuuuagugaacacaauaccggcuuuggccuacaagucuagccaacuucaaaugggacaaaaaaagaauucaaagcaagaugccaagacaacagauaaugacugcucaaugguugcucuaggaaagcagcauucugaagaggcuucuaaagacaauagcgacggagugaaugaaaaggugagcugugugugauaggcuaguugccguggcaacuguggaaggcacacugagcaaguuuucaccuaucuggaaaaaaaaaaauaugagauuggaaaaaauuagacaagucuaguggaaccaacgaucauaucuguaugccucauuuuauucugucaaugaaaagcgggguucaaugcuacaaaaugugugcuuggaaaauguucugacagcauuucagcugugagcuuucugauacuuauuuauaacauuguaaaugauaugucuuuaaaaugauucacuuuuauuguauaauuaugaagcccuaaguaaaucuaaauuaacuucuauuuucaaguggaaaccuugcugcuaugcuguucauugaugacaugggauugaguugguuaccuauugcuguaaauaaaaauagcuauaaauagugaaaauuuuauugaauauaauggccucuuaaaaauuaucuuuaaaacuuacuaugguauauauuuugaaaggagaaaaaaaaagccacuaaggucaguguuauaaaaucuguauugcuaagauaauuaaaugaaauacuugacaacauuuuucauuccugcuuuuucauagauaccauuuugaaauauucacaagguugcuggcauuugcugcauuucaaguuaauucucagaagugaaaaagacuucaaauguuauucaauaacuauugcugcuuucucuucuacuucuugugcuuuacucugaauuuccaguguggucuuguuuaauauuuguuccucuagguaaacuagcaaaaggaugauuuaacauuaccaaaugccuuucuagcaauugcuucucuaaaacagcacuaucgagguauuugguaacuugcugugaaaugacugcaucaugcaugcacucuuuugagcaguaaauguauauugauguaacugugucaggauugaggaugaacucagguuuccggcuacugacagugguagaguccuaggacaucucuguaaaaagcaggugacuuuccuaugacacucaucagguaaacugaugcuuucagauccaucgguuuauacuauuuauuaaaaccauucugcuugguuccacaaucaucuauugaguguacauuuaugugugaagcaaauuucuagauaugagaaauauaaaaauaauuaaaacaaaauccuugccuucaaacgaaauggcucggccaggcacggaggcucgugcauguaauccuagcacuuugggaggcugagaugggaggaucacuugaggccaagaguuugagaccaaccuggguaacaaagugagaccucccugucucuacaaaaaaaaucaaaaaauuaucugauccuuguggcacacaacuguggucccagcuacaggggaggcugagacgcaaggaucacuugagcccagaagcucaaggcugcagugagccaaguucacaccacugccauuuccuccugggcaacagagugagacccuaucacaaaaaaaaagaaagaaaaaaagaaaaugacuaaacugaaauggucuuagucaaaaaauauauacagacuuauuuuuagaaucauuucaacuuauuuccuucuuaccugguucagaaacaaguuuguuucaacugugagaguaguuuuaaaaauaaguuuuccugauugguuaccaauaaacaccaaacaaauaaaggagggggaaaauaucacuugaggcuuaacauugcaauuaauuuagaaauacgcaccucaaucucaagccaguuuauaacuaccauaguuuugagcaaaauuuuucuucuauuucuccaaaaacucacucauuucaguauuacucauugacacugggcucccuaauagaaaagcagauauuauaauauuucauugaaaugguuacuaaagagaaaaagagauuaacaacaguuaauccaccuugauauuauaacacucaagguuaaauugcauguuguuauuagaaucaauguuauauauaugauuacaaugcugaagcauuucuauugaaugagaaauaguguuugcccuaaaccuuugggaaaaaaauacagauuugaaaacuaaagaaugugggaagugucauuguguaauuuggaaaugcauaaaauucucauuauguccucauagauauguugaaagaugguucacuguuucaaagauagaauguauaauaugcugcuaguaccaaggcugcauuugaacugaacuacuccuuaugauauaccauacuaacugaugaaaauauaaauguuaaaaugucuccacucuaauguauuuugccuucacuuguaacucugcacauuucauuauauauuagggaaaagauuacagauucuacccccauauacucaaucuugaucagaauguacauuaauguugccugauagaugucuuuugacuaccuauauauuacaaauguguauguguuuuuuucuuccauaaguaucuauucaaguguauacuguauaggcaaauguguagcuguauauauguacagacauguucucaugcacuuuauucaauauuuauauauuuauauggacgaaaagcaagucaaugaaaacacucaguauauacacaugugugcauguuuguaucauguuuauuuaaaugugaauucauguucuaauauuucuaugaccuuccauugcugcaaagcagauuaaaguguaacaaaaauaucagcucuuuaaauaaacuggaaauuaaucuaauuuuguggaagaacuuuaagacaagucuaugaggauggcgaaaacaaugguccacauauauaguuucuugauccuaaauaaaaugguaccuuugggcccuuuguuuagucuggcaauuuucugacuacuauggcuuuuuaaacagacucacaccuaagugaaacacaguugucaaaugauuuauggaucugugauuaaaggauucacuuuuaaugaauacuauuaaaacggaguaacaaaccuugcucuacuuuaaguugaaugauagauuccucuauauucaaaaaaaagcaauuaacugccauaauuagggaaaacuguuuaaagcauacugcauuuacuuggccacagcgccaucucaugguaauuuuuuaaagugcuaccguagccaaauugaacuaagucacuguacugcuuucagcaaagggugcuccucccauuugugcaucaaugagacauauuuauaaagugcuaaauuauucugugccauauguaacaaauacagugaagauuauuuuaugaacuuauuuuaaucaaggcgaugcuaaaaguuuucaagaaaggauaaauaacuguaaauaaaguagacucaaaaaugaa

SEQ ID NO: 53 - RNA, 2770 nt, Homo sapiens
  mRNA (1)..(2770): mRNA
gauagagagacaaagaggaaaagagagcgagguagaaaacggauacugccuaugccuacuccaucccucuuucagcaccaaggacagaaccucugagcggcugacccaagcaacgcucaguuuagggucccucccaaauccucuaaagaaaacggauacauucgaaagcagcuaugaaacaugcacuaaggucuaauagggaagcuggaaaagcagcacucaaguaauuucaccuuagaggcaaaaaugggugauuucuuucuguucauuucauaguuucugaguccugagaaaggcaaaguuugcuuugcuuggguaugucugcugucaguaaauggcugcaggagccgaagugguaaacuccucggucuccagaaaucagaagaaaauuuuagggaagccccuuggcaucacgcaccucccucugggcuauggcaucucugagccagcugaguggccaccugaacuacaccuguggggcagagaacuccacaggugccagccaggcccgcccacaugccuacuaugcccucuccuacugcgcgcucauccuggccaucgucuucggcaauggccuggugugcauggcugugcugaaggagcgggcccugcagacuaccaccaacuacuuaguagugagccuggcuguggcagacuugcugguggccaccuuggugaugcccugggugguauaccuggaggugacagguggagucuggaauuucagccgcauuugcugugauguuuuugucacccuggaugucaugauguguacagccagcauccuuaaucucugugccaucagcauagacagguacacugcaguggucaugcccguucacuaccagcauggcacgggacagagcuccugucggcgcguggcccucaugaucacggccgucuggguacuggccuuugcuguguccugcccucuucuguuuggcuuuaauaccacaggggaccccacugucugcuccaucuccaacccugauuuugucaucuacucuucagugguguccuucuaccugcccuuuggagugacuguccuugucuaugccagaaucuauguggugcugaaacaaaggagacggaaaaggauccucacucgacagaacagucagugcaacagugucaggccuggcuucccccagcaaacccucucuccugacccggcacaucuggagcugaagcguuacuacagcaucugccaggacacugccuuggguggaccaggcuuccaagaaagaggaggagaguugaaaagagaggagaagacucggaauucccugagucccaccauagcgcccaagcucagcuuagaaguucgaaaacucagcaauggcagauuaucgacaucuuugaagcuggggccccugcaaccucggggagugccacuucgggagaagaaggcaacccaaaugguggccauugugcuuggggccuucauugucugcuggcugcccuucuucuugacccauguucucaauacccacugccagacaugccacguguccccagagcuuuacagugccacgacauggcugggcuacgugaauagcgcccucaacccugugaucuauaccaccuucaauaucgaguuccggaaagccuuccucaagauccugucuugcugagggagcagaagagggaacacucuuuguacccauuucuagcugccaggcuguuggcccacucagaaagacugugcaguguccuccggcaugcaguaggaagaagauuccuggagccaggaugucccugcuugucagguauaaccagauguguccuccaaaacagaauauucuaccuccccagcuaaaaucugacuucauucugacaaucuucaaaugacuggggagcuuccaagugauaagcaauaauucagaaagaagcugauaaaacaugacucacacaaaauugucaucagaggcaugauucauuacacugaaggaaaaugaacagaucucaucauucucaacuugcagggguccuugaaaucagccaguaaaccaccuucuagaacagccagucugucuuccuucaaaccucuaauuaacugucaggcuaaccuuguacuuuuacgggcccauuccucuuggucauuucuuuccuucacuguaucuggaacugugaauggcaaggccagugcuucuaccucucuguagaauccgaugggcuacauuuggugaggugaauuucucacaccaagagaaacacugucccacauugggaugggggcuucauuucaaacuuugcuacccuuguauuuuagcuuuagcuaagaaggcuuucugauuccauuccuuuacucucuagaggaaaucucagagucucaacuuccagaaucauucaagauggggauauuuucugaagacggaaugacacaucccauuguaguuucacuguugaaacuuaugguucagaacauaccuuagauaugcuuuucauaacacuaccaaauuuuccuacccagggcaucuccauuucugcuuaacagggacccaauauaguggaccacaucaugacauuuugggcaacaguggaccacauauacaacgguggucucauaagauaauaauggagcugaagaauuucuaucacuuaaugaugucauagcuguugcaacaucauagcucaaugcauuauuuacaugucuguggugaugcugguauaaacaaaaugaugcugccagucguguaaaaguauacaauuguguacaguacguaauacuuaauauugauaauaaaugacuauguuacugguuuaugua

SEQ ID NO: 54 - RNA, 2808 nt, Homo sapiens
  mRNA (1)..(2808): mRNA
agugccgaaccggcagccucacgcgcgcaccgcgccgccuccgccccguccccgcgcucccuccugcccgcccgccccgcgcccggccccgccccgccccgccccgccgcggcccguccacugcuccccgcgggccagagccggccgagcugcugcccgccggggcucugaacggcgcggcggggccgggagccagggaccggccgaggagaguggcggccccggacggcugccggaggggcggccgcgcguggaugcggcgggagcuggaagccucaagcagccggcgccgucucugcccccgggcgcccuauggcuugaagagccuggccacccaguggcuccaccgcccugauggauccacugaaucuguccugguaugaugaugaucuggagaggcagaacuggagccggcccuucaacgggucagacgggaaggcggacagaccccacuacaacuacuaugccacacugcucacccugcucaucgcugucaucgucuucggcaacgugcuggugugcauggcugugucccgcgagaaggcgcugcagaccaccaccaacuaccugaucgucagccucgcaguggccgaccuccucgucgccacacuggucaugcccuggguugucuaccuggaggugguaggugaguggaaauucagcaggauucacugugacaucuucgucacucuggacgucaugaugugcacggcgagcauccugaacuugugugccaucagcaucgacagguacacagcuguggccaugcccaugcuguacaauacgcgcuacagcuccaagcgccgggucaccgucaugaucuccaucgucuggguccuguccuucaccaucuccugcccacuccucuucggacucaauaacgcagaccagaacgagugcaucauugccaacccggccuucguggucuacuccuccaucgucuccuucuacgugcccuucauugucacccugcuggucuacaucaagaucuacauuguccuccgcagacgccgcaagcgagucaacaccaaacgcagcagccgagcuuucagggcccaccugagggcuccacuaaagggcaacuguacucaccccgaggacaugaaacucugcaccguuaucaugaagucuaaugggaguuucccagugaacaggcggagaguggaggcugcccggcgagcccaggagcuggagauggagaugcucuccagcaccagcccacccgagaggacccgguacagccccaucccacccagccaccaccagcugacucuccccgacccgucccaccauggucuccacagcacucccgacagccccgccaaaccagagaagaaugggcaugccaaagaccaccccaagauugccaagaucuuugagauccagaccaugcccaauggcaaaacccggaccucccucaagaccaugagccguaggaagcucucccagcagaaggagaagaaagccacucagaugcucgccauuguucucggcguguucaucaucugcuggcugcccuucuucaucacacacauccugaacauacacugugacugcaacaucccgccuguccuguacagcgccuucacguggcugggcuaugucaacagcgccgugaaccccaucaucuacaccaccuucaacauugaguuccgcaaggccuuccugaagauccuccacugcugacucugcugccugcccgcacagcagccugcuucccaccucccugcccaggccggccagccucacccuugcgaaccgugagcaggaaggccuggguggaucggccuccucuucaccccggcaggcccugcaguguucgcuuggcuccaugcuccucacugcccgcacacccucacucugccagggcagugcuagugagcugggcaugguaccagcccuggggcugggccccccagcucaggggcagcucauagaguccccccucccaccuccagucccccuauccuuggcaccaaagaugcagccgccuuccuugaccuuccucuggggcucuaggguugcuggagccugagucagggcccagaggcugaguuuucucuuuguggggcuuggcguggagcaggcgguggggagagauggacaguucacacccugcaaggcccacaggaggcaagcaagcucucuugccgaggagccaggcaacuucaguccugggagacccauguaaauaccagacugcagguuggaccccagagauucccaagccaaaaaccuuagcucccucccgcaccccgauguggaccucuacuuuccaggcuaguccggacccaccucaccccguuacagcuccccaagugguuuccacaugcucugagaagaggagcccucaucuugaagggcccaggagggucuauggggagaggaacuccuuggccuagcccacccugcugccuucugacggcccugcaauguaucccuucucacagcacaugcuggccagccuggggccuggcagggaggucaggcccuggaacucuaucugggccugggcuaggggacaucagagguucuuugagggacugccucugccacacucugacgcaaaaccacuuuccuuuucuauuccuucuggccuuuccucucuccuguuucccuucccuuccacugccucugccuuagaggagcccacggcuaagaggcugcugaaaaccaucuggccuggccuggcccugcccugaggaaggaggggaagcugcagcuugggagagccccuggggccuagacucuguaacaucacuauccaugcaccaaacuaauaaaacuuugacgagucaccuuccagga

SEQ ID NO: 55 - RNA, 1399 nt, Homo sapiens
  mRNA (1)..(1399): mRNA
guuguccgcggugcucagcgcccgcccgggcgcgccauggggaaccgcagcaccgcggacgcggacgggcugcuggcugggcgcgggccggccgcgggggcaucugcgggggcaucugcggggcuggcugggcagggcgcggcggcgcugguggggggcgugcugcucaucggcgcggugcucgcggggaacucgcucgugugcgugagcguggccaccgagcgcgcccugcagacgcccaccaacuccuucaucgugagccuggcggccgccgaccuccuccucgcucuccuggugcugccgcucuucgucuacuccgagguccaggguggcgcguggcugcugagcccccgccugugcgacgcccucauggccauggacgucaugcugugcaccgccuccaucuucaaccugugcgccaucagcguggacagguucguggccguggccgugccgcugcgcuacaaccggcagggugggagccgccggcagcugcugcucaucggcgccacguggcugcuguccgcggcgguggcggcgcccguacugugcggccucaacgacgugcgcggccgcgaccccgccgugugccgccuggaggaccgcgacuacguggucuacucguccgugugcuccuucuuccuacccugcccgcucaugcugcugcucuacugggccacguuccgcggccugcagcgcugggagguggcacgucgcgccaagcugcacggccgcgcgccccgccgacccagcggcccuggcccgccuucccccacgccacccgcgccccgccucccccaggaccccugcggccccgacugugcgccccccgcgcccggccuuccccggggucccugcggccccgacugugcgcccgccgcgcccagccucccccaggaccccugcggccccgacugugcgccccccgcgcccggccuccccccggaccccugcggcuccaacugugcuccccccgacgccgucagagccgccgcgcucccaccccagacuccaccgcagacccgcaggaggcggcgugccaagaucaccggccgggagcgcaaggccaugaggguccugccggugguggucggggccuuccugcugugcuggacgcccuucuucguggugcacaucacgcaggcgcuguguccugccugcuccgugcccccgcggcuggucagcgccgucaccuggcugggcuacgucaacagcgcccucaaccccgucaucuacacugucuucaacgccgaguuccgcaacgucuuccgcaaggcccugcgugccugcugcugagccgggcacccccggacgccccccggccugauggccaggccucagggaccaaggagauggggagggcgcuuuuguacguuaauuaaacaaauuccuucccaaa

SEQ ID NO: 56 - RNA, 2507 nt, Homo sapiens
  mRNA (1)..(2507): mRNA
agucagcccagaagcggcucauugaaagcagacccucuucggcgcucgcugggcggaggacgcgccgcgguccgcagacccgagcgagcugggcaccgccgggcgcccccggcccugccgcccccuccuccccgccgcucccccgcgagcccggccaggcgcgccugacguggaccauuaaacuuggagcugccgccucguccccucuccuccuccuccucccucugacaggcgagcgagcgacucggugcaggcaggagacgugcugccgggcugggcugcccgggggagaugacuccucgccaggagggcgccucugggaagaagaccacgggggaagcaaaguuucagggcagcugaggagccuucgccgcagcccuuccgagcccaaucaucccccuggcuauggagggcggacucuaagaugaaucccgaccuggacaccggccacaacacaucagcaccugcccacuggggagaguugaaaaaugccaacuucacuggccccaaccagaccucgagcaacuccacacugccccagcuggacaucaccagggccaucucugugggccuggugcugggcgccuucauccucuuugccaucgugggcaacauccuagucaucuugucuguggccugcaaccggcaccugcggacgcccaccaacuacuucauugucaaccuggccauggccgaccugcuguugagcuucaccguccugcccuucucagcggcccuagaggugcucggcuacugggugcuggggcggaucuucugugacaucugggcagccguggauguccugugcugcacagcguccauucugagccugugcgccaucuccaucgaucgcuacaucggggugcgcuacucucugcaguaucccacgcuggucacccggaggaaggccaucuuggcgcugcucagugucugggucuuguccaccgucaucuccaucgggccucuccuuggguggaaggagccggcacccaacgaugacaaggagugcggggucaccgaagaacccuucuaugcccucuucuccucucugggcuccuucuacaucccucuggcggucauucuagucauguacugccgugucuauauaguggccaagagaaccaccaagaaccuagaggcaggagucaugaaggagauguccaacuccaaggagcugacccugaggauccauuccaagaacuuucacgaggacacccuuagcaguaccaaggccaagggccacaaccccaggaguuccauagcugucaaacuuuuuaaguucuccagggaaaagaaagcagcuaagacguugggcauuguggucgguauguucaucuugugcuggcuacccuucuucaucgcucuaccgcuuggcuccuuguucuccacccugaagccccccgacgccguguucaaggugguguucuggcugggcuacuucaacagcugccucaaccccaucaucuacccaugcuccagcaaggaguucaagcgcgcuuucgugcgcauccucgggugccagugccgcggccgcggccgccgccgacgccgccgccgccgucgccugggcggcugcgccuacaccuaccggccguggacgcgcggcggcucgcuggagcgcucgcagucgcgcaaggacucgcuggacgacagcggcagcugccugagcggcagccagcggacccugcccucggccucgccgagcccgggcuaccugggccgcggcgcgccaccgccagucgagcugugcgccuuccccgaguggaaggcgcccggcgcccuccugagccugcccgcgccugagccccccggccgccgcggccgccacgacucgggcccgcucuucaccuucaagcuccugaccgagcccgagagccccgggaccgacggcggcgccagcaacggaggcugcgaggccgcggccgacguggccaacgggcagccgggcuucaaaagcaacaugccccuggcgcccgggcaguuuuagggcccccgugcgcagcuuucuuucccuggggaggaaaacaucgugggggggaggggagggcggggcggaggggggaggggagcguccacgccggguagacgcgcgccccaaggggaaccggggggagggccggggagaggggcagcugcuuuucuggcaggggcaugggugccagguaccacgcggaaagccgggccgagcaugcugagagccugggggacccgacgccgccgggauuuaccucucucucucccucuguguauauauaaacgagucccucucuacuuguauuuaaccgugggugcacgugagugugacugugcggugugcguguguuccgcuugugugugugcguggggccgcccugugagggcgcggcgacugugcgcccaggaggcaaccgggggcguugugugugucgugacuucguaccucucaagccccuccuuguacuucacugaaggauggcacuuuggugggguuggaaacaaagucgacauuaaaggucauuucuccuguu

SEQ ID NO: 57 - RNA, 2942 nt, Homo sapiens
  mRNA (1)..(2942): mRNA
gggagcgccgcaguggccgcugccugcaccgcccggccccggagccgccgcuuugcccgcuugccgggcacgcucagagcgccgcgcgcguagcgggccccggcucuucggugcuacuccacggccggccucggggagcccggccgccgcccgggccaggcagcuccgcucucggacagccgcgcuccgcgucacaggaacuugggcaggaccccgacgggacccgugcgcggagcugcaucuggagccccgcggcuaugcccugugcuccccucccugccggccgcucguucugugccccggcccggccaccgacggccgcgcguugagaugacuuuccgcgaucuccugagcgucaguuucgagggaccccgcccggacagcagcgcagggggcuccagcgcgggcggcggcgggggcagcgcgggcggcgcggcccccucggagggcccggcggugggcggcgugccggggggcgcgggcggcggcggcggcguggugggcgcaggcagcggcgaggacaaccggagcuccgcgggggagccggggagcgcgggcgcgggcggcgacgugaauggcacggcggccgucgggggacugguggugagcgcgcagggcgugggcgugggcgucuuccuggcagccuucauccuuauggccguggcagguaaccugcuugucauccucucaguggccugcaaccgccaccugcagaccgucaccaacuauuucaucgugaaccuggccguggccgaccugcugcugagcgccaccguacugcccuucucggccaccauggagguucugggcuucugggccuuuggccgcgccuucugcgacguaugggccgccguggacgugcugugcugcacggccuccauccucagccucugcaccaucuccguggaccgguacgugggcgugcgccacucacucaaguacccagccaucaugaccgagcgcaaggcggccgccauccuggcccugcucugggucguagcccuggugguguccguagggccccugcugggcuggaaggagcccgugcccccugacgagcgcuucugcgguaucaccgaggaggcgggcuacgcugucuucuccuccgugugcuccuucuaccugcccauggcggucaucguggucauguacugccgcguguacguggucgcgcgcagcaccacgcgcagccucgaggcgggcgucaagcgcgagcgaggcaaggccuccgagguggugcugcgcauccacugucgcggcgcggccacgggcgccgacggggcgcacggcaugcgcagcgccaagggccacaccuuccgcagcucgcucuccgugcgccugcucaaguucucccgugagaagaaagcggccaagacucuggccaucgucgugggugucuucgugcucugcugguucccuuucuucuuuguccugccgcucggcuccuuguucccgcagcugaagccaucggagggcgucuucaaggucaucuucuggcucggcuacuucaacagcugcgugaacccgcucaucuaccccuguuccagccgcgaguucaagcgcgccuuccuccgucuccugcgcugccagugccgucgucgccggcgccgccgcccucucuggcgugucuacggccaccacuggcgggccuccaccagcggccugcgccaggacugcgccccgaguucgggcgacgcgccccccggagcgccgcuggcccucaccgcgcuccccgaccccgaccccgaacccccaggcacgcccgagaugcaggcuccggucgccagccgucgaaagccacccagcgccuuccgcgaguggaggcugcuggggccguuccggagacccacgacccagcugcgcgccaaagucuccagccugucgcacaagauccgcgccgggggcgcgcagcgcgcagaggcagcgugcgcccagcgcucagagguggaggcugugucccuaggcgucccacacgagguggccgagggcgccaccugccaggccuacgaauuggccgacuacagcaaccuacgggagaccgauauuuaaggaccccagagcuaggccgcggagugugcugggcuuggggguaagggggaccagagaggcgggcugguguucuaagagcccccgugcaaaucggagacccggaaacugaucagggcagcugcucugugacaucccugaggaacugggcagagcuugaggcuggagcccuugaaaggugaaaaguaguggggcccccugcuggacucaggugcccagaacucuuuucuuagaagggagaggcugcgggcuccguggggccuuuugcucccaaucccuauuugagaaacacugccccauccuccaugcccugaacccugaguagacagccccaagcauggccaggaaggccugccccuccucuucugggaaauaggcacagaagcuuuggccggcuaccccagugccucccuuagggaaaaagucagugggaccagggcuccucaguggacaaauuucuccuucuuacugccaacuggaggaacccaguggaccuaccaccacccacaagcuuugguggcagauuguauagccgucauacaagccugcuguauuuauccaugggcccgcuguccgccacccaggcuccuccgugcacaccccaucaccuggccccuaccccuuauuacuuccccucaagggccuuacuguuuacacucuguacauagcucaucgugccugugcccaucacuucuugcugggauuccaagccaccaauuggagagcuuuauuugucauuuugagacauauuuauugguaagaguacuucuauuuuguccaagcugugcaaacugucccucuuuagcccauaacccauuaaaaaaaaaauacuuuuuccccuaaagucugucaagaacuaaaggcccgcaacagaauagauugggucuacauuugcc

SEQ ID NO: 58 - RNA, 3770 nt, Homo sapiens
  mRNA (1)..(3770): mRNA
gaguuacgucagcgagagcugcaaaguuccuugcuauucuuuucuggugucggggagcugaauauuaaaagggugauuguggaguuaccgguuaucugcauuuuuuuuucuuuucuuauuuugacucuuuuuaaaaaaugcagguaaagugacagcgguucaggagcuuaaagacaucagugguggaggggugagucagcgggugcaaaaggacaaggauuuggugccucggagacacgguccccucuccgccuccagagaagagcaggcaggcagcucccgggaccgaagccggguccacaucccccgcgcgcgagcugguggcucagcagcggcgcuucagguggacgcggucaaacgaugccccgcagccuccugggucucagcacauauuccacaccuacguccccugaccugugcuccuagaagcuggagagagcaggagccuucgguggggcagcucaaaauguagguaacugcgggccaggagcagcgcccaaccuguagcgcugcgcuacccaaccaucggucccugccuuugagcgucgacggcugaucuuuugguuugagggagagacuggcgcuggaguuuugaauuccgaaucaugugcagaaugcugaaucuucccccagccaggacgaauaagacagcgcggaaaagcagauucucguaauucuggaauugcauguugcaaggagucuccuggaucuucgcacccagcuucggguagggagggaguccgggucccgggcuaggccagcccggcagguggagaggguccccggcagccccgcgcgccccuggccaugucuuuaaugcccugccccuucauguggccuucugaggguucccagggcuggccaggguuguuucccacccgcgcgcgcgcucucacccccagccaaacccaccuggcagggcucccuccagccgagaccuuuugauucccggcucccgcgcucccgccuccgcgccagcccgggagguggcccuggacagccggaccucgcccggccccggcugggaccaugguguuucucucgggaaaugcuuccgacagcuccaacugcacccaaccgccggcaccggugaacauuuccaaggccauucugcucggggugaucuuggggggccucauucuuuucggggugcuggguaacauccuagugauccucuccguagccugucaccgacaccugcacucagucacgcacuacuacaucgucaaccuggcgguggccgaccuccugcucaccuccacggugcugcccuucuccgccaucuucgagguccuaggcuacugggccuucggcagggucuucugcaacaucugggcggcaguggaugugcugugcugcaccgcguccaucaugggccucugcaucaucuccaucgaccgcuacaucggcgugagcuacccgcugcgcuacccaaccaucgucacccagaggaggggucucauggcucugcucugcgucugggcacucucccuggucauauccauuggaccccuguucggcuggaggcagccggcccccgaggacgagaccaucugccagaucaacgaggagccgggcuacgugcucuucucagcgcugggcuccuucuaccugccucuggccaucauccuggucauguacugccgcgucuacgugguggccaagagggagagccggggccucaagucuggccucaagaccgacaagucggacucggagcaagugacgcuccgcauccaucggaaaaacgccccggcaggaggcagcgggauggccagcgccaagaccaagacgcacuucucagugaggcuccucaaguucucccgggagaagaaagcggccaaaacgcugggcaucguggucggcugcuucguccucugcuggcugccuuuuuucuuagucaugcccauugggucuuucuucccugauuucaagcccucugaaacaguuuuuaaaauaguauuuuggcucggauaucuaaacagcugcaucaaccccaucauauacccaugcuccagccaagaguucaaaaaggccuuucagaaugucuugagaauccagugucucugcagaaagcagucuuccaaacaugcccugggcuacacccugcacccgcccagccaggccguggaagggcaacacaaggacauggugcgcauccccgugggaucaagagagaccuucuacaggaucuccaagacggauggcguuugugaauggaaauuuuucucuuccaugccccguggaucugccaggauuacaguguccaaagaccaauccuccuguaccacagcccgggugagaaguaaaagcuuuuugcaggucugcugcuguguagggcccucaacccccagccuugacaagaaccaucaaguuccaaccauuaagguccacaccaucucccucagugagaacggggaggaagucuaggacaggaaagaugcagaggaaaggggaauaaucuuagguacccaccccacuuccuucucggaaggccagcucuucuuggaggacaagacaggaccaaucaaagaggggaccugcugggaauggggugggugguagacccaacucaucaggcagcggguagggcacagggaagagggagggugucucacaaccaaccaguucagaaugauacggaacagcauuucccugcagcuaaugcuuucuuggucacucugugcccacuucaacgaaaaccaccaugggaaacagaauuucaugcacaauccaaaagacuauaaauauaggauuaugauuucaucaugaauauuuugagcacacacucuaaguuuggagcuauuucuugauggaagugaggggauuuuauuuucaggcucaaccuacugacagccacauuugacauuuauggggagagggccuagagaggaagagcauuaagaugguggccgagauccagacacauuauuuuuagagcaaguccugaaacuccacccuuucugauacauccaauuucucagcuugagggaaacuuggcagugagguuuaccuuugacugcacuuugaauggaugcugcuccucauuucccaaagaugagagugaggggcacucaugaugucuuggaauguaguguucucuauccuugggucaggagaggguugaaagaaccaaccagggugguauggcuuuucccaagagaaaacaauagaccugagcaucauucuuucaauuuaacuacuagagagaaaauccauuaaacaguuccaagaucagaugaugauaggcuuucucucuaauaucuuuaaagagcaagagguugguguggcuguuaauuucuugcaaagauagaggcuguucacuuacugcacagccauuucaacauggcuuacaaaagccuuucuugacaaaucacuuaccuguuccagaacucuguuaugaaaauccagagcuuauaauauuuugaaaggcaaaagauugucucccauuacuucuuaucugcuucacuauugcauaaugaaugagcuucaccuguggcauguuggaaugagccuuaugauccaaguacauuauucccgaacuuugaaaaauacuaaugcuuagcuucagacaauacugauggaucaccaaagaacugucuaacgcaggaguuugcaaacauuuuuugauagaaggccauuuguucugguaaaagauccaguagucaacucaacuucaucuauuccuacuuuucugcaagagcuuggggaacaugcuauauuuugcuuuauguauaccaaauuuugguaaaccauaauaacucaguaca

SEQ ID NO: 59 - RNA, 3696 nt, Homo sapiens
  mRNA (1)..(3696): mRNA
acuccccugcaggcgcggcuggggcgaaagccugcgagcugagcgggcgcaagguccuccgcgccuccuuuaagaaccggcccagcccggcccgcgcccccagagcguacggcauccgcguggcgggagggcgcgacuuucuccggucccgggcgggacggggacggcggcgggacaacuugggaaacuucucuggggcggacggcagggaccccgggcaccgguggaggaggauguaggagggcggcugcugguccuggguguucccgaccuccuaggccccgcucguccaggccauggggcuccagcgcccucggcgccgcccgaggggcgacgcucuugucuagccgagccgggcagcgcugucguccacggugcgcacugggcgggcagcgcucccucugcccaccucccgccccgucauggaccaccaggaccccuacuccgugcaggccacagcggccauagcggcggccaucaccuuccucauucucuuuaccaucuucggcaacgcucuggucauccuggcuguguugaccagccgcucgcugcgcgccccucagaaccuguuccuggugucgcuggccgccgccgacauccugguggccacgcucaucaucccuuucucgcuggccaacgagcugcugggcuacugguacuuccggcgcacguggugcgagguguaccuggcgcucgacgugcucuucugcaccucguccaucgugcaccugugcgccaucagccuggaccgcuacugggccgugagccgcgcgcuggaguacaacuccaagcgcaccccgcgccgcaucaagugcaucauccucacuguguggcucaucgccgccgucaucucgcugccgccccucaucuacaagggcgaccagggcccccagccgcgcgggcgcccccagugcaagcucaaccaggaggccugguacauccuggccuccagcaucggaucuuucuuugcuccuugccucaucaugauccuugucuaccugcgcaucuaccugaucgccaaacgcagcaaccgcagaggucccagggccaagggggggccugggcagggugaguccaagcagccccgacccgaccaugguggggcuuuggccucagccaaacugccagcccuggccucuguggcuucugccagagaggucaacggacacucgaaguccacuggggagaaggaggagggggagaccccugaagauacugggacccgggccuugccacccaguugggcugcccuucccaacucaggccagggccagaaggaggguguuuguggggcaucuccagaggaugaagcugaagaggaggaagaggaggaggaggaggaggaagagugugaaccccaggcagugccagugucuccggccucagcuugcagccccccgcugcagcagccacagggcucccgggugcuggccacccuacguggccaggugcuccugggcaggggcgugggugcuauaggugggcagugguggcgucgacgggcgcagcugacccgggagaagcgcuucaccuucgugcuggcuguggucauuggcguuuuugugcucugcugguuccccuucuucuucagcuacagccugggagccaucugcccgaagcacugcaaggugccccauggccucuuccaguucuucuucuggaucggcuacugcaacagcucacugaacccuguuaucuacaccaucuucaaccaggacuuccgccgugccuuccggaggauccugugccgcccguggacccagacggccuggugagcccgccugcgcugccccugugggguuggugcgguggcgccggggucacccugcuucuugcccugcuguguguggcugccuccccugggcuuucugcucccugcccagauccuguaggccucaucuuaggaaccccuugggaggggugggcaggggggcugcuagcaagggucccagugaagcuuccccuugccggcuuagcugugggggaccccuucuccacccucucccugagcacaggccgauggaggugguucaaauccucuggaacauagccaagaccaggagaagagagagcacuuucuucccagagccccaugcucuccagaccaaugucugggcuucccuuucuugaggaccuuguguuccuggcaggucacuugcuugugguguuuucguuucuuuuucaucucccccccacccacaaagagcacggagccagccuuccacuuuucccaguggggccugcugcugagggggaggaagaaacgaagacugaucacccacgcuaggcacucgcggucccuggcaggcgcugggaugggggcuuaugggguggcaucgucucugggcccuccuuucccccuuugccuguuuucggaucugugguuccuuugaaagccagaacaauggaucggcuuccuuacccagcaccccuccgguagguggguggccacguggaugccucgcuggggcggucuuggaggccuggucucugccucgacgggagauccccgaucacuggcauucacccccugcaaaaaucggggcgacaauagcucacugccuacuugcugcagggagaugaaaggcuuugcagaaagcuuugagcucugugggggaacacacuagagaaccaaaaaugugauuauauggugauauaaaaaucccuuuccucuguguuuaccaccaccugucuuccuguagacuuuuguucugucccuggggugugugaauuccuaccccgaacuggaagccgggaguggcagacagaaucacuauuucaaguuaaaggaucucuuugagaauguguucuucuggcugcaaaggucugaguuauuacgcuacaugacaacguuucgacauuucaccggcaacaccaagaggguuuuuaguggcuugggucuccccagugggggauaagucuuuugucaucaaggaggcaaauugucuccccaagacagcucaaaauauccacaccucggcaacagucuaagaugagagccugugacagguggcagcgcccccaggugggguacuggcaucagagccuggugcgccccuaggggagccucccacuggagugcccggccaggucuccaagccccaaaugaguccuugugaaccacaacugauccccccaggugggugcuuguggacugccucggacccagccacgcugcuccccgcaaugcugauggggcugugcauugaggaccccugcuuccugguucucagucccaccccaaaaccuggcacccagaacaguuggaaguguggaaaggagguuuaucggccuucccuuggagagggccuggcuucaacauugggccaguaggcaucuuagcuuggcaggugucgggggaaugggccagauggaccugcuagauuuggaagggcaccgagggaguuuucuggguguagagagaauggaggggaccaaaaagaguccuuccuggggugugggaggcuucccagcuugguccucaguggguuguugaggccagaguaucgcccugggaugugguggggagcugggccaggagagggacugacugugacccucugcuggccggucuugugugcgccccaugggacccccaguguucuugccugugaccucuuauugcgacaugcaggugguguuuuuuuuuuuuuuaaacucugagcuauuuuaucaauaaaggauauuuuguaauaa

SEQ ID NO: 60 - RNA, 3879 nt, Homo sapiens
  mRNA (1)..(3879): mRNA
gagcagcagcagcuccagcucggugcagaagcccagcagccggcgugccgccgcccggccacuccagcgccuucuuccccgccuugcgcuccugccccaacucgcgcugucgucggaccccggcccauccagcagcgcucggcgcccaccaggcggacgcccaggagaaccccugccuccgucgcggcuccuggagagcugaucguucaccugccccggcccgccugaggacgggggugccuucaugcggcccccacacuccucaccccgccgccgccgccgucccggagcuccgcacagugugccccagccccagcagggcgcacaacuuuggaagucucgcggcgcuccgagaggcggcagaguccgcgccccagccccgggccgggccgggccagaaccgcagcgucugggggaagccagagagucgguaaucgcuucggggauguaaggcgacagacauaggacccccgagcucgcaucagcacccuucggcugccucccggggugggggcgggccccgcacacgguaagaccucuugcuuucgcucaggcucaagauucaagauacagauauugauauguauauauauauuuaauuuccugucauccuuccaaguuaucaggccaccgaugauuuuuguucucccuucuugaagaauaaaucucucuuuacccaucggcucucccuacucucucccgccgcuuagaaauaaaacuuggcuguauuaggagcucggagcaagaaggcgcccaccgagagcgucugaagcgcgagccaggcgcaguucgcgggacccgggccaugggccgcuagcgguccuccaguucgggcccggccucccugcggcccccucccuaugugagccgcagccaggcgagcggggcgccggaggaagaggaggacccacgggcgccgggccggaaggcagcuggcagcaggcccaggccagcgggcgcccgcguucauguuccgccaggagcagccguuggccgagggcagcuuugcgcccaugggcucccugcagccggacgcgggcaacgcgagcuggaacgggaccgaggcgccggggggcggcgcccgggccaccccuuacucccugcaggugacgcugacgcuggugugccuggccggccugcucaugcugcucaccguguucggcaacgugcucgucaucaucgccguguucacgagccgcgcgcucaaggcgccccaaaaccucuuccuggugucucuggccucggccgacauccugguggccacgcucgucaucccuuucucgcuggccaacgaggucaugggcuacugguacuucggcaaggcuuggugcgagaucuaccuggcgcucgacgugcucuucugcacgucguccaucgugcaccugugcgccaucagccuggaccgcuacugguccaucacacaggccaucgaguacaaccugaagcgcacgccgcgccgcaucaaggccaucaucaucaccgugugggucaucucggccgucaucuccuucccgccgcucaucuccaucgagaagaagggcggcggcggcggcccgcagccggccgagccgcgcugcgagaucaacgaccagaagugguacgucaucucgucgugcaucggcuccuucuucgcucccugccucaucaugauccuggucuacgugcgcaucuaccagaucgccaagcgucgcacccgcgugccacccagccgccgggguccggacgccgucgccgcgccgccggggggcaccgagcgcaggcccaacggucugggccccgagcgcagcgcgggcccggggggcgcagaggccgaaccgcugcccacccagcucaacggcgccccuggcgagcccgcgccggccgggccgcgcgacaccgacgcgcuggaccuggaggagagcucgucuuccgaccacgccgagcggccuccagggccccgcagacccgagcgcgguccccggggcaaaggcaaggcccgagcgagccaggugaagccgggcgacagccugccgcggcgcgggccgggggcgacggggaucgggacgccggcugcagggccgggggaggagcgcgucggggcugccaaggcgucgcgcuggcgcgggcggcagaaccgcgagaagcgcuucacguucgugcuggccguggucaucggaguguucguggugugcugguuccccuucuucuucaccuacacgcucacggccgucgggugcuccgugccacgcacgcucuucaaauucuucuucugguucggcuacugcaacagcucguugaacccggucaucuacaccaucuucaaccacgauuuccgccgcgccuucaagaagauccucugucggggggacaggaagcggaucgugugagguuuccgcuggcgcccgcguagacucacgcugacugcaggcagcggggggcaucgaggggugcuuagccccagggcacucagaaacccgggcgcugccugcucugcguuuccucgucugggguggcucugcagccuccugcgggcgggcgucugcugcuccuacaagggaagcuucuugcugccaggcccacacauccccaguuguugguuuggccacucuugaccuggagccaucuuccuagugggccaccccuaaucacuauugcuuccuaaagguauuuucacccucuucgccugguacagcccucacagcucuucagagcaagcacuggacuacaagggcauggcucacaaaagguuaauggauggggguuaccuagcccuggcuaauuccccuuccauucccaacucucucucucuuuuuaaagaaaaaugcuaagggcagcccugccugcccuccccaucccccgcuguaaauauacacuauuuuugauagcacacauggggcccccauaucucuuggccuugguuuugauguugaaauccuggccuugggagagaugccuuccaggcagacacagcugucugguucaggccaagccccuuugcaaugcaagcccuuucugguguuaugaagucccucuaugucgucguuuucaccagcaacuggugacugucccuucgacacggaccugcuuugagauuuccugacagggaaaagauuucuguccauuuuuuuccugugccuaacagcauaauugccuuuuccuauguaaauauuaugaugguggaucaagacauaaguaaaugagccuuucugccucacaucagcccuguguauaaagccauuauucucugaugcacuguuugccccaguaacucacuuuaaaaccucucuuuccaguguucccucucucccuccagggccacugcuugaagaagaauauguauguuucuaucuuguaugucugugugccccuccugccccgaaagugcugacuauggggaaaucuuuuagcugcuguuuuuagacuccaaggaguggaaauuauguggaagaagcaaaccugauacaauuugcccaagguaaacaguuugaaaagacaaaugggccugccaaacuguacaguuucuuccccaagagcuguuagguaucaaaauguuguccuuuccccccuccgugcuuuucugguugagaucaugucauugaugaacugccaaagucaggggaggagggcagagacuuuguguuuacaucugcauuucuacauguuuuagacagagacaauuuaaggccugcacucuuauuucacuaaagaaaaacuaaugucagcacauguugcuaaugacaguggauuuuuuuuuaaauaaaaaaguuuacagaucaaaugugaaauaaauaugaauggagugguccucuugucuguuaucugaguuuucaaaagcuuuaagacucugggaacaucugauuuuauggauuuuuuaaaaauaaaaaauguacauuauaaaaa

SEQ ID NO: 61 - RNA, 2142 nt, Homo sapiens
  mRNA (1)..(2142): mRNA
acuuacacgcucggcagcugcggggagcccggcagccacgcucuccggcgcgccgcccgcggagccaccacggccgagggccggcugcugggcgccgcgguccccggcgggcgcgcccgagcagcaggcggcgaugcgggcgccgaccccggcuggggggcgcccgagcugccgcggcugcgccccggcuccaggagggacggcguagcucgcgggaggaccauggcguccccggcgcuggcggcggcgcuggcgguggcggcagcggcgggccccaaugcgagcggcgcgggcgagaggggcagcggcgggguugccaaugccucgggggcuuccugggggccgccgcgcggccaguacucggcgggcgcgguggcagggcuggcugccguggugggcuuccucaucgucuucaccguggugggcaacgugcugguggugaucgccgugcugaccagccgggcgcugcgcgcgccacagaaccucuuccuggugucgcuggccucggccgacauccugguggccacgcuggucaugcccuucucguuggccaacgagcucauggccuacugguacuucgggcagguguggugcggcguguaccuggcgcucgaugugcuguuuugcaccucgucgaucgugcaucugugugccaucagccuggaccgcuacuggucggugacgcaggccgucgaguacaaccugaagcgcacaccacgccgcgucaaggccaccaucguggccguguggcucaucucggccgucaucuccuucccgccgcuggucucgcucuaccgccagcccgacggcgccgccuacccgcagugcggccucaacgacgagaccugguacauccuguccuccugcaucggcuccuucuucgcgcccugccucaucaugggccuggucuacgcgcgcaucuaccgaguggccaagcugcgcacgcgcacgcucagcgagaagcgcgcccccgugggccccgacggugcguccccgacuaccgaaaacgggcugggcgcggcggcaggcgcaggcgagaacgggcacugcgcgcccccgcccgccgacguggagccggacgagagcagcgcagcggccgagaggcggcggcgccggggcgcguugcggcggggcgggcggcggcgagcgggcgcggaggggggcgcgggcggugcggacgggcagggggcggggccgggggcggcugagucgggggcgcugaccgccuccagguccccggggcccgguggccgccugucgcgcgccagcucgcgcuccgucgaguucuuccugucgcgccggcgccgggcgcgcagcagcgugugccgccgcaagguggcccaggcgcgcgagaagcgcuucaccuuugugcuggcuguggucaugggcguguucgugcucugcugguuccccuucuucuucagcuacagccuguacggcaucugccgcgaggccugccaggugcccggcccgcucuucaaguucuucuucuggaucggcuacugcaacagcucgcucaacccggucaucuacacggucuucaaccaggauuuccggcgauccuuuaagcacauccucuuccgacggaggagaaggggcuucaggcagugacucgcacccgucugggaauccuggacagcuccgcgcucggggcugggcagaaggggcggcccggacgggggagcuuucccagagacccggggauggauuggccuccagggcgcaggggagggugcggcagggcaggagcuuggcagagagauagccgggcuccagggaguggggaggagagagggggagaccccuuugccuuccccccucagcaaggggcugcuucuggggcucccugccuggauccagcucugggagcccugccgagguguggcugugaggucaggguuuuagagagcaguggcagagguagcccccuaaaugggcaagcaaggagccccccaaagacacuaccacuccccauccccgucugaccaagggcugacuucuccaggaccuagucgggggguggcugccagggggcaaggagaaagcaccgacaaucuuugauuacugaaaguauuuaaauguuugccaaaaacaacagccaaaacaaccaaacuauuuucuaaauaaaccuuuguaaucuaa

SEQ ID NO: 62 - RNA, 3800 nt, Homo sapiens
  mRNA (1)..(3800): mRNA
gcacccggccucuccuccgcgcgugcgggccccucgcggcgccucccgggcucgggagacccggucccgccgcccgcccggcccgcggccgccucuuccucucgcagccccgcggcgcuugccgcagucgcaggcggcggagcagccagagcgccccagguaccagugcgggcgggagacggaggcgacccugcugccggguggggagggggcgccgcgcggccgcugcccuccucuucccgcggggccuggcggccccggcgaacccccgcgccgcacaggccgugugcgccggaucccggugccuccgcgccagcugagaccugccuuucccaggggcggcuuugcuccggccccuaggcccccgcccugguaccccacacgcccaccggccgcggggagccugcuccggcccgagagcgcccauucaccccccucacccaccuccccgcguucccacuuccccgcacucugacccggccggacgccccuccccuaucuugccgcccgcccccuccagggggcucugcucccaccccagggagcccauccgaccucugcuugacuucccgccgcuuccuucaggggccucggcucaucgggugccccuccccaaacuuccaacccguuugcuccaggaguuccugccccauccccgagggcgcccaaauagccacacuguguccuccuguagucgcugcccccugaccuagcgcgacccagcgcccccgcccauguccccccacucaccucccccggggggcguggugagucgcggucuguucucacggacgguccccguccagccugcgcuucgccggggcccucaucugcuuucccgccacccuaucacucccuugccguccacccucgguccucauggucccagagccgggcccaaccgccaauagcaccccggccuggggggcagggccgccgucggccccggggggcagcggcuggguggcggccgcgcugugcguggucaucgcgcugacggcggcggccaacucgcugcugaucgcgcucaucugcacucagcccgcgcugcgcaacacguccaacuucuuccuggugucgcucuucacgucugaccugaugguggggcugguggugaugccgccggccaugcugaacgcgcuguacgggcgcugggugcuggcgcgcggccucugccugcucuggaccgccuucgacgugaugugcugcagcgccuccauccucaaccucugccucaucagccuggaccgcuaccugcucauccucucgccgcugcgcuacaagcugcgcaugacgccccugcgugcccuggcccuaguccugggcgccuggagccucgccgcucucgccuccuuccugccccugcugcugggcuggcacgagcugggccacgcacggccacccgucccuggccagugccgccugcuggccagccugccuuuuguccuuguggcgucgggccucaccuucuuccugcccucgggugccauaugcuucaccuacugcaggauccugcuagcugcccgcaagcaggccgugcagguggccucccucaccaccggcauggccagucaggccucggagacgcugcaggugcccaggaccccacgcccagggguggagucugcugacagcaggcgucuagccacgaagcacagcaggaaggcccugaaggccagccugacgcugggcauccugcugggcauguucuuugugaccugguugcccuucuuuguggccaacauaguccaggccgugugcgacugcaucuccccaggccucuucgauguccucacauggcuggguuacuguaacagcaccaugaaccccaucaucuacccacucuucaugcgggacuucaagcgggcgcugggcagguuccugccauguccacgcuguccccgggagcgccaggccagccuggccucgccaucacugcgcaccucucacagcggcccccggcccggccuuagccuacagcaggugcugccgcugccccugccgccggacucagauucggacucagacgcaggcucaggcggcuccucgggccugcggcucacggcccagcugcugcuuccuggcgaggccacccaggaccccccgcugcccaccagggccgcugccgccgucaauuucuucaacaucgaccccgcggagcccgagcugcggccgcauccacuuggcauccccacgaacugacccgggcuuggggcuggccaauggggagcuggauugagcagaacccagacccugaguccuugggccagcucuuggcuaagaccaggaggcugcaagucuccuagaagcccucugagcuccagaggggugcgcagagcugacccccugcugccaucuccaggccccuuaccugcagggaucauagcugacucagauaucguguucugaaggaugcuccacuguugaguguaacuugugugugcagaggaugggcuggaggacucuggauguuggggagaaggaccucuugguucagguuaggaugaaaacaaucaugaccuuugcccauucugucaggcuggacgggagggaaugcccccaccugcaggcauggauuaaugcugggcugaaccccuugcuacucacaguguaguccauggacaaucggcauugccgucaccuuugggaguuugugaaaaaugcacaucucugccccugcccagaccugcugagucagaacugcauguuaccaagaucccaggugaaucaugugcccauuacaguuugagaugugcugccuuagaucaucuuuaacccucagaaugauuugcuggaugguuuuuggaccugucagucuccccucccugguauauauguggcagcagcugcaccugggaggcaggcaggcaugcaugugggccucugcucuaccaccuaugagcucuggggccugacacgaguuauuuaaccucuccgagccucgguuaucccauguguaagguagagaaaugaugccuaucuugcagguugucuugagaauuaagacaaugugugcccagcacccaguacacggucaguuaauugguacguucuguuguuacgacccccggcuauccagcccccuggccucuccggcugugugucaggaagggcucuuugcucccguuguucucuuuccauuugccucccccucuucagcccuggccuagccucccaaagccugacucugguuccccaacucccuguucuggcuccuggguggggaccccaucucucagggacccucccagcucccaagcugcugcccugccuuuccugggggcaaaaggggcagguggcaguuucuuuuucagccucagaacugguuuuccucaauccugggggguccuuggaccuagaagggaacaacugauggggauaaacccagccccuugcccaccugccucuggccuucccuggggucagugagagaaaugggaaugguagcuacuugggucagcagaguagagucaaagagcuguuccuggugugagguggggcccacgaggcaggcagguggacucuggcacaggggccuugguuucugggggcaggggguuggggaacucuuggucgccaguuuaccugccauaggucccuuuucagcccugccucccccaaguuccaucaacacaaacucccaggggguccuccaugccuggccuggcccucuccuuggugcugaugagacacaacucuggccccggcggcuggccucugaggggccauggugcuugcggggcuuccccuaucccccaggucugcccaaggggacucucaauaaacucuagcugaaggca

SEQ ID NO: 63 - RNA, 2170 nt, Homo sapiens
  mRNA (1)..(2170): mRNA
agucacagcagcaagcaagucuagugaacagauaagaugacaugcucagcaaaauaacaacgaaaccagagggggaacucucuggcaugcaaguucaaacacgacucuacaacuacggcagaaaaagagagagagagaaacuaaaaauauauauauauccuauuuuuuucacagcuaucaguuucuuucacugagcuuuccuaaauuuaagccucuagaaaauaauaaauacuuggauaucuuaccuacaaacauggacagauguguguaugcgcucauuuuagagaacuugaauuuuuuuuuuuaaaggaaggugucaacuuuggcuuuugaguguuuggcaugguuacaaugccuuaaaaaaacagaugagcagcuuagcuacuaaccaugcugaccacuguucggaacgggauugaaucacagaaaaacagcaaauggcucucucuuacagagugucugaacuucaaagcacaauuccugagcacauuuugcagagcaccuuuguucacguuaucucuucuaacuggucuggauuacagacagaaucaauaccagaggaaaugaaacagauuguugaggaacagggaaauaaacugcacugggcagcucuucugauacucauggugauaauacccacaauugguggaaauacccuuguuauucuggcuguuucacuggagaagaagcugcaguaugcuacuaauuacuuucuaauguccuuggcgguggcugauuugcugguuggauuguuugugaugccaauugcccucuugacaauaauguuugaggcuauguggccccucccacuuguucuauguccugccugguuauuucuugacguucucuuuucaaccgcauccaucaugcaucucugugccauuucaguggaucguuacauagccaucaaaaagccaauccaggccaaucaauauaacucacgggcuacagcauucaucaagauuacaguggugugguuaauuucaauaggcauugccauuccagucccuauuaaagggauagagacugauguggacaacccaaacaauaucacuugugugcugacaaaggaacguuuuggcgauuucaugcucuuuggcucacuggcugccuucuucacaccucuugcaauuaugauugucaccuacuuucucacuauccaugcuuuacagaagaaggcuuacuuagucaaaaacaagccaccucaacgccuaacaugguugacugugucuacaguuuuccaaagggaugaaacaccuugcucgucaccggaaaagguggcaaugcuggaugguucucgaaaggacaaggcucugcccaacucaggugaugaaacacuuaugcgaagaacauccacaauugggaaaaagucagugcagaccauuuccaacgaacagagagccucaaagguccuagggauuguguuuuuccucuuuuugcuuauguggugucccuucuuuauuacaaauauaacuuuaguuuuaugugauuccuguaaccaaacuacucuccaaaugcuccuggagauauuuguguggauaggcuauguuuccucaggagugaauccuuuggucuacacccucuucaauaagacauuucgggaugcauuuggccgauauaucaccugcaauuaccgggccacaaagucaguaaaaacucucagaaaacgcuccaguaagaucuacuuccggaauccaauggcagagaacucuaaguuuuucaagaaacauggaauucgaaaugggauuaacccugccauguaccagaguccaaugaggcuccgaaguucaaccauucagucuucaucaaucauucuacuagauacgcuucuccucacugaaaaugaaggugacaaaacugaagagcaaguuaguuauguauagcagaacuggcaguugucaucaaacauaaugaugaguaagaugaugaaugagauguaaaugugccaagaauauauuauauaaagaauuuuaugucauauaucaaaucaucucuuuaaccuaagauguaaguauuaagaauaucuaauuuuccuaauuuggacaagauuauuccaugaggaaaauaauuuuauauagcuacaaaugaaaacaauccagcacucugguuaaauuuuaagguauucgaaugaaauaaagucaaaucaauaaauuucaggcuuuaaaaagaa

SEQ ID NO: 64 - RNA, 4774 nt, Homo sapiens
  mRNA (1)..(4774): mRNA
acccgcgcgagguaggcgcucuggugcuugccgaggacgcuuccuuccucagaugcaccgaucuucccgauacugccuuuggagcggcuagauugcuagccuuggcugcuccauuggccugccuugccccuuaccugccgauugcauaugaacucuucuucugucuguacaucguugucgucggagucgucgcgaucgucguggcgcucgugugauggccuucguccguuuagaguaguguaguuaguuaggggccaacgaagaagaaagaagacgcgauuagugcagagaugcuggagguggucaguuacuaagcuagaguaagauagcggagcgaaaagagccaaaccuagccggggggcgcacggucacccaaaggaggucgacucgccggcgcuuccuaucgcgccgagcucccuccauuccucucccuccgccgaggcgcgagguugcggcgcgcagcgcagcgcagcucagcgcaccgacugccgcgggcuccgcugggcgauugcagccgaguccguuucucgucuagcugccgccgcggcgaccgcugccuggucuuccucccggacgcuaguggguuaucagcuaacacccgcgagcaucuauaacauaggccaacugacgccauccuucaaaaacaacuaaaggaugauaugaugaaccuagccuguuaauuucgucuucucaauuuuaaacuuugguugcuuaagacugaagcaaucauggugaaccugaggaaugcggugcauucauuccuugugcaccuaauuggccuauugguuuggcaaugugauauuucugugagcccaguagcagcuauaguaacugacauuuucaauaccuccgaugguggacgcuucaaauucccagacgggguacaaaacuggccagcacuuucaaucgucaucauaauaaucaugacaauagguggcaacauccuugugaucauggcaguaagcauggaaaagaaacugcacaaugccaccaauuacuucuuaaugucccuagccauugcugauaugcuagugggacuacuugucaugccccugucucuccuggcaauccuuuaugauuaugucuggccacuaccuagauauuugugccccgucuggauuucuuuagauguuuuauuuucaacagcguccaucaugcaccucugcgcuauaucgcuggaucgguauguagcaauacguaauccuauugagcauagccguuucaauucgcggacuaaggccaucaugaagauugcuauuguuugggcaauuucuauagguguaucaguuccuaucccugugauuggacugagggacgaagaaaagguguucgugaacaacacgacgugcgugcucaacgacccaaauuucguucuuauuggguccuucguagcuuucuucauaccgcugacgauuauggugauuacguauugccugaccaucuacguucugcgccgacaagcuuugauguuacugcacggccacaccgaggaaccgccuggacuaagucuggauuuccugaagugcugcaagaggaauacggccgaggaagagaacucugcaaacccuaaccaagaccagaacgcacgccgaagaaagaagaaggagagacguccuaggggcaccaugcaggcuaucaacaaugaaagaaaagcuucgaaaguccuugggauuguuuucuuuguguuucugaucauguggugcccauuuuucauuaccaauauucugucuguucuuugugagaaguccuguaaccaaaagcucauggaaaagcuucugaauguguuuguuuggauuggcuauguuuguucaggaaucaauccucugguguauacucuguucaacaaaauuuaccgaagggcauucuccaacuauuugcguugcaauuauaagguagagaaaaagccuccugucaggcagauuccaagaguugccgccacugcuuugucugggagggagcuuaauguuaacauuuaucggcauaccaaugaaccggugaucgagaaagccagugacaaugagcccgguauagagaugcaaguugagaauuuagaguuaccaguaaaucccuccagugugguuagcgaaaggauuagcagugugugagaaagaacagcacagucuuuuccuacgguacaagcuacauauguaggaaaauuuucuucuuuaauuuuucuguuggucuuaacuaauguaaauauugcugucugaaaaaguguuuuuacauauagcuuugcaaccuuguacuuuacaaucaugccuacauuagugagauuuaggguucuauauuuacuguuuauaauagguggagacuaacuuauuuugauuguuugaugaauaaaauguuuauuuuugcucucccucccuucuuuccuuccuuuuuuccuuucuuccuuccuuucucucuuucuuuugugcauauggcaacguucauguucaucucagguggcauuugcaggugaccagaaugaggcacaugacagugguuauauuucaaccacaccuaaauuaacaaauucaguggacauuuguucuggguuaacaguaaauauacacuuuacauucuugcucugcucaucuacacauauaaacacaguaagauagguucugcuuucugauacaucugucagugagucagaggcagaaccuagucuuguuguucauauaggggcaaaaauuugacauugucagaauguuguguugguauuuacugcaaugucugucccuaaacauagugguauuuuaacauagcagcugguuaaccgggacuacagaaguggaaggauaaugagauguaauacaccaaauagcuuuucacuucuuaaggacaguguucaaauucugauuauuacaacaagcaaacugaaauuaguguuuucauucugguccuuaguaaauuccuaauucuaugauuaaacugggaaaugagaucccagaguuauuucccaacccaggauucaacaucaauuggguuuugaucucagcauccuggaaauuugugugcuucacacaaagugaaauuaguauuuugagccuuauuaaaauauuuucuuaauuaugguaccucugucuauaggacuuaauuuagcaguccauuuuugaguaaaacuuguauuggaaguauagaugguagaaacuuuggaaguuuuacuugauuaaggacuacagaauugggcccuuagaaugugaaaaaaaaaaguaauuaaaaagacacuuuuaccgaacucgggauuacagaaacacggaguuuccauuuggauuuuaaacaaaauuuaugucauuuucagauccuuccaaacucucuagugcaggaaaaggcugcagcuaauuugugaaaguggcaagcucuucauugcacugcaguuauuuaccagaaguuuaaaucuuuguuaaaauauaguguuguguuacaauaaguguuggccaucauuucauucgugggccugcugcucucuaagaauucaguagcauuuuaauaguuucuaaaccaugaaaaguuuucaagcauugcuaaagucaggccauucagucuaugcugugugcagaguauacaaguguuucuaguaacaguauuuccauacgugcccauuucacacaacuguggauaaauuuuggaagaauucaugaugcuaguucuuacgcuugacaguuacuuacacaccugagaaugugccucucaguaucuuaaaauugguuaaugaaaaaucugaauuucuaaaacccuuggucuguguucucaacacacaguauagauaaauccaauagucugccacaagggcaguggaagagcugcuguauuugaggaaacucauacagucucuauuugauuugcaacacugccaaacaucagucaauugcuugagcaugcccaaauauaacaugaaagucaagucuaccugccuugccuguuaggucuguugaagugcauguuaaaauaauuauaugaagcagaaugagaugauuuaauucuuaccgaaaugaaaauggcugaagaaacacagcaugcauuuagcaugaguucugcacauacagaugguguccugcauguaugccauguauguugcaugaauccaucgauuuguauuaauguagggcagaauagcugauagaagaaggacugaagaaaauccuucagcaauccuuaaaaagaccaugcauucagaucugaaguagugugaguguuagaaaaaacuggaaacaucugauuucugaacuaucagggcaagcucauagcacauguuuuacaaagaaacaaaauauaaaucacagauuuccaaaaguacuagcaauaaguugaaugauaauagcucacagcacauuuguuaaugauucuugugucaucaaguaguaguacuuaauaguacccaaccugguaauuauccucaaguugugugcuauucguaaguucugugcaguuugguaugaaacaaauauacucauuuggauauaaaucuuacccuucaauguuaaaucuacaaacuuuuauaaauguuuuaaagaaguccaugugauaauuguaaaggugaugaauuuaccaucaaacaaaucauuuugauguauuauuauauauguauaucuguguaagacacgugcaacagacugccuuauauuauuuucuguaauucuucuccuuugucaaaugguauuuuuugugaaugguugcaaaguguugucuuauuccuaauuccuguauguuauccacuacagguuuuaugagacuuccuauuaauuuauuaaauuuauuaaauguugaaaaaaaaaaaaaaaaaaaaaaa

SEQ ID NO: 65 - RNA, 2376 nt, Homo sapiens
  mRNA (1)..(2376): mRNA
agugccagccuggcgcccgcgacugccugccccagccccucaguggcggcuugcucucuucucucgcuccgaaccagacacagccgcugccgcugccguccggcgcgcuacagacucccgagaacagcccuggcugucagcgagcaccagccgcuuccuguccccaucgcggagacuggaggggcgcaccacggccauggagccagaggcgcuucaggaggcaagagaaguccccgcgcgcuccgcagcccggcgcagcucauggugagcgcccucuggggcucgagggucccuuggcugagggggcgcauccucggggugcccgauggggcugccugggggucgcagggcugaaguugggaccgcgcacagaccgccccugcaguccagcccgaaaugcugccgccaggcagcaacggcaccgcguacccggggcaguucgcucuauaccagcagcuggcgcaggggaacgccguggggggcucggcgggggcaccgccacuggggcccucacagguggucaccgccugccugcugacccuacucaucaucuggacccugcugggcaacgugcuggugugcgcagccaucgugcggagccgccaccugcgcgccaacaugaccaacgucuucaucgugucucuggccgugucagaccuuuucguggcgcugcuggucaugcccuggaaggcagucgccgagguggccgguuacuggcccuuuggagcguucugcgacgucuggguggccuucgacaucaugugcuccacugccuccauccugaaccugugcgucaucagcguggaccgcuacugggccaucuccaggcccuuccgcuacaagcgcaagaugacucagcgcauggccuuggucauggucggccuggcauggaccuuguccauccucaucuccuucauuccgguccagcucaacuggcacagggaccaggcggccucuuggggcgggcuggaccugccaaacaaccuggccaacuggacgcccugggaggaggacuuuugggagcccgacgugaaugcagagaacugugacuccagccugaaucgaaccuacgccaucucuuccucgcucaucagcuucuacauccccguugccaucaugaucgugaccuacacgcgcaucuaccgcaucgcccaggugcagauccgcaggauuuccucccuggagagggccgcagagcacgcgcagagcugccggagcagcgcagccugcgcgcccgacaccagccugcgcgcuuccaucaagaaggagaccaagguucucaagacccugucggugaucaugggggucuucguguguugcuggcugcccuucuucauccuuaacugcauggucccuuucugcaguggacaccccgaaggcccuccggccggcuuccccugcgucagugagaccaccuucgacgucuucgucugguucggcugggcuaacuccucacucaaccccgucaucuaugccuucaacgccgacuuucagaagguguuugcccagcugcuggggugcagccacuucugcucccgcacgccgguggagacggugaacaucagcaaugagcucaucuccuacaaccaagacaucgucuuccacaaggaaaucgcagcugccuacauccacaugaugcccaacgccguuacccccggcaaccgggagguggacaacgacgaggaggaggguccuuucgaucgcauguuccagaucuaucagacguccccagauggugacccuguugcugagucugucugggagcuggacugcgagggggagauuucuuuagacaaaauaacaccuuucaccccgaauggauuccauuaaacugcauuaagaaacccccucauggaucugcauaaccgcacagacauugacaagcacgcacacacacgcaaauacaugccuuuccagugcugcucccuuuaucauguguuucuguguaguagcucgugugcuuagaaaccucaccccauugauugguaguucgaagaauuggcagaagcaguugcaauaaacucagucaaauguacccagccuaccagagauggaccaacgauccuaugagagaagagaguauggugcuggguccuuaaaaaaaaaaaugauacuugguccuuaaaaaauaugcucuccccucccuuuuuaaacaaauggcuuguucagucacuuguuuguguuugaauugauuuuuaaacagcagguugugugugugugcagugauguggugggagcacagcuuuccugggucuggauucccguggcuuugugcuuaugucauuucuucucucugugcuggugggggccucuuuaccauagcuuaagaaguaucccugauuuauucuggugucuaauaaacacagauuauuugua

SEQ ID NO: 66 - RNA, 4031 nt, Homo sapiens
  mRNA (1)..(4031): mRNA
gugcgcuugcuggugccagcucggcucgcugccucgcauugccacaggcuccugagaggucgcgggcagugccugcggggaggcgcggggcccugcucuguagggcugaaggccgcccgagguucgccaaggcucugggcucucgaaaggaagccaagaaaagaagcugcccaggugaccaguccugggagugcucucucccaaggaagcuccgagcgcccaggagcccuuagccggggucuagugcccuuugaacaaucuccagcucuucaaggaagugggcugccgccgccucucuugggaccuggccugggauccuuuccccaaacgcaccccggcgauuuuugcgcaccgggagccgaaccccugcugcgcgcagcuggcugggcucaggcgcgcuuccucaacguuucggagccgcugcccccagcgaaguccacauuccaagcuccaggggcuuugagagagacgaccccaaggcaaggcguuuggagagcugcugaggagccaggggcuuggaggagcgagaagacauguauuuucagcugagucucagaaggggagaaucuccugucaccaccagaaaagcaacagccccgaaaugugauugcaacugacuagcagagcagaggcccaggagucacuggauugaugauuuagaauaugcuaaaaagccagugcuuuauuuggggaauucaggggcuuucuggugcccaagacagugaccugcagcaagggagucagaagacagauguagaaaucaagagugaccauccacgggauugacuuggauugccacucaagcgguccucucauggaauguuggugaggcccucugccagggaagcaaucuggcugugcaaagugcugccugguggggaggacuccuggaaaucugacugaccccuauucccugcuugggaacuugaggggugucagagccccugaugugcuuucucuuaggaagaugaggacucugaacaccucugccauggacgggacugggcuggugguggagagggacuucucuguucguauccucacugccuguuuccugucgcugcucauccuguccacgcuccuggggaacacgcuggucugugcugccguuaucagguuccgacaccugcgguccaaggugaccaacuucuuugucaucuccuuggcugugucagaucucuugguggccguccuggucaugcccuggaaggcaguggcugagauugcuggcuucuggcccuuuggguccuucuguaacaucuggguggccuuugacaucaugugcuccacugcauccauccucaaccucugugugaucagcguggacagguauugggcuaucuccagcccuuuccgguaugagagaaagaugacccccaaggcagccuucauccugaucaguguggcauggaccuugucuguacucaucuccuucaucccagugcagcucagcuggcacaaggcaaaacccacaagccccucugauggaaaugccacuucccuggcugagaccauagacaacugugacuccagccucagcaggacauaugccaucucauccucuguaauaagcuuuuacaucccuguggccaucaugauugucaccuacaccaggaucuacaggauugcucagaaacaaauacggcgcauugcggccuuggagagggcagcaguccacgccaagaauugccagaccaccacagguaauggaaagccugucgaauguucucaaccggaaaguucuuuuaagauguccuucaaaagagaaacuaaaguccugaagacucugucggugaucauggguguguuugugugcuguuggcuaccuuucuucaucuugaacugcauuuugcccuucugugggucuggggagacgcagcccuucugcauugauuccaacaccuuugacguguuugugugguuugggugggcuaauucauccuugaaccccaucauuuaugccuuuaaugcugauuuucggaaggcauuuucaacccucuuaggaugcuacagacuuugcccugcgacgaauaaugccauagagacggugaguaucaauaacaauggggccgcgauguuuuccagccaucaugagccacgaggcuccaucuccaaggagugcaaucugguuuaccugaucccacaugcugugggcuccucugaggaccugaaaaaggaggaggcagcuggcaucgccagacccuuggagaagcuguccccagcccuaucagucauauuggacuaugacacugacgucucucuggagaagauccaacccaucacacaaaacggucagcacccaaccugaacucgcagaugaauccugccacacaugcucaucccaaaagcuagaggagauugcucuggggcuugcuauuaagaaacuaagguacggugagacucugaggugucaggagagcccucugcugcuuuccaacacacaauuaacuccguuuccaaauacauuccaguguauuuucuguguuguucauagucaaucaaacagggacacuacaaacauggggagccauaagggacaugucuuuggcuucagaauuguuuuuagaaauuuauucuuaucuuaggauuuaccaaauagggcaaagaaucaacagugaacagcuucacuuaaaaucaaauuuuucugggaagaaaaugagauggguugaguuugcuguauacaaacaggugcuaacacuguucccagcaaaguuuucagauuguaaagguaggugcaugccuucauaaauuauuucuaaaacauuaauugaggcuuacaguaggagugagaaauuuuuuuccagaauugagagauguuuuguugauauugguucuauuuauuuauuguauauauggauauuuuuaauuuaugauauaauaaauauauauuuaucauauuuaauaggauaaauuaaugaguuuuauccaagaccuuacaaccacauuucuggccauuuaacuagcacuuuauaagccaaugaagcaaacacacagacucugugagauucuaaauguucauguguaacuucuagaaacacagcagaaacugauagauaagggaauaaaguugaaaugauuccuuaaaauucauggacacagauaaaugcaaggugagaauugacaaaugcuauaaaugcuuucuuuuucugaaaagauuuugaaaaauuuaaaaaaguauagcuacuacuguguucaaaacguuuuaaaugacaaaugacuuucccaggggaauuugcaguucuguaaauaucuuaaauaaaagccaacuuaagaagagcccagcauuaaauuuacgaucuuaggugguaaugaaaaguauaugcugcuuuguauuuauguaaaauaauuggcccucuccaucuuuucucauuucaugugucagguaguuuuucugaaccacacaaauggcuuuccuggagagagaucuguagcacagacaguggguuacagcagccccacugagggaccaaacucaaacccugcauuuccaucuuaccaggucaaaccaaaccagucaguggggcuacuuuuuauagugcuuuaaucugaauuuagagcugauuuuuaaaggagucuuuaaauguuaaugguauacuaacuaacgaauagugccucauuaucauucuugagucagauacuucuguugaugggagaaacagaagaauccuucccuuuggguguguugagcucccccaaagccaucagcaucucuuuugacaaaugcuaguccuuucucugugcuuuggaaucagguuccugcaucaucacccggacuguaaaaaguaucauaagccucccuugccagaugccaacucguggggcauuucaacagaguuucuuugaaauguuuacaacguauucuucuugauaagcaaugaacuuaacauuuagaugcaauccgugaaaagaaaaaaaaaucugaaaaauaucuccugcaucaggucuguguuauuuauguauugugaauguuuucuuaauuuuauuggcuguaugcuuucuuacacauaauaaaaauauuuugugaacucaaa

SEQ ID NO: 67 - RNA, 3336 nt, Homo sapiens
  mRNA (1)..(3336): mRNA
agaugagcagcgcuccggccagggagggcgcgugcuggccagcccggaacgggugagaucgcuccagcccgccucugugcccacuccccugugcgcccugccggcagagcggcacccccgcugucccuggcgcccagugggggcggcagcgggcagggcagcggggaccaccggggcugggggcuguugagcccguggaguccggcucgguuggggagaaggacgaugcgcggcgagcccaggugauccggggcugggguccugcgcccgcagccccuggcccgcgccucacgcucgccggcuccccagcgcgggggcuggagcccgcacccgagggggcggcagggagggcgcaggcagcggcugggagcgcagcggcagcggcggcagcagaagcucggcucagcggcuggggguggccgcucgaaucugccagggcaccucgcucccucgccucuggcagcgggacccugugggcauugaaauccaacucacucaugcuuauuuccuguaauggacaaacuugaugcuaaugugaguucugaggaggguuucgggucaguggagaagguggugcugcucacguuucucucgacgguuauccugauggccaucuuggggaaccugcuggugaugguggcugugugcugggacaggcagcucaggaaaauaaaaacaaauuauuucauuguaucucuugcuuuugcggaucugcugguuucggugcuggugaugcccuuuggugccauugagcugguucaagacaucuggauuuauggggagguguuuugucuuguucggacaucucuggacguccugcucacaacggcaucgauuuuucaccugugcugcauuucucuggauagguauuacgccaucugcugccagccuuuggucuauaggaacaagaugaccccucugcgcaucgcauuaaugcugggaggcugcugggucauccccacguuuauuucuuuucucccuauaaugcaaggcuggaauaacauuggcauaauugauuugauagaaaagaggaaguucaaccagaacucuaacucuacguacugugucuucauggucaacaagcccuacgccaucaccugcucugugguggccuucuacaucccauuucuccucauggugcuggccuauuaccgcaucuaugucacagcuaaggagcaugcccaucagauccagauguuacaacgggcaggagccuccuccgagagcaggccucagucggcagaccagcauagcacucaucgcaugaggacagagaccaaagcagccaagacccugugcaucaucauggguugcuucugccucugcugggcaccauucuuugucaccaauauuguggauccuuucauagacuacacugucccugggcagguguggacugcuuuccucuggcucggcuauaucaauuccggguugaacccuuuucucuacgccuucuugaauaagucuuuuagacgugccuuccucaucauccucugcugugaugaugagcgcuaccgaagaccuuccauucugggccagacugucccuuguucaaccacaaccauuaauggauccacacauguacuaagggaugcaguggagugugguggccagugggagagucagugucacccgccagcaacuucuccuuugguggcugcucagcccagugacacuuaggccccugggacaaugacccagaagacagccaugccuccgaaagagggccagguccuaagcugcugcuugugcgcgacugcacccgguauucucuucaccugaggcuuuccguccgccagugcaggaacccggugcucgcugggcuuuuccucugagauuccagcagguggcgcuggagggagucaggggacauaauggccuccuuguucacuuuuuauuucccaacacucccucuucccagauucucugcuuuugccgcguggucucuggugucucugacauguucccgucacucacugugucugaucugucucacucacguucuaagcacgauagcuugcugcguccuaucucggucacugaugucuccaaauacgccuucuuugcugugugggcuuccagauaucaacaaaauacucccucucuguuccugucuuagccagaggcgucgcuccugucuugcuuacuugcuuggucacguccgucacagccauguucaagcuauccccugccucuucuucgcccuguucugugccagacgcuaacacacuuuucucccugugucuggguccaaacgugcauccucuugguuggcccccuugucagucccagacguccacuugcucuuccuuauguccaaugucuguccuccucuucaggacaugcuccauuuauuuuccauggaagacacagggucagagaucugucagaggaaaauggucuccacagcaacucuggagggaagcugaagacagauagcuuccuccugguguccuggccgcuggcgugucugcccugagaugcucgcucuccugcugggcuagaacgggacgucuucuaaaaagccuugggcagggcuagcaacaucguagguguuuauuaggcuucaggaucuuucccaugaacuugugauaucauuuugaaacccuucaaucuggcuuuguugcugccaaaggagauaacuguuggaaguucacacuggggaaacucauuuuccccugcagcucccccaaggugcaggagggcaggauccagcagcacuggggaugccgaggcaaugcuguacuugaagccagaggacccaaauugccgcccuggcugugcugcaaacaagcugugagaucuugaauaguguucuuucaucucuggaccccacuuuccacaucugugcacucaagcuuggacuagaggaccuuuaaguuuucuuucuguuucuaagacaacaguaugcugcuauagccgucugcuaccucgagaagccauggccugccugccuuuuggcuuuguuucaaaggaacgacccuccucauuugggccuuuuagaccccugaccuuuccagcuuuguccuucagauuaaucuggaugucuuuuauucaaagcugagucaguggcucuugcgaaugccccauagcagaagucccgggaagguaaaugccaaacccagagccaugugagauucugacuuugaucuuggcugugguuuaauagcaucucaggauuauuuaucauauucucuuuuuuucuuauugcuuauaaggugucaguuauuguuuccauuguuguuuacuuuaguucuggaacugaaaccgacaggaagaaacuuuggaauaaggaagagaagauugaccaaaccauccaaaugccaaaaagaaaaa

SEQ ID NO: 68 - RNA, 2585 nt, Homo sapiens
  mRNA (1)..(2585): mRNA
gggacagcuagagaagauggcccaggcuggggaagucgcucucaugccuugcuguccccuccccugagccaggugauuugggagacccccuccuuccuucuuucccuaccgccccacgcgcgacccggggauggcuccguggccucacgagaacagcucucuugccccauggccggaccuccccacccuggcgcccaauaccgccaacaccagugggcugccagggguuccgugggaggcggcccuagccggggcccugcuggcgcuggcggugcuggccaccgugggaggcaaccugcuggucaucguggccaucgccuggacuccgagacuccagaccaugaccaacguguucgugacuucgcuggccgcagccgaccuggugaugggacuccugguggugccgccggcggccaccuuggcgcugacuggccacuggccguugggcgccacuggcugcgagcuguggaccucgguggacgugcugugugugaccgccagcaucgaaacccugugcgcccuggccguggaccgcuaccuggcugugaccaacccgcugcguuacggcgcacuggucaccaagcgcugcgcccggacagcugugguccuggugugggucgugucggccgcggugucguuugcgcccaucaugagccagugguggcgcguaggggccgacgccgaggcgcagcgcugccacuccaacccgcgcugcugugccuucgccuccaacaugcccuacgugcugcuguccuccuccgucuccuucuaccuuccucuucucgugaugcucuucgucuacgcgcggguuuucgugguggcuacgcgccagcugcgcuugcugcgcggggagcugggccgcuuuccgcccgaggagucuccgccggcgccgucgcgcucucuggccccggccccgguggggacgugcgcuccgcccgaaggggugcccgccugcggccggcggcccgcgcgccuccugccucuccgggaacaccgggcccugugcaccuugggucucaucaugggcaccuucacucucugcugguugcccuucuuucuggccaacgugcugcgcgcccuggggggccccucucuagucccgggcccggcuuuccuugcccugaacuggcuagguuaugccaauucugccuucaacccgcucaucuacugccgcagcccggacuuucgcagcgccuuccgccgucuucugugccgcugcggccgucgccugccuccggagcccugcgccgccgcccgcccggcccucuuccccucgggcguuccugcggcccggagcagcccagcgcagcccaggcuuugccaacggcucgacggggcuucuuggggaguuucuuaggccugaaggacaagaagcaacaacucuguugaucagaaccuguggaaaaccucuggccucuguucagaaugagucccaugggauuccccggcugugacacucuacccuccagaaccugacgacugggccaugugacccaaggagggauccuuaccaaguggguuuucaccauccucuugcucucugucugagagauguuuucuaaaccccagccuugaacuucacuccucccucagugguaguguccaggugccguggagcagcaggcuggcuuugguaggggcacccaucacccggcuugccugugcagucagugagugcuuagggcaaagagagcuccccugguuccauuccuucugccacccaaacccugaugagaccuuaguguucuccaggcucuguggcccaggcugagagcagcaggguagaaaagaccaagauuugggguuuuaucucugguucccuuauuacugcucucaagcaguggccucucucacuuuagccauggaauggcuccgaucuaccucacagcagugucagaaggacuucgccaggguuuugggagcuccaggguucauaagaaggugaaccauuagaacagaucccuucuuuuccuuuugcaaucagauaaauaaauaucacugaaugcaguucauccucggcccccuuucccuccguuuguuuucuuuucauaauccacuuacucccuucccuucuacucugcgcuggcuuuugacagaggcaguaaauuaggccuaauccucacucuuuucuuccuaaucuucaucaaacaaaaaaugaaaagucugucuggacgaaggggagugagcuugagccuuugauaucuugcucccccacccuuccugaaacucuugaaauccaguugccauugaguagcaaagccacgcuccccacaggacuuggacagagggcccacagggggaugggcuggcuguggccagguuuagggcagggggcauuuguccccuccaugcuauaauccaguggugccuuacauggugugugugugugugugugcgugugugugugugugugugugucuggaggcacaggcacaaagcauugcuuggguuggucaaaugucuugugucauaaauauauucugauguuucccagccuuuccacaaccucuaccuucccacucaccuuccccagcuacaaaaaucuguauuauccucuuaaaguaaaacuggaguuacaaauuuga

SEQ ID NO: 69 - RNA, 3039 nt, Homo sapiens
  mRNA (1)..(3039): mRNA
agaaacaugcugaagucccggcggcucuuccagcagcggcagcggcuccagcagcagcggcggcggcggcggcggcggcagcggcagcgacagcgcucggcuccugcgggaaaggcgcccggcgcccaugccuccggccccgcgccgcggcugcccugacccggccgcgaccucccucugcgcaccacgccgcccgggcuucugggguguuccccaaccacggcccagcccugccacaccccccgcccccggccuccgcagcucggcaugggcgcgggggugcucguccugggcgccuccgagcccgguaaccugucgucggccgcaccgcuccccgacggcgcggccaccgcggcgcggcugcuggugcccgcgucgccgcccgccucguugcugccucccgccagcgaaagccccgagccgcugucucagcaguggacagcgggcaugggucugcugauggcgcucaucgugcugcucaucguggcgggcaaugugcuggugaucguggccaucgccaagacgccgcggcugcagacgcucaccaaccucuucaucaugucccuggccagcgccgaccuggucauggggcugcugguggugccguucggggccaccaucgugguguggggccgcugggaguacggcuccuucuucugcgagcuguggaccucaguggacgugcugugcgugacggccagcaucgagacccugugugucauugcccuggaccgcuaccucgccaucaccucgcccuuccgcuaccagagccugcugacgcgcgcgcgggcgcggggccucgugugcaccgugugggccaucucggcccugguguccuuccugcccauccucaugcacugguggcgggcggagagcgacgaggcgcgccgcugcuacaacgaccccaagugcugcgacuucgucaccaaccgggccuacgccaucgccucguccguagucuccuucuacgugccccugugcaucauggccuucguguaccugcggguguuccgcgaggcccagaagcaggugaagaagaucgacagcugcgagcgccguuuccucggcggcccagcgcggccgcccucgcccucgcccucgcccguccccgcgcccgcgccgccgcccggacccccgcgccccgccgccgccgccgccaccgccccgcuggccaacgggcgugcggguaagcggcggcccucgcgccucguggcccugcgcgagcagaaggcgcucaagacgcugggcaucaucaugggcgucuucacgcucugcuggcugcccuucuuccuggccaacguggugaaggccuuccaccgcgagcuggugcccgaccgccucuucgucuucuucaacuggcugggcuacgccaacucggccuucaaccccaucaucuacugccgcagccccgacuuccgcaaggccuuccagggacugcucugcugcgcgcgcagggcugcccgccggcgccacgcgacccacggagaccggccgcgcgccucgggcugucuggcccggcccggacccccgccaucgcccggggccgccucggacgacgacgacgacgaugucgucggggccacgccgcccgcgcgccugcuggagcccugggccggcugcaacggcggggcggcggcggacagcgacucgagccuggacgagccgugccgccccggcuucgccucggaauccaagguguagggcccggcgcggggcgcggacuccgggcacggcuucccaggggaacgaggagaucuguguuuacuuaagaccgauagcaggugaacucgaagcccacaauccucgucugaaucauccgaggcaaagagaaaagccacggaccguugcacaaaaaggaaaguuugggaagggaugggagaguggcuugcugauguuccuuguuguuuuuuuuuucuuuucuuuucuuucuucuucuuuuuuuuuuuuuuuuuuuuuucuguuugugguccggccuucuuuugugugugcgugugaugcaucuuuagauuuuuuucccccaccaggugguuuuugacacucucugagaggaccggaguggaagauggguggguuaggggaagggagaagcauuaggaggggauuaaaaucgaucaucguggcucccaucccuuucccgggaacaggaacacacuaccagccagagagaggagaaugacaguuugucaagacauauuuccuuuugcuuuccagagaaauuucauuuuaauuucuaaguaaugauuucugcuguuaugaaagcaaagagaaaggauggaggcaaaauaaaaaaaaaucacguuucaagaaauguuaagcucuucuuggaacaagccccaccuugcuuuccuuguguagggcaaacccgcugucccccgcgcgccuggguggucaggcugagggauuucuaccucacacugugcauuugcacagcagauagaaagacuuguuuauauuaaacagcuuauuuauguaucaauauuaguuggaaggaccaggcgcagagccucucucugugacaugugacucugucaauugaagacaggacauuaaaagagagcgagagagagaaacaguucagauuacugcacauguggauaaaaacaaaaacaaaaaaaaggagugguucaaaaugccauuuuugcacaguguuaggaauuacaaaauccacagaagauguuacuugcacaaaaagaaauuaaauauuuuuuaaagggagaggggcugggcagaucuuaaauaaaauucaaacucuacuucuguugucuaguauguuauugagcuaaugauucauugggaaaauaccuuuuuauacuccuuuaucaugguacuguaacuguauccauauuauaaauauaauuaucuuaaggauuuuuuauuuuuuuuuauguccaagugcccacgugaauuugcuggugaaaguuagcacuuguguguaaauucuacuuccucuuguguguuuuaccaaguauuuauacucuggugcaacuaacuacugugugaggaauugguccaugugcaauaaauaccaaugaagcacaa

SEQ ID NO: 70 - RNA, 2013 nt, Homo sapiens
  mRNA (1)..(2013): mRNA
gcacugcgaagcggcuucuucagagcacgggcuggaacuggcaggcaccgcgagccccuagcacccgacaagcugagugugcaggacgaguccccaccacacccacaccacagccgcugaaugaggcuuccaggcguccgcucgcggcccgcagagccccgccguggguccgcccgcugaggcgcccccagccagugcgcucaccugccagacugcgcgccauggggcaacccgggaacggcagcgccuucuugcuggcacccaauggaagccaugcgccggaccacgacgucacgcaggaaagggacgagguguggguggugggcaugggcaucgucaugucucucaucguccuggccaucguguuuggcaaugugcuggucaucacagccauugccaaguucgagcgucugcagacggucaccaacuacuucaucacuucacuggccugugcugaucuggucaugggccuggcaguggugcccuuuggggccgcccauauucuuaugaaaauguggacuuuuggcaacuucuggugcgaguuuuggacuuccauugaugugcugugcgucacggccagcauugagacccugugcgugaucgcaguggaucgcuacuuugccauuacuucaccuuucaaguaccagagccugcugaccaagaauaaggcccgggugaucauucugaugguguggauugugucaggccuuaccuccuucuugcccauucagaugcacugguaccgggccacccaccaggaagccaucaacugcuaugccaaugagaccugcugugacuucuucacgaaccaagccuaugccauugccucuuccaucguguccuucuacguuccccuggugaucauggucuucgucuacuccagggucuuucaggaggccaaaaggcagcuccagaagauugacaaaucugagggccgcuuccauguccagaaccuuagccagguggagcaggaugggcggacggggcauggacuccgcagaucuuccaaguucugcuugaaggagcacaaagcccucaagacguuaggcaucaucaugggcacuuucacccucugcuggcugcccuucuucaucguuaacauugugcaugugauccaggauaaccucauccguaaggaaguuuacauccuccuaaauuggauaggcuaugucaauucugguuucaauccccuuaucuacugccggagcccagauuucaggauugccuuccaggagcuucugugccugcgcaggucuucuuugaaggccuaugggaauggcuacuccagcaacggcaacacaggggagcagaguggauaucacguggaacaggagaaagaaaauaaacugcugugugaagaccucccaggcacggaagacuuugugggccaucaagguacugugccuagcgauaacauugauucacaagggaggaauuguaguacaaaugacucacugcuguaaagcaguuuuucuacuuuuaaagacccccccccccaacagaacacuaaacagacuauuuaacuugaggguaauaaacuuagaauaaaauuguaaaauuguauagagauaugcagaaggaagggcauccuucugccuuuuuuauuuuuuuaagcuguaaaaagagagaaaacuuauuugagugauuauuuguuauuuguacaguucaguuccucuuugcauggaauuuguaaguuuaugucuaaagagcuuuaguccuagaggaccugagucugcuauauuuucaugacuuuuccauguaucuaccucacuauucaaguauuagggguaauauauugcugcugguaauuuguaucugaaggagauuuuccuuccuacacccuuggacuugaggauuuugaguaucucggaccuuucagcugugaacauggacucuucccccacuccucuuauuugcucacacgggguauuuuaggcagggauuugaggagcagcuucaguuguuuucccgagcaaagucuaaaguuuacaguaaauaaauuguuugaccaug

SEQ ID NO: 71 - DNA, 1440 bp, Artificial Sequence
  Nucleotide sequence
atggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactacgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctgccttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctagccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggtag

SEQ ID NO: 72 - protein, 41 aa, Artificial Sequence
  HUMAN Muscarinic Acetylcholine Receptor M4 OS Homo Sapiens OX 9606 GN CHRM4 PE 1 SV 2
VNNYFLFSLACADLIIGAFSMNLYTVYIIKGYWPLGAVVCD

SEQ ID NO: 73 - protein, 41 aa, Artificial Sequence
  HUMAN Muscarinic Acetylcholine Receptor M1 OS Homo Sapiens OX 9606 GN CHRM1 PE 1 SV 2
VNNYFLLSLACADLIIGTFSMNLYTTYLLMGHWALGTLACD

SEQ ID NO: 74 - protein, 41 aa, Artificial Sequence
  HUMAN Muscarinic Acetylcholine Receptor M2 OS Homo Sapiens OX 9606 GN CHRM2 PE 1 SV 1
VNNYFLFSLACADLIIGVFSMNLYTLYTVIGYWPLGPVVCD

SEQ ID NO: 75 - protein, 41 aa, Artificial Sequence
  HUMAN Muscarinic Acetylcholine Receptor M3 OS Homo Sapiens OX 9606 GN CHRM3 PE 1 SV 1
VNNYFLLSLACADLIIGVISMNLFTTYIIMNRWALGNLACD

SEQ ID NO: 76 - protein, 41 aa, Artificial Sequence
  HUMAN Muscarinic Acetylcholine Receptor M5 OS Homo Sapiens OX 9606 GN CHRM5 PE 1 SV 2
VNNYYLLSLACADLIIGIFSMNLYTTYILMGRWALGSLACD

SEQ ID NO: 77 - protein, 41 aa, Artificial Sequence
  HUMAN Histamine H1 receptor OS homo sapiens OX 9606 GN HRH1 PE 1 SV 1
VGNLYIVSLSVADLIVGAVVMPMNILYLLMSKWSLGRPLCL

SEQ ID NO: 78 - protein, 41 aa, Artificial Sequence
  HUMAN Histamine H2 receptor OS homo sapiens OX 9606 GN HRH2 PE 2 SV 1
LTNCFIVSLAITDLLLGLLVLPFSAIYQLSCKWSFGKVFCN

SEQ ID NO: 79 - protein, 41 aa, Artificial Sequence
  HUMAN Histamine H3 receptor OS homo sapiens OX 9606 GN HRH3 PE 1 SV 2
QNNFFLLNLAISDFLVGAFCIPLYVPYVLTGRWTFGRGLCK

SEQ ID NO: 80 - protein, 40 aa, Artificial Sequence
  HUMAN Histamine H4 receptor OS homo sapiens OX 9606 GN HRH4 PE 1 SV 2
RSSYFFLNLAISDFFVGVISIPLYIPHTLFEWDFGKEICV

SEQ ID NO: 81 - protein, 41 aa, Artificial Sequence
  HUMAN 5-hydroxytryptamine receptor 1F OS homo sapiens OX 9606 GN HTR1F PE 2 SV 1
PANYLICSLAVTDFLVAVLVMPFSIVYIVRESWIMGQVVCD

SEQ ID NO: 82 - protein, 41 aa, Artificial Sequence
  HUMAN 5-hydroxytryptamine receptor 1E OS homo sapiens OX 9606 GN HTR1E PE 1 SV 1
PANYLICSLAVTDLLVAVLVMPLSIIYIVMDRWKLGYFLCE

SEQ ID NO: 83 - protein, 41 aa, Artificial Sequence
  HUMAN 5-hydroxytryptamine receptor 1D OS homo sapiens OX 9606 GN HTR1D PE 1 SV 1
PANYLIGSLATTDLLVSILVMPISIAYTITHTWNFGQILCD

SEQ ID NO: 84 - protein, 41 aa, Artificial Sequence
  HUMAN 5-hydroxytryptamine receptor 1B OS homo sapiens OX 9606 GN HTR1B PE 1 SV 1
PANYLIASLAVTDLLVSILVMPISTMYTVTGRWTLGQVVCD

SEQ ID NO: 85 - protein, 42 aa, Artificial Sequence
  HUMAN 5-hydroxytryptamine receptor 5A OS homo sapiens OX 9606 GN HTR5A PE 2 SV 1
VPHNLVASMAVSDVLVAALVMPLSLVHELSGRRWQLGRRLCQ

SEQ ID NO: 86 - protein, 42 aa, Artificial Sequence
  HUMAN 5-hydroxytryptamine receptor 7 OS homo sapiens OX 9606 GN HTR7 PE 1 SV 2
PSNYLIVSLALADLSVAVAVMPFVSVTDLIGGKWIFGHFFCN

SEQ ID NO: 87 - protein, 41 aa, Artificial Sequence
  HUMAN 5-hydroxytryptamine receptor 1A OS homo sapiens OX 9606 GN HTR1A PE 1 SV 3
VANYLIGSLAVTDLMVSVLVLPMAALYQVLNKWTLGQVTCD

SEQ ID NO: 88 - protein, 42 aa, Artificial Sequence
  HUMAN 5-hydroxytryptamine receptor 2A OS homo sapiens OX 9606 GN HTR2A PE 1 SV 2
ATNYFLMSLAIADMLLGFLVMPVSMLTILYGYRWPLPSKLCA

SEQ ID NO: 89 - protein, 42 aa, Artificial Sequence
  HUMAN (D3) Dopamine receptor OS homo sapiens OX 9606 GN DRD3 PE 1 SV 2
TTNYLVVSLAVADLLVATLVMPWVVYLEVTGGVWNFSRICCD

SEQ ID NO: 90 - protein, 41 aa, Artificial Sequence
  HUMAN (D2) Dopamine receptor OS homo sapiens OX 9606 GN DRD2 PE 1 SV 2
TTNYLIVSLAVADLLVATLVMPWVVYLEVVGEWKFSRIHCD

SEQ ID NO: 91 - protein, 42 aa, Artificial Sequence
  HUMAN (D4) Dopamine receptor OS homo sapiens OX 9606 GN DRD4 PE 1 SV 3
PTNSFIVSLAAADLLLALLVLPLFVYSEVQGGAWLLSPRLCD

SEQ ID NO: 92 - protein, 41 aa, Artificial Sequence
  HUMAN Alpha-1B adrenergic receptor OS Homo sapiens OX 9606 GN ADRA1B PE 1 SV 3
PTNYFIVNLAMADLLLSFTVLPFSAALEVLGYWVLGRIFCD

SEQ ID NO: 93 - protein, 41 aa, Artificial Sequence
  HUMAN Alpha-1D adrenergic receptor OS Homo sapiens OX 9606 GN ADRA1D PE 1 SV 2
VTNYFIVNLAVADLLLSATVLPFSATMEVLGFWAFGRAFCD

SEQ ID NO: 94 - protein, 41 aa, Artificial Sequence
  HUMAN Alpha-1A adrenergic receptor OS Homo sapiens OX 9606 GN ADRA1A PE 1 SV 2
VTHYYIVNLAVADLLLTSTVLPFSAIFEVLGYWAFGRVFCN

SEQ ID NO: 95 - protein, 41 aa, Artificial Sequence
  HUMAN Alpha-2B adrenergic receptor OS Homo sapiens OX 9606 GN ADRA2B PE 1 SV 4
PQNLFLVSLAAADILVATLIIPFSLANELLGYWYFRRTWCE

SEQ ID NO: 96 - protein, 41 aa, Artificial Sequence
  HUMAN Alpha-2A adrenergic receptor OS Homo sapiens OX 9606 GN ADRA2A PE 1 SV 4
PQNLFLVSLASADILVATLVIPFSLANEVMGYWYFGKAWCE

SEQ ID NO: 97 - protein, 41 aa, Artificial Sequence
  HUMAN Alpha-2C adrenergic receptor OS Homo sapiens OX 9606 GN ADRA2C PE 1 SV 2
PQNLFLVSLASADILVATLVMPFSLANELMAYWYFGQVWCG

SEQ ID NO: 98 - protein, 41 aa, Artificial Sequence
  HUMAN 5-hydroxytryptamine receptor 6 OS homo sapiens OX 9606 GN HTR6 PE 1 SV 1
TSNFFLVSLFTSDLMVGLVVMPPAMLNALYGRWVLARGLCL

SEQ ID NO: 99 - protein, 42 aa, Artificial Sequence
  HUMAN 5-hydroxytryptamine receptor 2B OS homo sapiens OX 9606 GN HTR2B PE 1 SV 1
ATNYFLMSLAVADLLVGLFVMPIALLTIMFEAMWPLPLVLCP

SEQ ID NO: 100 - protein, 42 aa, Artificial Sequence
  HUMAN 5-hydroxytryptamine receptor 2C OS homo sapiens OX 9606 GN HTR2C PE 1 SV 1
ATNYFLMSLAIADMLVGLLVMPLSLLAILYDYVWPLPRYLCP

SEQ ID NO: 101 - protein, 41 aa, Artificial Sequence
  HUMAN D(1B) dopamine receptor OS homo sapiens OX 9606 GN DRD5 PE 1 SV 2
NMTNVFIVSLAVSDLFVALLVMPWKAVAEVAGYWPFGAFCD

SEQ ID NO: 102 - protein, 41 aa, Artificial Sequence
  HUMAN D(1A) dopamine receptor OS homo sapiens OX 9606 GN DRD1 PE 1 SV 1
KVTNFFVISLAVSDLLVAVLVMPWKAVAEIAGFWPFGSFCN

SEQ ID NO: 103 - protein, 42 aa, Artificial Sequence
  HUMAN 5-hydroxytryptamine receptor 4 OS homo sapiens OX 9606 GN HTR4 PE 1 SV 2
IKTNYFIVSLAFADLLVSVLVMPFGAIELVQDIWIYGEVFCL

SEQ ID NO: 104 - protein, 41 aa, Artificial Sequence
  HUMAN Beta-3 adrenergic receptor OS homo sapiens OX 9606 GN ADRB3 PE 1 SV 3
MTNVFVTSLAAADLVMGLLVVPPAATLALTGHWPLGATGCE

SEQ ID NO: 105 - protein, 41 aa, Artificial Sequence
  HUMAN Beta-1 adrenergic receptor OS homo sapiens OX 9606 GN ADRB1 PE 1 SV 2
LTNLFIMSLASADLVMGLLVVPFGATIVVWGRWEYGSFFCE

SEQ ID NO: 106 - protein, 41 aa, Artificial Sequence
  HUMAN Beta-2 adrenergic receptor OS homo sapiens OX 9606 GN ADRB2 PE 1 SV 3
VTNYFITSLACADLVMGLAVVPFGAAHILMKMWTFGNFWCE

SEQ ID NO: 107 - protein, 39 aa, Artificial Sequence
  HUMAN Muscarinic Acetylcholine Receptor M4 OS Homo Sapiens OX 9606 GN CHRM4 PE 1 SV 2
LWLALDYVVSNASVMNLLIISFDRYFCVTKPLTYPARRT

SEQ ID NO: 108 - protein, 39 aa, Artificial Sequence
  HUMAN Muscarinic Acetylcholine Receptor M1 OS Homo Sapiens OX 9606 GN CHRM1 PE 1 SV 2
LWLALDYVASNASVMNLLLISFDRYFSVTRPLSYRAKRT

SEQ ID NO: 109 - protein, 39 aa, Artificial Sequence
  HUMAN Muscarinic Acetylcholine Receptor M2 OS Homo Sapiens OX 9606 GN CHRM2 PE 1 SV 1
LWLALDYVVSNASVMNLLIISFDRYFCVTKPLTYPVKRT

SEQ ID NO: 110 - protein, 39 aa, Artificial Sequence
  HUMAN Muscarinic Acetylcholine Receptor M3 OS Homo Sapiens OX 9606 GN CHRM3 PE 1 SV 1
LWLAIDYVASNASVMNLLVISFDRYFSITRPLTYRAKRT

SEQ ID NO: 111 - protein, 39 aa, Artificial Sequence
  HUMAN Muscarinic Acetylcholine Receptor M5 OS Homo Sapiens OX 9606 GN CHRM5 PE 1 SV 2
LWLALDYVASNASVMNLLVISFDRYFSITRPLTYRAKRT

SEQ ID NO: 112 - protein, 39 aa, Artificial Sequence
  HUMAN Histamine H1 receptor OS homo sapiens OX 9606 GN HRH1 PE 1 SV 1
FWLSMDYVASTASIFSVFILCIDRYRSVQQPLRYLKYRT

SEQ ID NO: 113 - protein, 39 aa, Artificial Sequence
  HUMAN Histamine H2 receptor OS homo sapiens OX 9606 GN HRH2 PE 2 SV 1
IYTSLDVMLCTASILNLFMISLDRYCAVMDPLRYPVLVT

SEQ ID NO: 114 - protein, 40 aa, Artificial Sequence
  HUMAN Histamine H3 receptor OS homo sapiens OX 9606 GN HRH3 PE 1 SV 2
LWLVVDYLLCTSSAFNIVLISYDRFLSVTRAVSYRAQQGD

SEQ ID NO: 115 - protein, 40 aa, Artificial Sequence
  HUMAN Histamine H4 receptor OS homo sapiens OX 9606 GN HRH4 PE 1 SV 2
FWLTTDYLLCTASVYNIVLISYDRYLSVSNAVSYRTQHTG

SEQ ID NO: 116 - protein, 39 aa, Artificial Sequence
  HUMAN 5-hydroxytryptamine receptor 1F OS homo sapiens OX 9606 GN HTR1F PE 2 SV 1
IWLSVDITCCTCSILHLSAIALDRYRAITDAVEYARKRT

SEQ ID NO: 117 - protein, 39 aa, Artificial Sequence
  HUMAN 5-hydroxytryptamine receptor 1E OS homo sapiens OX 9606 GN HTR1E PE 1 SV 1
VWLSVDMTCCTCSILHLCVIALDRYWAITNAIEYARKRT

SEQ ID NO: 118 - protein, 39 aa, Artificial Sequence
  HUMAN 5-hydroxytryptamine receptor 1D OS homo sapiens OX 9606 GN HTR1D PE 1 SV 1
IWLSSDITCCTASILHLCVIALDRYWAITDALEYSKRRT

SEQ ID NO: 119 - protein, 39 aa, Artificial Sequence
  HUMAN 5-hydroxytryptamine receptor 1B OS homo sapiens OX 9606 GN HTR1B PE 1 SV 1
FWLSSDITCCTASILHLCVIALDRYWAITDAVEYSAKRT

SEQ ID NO: 120 - protein, 39 aa, Artificial Sequence
  HUMAN 5-hydroxytryptamine receptor 5A OS homo sapiens OX 9606 GN HTR5A PE 2 SV 1
LWIACDVLCCTASIWNVTAIALDRYWSITRHMEYTLRTR

SEQ ID NO: 121 - protein, 39 aa, Artificial Sequence
  HUMAN 5-hydroxytryptamine receptor 7 OS homo sapiens OX 9606 GN HTR7 PE 1 SV 2
VFIAMDVMCCTASIMTLCVISIDRYLGITRPLTYPVRQN

SEQ ID NO: 122 - protein, 39 aa, Artificial Sequence
  HUMAN 5-hydroxytryptamine receptor 1A OS homo sapiens OX 9606 GN HTR1A PE 1 SV 3
LFIALDVLCCTSSILHLCAIALDRYWAITDPIDYVNKRT

SEQ ID NO: 123 - protein, 39 aa, Artificial Sequence
  HUMAN 5-hydroxytryptamine receptor 2A OS homo sapiens OX 9606 GN HTR2A PE 1 SV 2
VWIYLDVLFSTASIMHLCAISLDRYVAIQNPIHHSRFNS

SEQ ID NO: 124 - protein, 42 aa, Artificial Sequence
  HUMAN (D3) Dopamine receptor OS homo sapiens OX 9606 GN DRD3 PE 1 SV 2
VFVTLDVMMCTASILNLCAISIDRYTAVVMPVHYQHGTGQSS

SEQ ID NO: 125 - protein, 40 aa, Artificial Sequence
  HUMAN (D2) Dopamine receptor OS homo sapiens OX 9606 GN DRD2 PE 1 SV 2
IFVTLDVMMCTASILNLCAISIDRYTAVAMPMLYNTRYSS

SEQ ID NO: 126 - protein, 39 aa, Artificial Sequence
  HUMAN (D4) Dopamine receptor OS homo sapiens OX 9606 GN DRD4 PE 1 SV 3
ALMAMDVMLCTASIFNLCAISVDRFVAVAVPLRYNRQGG

SEQ ID NO: 127 - protein, 39 aa, Artificial Sequence
  HUMAN Alpha-1B adrenergic receptor OS Homo sapiens OX 9606 GN ADRA1B PE 1 SV 3
IWAAVDVLCCTASILSLCAISIDRYIGVRYSLQYPTLVT

SEQ ID NO: 128 - protein, 39 aa, Artificial Sequence
  HUMAN Alpha-1D adrenergic receptor OS Homo sapiens OX 9606 GN ADRA1D PE 1 SV 2
VWAAVDVLCCTASILSLCTISVDRYVGVRHSLKYPAIMT

SEQ ID NO: 129 - protein, 39 aa, Artificial Sequence
  HUMAN Alpha-1A adrenergic receptor OS Homo sapiens OX 9606 GN ADRA1A PE 1 SV 2
IWAAVDVLCCTASIMGLCIISIDRYIGVSYPLRYPTIVT

SEQ ID NO: 130 - protein, 39 aa, Artificial Sequence
  HUMAN Alpha-2B adrenergic receptor OS Homo sapiens OX 9606 GN ADRA2B PE 1 SV 4
VYLALDVLFCTSSIVHLCAISLDRYWAVSRALEYNSKRT

SEQ ID NO: 131 - protein, 39 aa, Artificial Sequence
  HUMAN Alpha-2A adrenergic receptor OS Homo sapiens OX 9606 GN ADRA2A PE 1 SV 4
IYLALDVLFCTSSIVHLCAISLDRYWSITQAIEYNLKRT

SEQ ID NO: 132 - protein, 39 aa, Artificial Sequence
  HUMAN Alpha-2C adrenergic receptor OS Homo sapiens OX 9606 GN ADRA2C PE 1 SV 2
VYLALDVLFCTSSIVHLCAISLDRYWSVTQAVEYNLKRT

SEQ ID NO: 133 - protein, 39 aa, Artificial Sequence
  HUMAN 5-hydroxytryptamine receptor 6 OS homo sapiens OX 9606 GN HTR6 PE 1 SV 1
LWTAFDVMCCSASILNLCLISLDRYLLILSPLRYKLRMT

SEQ ID NO: 134 - protein, 39 aa, Artificial Sequence
  HUMAN 5-hydroxytryptamine receptor 2B OS homo sapiens OX 9606 GN HTR2B PE 1 SV 1
AWLFLDVLFSTASIMHLCAISVDRYIAIKKPIQANQYNS

SEQ ID NO: 135 - protein, 39 aa, Artificial Sequence
  HUMAN 5-hydroxytryptamine receptor 2C OS homo sapiens OX 9606 GN HTR2C PE 1 SV 1
VWISLDVLFSTASIMHLCAISLDRYVAIRNPIEHSRFNS

SEQ ID NO: 136 - protein, 39 aa, Artificial Sequence
  HUMAN D(1B) dopamine receptor OS homo sapiens OX 9606 GN DRD5 PE 1 SV 2
VWVAFDIMCSTASILNLCVISVDRYWAISRPFRYKRKMT

SEQ ID NO: 137 - protein, 39 aa, Artificial Sequence
  HUMAN D(1A) dopamine receptor OS homo sapiens OX 9606 GN DRD1 PE 1 SV 1
IWVAFDIMCSTASILNLCVISVDRYWAISSPFRYERKMT

SEQ ID NO: 138 - protein, 40 aa, Artificial Sequence
  HUMAN 5-hydroxytryptamine receptor 4 OS homo sapiens OX 9606 GN HTR4 PE 1 SV 2
VRTSLDVLLTTASIFHLCCISLDRYYAICCQPLVYRNKMT

SEQ ID NO: 139 - protein, 39 aa, Artificial Sequence
  HUMAN Beta-3 adrenergic receptor OS homo sapiens OX 9606 GN ADRB3 PE 1 SV 3
LWTSVDVLCVTASIETLCALAVDRYLAVTNPLRYGALVT

SEQ ID NO: 140 - protein, 39 aa, Artificial Sequence
  HUMAN Beta-1 adrenergic receptor OS homo sapiens OX 9606 GN ADRB1 PE 1 SV 2
LWTSVDVLCVTASIETLCVIALDRYLAITSPFRYQSLLT

SEQ ID NO: 141 - protein, 39 aa, Artificial Sequence
  HUMAN Beta-2 adrenergic receptor OS homo sapiens OX 9606 GN ADRB2 PE 1 SV 3
FWTSIDVLCVTASIETLCVIAVDRYFAITSPFKYQSLLT

SEQ ID NO: 142 - protein, 40 aa, Artificial Sequence
  HUMAN Muscarinic Acetylcholine Receptor M4 OS Homo Sapiens OX 9606 GN CHRM4 PE 1 SV 2
NQCFIQFLSNPAVTFGTAIAAFYLPVVIMTVLYIHISLAS

SEQ ID NO: 143 - protein, 40 aa, Artificial Sequence
  HUMAN Muscarinic Acetylcholine Receptor M1 OS Homo Sapiens OX 9606 GN CHRM1 PE 1 SV 2
GQCYIQFLSQPIITFGTAMAAFYLPVTVMCTLYWRIYRET

SEQ ID NO: 144 - protein, 40 aa, Artificial Sequence
  HUMAN Muscarinic Acetylcholine Receptor M2 OS Homo Sapiens OX 9606 GN CHRM2 PE 1 SV 1
GECYIQFFSNAAVTFGTAIAAFYLPVIIMTVLYWHISRAS

SEQ ID NO: 145 - protein, 40 aa, Artificial Sequence
  HUMAN Muscarinic Acetylcholine Receptor M3 OS Homo Sapiens OX 9606 GN CHRM3 PE 1 SV 1
GECFIQFLSEPTITFGTAIAAFYMPVTIMTILYWRIYKET

SEQ ID NO: 146 - protein, 40 aa, Artificial Sequence
  HUMAN Muscarinic Acetylcholine Receptor M5 OS Homo Sapiens OX 9606 GN CHRM5 PE 1 SV 2
DECQIQFLSEPTITFGTAIAAFYIPVSVMTILYCRIYRET

SEQ ID NO: 147 - protein, 40 aa, Artificial Sequence
  HUMAN Histamine H1 receptor OS homo sapiens OX 9606 GN HRH1 PE 1 SV 1
DKCETDFYDVTWFKVMTAIINFYLPTLLMLWFYAKIYKAV

SEQ ID NO: 148 - protein, 38 aa, Artificial Sequence
  HUMAN Histamine H2 receptor OS homo sapiens OX 9606 GN HRH2 PE 2 SV 1
SKCKVQVNEVYGLVDGLVTFYLPLLIMCITYYRIFKVA

SEQ ID NO: 149 - protein, 40 aa, Artificial Sequence
  HUMAN Histamine H3 receptor OS homo sapiens OX 9606 GN HRH3 PE 1 SV 2
GHCYAEFFYNWYFLITASTLEFFTPFLSVTFFNLSIYLNI

SEQ ID NO: 150 - protein, 40 aa, Artificial Sequence
  HUMAN Histamine H4 receptor OS homo sapiens OX 9606 GN HRH4 PE 1 SV 2
SECEPGFFSEWYILAITSFLEFVIPVILVAYFNMNIYWSL

SEQ ID NO: 151 - protein, 39 aa, Artificial Sequence
  HUMAN 5-hydroxytryptamine receptor 1F OS homo sapiens OX 9606 GN HTR1F PE 2 SV 1
DECIIKHDHIVSTIYSTFGAFYIPLALILILYYKIYRAA

SEQ ID NO: 152 - protein, 39 aa, Artificial Sequence
  HUMAN 5-hydroxytryptamine receptor 1E OS homo sapiens OX 9606 GN HTR1E PE 1 SV 1
SQCTIQHDHVIYTIYSTLGAFYIPLTLILILYYRIYHAA

SEQ ID NO: 153 - protein, 39 aa, Artificial Sequence
  HUMAN 5-hydroxytryptamine receptor 1D OS homo sapiens OX 9606 GN HTR1D PE 1 SV 1
SDCLVNTSQISYTIYSTCGAFYIPSVLLIILYGRIYRAA

SEQ ID NO: 154 - protein, 39 aa, Artificial Sequence
  HUMAN 5-hydroxytryptamine receptor 1B OS homo sapiens OX 9606 GN HTR1B PE 1 SV 1
SECVVNTDHILYTVYSTVGAFYFPTLLLIALYGRIYVEA

SEQ ID NO: 155 - protein, 38 aa, Artificial Sequence
  HUMAN 5-hydroxytryptamine receptor 5A OS homo sapiens OX 9606 GN HTR5A PE 2 SV 1
EECQVSREPSYAVFSTVGAFYLPLCVVLFVYWKIYKAA

SEQ ID NO: 156 - protein, 38 aa, Artificial Sequence
  HUMAN 5-hydroxytryptamine receptor 7 OS homo sapiens OX 9606 GN HTR7 PE 1 SV 2
KVCLISQDFGYTIYSTAVAFYIPMSVMLFMYYQIYKAA

SEQ ID NO: 157 - protein, 38 aa, Artificial Sequence
  HUMAN 5-hydroxytryptamine receptor 1A OS homo sapiens OX 9606 GN HTR1A PE 1 SV 3
DACTISKDHGYTIYSTFGAFYIPLLLMLVLYGRIFRAA

SEQ ID NO: 158 - protein, 37 aa, Artificial Sequence
  HUMAN 5-hydroxytryptamine receptor 2A OS homo sapiens OX 9606 GN HTR2A PE 1 SV 2
GSCLLADDNFVLIGSFVSFFIPLTIMVITYFLTIKSL

SEQ ID NO: 159 - protein, 37 aa, Artificial Sequence
  HUMAN (D3) Dopamine receptor OS homo sapiens OX 9606 GN DRD3 PE 1 SV 2
TVCSISNPDFVIYSSVVSFYLPFGVTVLVYARIYVVL

SEQ ID NO: 160 - protein, 37 aa, Artificial Sequence
  HUMAN (D2) Dopamine receptor OS homo sapiens OX 9606 GN DRD2 PE 1 SV 2
NECIIANPAFVVYSSIVSFYVPFIVTLLVYIKIYIVL

SEQ ID NO: 161 - protein, 37 aa, Artificial Sequence
  HUMAN (D4) Dopamine receptor OS homo sapiens OX 9606 GN DRD4 PE 1 SV 3
AVCRLEDRDYVVYSSVCSFFLPCPLMLLLYWATFRGL

SEQ ID NO: 162 - protein, 38 aa, Artificial Sequence
  HUMAN Alpha-1B adrenergic receptor OS Homo sapiens OX 9606 GN ADRA1B PE 1 SV 3
KECGVTEEPFYALFSSLGSFYIPLAVILVMYCRVYIVA

SEQ ID NO: 163 - protein, 38 aa, Artificial Sequence
  HUMAN Alpha-1D adrenergic receptor OS Homo sapiens OX 9606 GN ADRA1D PE 1 SV 2
RFCGITEEAGYAVFSSVCSFYLPMAVIVVMYCRVYVVA

SEQ ID NO: 164 - protein, 38 aa, Artificial Sequence
  HUMAN Alpha-1A adrenergic receptor OS Homo sapiens OX 9606 GN ADRA1A PE 1 SV 2
TICQINEEPGYVLFSALGSFYLPLAIILVMYCRVYVVA

SEQ ID NO: 165 - protein, 38 aa, Artificial Sequence
  HUMAN Alpha-2B adrenergic receptor OS Homo sapiens OX 9606 GN ADRA2B PE 1 SV 4
PQCKLNQEAWYILASSIGSFFAPCLIMILVYLRIYLIA

SEQ ID NO: 166 - protein, 38 aa, Artificial Sequence
  HUMAN Alpha-2A adrenergic receptor OS Homo sapiens OX 9606 GN ADRA2A PE 1 SV 4
PRCEINDQKWYVISSCIGSFFAPCLIMILVYVRIYQIA

SEQ ID NO: 167 - protein, 38 aa, Artificial Sequence
  HUMAN Alpha-2C adrenergic receptor OS Homo sapiens OX 9606 GN ADRA2C PE 1 SV 2
PQCGLNDETWYILSSCIGSFFAPCLIMGLVYARIYRVA

SEQ ID NO: 168 - protein, 38 aa, Artificial Sequence
  HUMAN 5-hydroxytryptamine receptor 6 OS homo sapiens OX 9606 GN HTR6 PE 1 SV 1
GQCRLLASLPFVLVASGLTFFLPSGAICFTYCRILLAA

SEQ ID NO: 169 - protein, 40 aa, Artificial Sequence
  HUMAN 5-hydroxytryptamine receptor 2B OS homo sapiens OX 9606 GN HTR2B PE 1 SV 1
ITCVLTKERFGDFMLFGSLAAFFTPLAIMIVTYFLTIHAL

SEQ ID NO: 170 - protein, 37 aa, Artificial Sequence
  HUMAN 5-hydroxytryptamine receptor 2C OS homo sapiens OX 9606 GN HTR2C PE 1 SV 1
TTCVLNDPNFVLIGSFVAFFIPLTIMVITYCLTIYVL

SEQ ID NO: 171 - protein, 41 aa, Artificial Sequence
  HUMAN D(1B) dopamine receptor OS homo sapiens OX 9606 GN DRD5 PE 1 SV 2
VNAENCDSSLNRTYAISSSLISFYIPVAIMIVTYTRIYRIA

SEQ ID NO: 172 - protein, 41 aa, Artificial Sequence
  HUMAN D(1A) dopamine receptor OS homo sapiens OX 9606 GN DRD1 PE 1 SV 1
ETIDNCDSSLSRTYAISSSVISFYIPVAIMIVTYTRIYRIA

SEQ ID NO: 173 - protein, 41 aa, Artificial Sequence
  HUMAN 5-hydroxytryptamine receptor 4 OS homo sapiens OX 9606 GN HTR4 PE 1 SV 2
SNSTYCVFMVNKPYAITCSVVAFYIPFLLMVLAYYRIYVTA

SEQ ID NO: 174 - protein, 38 aa, Artificial Sequence
  HUMAN Beta-3 adrenergic receptor OS homo sapiens OX 9606 GN ADRB3 PE 1 SV 3
RCCAFASNMPYVLLSSSVSFYLPLLVMLFVYARVFVVA

SEQ ID NO: 175 - protein, 38 aa, Artificial Sequence
  HUMAN Beta-1 adrenergic receptor OS homo sapiens OX 9606 GN ADRB1 PE 1 SV 2
KCCDFVTNRAYAIASSVVSFYVPLCIMAFVYLRVFREA

SEQ ID NO: 176 - protein, 38 aa, Artificial Sequence
  HUMAN Beta-2 adrenergic receptor OS homo sapiens OX 9606 GN ADRB2 PE 1 SV 3
TCCDFFTNQAYAIASSIVSFYVPLVIMVFVYSRVFQEA

SEQ ID NO: 177 - protein, 35 aa, Artificial Sequence
  HUMAN Muscarinic Acetylcholine Receptor M4 OS Homo Sapiens OX 9606 GN CHRM4 PE 1 SV 2
ERKVTRTIFAILLAFILTWTPYNVMVLVNTFCQSC

SEQ ID NO: 178 - protein, 35 aa, Artificial Sequence
  HUMAN Muscarinic Acetylcholine Receptor M1 OS Homo Sapiens OX 9606 GN CHRM1 PE 1 SV 2
EKKAARTLSAILLAFILTWTPYNIMVLVSTFCKDC

SEQ ID NO: 179 - protein, 35 aa, Artificial Sequence
  HUMAN Muscarinic Acetylcholine Receptor M2 OS Homo Sapiens OX 9606 GN CHRM2 PE 1 SV 1
EKKVTRTILAILLAFIITWAPYNVMVLINTFCAPC

SEQ ID NO: 180 - protein, 35 aa, Artificial Sequence
  HUMAN Muscarinic Acetylcholine Receptor M3 OS Homo Sapiens OX 9606 GN CHRM3 PE 1 SV 1
EKKAAQTLSAILLAFIITWTPYNIMVLVNTFCDSC

SEQ ID NO: 181 - protein, 35 aa, Artificial Sequence
  HUMAN Histamine H1 receptor OS homo sapiens OX 9606 GN HRH1 PE 1 SV 1
ERKAAQTLSAILLAFIITWTPYNIMVLVSTFCDKC

SEQ ID NO: 182 - protein, 35 aa, Artificial Sequence
  HUMAN Histamine H2 receptor OS homo sapiens OX 9606 GN HRH2 PE 2 SV 1
ERKAAKQLGFIMAAFILCWIPYFIFFMVIAFCKNC

SEQ ID NO: 183 - protein, 36 aa, Artificial Sequence
  HUMAN Histamine H3 receptor OS homo sapiens OX 9606 GN HRH3 PE 1 SV 2
EHKATVTLAAVMGAFIICWFPYFTAFVYRGLRGDDA

SEQ ID NO: 184 - protein, 35 aa, Artificial Sequence
  HUMAN Histamine H3 receptor OS homo sapiens OX 9606 GN HRH3 PE 1 SV 2
DRKVAKSLAVIVSIFGLCWAPYTLLMIIRAACHGH

SEQ ID NO: 185 - protein, 35 aa, Artificial Sequence
  HUMAN Histamine H4 receptor OS homo sapiens OX 9606 GN HRH4 PE 1 SV 2
ARRLAKSLAILLGVFAVCWAPYSLFTIVLSFYSSA

SEQ ID NO: 186 - protein, 38 aa, Artificial Sequence
  HUMAN 5-hydroxytryptamine receptor 1F OS homo sapiens OX 9606 GN HTR1F PE 2 SV 1
ERKAERKAATTLGLILGAFVICWLPFFVKELVVNVCDK

SEQ ID NO: 187 - protein, 35 aa, Artificial Sequence
  HUMAN 5-hydroxytryptamine receptor 1E OS homo sapiens OX 9606 GN HTR1E PE 1 SV 1
ERKAARILGLILGAFILSWLPFFIKELIVGLSIYT

SEQ ID NO: 188 - protein, 35 aa, Artificial Sequence
  HUMAN 5-hydroxytryptamine receptor 1D OS homo sapiens OX 9606 GN HTR1D PE 1 SV 1
ERKATKILGIILGAFIICWLPFFVVSLVLPICRDS

SEQ ID NO: 189 - protein, 35 aa, Artificial Sequence
  HUMAN 5-hydroxytryptamine receptor 1B OS homo sapiens OX 9606 GN HTR1B PE 1 SV 1
ERKATKTLGIILGAFIVCWLPFFIISLVMPICKDA

SEQ ID NO: 190 - protein, 35 aa, Artificial Sequence
  HUMAN 5-hydroxytryptamine receptor 5A OS homo sapiens OX 9606 GN HTR5A PE 2 SV 1
EQRAALMVGILIGVFVLCWIPFFLTELISPLCSCD

SEQ ID NO: 191 - protein, 36 aa, Artificial Sequence
  HUMAN 5-hydroxytryptamine receptor 7 OS homo sapiens OX 9606 GN HTR7 PE 1 SV 2
EQKAATTLGIIVGAFTVCWLPFFLLSTARPFICGTS

SEQ ID NO: 192 - protein, 35 aa, Artificial Sequence
  HUMAN 5-hydroxytryptamine receptor 1A OS homo sapiens OX 9606 GN HTR1A PE 1 SV 3
ERKTVKTLGIIMGTFILCWLPFFIVALVLPFCESS

SEQ ID NO: 193 - protein, 35 aa, Artificial Sequence
  HUMAN 5-hydroxytryptamine receptor 2A OS homo sapiens OX 9606 GN HTR2A PE 1 SV 2
EQKACKVLGIVFFLFVVMWCPFFITNIMAVICKES

SEQ ID NO: 194 - protein, 34 aa, Artificial Sequence
  HUMAN (D3) Dopamine receptor OS homo sapiens OX 9606 GN DRD3 PE 1 SV 2
EKKATQMVAIVLGAFIVCWLPFFLTHVLNTHCQT

SEQ ID NO: 195 - protein, 33 aa, Artificial Sequence
  HUMAN (D2) Dopamine receptor OS homo sapiens OX 9606 GN DRD2 PE 1 SV 2
EKKATQMLAIVLGVFIICWLPFFITHILNIHCD

SEQ ID NO: 196 - protein, 34 aa, Artificial Sequence
  HUMAN (D4) Dopamine receptor OS homo sapiens OX 9606 GN DRD4 PE 1 SV 3
ERKAMRVLPVVVGAFLLCWTPFFVVHITQALCPA

SEQ ID NO: 197 - protein, 36 aa, Artificial Sequence
  HUMAN Alpha-1B adrenergic receptor OS Homo sapiens OX 9606 GN ADRA1B PE 1 SV 3
EKKAAKTLGIVVGMFILCWLPFFIALPLGSLFSTLK

SEQ ID NO: 198 - protein, 36 aa, Artificial Sequence
  HUMAN Alpha-1D adrenergic receptor OS Homo sapiens OX 9606 GN ADRA1D PE 1 SV 2
EKKAAKTLAIVVGVFVLCWFPFFFVLPLGSLFPQLK

SEQ ID NO: 199 - protein, 36 aa, Artificial Sequence
  HUMAN Alpha-1A adrenergic receptor OS Homo sapiens OX 9606 GN ADRA1A PE 1 SV 2
EKKAAKTLGIVVGCFVLCWLPFFLVMPIGSFFPDFK

SEQ ID NO: 200 - protein, 35 aa, Artificial Sequence
  HUMAN Alpha-2B adrenergic receptor OS Homo sapiens OX 9606 GN ADRA2B PE 1 SV 4
EKRFTFVLAVVIGVFVLCWFPFFFSYSLGAICPKH

SEQ ID NO: 201 - protein, 34 aa, Artificial Sequence
  HUMAN Alpha-2A adrenergic receptor OS Homo sapiens OX 9606 GN ADRA2A PE 1 SV 4
EKRFTFVLAVVIGVFVVCWFPFFFTYTLTAVGCS

SEQ ID NO: 202 - protein, 35 aa, Artificial Sequence
  HUMAN Alpha-2C adrenergic receptor OS Homo sapiens OX 9606 GN ADRA2C PE 1 SV 2
EKRFTFVLAVVMGVFVLCWFPFFFSYSLYGICREA

SEQ ID NO: 203 - protein, 34 aa, Artificial Sequence
  HUMAN 5-hydroxytryptamine receptor 6 OS homo sapiens OX 9606 GN HTR6 PE 1 SV 1
ALKASLTLGILLGMFFVTWLPFFVANIVQAVCDC

SEQ ID NO: 204 - protein, 34 aa, Artificial Sequence
  HUMAN 5-hydroxytryptamine receptor 2B OS homo sapiens OX 9606 GN HTR2B PE 1 SV 1
EQRASKVLGIVFFLFLLMWCPFFITNITLVLCDS

SEQ ID NO: 205 - protein, 35 aa, Artificial Sequence
  HUMAN 5-hydroxytryptamine receptor 2C OS homo sapiens OX 9606 GN HTR2C PE 1 SV 1
ERKASKVLGIVFFVFLIMWCPFFITNILSVLCEKS

SEQ ID NO: 206 - protein, 43 aa, Artificial Sequence
  HUMAN D(1B) dopamine receptor OS homo sapiens OX 9606 GN DRD5 PE 1 SV 2
ETKVLKTLSVIMGVFVCCWLPFFILNCMVPFCSGHPEGPPAGF

SEQ ID NO: 207 - protein, 39 aa, Artificial Sequence
  HUMAN D(1A) dopamine receptor OS homo sapiens OX 9606 GN DRD1 PE 1 SV 1
ETKVLKTLSVIMGVFVCCWLPFFILNCILPFCGSGETQP

SEQ ID NO: 208 - protein, 35 aa, Artificial Sequence
  HUMAN 5-hydroxytryptamine receptor 4 OS homo sapiens OX 9606 GN HTR4 PE 1 SV 2
ETKAAKTLCIIMGCFCLCWAPFFVTNIVDPFIDYT

SEQ ID NO: 209 - protein, 36 aa, Artificial Sequence
  HUMAN Beta-3 adrenergic receptor OS homo sapiens OX 9606 GN ADRB3 PE 1 SV 3
EHRALCTLGLIMGTFTLCWLPFFLANVLRALGGPSL

SEQ ID NO: 210 - protein, 35 aa, Artificial Sequence
  HUMAN Beta-1 adrenergic receptor OS homo sapiens OX 9606 GN ADRB1 PE 1 SV 2
EQKALKTLGIIMGVFTLCWLPFFLANVVKAFHREL

SEQ ID NO: 211 - protein, 35 aa, Artificial Sequence
  HUMAN Beta-2 adrenergic receptor OS homo sapiens OX 9606 GN ADRB2 PE 1 SV 3
EHKALKTLGIIMGTFTLCWLPFFIVNIVHVIQDNL

SEQ ID NO: 212 - protein, 9 aa, Artificial Sequence
  hydrophilic residues
GPSNDQERK